SEQUENCE LISTING (158 sequences)

SEQ ID NO: 1 - protein, 133 aa, Homo sapiens [sequence omitted]

SEQ ID NO: 2 - protein, 132 aa, synthetic construct
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 3 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 4 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 5 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXYKFYXPKKATELKHLQCLEEELKPLEEVLXYAQSKNFHLRPRDLISNINVIVLELKGSETTFXCEYADETATIVEFLNRWITFSQSIISTLT

SEQ ID NO: 6 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFKFYXPKKATELKHLQCLEEELKPLEEVLXYAQSKNFHLRPRDLISNINVIVLELKGSETTFXCEYADETATIVEFLNRWITFSQSIISTLT

SEQ ID NO: 7 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXYKFYXPKKATELKHLQCLEEELKPLEEVLXGAQSKNFHLRPRDLISNINVIVLELKGSETTFXCEYADETATIVEFLNRWITFSQSIISTLT

SEQ ID NO: 8 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXYKFYXPKKATELKHLQCLEEELKYLEEVLXLAQSKNFHLRPRDLISNINVIVLELKGSETTFXCEYADETATIVEFLNRWITFSQSIISTLT

SEQ ID NO: 9 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFKFYXPKKATELKHLQCLEEELKYLEEVLXLAQSKNFHLRPRDLISNINVIVLELKGSETTFXCEYADETATIVEFLNRWITFSQSIISTLT

SEQ ID NO: 10 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTYXLXYKFYXPKKATELKHLQCLEEYLKYLEEVLXLAQSKNFHLRPRDLISNINVIVLELKGSETTFXCEYADETATIVEFLNRWITFSQSIISTLT

SEQ ID NO: 11 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFKFYXPKKATELKHLQCLEEYLKYLEEVLXLAQSKNFHLRPRDLISNINVIVLELKGSETTFXCEYADETATIVEFLNRWITFSQSIISTLT

SEQ ID NO: 12 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFKFYXPKKATELKHLQCLEEYLKPLEEVLXLAQSKNFHLRPRDLISNINVIVLELKGSETTFXCEYADETATIVEFLNRWITFSQSIISTLT

SEQ ID NO: 13 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFYFYXPKKATELKHLQCLEEELKPLEEVLXLAQSKNFHLRPRDLISNINVIVLELKGSETTFXCEYADETATIVEFLNRWITFSQSIISTLT

SEQ ID NO: 14 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFKYYXPKKATELKHLQCLEEELKPLEEVLXLAQSKNFHLRPRDLISNINVIVLELKGSETTFXCEYADETATIVEFLNRWITFSQSIISTLT

SEQ ID NO: 15 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPYLTRXLXFKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 16 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEYLLLDLQXILNGINNYKNPKLTRXLXFKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 17 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEYLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 18 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLYLQXILNGINNYKNPKLTRXLXFKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 19 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLYLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 20 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPYLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 21 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXYKFYXPKKATELKHLQCLEEYLKPLEEVLXLAQSKNFHLRPRDLISNINVIVLELKGSETTFXCEYADETATIVEFLNRWITFSQSIISTLT

SEQ ID NO: 22 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXYKFYXPKKATELKHLQCLEEYLKYLEEVLXLAQSKNFHLRPRDLISNINVIVLELKGSETTFXCEYADETATIVEFLNRWITFSQSIISTLT

SEQ ID NO: 23 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFKFYXPKKATELKHLQCLEEELKPLEEVLXLAQSKNFHLRPRDLISNINVIVLELKGSETTFXCEYADETATIVEFLNRWITFCQSIISTLT

SEQ ID NO: 24 - protein, 20 aa, synthetic construct
  VARIANT 1..20: note = This sequence may encompass 1-10 GS repeating units
GSGSGSGSGSGSGSGSGSGS

SEQ ID NO: 25 - protein, 30 aa, synthetic construct
  VARIANT 1..30: note = This sequence may encompass 1-10 GGS repeating units
GGSGGSGGSGGSGGSGGSGGSGGSGGSGGS

SEQ ID NO: 26 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 27 - protein, 40 aa, synthetic construct
  VARIANT 1..40: note = This sequence may encompass 1-10 GGSG repeating units
GGSGGGSGGGSGGGSGGGSGGGSGGGSGGGSGGGSGGGSG

SEQ ID NO: 28 - protein, 50 aa, synthetic construct
  VARIANT 1..50: note = This sequence may encompass 1-10 GGSGG repeating units
GGSGGGGSGGGGSGGGGSGGGGSGGGGSGGGGSGGGGSGGGGSGGGGSGG

SEQ ID NO: 29 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 30 - protein, 15 aa, synthetic construct
GGGGSGGGGSGGGGS

SEQ ID NO: 31 - protein, 20 aa, synthetic construct
GGGGSGGGGSGGGGSGGGGS

SEQ ID NO: 32 - protein, 445 aa, synthetic construct
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 33 - protein, 214 aa, synthetic construct
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 447 aa, synthetic construct
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 214 aa, synthetic construct
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 452 aa, synthetic construct
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 219 aa, synthetic construct
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 443 aa, synthetic construct
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - protein, 214 aa, synthetic construct
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 444 aa, synthetic construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 214 aa, synthetic construct
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42-45 - skipped (4 entries)

SEQ ID NO: 46 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 119 aa, synthetic construct
QVQLVQSGVEVKKPGASVKVSCKASGYTFTNYYMYWVRQAPGQGLEWMGGFPSNGGTNFNEKFKNRVTLTTDSSTTTAYMELKSLQFDDTAVYYCARRDYRFDMGFDYWGQGTTVTVSS

SEQ ID NO: 49 - protein, 111 aa, synthetic construct [sequence omitted]

SEQ ID NO: 50 - protein, 113 aa, synthetic construct [sequence omitted]

SEQ ID NO: 51 - protein, 107 aa, synthetic construct [sequence omitted]

SEQ ID NO: 52 - protein, 459 aa, synthetic construct
QVQLVQSGGGLVQPGGSLRLSCAASGFTFSSYWMYWVRQVPGKGLEWVSAIDTGGGRTYYADSVKGRFAISRVNAKNTMYLQMNSLRAEDTAVYYCARDEGGGTGWGVLKDWPYGLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 214 aa, synthetic construct
QPVLTQPLSVSVALGQTARITCGGNNIGSKNVHWYQQKPGQAPVLVIYRDSNRPSGIPERFSGSNSGNTATLTISRAQAGDEADYYCQVWDSSTAVFGTGTKLTVLQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 439 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASNGDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 55 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 443 aa, synthetic construct
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 214 aa, synthetic construct
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 442 aa, synthetic construct
OVQLVESGGGLVKPGGSLRLSCAASGFTFSNYGMSWIROAPGKGLEWVSTISGGGSNIYYADSVKGRFTISRDNAKNSLYLQMNSLRAEDTAVYYCVSYYYGIDFWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 214 aa, synthetic construct
DIQMTQSPSSLSASVGDRVTITCKASQDVTTAVAWYQQKPGKAPKLLIYWASTRHTGVPSRFSGSGSGTDFTLTISSLQPEDFATYYCOOHYTIPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 445 aa, synthetic construct
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 61 - protein, 218 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 444 aa, synthetic construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 63 - protein, 220 aa, synthetic construct
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 443 aa, synthetic construct
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 65 - protein, 220 aa, synthetic construct
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 450 aa, synthetic construct
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 67 - protein, 214 aa, synthetic construct
EIVLTQSPATLSLSPGERATLSCRASQSVRSYLAWYQQKPGQAPRLLIYDASNRATGIPARFSGSGSGTDFTLTISSLEPEDFAVYYCQQRNYWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 496 aa, synthetic construct
DIQMTQSPSSLSASVGDRVTITCRASQDVSSVVAWYQQKPGKAPKLLIYSASYRYTGVPSRFSGSGSGTDFTLTISSLQPEDFATYYCQQHYSTPWTFGGGTKLEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGEVAACEKEVAALEKEVAALEKEVAALEKESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 69 - protein, 271 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVQSGAEVKKPGASVKVSCKASGYTFTDYNMDWVRQAPGQGLEWMGDINPDNGVTIYNQKFEGRVTMTTDTSTSTAYMELRSLRSDDTAVYYCAREADYFYFDYWGQGTTLTVSSGGCGGGKVAACKEKVAALKEKVAALKEKVAALKE

SEQ ID NO: 70 - protein, 447 aa, synthetic construct
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 213 aa, synthetic construct
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 113 aa, synthetic construct
QVQLVESGGGVVQPGRSLRLTCKASGLTFSSSGMHWVRQAPGKGLEWVAVIWYDGSKRYYADSVKGRFTISRDNSKNTLFLQMNSLRAEDTAVYYCATNNDYWGQGTLVTVSS

SEQ ID NO: 73 - protein, 107 aa, synthetic construct
EIVLTQSPATLSLSPGERATLSCRASQSVSSYLAWYQQKPGQAPRLLIYTASNRATGIPARFSGSGSGTDFTLTISSLEPEDFAVYYCQQYSNWPRTFGQGTKVEIK

SEQ ID NO: 74 - protein, 120 aa, synthetic construct
EVQLQQSGPVLVKPGASVKMSCKASGYTFTSYYMYWVKQSHGKSLEWIGGVNPSNGGTNFNEKFKSKATLTVDKSSSTAYMELNSLTSEDSAVYYCARRDYRYDMGFDYWGQGTTLTVSS

SEQ ID NO: 75 - protein, 111 aa, synthetic construct
QIVLTQSPAIMSASPGEKVTMTCRASKGVSTSGYSYLHWYQQKPGSSPRLLIYLASYLESGVPVRFSGSGSGTSYSLTISRMEAEDAATYYCQHSRELPLTFGTGTRLEIK

SEQ ID NO: 76 - protein, 120 aa, synthetic construct
QVQLVQSGAEVKKPGASVKVSCKASGYTFTSYYMYWVRQAPGQGLEWMGGVNPSNGGTNFNEKFKSRVTITADKSTSTAYMELSSLRSEDTAVYYCARRDYRYDMGFDYWGQGTTVTVSS

SEQ ID NO: 77 - protein, 111 aa, synthetic construct
EIVLTQSPATLSLSPGERATISCRASKGVSTSGYSYLHWYQQKPGQAPRLLIYLASYLESGVPARFSGSGSGTDFTLTISSLEPEDFATYYCQHSRELPLTFGTGTKVEIK

SEQ ID NO: 78 - protein, 448 aa, synthetic construct
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 219 aa, synthetic construct
DVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 5 aa, synthetic construct
NYYMY

SEQ ID NO: 81 - protein, 17 aa, synthetic construct
GINPSNGGTNFNEKFKN

SEQ ID NO: 82 - protein, 11 aa, synthetic construct
RDYRFDMGFDY

SEQ ID NO: 83 - protein, 15 aa, synthetic construct
RASKGVSTSGYSYLH

SEQ ID NO: 84 - protein, 7 aa, synthetic construct
LASYLES

SEQ ID NO: 85 - protein, 9 aa, synthetic construct
QHSRDLPLT

SEQ ID NO: 86 - protein, 5 aa, synthetic construct
NSGMH

SEQ ID NO: 87 - protein, 17 aa, synthetic construct
VIWYDGSKRYYADSVKG

SEQ ID NO: 88 - protein, 4 aa, synthetic construct
NDDY

SEQ ID NO: 89 - protein, 11 aa, synthetic construct
RASQSVSSYLA

SEQ ID NO: 90 - protein, 7 aa, synthetic construct
DASNRAT

SEQ ID NO: 91 - protein, 9 aa, synthetic construct
QQSSNWPRT

SEQ ID NO: 92 - protein, 9 aa, synthetic construct
FTFSNYGMS

SEQ ID NO: 93 - protein, 10 aa, synthetic construct
TISGGGSNIY

SEQ ID NO: 94 - protein, 9 aa, synthetic construct
VSYYYGIDF

SEQ ID NO: 95 - protein, 11 aa, synthetic construct
KASQDVTTAVA

SEQ ID NO: 96 - protein, 7 aa, synthetic construct
WASTRHT

SEQ ID NO: 97 - protein, 9 aa, synthetic construct
QQHYTIPWT

SEQ ID NO: 98 - protein, 8 aa, synthetic construct
GLTFSSSG

SEQ ID NO: 99 - protein, 8 aa, synthetic construct
IWYDGSKR

SEQ ID NO: 100 - protein, 6 aa, synthetic construct
ATNNDY

SEQ ID NO: 101 - protein, 11 aa, synthetic construct
RASQSVSSYLA

SEQ ID NO: 102 - protein, 7 aa, synthetic construct
TASNRAT

SEQ ID NO: 103 - protein, 9 aa, synthetic construct
QQYSNWPRT

SEQ ID NO: 104 - protein, 831 aa, synthetic construct
MQIPQAPWPWWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQSKYGPPCPSCPAPEFLGGPSVFLFPPKPKDTLMISRTPEVTCWVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMDQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLMQIPQAPWPWWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 105 - protein, 230 aa, synthetic construct
  VARIANT 18: note = Any naturally occurring amino acid
  VARIANT 19: note = Any naturally occurring amino acid
  VARIANT 21: note = Any naturally occurring amino acid
  VARIANT 140: note = Any naturally occurring amino acid
  VARIANT 142: note = Any naturally occurring amino acid
  VARIANT 177: note = Any naturally occurring amino acid
PKSCDKTHTCPPCPAPEXXGXPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKXTPPVLDSDGSFFLYSKLTVDKSRWQQGNVFSCSVMHEALHNHYTQKSLSLSPG

SEQ ID NO: 106 - protein, 48 aa, synthetic construct
QETNPTENLYFQQKNMQCQRRFYEALHDPNLNEEQRNARIRSIRDDDC

SEQ ID NO: 107 - protein, 61 aa, synthetic construct
QTADNQKNMQCQRRFYEALHDPNLNEEQRNARIRSIRDDCSQSANLLAEAQQLNDAQAPQA

SEQ ID NO: 108 - protein, 38 aa, synthetic construct
QETKNMQCQRRFYEALHDPNLNEEQRNARIRSIRDDDC

SEQ ID NO: 109 - protein, 38 aa, synthetic construct
QETFNKQCQRRFYEALHDPNLNEEQRNARIRSIRDDDC

SEQ ID NO: 110 - protein, 38 aa, synthetic construct
QETFNMQCQRRFYEALHDPNLNKEQRNARIRSIRDDDC

SEQ ID NO: 111 - protein, 37 aa, synthetic construct
QETFNMQCQRRFYEALHDPNLNEEQRNARIRSIKDDC

SEQ ID NO: 112 - protein, 35 aa, synthetic construct
QETMQCQRRFYEALHDPNLNEEQRNARIRSIKDDC

SEQ ID NO: 113 - protein, 34 aa, synthetic construct
QETQCQRRFYEALHDPNLNEEQRNARIRSIKDDC

SEQ ID NO: 114 - protein, 33 aa, synthetic construct
QETCQRRFYEALHDPNLNEEQRNARIRSIKDDC

SEQ ID NO: 115 - protein, 20 aa, synthetic construct
QETRGNCAYHKGQLVWCTYH

SEQ ID NO: 116 - protein, 20 aa, synthetic construct
QETRGNCAYHKGQIIWCTYH

SEQ ID NO: 117 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 118 - protein, 170 aa, Homo sapiens
YFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 119 - skipped

SEQ ID NO: 120 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 121 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123-152 - skipped (30 entries)

SEQ ID NO: 153 - protein, 6 aa, synthetic construct
  VARIANT 6: note = Alanine or Proline
LLQGPX

SEQ ID NO: 154 - protein, 8 aa, synthetic construct
GGLLQGPP

SEQ ID NO: 155 - protein, 17 aa, synthetic construct
  SITE 1: note = Acetyl arginine
  DISULFID 4..14: note = Intrachain disulfide bridge
  SITE 8: note = Lysine-3-((3-((2,5-dioxopyrrolidin-1-yl)oxy)-3-oxop ropyl)disulfaneyl)propanoate
  SITE 17: note = Histidine amide
RGNCAYHKGQLVWCTYH

SEQ ID NO: 156 - protein, 5 aa, synthetic construct
  SITE 1: note = 5-oxaproline
  SITE 2: note = N-(32-azido-3,6,9,12,15,18,21,24,27,30-decaoxadotri acontyl)-2-acetamide tyrosine
  SITE 5: note = N-(29-amino-3,6,9,12,15,18,21,24,27-nonaoxanonacosy l)-2-acetamide tyrosine
XYKFY

SEQ ID NO: 157 - protein, 5 aa, synthetic construct
  SITE 1: note = Alanyl-prolyl-threonyl-seryl-seryl-seryl-threonyl-l ysyl-lysyl-threonyl-glutaminyl-leucyl-glutaminyl-leucyl-gl utamyl-histidyl-leucyl-leucyl-leucyl-aspartyl-glutaminyl-n orleucyl-isoleucyl-leucyl-asparaginyl-glycyl-isoleucyl-asp araginyl-asparaginyl-tyrosyl-lysyl-asparaginyl-prolyl-lysy l-threonyl-arginyl-norleucyl-leucine Homoserine
  SITE 2: note = N-(32-azido-3,6,9,12,15,18,21,24,27,30-decaoxadotri acontyl)-2-acetamide tyrosine
  SITE 5: note = N-(29-amino-3,6,9,12,15,18,21,24,27-nonaoxanonacosy l)-2-acetamide tyrosine
XYKFY

SEQ ID NO: 158 - protein, 6 aa, synthetic construct
  SITE 2: note = Homoserine
  SITE 3: note = N-(32-azido-3,6,9,12,15,18,21,24,27,30-decaoxadotri acontyl)-2-acetamide tyrosine
  SITE 6: note = N-(29-amino-3,6,9,12,15,18,21,24,27-nonaoxanonacosy l)-2-acetamide tyrosine
LXYKFY

(19) United States
(12) Patent Application Publication
(10) Pub. No.: US 2023/0250181 A1
(43) Pub. Date: Aug. 10, 2023

(54) MODIFIED CHECKPOINT INHIBITORS AND USES THEREOF

(71) Applicant: Bright Peak Therapeutics AG, Basel (CH)

(72) Inventors: Vijaya Raghavan PATTABIRAMAN, Volketswil (CH); Bertolt KREFT, Kleinmachnow (DE); Jean-Philippe CARRALOT, Saint-Louis (FR); Rubén ALVAREZ SANCHEZ, Rosenau (FR); Magali MULLER, Pulversheim (FR); Matilde ARÉVALO-RUIZ, Basel (CH)

(21) Appl. No.: 17/861,174

(22) Filed: Jul. 9, 2022

Related U.S. Application Data
(60) Provisional application No. 63/219,981, filed on Jul. 9, 2021.
(60) Provisional application No. 63/219,989, filed on Jul. 9, 2021.

(51) Int. Cl. C07K 16/28; A61K 38/20; A61K 31/395

(52) U.S. Cl. CPC C07K 16/2896; A61K 38/2013; A61K 31/5545

(57) ABSTRACT

The present disclosure relates to modified anti-PD-1 polypeptides, pharmaceutical compositions comprising modified anti-PD-1 polypeptides, methods of making anti-PD-1 polypeptides, and methods of using the modified anti-PD-1 polypeptides for treatment of diseases. In one aspect, the disclosure relates to methods of treating cancer in a subject using the modified anti-PD-1 polypeptides.

### CROSS REFERENCE

This application claims the benefit of U.S. Provisional Application No. 63/219,981 filed Jul. 9, 2021, and U.S. Provisional Application No. 63/219,989 filed Jul. 9, 2021, which applications are incorporated herein by reference in their entirety.

### SEQUENCE LISTING

The instant application contains a Sequence Listing which has been submitted electronically in XML file format and is hereby incorporated by reference in its entirety. Said XML copy, created on Aug. 16, 2022, is named 94917-0027_708201US_SL.xml and is 189,028 bytes in size.

### BACKGROUND

In 2021, an estimated 1.8 million new cases of cancer will be diagnosed in the United States, and over 600,000 people will die from the disease. Immunotherapies utilize the immune system of a subject to aid in the treatment of ailments. Immunotherapies can be designed to either activate or suppress the immune system depending on the nature of the disease being treated. A goal of various immunotherapies for the treatment of cancer is to stimulate the immune system so that it recognizes and destroys tumors or other cancerous tissue.

Programmed cell death protein 1 (PD-1) is a protein on the surface of cells that regulates the immune system's response to cells of the human body by downregulating the immune system and promoting self-tolerance by suppressing T cell inflammatory activity. Programmed cell death-ligand 1 (PD-L1) is a type 1 transmembrane protein that suppresses the adaptive arm of the immune system. The PD-1 and PD-L1 pathways represent adaptive immune system resistance mechanisms exerted by tumor cells in response to endogenous immune anti-tumor activity. PD-1 inhibitors, such as anti-PD-1 polypeptides and anti-PD-1 antigen binding fragments are checkpoint inhibitor anticancer agents that block the activity of PD-1 immune checkpoint proteins. Single agent therapies alone, however, in many instances are insufficient in achieving durable responses in cancer patients. Thus, there is a need for improved therapies to treat cancer.

### BRIEF SUMMARY

Described herein are anti-programmed cell death protein 1 (PD-1)-interleukin 2 (IL2) immunoconjugates and uses thereof.

In one aspect, described herein is a composition comprising: a polypeptide which selectively binds to programmed cell death protein 1 (PD-1); a modified IL-2 polypeptide; and a linker, wherein the linker comprises: a first point of attachment covalently attached to a non-terminal residue of the modified IL-2 polypeptide; and a second point of attachment covalently attached to the polypeptide which selectively binds to PD-1.

In one aspect, described herein is a composition comprising: a polypeptide which selectively binds to PD-1, a modified IL-2 polypeptide, and a linker; wherein the linker comprises: a first point of attachment covalently attached to the modified IL-2 polypeptide; and a second point of attachment covalently attached to a non-terminal residue of the polypeptide which selectively binds to PD-1.

In another aspect, described herein, is a composition comprising: a polypeptide which selectively binds to PD-1, a modified IL-2 polypeptide, and a chemical linker; wherein the chemical linker comprises: a first point of attachment covalently attached to the modified IL-2 polypeptide; and a second point of attachment covalently attached to the polypeptide which selectively binds to PD-1.

In another aspect, described herein, is composition comprising: a polypeptide which selectively binds to PD-1, a modified IL-2 polypeptide, and a chemical linker; wherein the chemical linker comprises: a first point of attachment covalently attached to the modified IL-2 polypeptide; and a second point of attachment covalently attached to the polypeptide which selectively binds to PD-1, wherein the modified IL-2 polypeptide is biased towards the IL-2 receptor beta subunit.

In another aspect, described herein is a composition comprising: an IL-2 polypeptide, wherein the IL-2 polypeptide comprises: a first polymer attached at amino acid residue 42, wherein amino acid residue position numbering of the modified IL-2 polypeptide is based on SEQ ID NO: 1 as a reference sequence; and a polypeptide which selectively binds to programmed cell death protein 1 (PD-1).

In another aspect, described herein is a composition comprising: (a) an antibody or an antigen binding fragment which selectively binds to programmed cell death protein 1 (PD-1) and that comprises an Fc region, the Fc region comprising an amino acid sequence with 90% or more identity to SEQ ID NO: 105; (b) one or more linkers covalently attached to the Fc region at an amino acid residue selected from the group consisting of: (i) positions 25 to 35 of SEQ ID NO: 105; (ii) positions 70 to 80 of SEQ ID NO: 105; and (iii) positions 95 to 105 of SEQ ID NO: 105; and (c) one or more cytokines covalently attached to the linker.

In another aspect, described herein, is a composition comprising: (a) an antibody or antigen binding fragment thereof which selectively binds to PD-1 and that comprises an Fc region; (b) one or more linkers covalently attached to the Fc region at an amino acid residue selected from the group consisting of K246, K248, K288, K290, and K317 (Eu numbering); and (c) one or more cytokines covalently attached to the one or more linkers

The polypeptide which selectively binds to PD-1 can be, for example, a recombinant protein, such as an antibody, or a synthetic protein.

In another aspect, described herein is a pharmaceutical composition comprising: a) a composition described herein; and b) one or more pharmaceutically acceptable carriers or excipients.

In another aspect, described herein is a method of treating cancer in a subject in need thereof, the method comprising administering to the subject an effective amount of any of the compositions described herein or a pharmaceutical composition described herein.

In another aspect, described herein is a method of making a composition described herein, the method comprising: a) covalently attaching a reactive group to a specific residue of a polypeptide which selectively binds PD-1; b) contacting the reactive group with a complementary reactive group attached to a cytokine; and c) forming the composition.

In another aspect, described herein is a method of creating a composition comprising: a polypeptide which selectively binds to programmed cell death protein 1 (PD-1); a modified IL-2 polypeptide; and a linker, wherein the linker comprises: a first point of attachment covalently attached to a non-terminal residue of the modified IL-2 polypeptide; and a second point of attachment covalently attached to the polypeptide which selectively binds to PD-1, the method comprising: a) providing an anti-PD-1 polypeptide having at least one acceptor amino acid residue that is reactive with a linker in the presence of a coupling enzyme; and b) reacting said anti-PD-1 polypeptide with a linker comprising a primary amine, wherein the linker comprises a reactive group (R), in the presence of an enzyme capable of causing the formation of a covalent bond between the at least one acceptor amino acid residue and the linker, wherein the covalent bond is not at the R moiety, and wherein the method is performed under conditions sufficient to cause the at least one acceptor amino acid residue to form a covalent bond to the reactive group via the linker, wherein the covalent bond comprises the second point of attachment of the linker.

In another aspect, described herein is a method of creating a composition comprising: a polypeptide which selectively binds to PD-1; a modified IL-2 polypeptide; and a linker, wherein the linker comprises: a first point of attachment covalently attached to a residue of the modified IL-2 polypeptide; and a second point of attachment covalently attached to the polypeptide which selectively binds to PD-1, the method comprising: a) providing a polypeptide which selectively binds to PD-1 having at least one acceptor amino acid residue that is reactive with a linker precursor in the presence of a functionalized Fc binding affinity peptide; and b) reacting said polypeptide which selectively binds to PD-1 with a linker precursor comprising a reactive group (R) capable of forming a bond with the acceptor amino acid residue, and wherein the method is performed under conditions sufficient to cause the at least one acceptor amino acid residue to form a covalent bond to the reactive group via the linker, wherein the covalent bond comprises the second point of attachment of the linker.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

FIG. 1A illustrates anti-PD1-IL2 immunocytokine of the disclosure and the interaction of the anti-PD1-IL2 immunocytokine with an activated T cell through IL2Rβ/γ upregulation and PD-1 inhibition.

FIG. 1B shows the structure of modified conjugatable cytokine Composition AB.

FIG. 2A shows site-selective modification of anti-PD1 antibody by chemical modification technology to introduce one or two conjugation handles.

FIG. 2B shows Q-TOF mass spectra of unmodified Pembrolizumab and Pembrolizumab with a DBCO conjugation handle.

FIG. 2C shows site-selective conjugation of modified IL2 cytokine to generate a PD1-IL2 with DAR1, DAR 2 or mixed DAR between 1 and 2.

FIG. 2D shows TIC chromatogram (top) and intact RP-HPLC (bottom) profile of crude Pembrolizumab and Composition AB conjugation reaction.

FIG. 2E shows Q-TOF mass spec profile of crude Pembrolizumab conjugated to Composition AB conjugation reaction showing the formation of drug-antibody ratio of 1 (DAR1) and drug-antibody ratio of 2 (DAR 2) species.

FIG. 2F shows intact RP-HPLC (Top left) profile of purified Pembrolizumab conjugated to Composition AB.

FIG. 2G shows Q-TOF mass spec profile of purified Pembrolizumab conjugated to Composition AB with mixed drug-antibody ratio (DAR).

FIG. 2H shows SEC-HPLC of purified immunocytokine comprising Pembrolizumab conjugated to Composition AB.

FIG. 3 shows plots measuring ability of the unmodified and of conjugated anti-PD1 antibodies to bind with PD1/CD279 ligand, with the figure showing ELISA signal on the y-axis and dosage of the biotinylated PD-1 protein on the x-axis. The unconjugated reference antibodies are Pembrolizumab, Nivolumab, and LZM-009. The conjugated antibodies tested in this figure are Compositions A, C, D, E, F, G, and H.

FIG. 4 shows plots measuring ability of the unmodified and of conjugated anti-PD1 antibodies to interfere with PD1/PDL1 pathway, with the figure showing mean luminescence intensity of effector cells NFAT-RE reporter on the y-axis and dosage of the unmodified and of conjugated anti-PD1 antibodies on the x-axis. The unconjugated reference antibody is Pembrolizumab, and the conjugated antibody tested in this figure is Composition B. The IL-2 polypeptides tested in this figure are Proleukin and Composition AA.

FIG. 5 shows plots measuring ability of the unmodified and of conjugated anti-PD1 antibodies to bind to human neonatal Fc receptor (FcRn) at pH 6, with the figure showing mean AlphaLISA® FcRn-IgG signal on the y-axis and dosage of the unmodified and of conjugated anti-PD1 antibodies on the x-axis. The unconjugated reference antibodies are Pembrolizumab and LZM-009. The conjugated antibodies tested are Compositions A, D, E, H, J, and K.

FIG. 6A shows plots measuring ability of the unmodified and of conjugated anti-PD1 antibodies to bind to human Fc gamma receptor I (CD64), human Fc gamma receptor IIa (CD32a), human Fc gamma receptor IIb (CD32b), and to human Fc gamma receptor Ma (CD16) with the figure showing mean ELISA signal on the y-axis and dosage of the unmodified and of conjugated anti-PD1 antibodies on the x-axis. The unconjugated reference antibodies are Pembrolizumab and LZM-009. The conjugated antibodies tested are Compositions A, C, D, and H.

FIG. 6B shows plots measuring ability of the conjugated anti-PD1 antibodies to bind to human Fc gamma receptor I (CD64), human Fc gamma receptor IIa (CD32a), human Fc gamma receptor lib (CD32b), and to human Fc gamma receptor Ma (CD16) with the figure showing mean ELISA signal on the y-axis and dosage of the conjugated anti-PD1 antibodies on the x-axis. The conjugated antibodies compositions tested from top to bottom are Compositions E, J, and K respectively.

FIG. 7A shows plots measuring the level of surface expression of PD-1/CD279 on parental non-transduced Mo7e (PDF) and stably transduced (PD1⁺) Mo7e cells.

FIG. 7B shows EC₅₀ values for phosphorylated signal transducer and activator of transcription 5 (pSTAT5) on the y-axis in parental PD1-Mo7e cells as “PDF” or Stable PD1+Mo7e cells as “PD1+” with treatments of modified IL-2 polypeptides or immunocytokines, as described on the x-axis. The measurement shown for PD1 negative cells are shown in black filled symbols, whereas PD1 positive cells are shown in grey open symbols. Unconjugated, modified IL-2 polypeptides tested in this figure are Proleukin, and Composition AB. The modified IL-2 anti-PD-1 immunocytokines are Compositions A, C, and H. Composition O, a Her2-targeted IL-2 immunocytokine, is shown as a negative control.

FIG. 8 shows plots measuring the effect of the modified IL-2 polypeptides unconjugated and conjugated to the anti-PD1 antibody on the inducement of Teff and Treg cells in human T-cells in vitro. Shown is the mean fluorescence intensity for phosphorylated signal transducer and activator of transcription5 (pSTAT5) on the y-axis as dose response to a modified IL-2 polypeptide or immunocytokines on the x-axis. The modified IL-2 polypeptide tested is Composition AA. The IL-2 antiPD-1 immunocytokines tested are Compositions A, B, and C.

FIG. 9A shows plots measuring the level of surface expression of PD-1/CD279 on resting memory (CD45RA−) and naïve (CD45RA+) CD8+ T_{eff} cells freshly isolated from peripheral blood of healthy donors, as well as cartoon depictions of the indicated cell types.

FIG. 9B shows the dose response effect of conjugating IL-2 to PD-1 on CD8+ Teff cells. Plots measuring the effect of the modified IL-2 polypeptides unconjugated and conjugated to the anti-PD1 antibody on the inducement of on resting memory (CD45RA−) and naïve (CD45RA+) CD8+ T_{eff} cells in an in vitro sample of human T-cells, with the figure showing mean fluorescence intensity for phosphorylated signal transducer and activator of transcription 5 (pSTAT5) on the y-axis and dosage of modified IL-2 polypeptide and immunocytokines on the x-axis. The modified IL-2 polypeptide tested in this figure is Composition AA. The IL-2 antiPD-1 immunocytokine tested in this figure is Composition B. The Her2-targeted immunocytokine Composition N (Trastuzumab antibody conjugated to IL-2 polypeptide) is shown as a negative control.

FIG. 10A shows plots measuring the effect of the modified IL-2 polypeptides unconjugated and conjugated to the anti-PD1 antibody on the inducement of resting naïve (CD45RA+) CD8+ T_{eff} cells in an in vitro sample of human T-cells in the presence or absence of excess amounts of unconjugated anti-PD1 antibody Pembrolizumab, with the figure showing mean fluorescence intensity for phosphorylated signal transducer and activator of transcription 5 (pSTAT5) on the y-axis and dosage of modified IL-2 polypeptide and immunocytokines on the x-axis. The modified IL-2 polypeptide tested in this figure is Composition AA and the immunocytokines tested in this figure are Composition B and HER2-targeted immunocytokine composition N (Trastuzumab antibody conjugated to IL-2 polypeptide) as a control

FIG. 10B shows plots measuring the effect of the modified IL-2 polypeptides unconjugated and conjugated to the anti-PD1 antibody on the inducement of resting memory (CD45RA−) CD8+T_{eff} cells in an in vitro sample of human T-cells in the presence or absence of excess amounts of unconjugated anti-PD1 antibody Pembrolizumab, with the figure showing mean fluorescence intensity for phosphorylated signal transducer and activator of transcription 5 (pSTAT5) on the y-axis and dosage of modified IL-2 polypeptide and immunocytokines on the x-axis. The modified IL-2 polypeptide tested in this figure is Composition AA and the immunocytokines tested in this figure are Composition B and Her2-targeted immunocytokine composition N (Trastuzumab antibody conjugated to IL-2 polypeptide) as a control.

FIG. 11A shows a plot describing the effect of PD-1 targeted and untargeted immunocytokines on the growth of CT26 syngeneic colon carcinoma tumors in hPD1 humanized BALB/c mice. The immunocytokine tested in this figure is Composition A tested as a single agent at 1, and 2.5 mg/kg after a single injection schedule. Control Her2-targeted immunocytokine Composition O (Trastuzumab antibody conjugated to IL-2 polypeptide) was also tested at 2.5 mg/kg. (mean±SEM).

FIG. 11B shows a bar chart describing the effect PD-1 targeted and untargeted immunocytokines on the growth of CT26 syngeneic colon carcinoma tumors in hPD1 humanized BALB/c mice 7 days after treatment. The immunocytokine tested in this figure is Composition A tested as a single agent at 1, and 2.5 mg/kg after a single injection schedule. Control Her2-targeted immunocytokine Composition O (Trastuzumab antibody conjugated to IL-2 polypeptide) was also tested at 2.5 mg/kg. (mean±SEM; **one-way ANOVA P-value<0.001).

FIG. 12A shows a plot describing the effect of PD-1 targeted and untargeted immunocytokines on the expansion of naïve (CD62L^{high} CD44^{low}) CD8+ T-cells in the blood and tumors of CT26 tumor bearing hPD1 humanized BALB/c mice 7 days after treatment. The immunocytokine tested in this figure is Composition A tested as a single agent at 1 and 2.5 mg/kg after a single injection schedule. Control Her2-targeted immunocytokine composition O (Trastuzumab antibody conjugated to IL-2 polypeptide) was also tested at 2.5 mg/kg. (n=3; mean±SEM).

FIG. 12B shows a plot describing the effect of PD-1 targeted and untargeted immunocytokines on the expansion of effector memory (CD62L^{negative} CD44^{high}) CD8+ T-cells in the blood and tumors of CT26 tumor bearing hPD1 humanized BALB/c mice 7 days after treatment. The immunocytokine tested in this figure is Composition A tested as a single agent at 1, and 2.5 mg/kg after a single injection schedule. Control Her2-targeted immunocytokine Composition O (Trastuzumab antibody conjugated to IL-2 polypeptide) was also tested at 2.5 mg/kg. (n=3; mean±SEM).

FIG. 13A shows a plot describing the effect of PD-1 targeting and untargeting of immunocytokines on their persistence in the blood and tumors of CT26 tumor bearing hPD1 humanized BALB/c mice, with the figure showing plasma or tumor concentration of PD-1 targeted and control immunocytokines on the y-axis and time on the x-axis. The immunocytokine tested in this figure is Composition A tested as a single agent at 1 and 2.5 mg/kg after a single injection schedule. Control Her2-targeted immunocytokine Composition O (Trastuzumab antibody conjugated to IL-2 polypeptide) was also tested at 2.5 mg/kg. (n=3; mean±SD).

FIG. 13B shows how PD-1 targeting results in a gradual accumulation of a PD1-IL2 immunocytokine within tumors over the course of 7d. In contrast, a non-targeted control immunocytokine (Her2-IL2) is cleared within 4d showing no intratumoral accumulation. The analysis was performed in CT26 tumor bearing hPD1 humanized BALB/c mice, and shown is the ratio of tumor/plasma concentrations of PD-1 targeted and control immunocytokines on the y-axis and time on the x-axis. The immunocytokine analysed is Composition A tested as a single agent at 1 and 2.5 mg/kg after a single application. Control Her2-targeted immunocytokine Composition O (Trastuzumab antibody conjugated to IL-2 polypeptide) was tested at 2.5 mg/kg. (n=3; mean±SEM).

FIG. 14A shows a plot describing the effect of a single injection of conjugated anti-PD1 antibody on the growth of MC38 syngeneic colon carcinoma tumors in hPD1 C57BL/6 mice. The immunocytokine tested in this figure is Composition H tested as a single agent at 1 mg/kg as a single injection. (n=8; mean±SEM).

FIG. 14B shows a bar chart describing the effect of a single injection of conjugated anti-PD1 antibody on the growth of MC38 syngeneic colon carcinoma tumors in hPD1 C57BL/6 mice after 7 days of treatment. The immunocytokine tested in this figure is Composition H tested as a single agent at 1 mg/kg a single injection. (n=8 animals; mean±SEM; **one-way ANOVA P-value<0.005).

### DETAILED DESCRIPTION

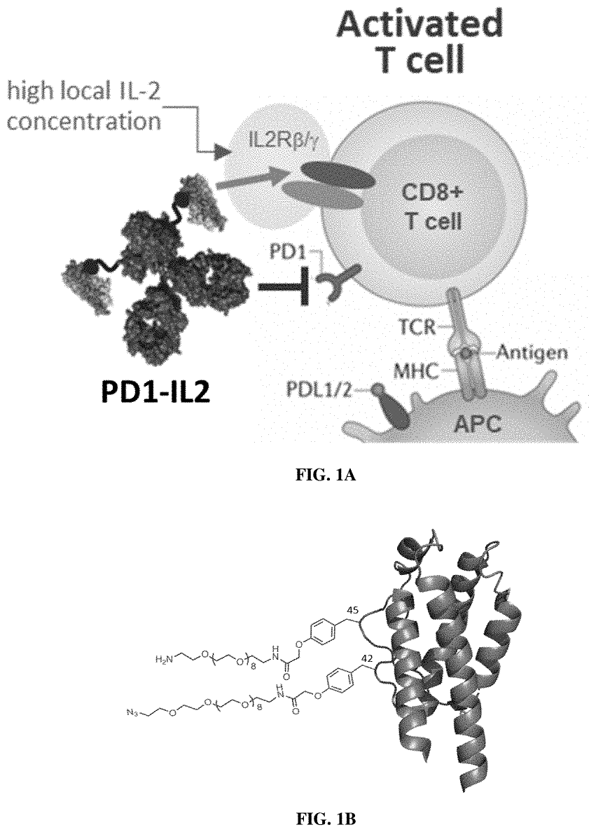

Disclosed herein are anti-PD-1 polypeptides. In some embodiments, the anti-PD-1 polypeptides are conjugated to a cell-signaling molecule, such as a cytokine. In some embodiments, the cytokine is IL-2. FIG. 1A illustrates an exemplary immunocytokine comprising an anti-PD-1 polypeptide conjugated to an IL-2 cytokine. The anti-PD-1 antibody/IL-2 immunocytokines (referred to herein as PD1-IL2s) of the disclosure can have superior efficacy and potentially improved tolerability by a subject. In some embodiments, the anti-PD-1-IL-2 immunocytokines of the disclosure can directly target tumor-infiltrating lymphocytes (TILs). In some embodiments, the anti-PD-1-IL-2 immunocytokines can significantly reduce the therapeutic dose of the anti-PD-1 polypeptide or IL-2 for a subject with a disease, such as cancer.

The anti-PD-1-IL-2 immunocytokines can act by one or more modes of action. In some embodiments, the anti-PD-1-IL-2 immunocytokines can inhibit PD-1 by targeting PD-1 and CD8+ T cells within tumors. In some embodiments, the anti-PD-1-IL-2 immunocytokines can activate T cells and NK cells via IL-2R βγ.

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment. The section headings used herein are for organizational purposes only and are not to be construed as limiting the subject matter described.

### Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise. In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Referred to herein are groups which are “attached” or “covalently attached” to residues of IL-2 polypeptides. As used herein, “attached” or “covalently attached” means that the group is tethered to the indicated residue, and such tethering can include a linking group (i.e., a linker). Thus, for a group “attached” or “covalently attached” to a residue, it is expressly contemplated that such linking groups are also encompassed.

Binding affinity refers to the strength of a binding interaction between a single molecule and its ligand/binding partner. A higher binding affinity refers to a higher strength bond than a lower binding affinity. In some instances, binding affinity is measured by the dissociation constant (K_{D}) between the two relevant molecules. When comparing K_{D} values, a binding interaction with a lower value will have a higher binding affinity than a binding interaction with a higher value. For a protein-ligand interaction, K_{D} is calculated according to the following formula:

$${K}_{D} = \frac{[ L ] [ P ]}{[ L P ]}$$

where [L] is the concentration of the ligand, [P] is the concentration of the protein, and [LP] is the concentration of the ligand/protein complex.

Referred to herein are certain amino acid sequences (e.g., polypeptide sequences) which have a certain percent sequence identity to a reference sequence or refer to a residue at a position corresponding to a position of a reference sequence. Sequence identity is measured by protein-protein BLAST algorithm using parameters of Matrix BLOSUM62, Gap Costs Existence:11, Extension:1, and Compositional Adjustments Conditional Compositional Score Matrix Adjustment. This alignment algorithm is also used to assess if a residue is at a “corresponding” position through an analysis of the alignment of the two sequences being compared.

The term “pharmaceutically acceptable” refers to approved or approvable by a regulatory agency of the Federal or a state government or listed in the U.S. Pharmacopeia or other generally recognized pharmacopeia for use in animals, including humans.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

A “pharmaceutically acceptable salt” suitable for the disclosure may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts include those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Certain formulas and other illustrations provided herein depict triazole reaction products resulting from azide-alkyne cycloaddition reactions. While such formulas generally depict only a single regioisomer of the resulting triazole formed in the reaction, it is intended that the formulas encompass both resulting regioisomers. Thus, while the formulas depict only a single regioisomer

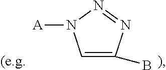

it is intended that the other regioisomer

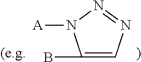

is also encompassed.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

The term “optional” or “optionally” denotes that a subsequently described event or circumstance can but need not occur, and that the description includes instances where the event or circumstance occurs and instances in which it does not.

The term “moiety” refers to a specific segment or functional group of a molecule. Chemical moieties are often recognized chemical entities embedded in or appended to a molecule.

As used herein, the term “number average molecular weight” (Mn) means the statistical average molecular weight of all the individual units in a sample, and is defined by Formula (1):

$$Mn = \frac{\sum {N}_{i} {M}_{i}}{\sum {N}_{i}} \qquad Formula ( 1 )$$

where Mᵢ is the molecular weight of a unit and Nᵢ is the number of units of that molecular weight.

As used herein, the term “weight average molecular weight” (Mw) means the number defined by Formula (2):

$$Mw = \frac{\sum {N}_{i} {M}_{i}^{2}}{\sum {N}_{i} {M}_{i}} \qquad Formula ( 2 )$$

where Mᵢ is the molecular weight of a unit and Nᵢ is the number of units of that molecular weight.

As used herein, “peak molecular weight” (Mp) means the molecular weight of the highest peak in a given analytical method (e.g., mass spectrometry, size exclusion chromatography, dynamic light scattering, analytical centrifugation, etc.).

As used herein, “non-canonical” amino acids can refer to amino acid residues in D- or L-form that are not among the 20 canonical amino acids generally incorporated into naturally occurring proteins.

As used herein, “conjugation handle” refers to a reactive group capable of forming a bond upon contacting a complementary reactive group. In some instances, a conjugation handle preferably does not have a substantial reactivity with other molecules which do not comprise the intended complementary reactive group. Non-limiting examples of conjugation handles, their respective complementary conjugation handles, and corresponding reaction products can be found in the table below. While table headings place certain reactive groups under the title “conjugation handle” or “complementary conjugation handle,” it is intended that any reference to a conjugation handle can instead encompass the complementary conjugation handles listed in the table (e.g., a trans-cyclooctene can be a conjugation handle, in which case tetrazine would be the complementary conjugation handle). In some instances, amine conjugation handles and conjugation handles complementary to amines are less preferable for use in biological systems owing to the ubiquitous presence of amines in biological systems and the increased likelihood for off-target conjugation.

Table of Conjugation Handles

| | | Reaction |
|---|---|---|
| Conjugation Handle | Complementary Conjugation Handle | Product |
| Sulfhydryl | alpha-halo-carbonyl (e.g., bromoacetamide), alpha- | thioether |
| | beta unsaturated carbonyl (e.g., maleimide, | |
| | acrylamide) | |
| Azide | alkyne (e.g, terminal alkyne, substituted | triazole |
| | cyclooctyne (e.g., dibenzocycloocytne (DBCO), | |
| | difluorocyclooctyne, bicyclo[6.1.0]nonyne, etc.) ) | |
| Phosphine | Azide/ester pair | amide |
| Tetrazine | trans-cyoclooctene | dihydropyridazine |
| Amine | Activated ester (e.g, N-hydroxysuccinimide ester, | amide |
| | pentaflurophenyl ester) | |
| isocyanate | amine | urea |
| epoxide | amine | alkyl-amine |
| hydroxyl amine | aldehyde, ketone | oxime |
| hydrazide | aldehyde, ketone | hydrazone |
| potassium acyl | O-substituted hydroxylamine (e.g., | amide |
| trifluoroborate | O-carbamoylhydroxylamine) | |

Throughout the instant application, prefixes are used before the term “conjugation handle” to denote the functionality to which the conjugation handle is linked. For example, a “protein conjugation handle” is a conjugation handle attached to a protein (either directly or through a linker), an “antibody conjugation handle” is a conjugation handle attached to an antibody (either directly or through a linker), and a “linker conjugation handle” is a conjugation handle attached to a linker group (e.g., a bifunctional linker used to link a synthetic protein and an antibody).

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, -propyl, 1-methyl ethyl, -butyl, -pentyl, 1,1-dimethyl ethyl, 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted. “Alkylene” or “alkylene chain” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—. Unless stated otherwise specifically in the specification, an alkylene group may be optionally substituted.

The term “alkenylene” or “alkenylene chain” refers to a straight or branched divalent hydrocarbon chain in which at least one carbon-carbon double bond is present linking the rest of the molecule to a radical group. In some embodiments, the alkenylene is —CH═CH—, —CH₂CH═CH—, or —CH═CHCH₂—. In some embodiments, the alkenylene is —CH═CH—. In some embodiments, the alkenylene is —CH₂CH═CH—. In some embodiments, the alkenylene is —CH═CHCH₂—.

The term “alkynyl” refers to a type of alkyl group in which at least one carbon-carbon triple bond is present. In one embodiment, an alkenyl group has the formula —C≡C—R^{X}, wherein Rₓ refers to the remaining portions of the alkynyl group. In some embodiments, R^{x} is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃, —C≡CCH₂CH, and —CH₂C^{∘}CH.

The term “aryl” refers to a radical comprising at least one aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted. In some embodiments, an aryl group comprises a partially reduced cycloalkyl group defined herein (e.g., 1,2-dihydronaphthalene). In some embodiments, an aryl group comprises a fully reduced cycloalkyl group defined herein (e.g., 1,2,3,4-tetrahydronaphthalene). When aryl comprises a cycloalkyl group, the aryl is bonded to the rest of the molecule through an aromatic ring carbon atom. An aryl radical can be a monocyclic or polycyclic (e.g., bicyclic, tricyclic, or tetracyclic) ring system, which may include fused, spiro or bridged ring systems.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopentyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted.

The term “heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkylene groups include, but are not limited to —CH₂—O—CH₂—, —CH₂—N(alkyl)-CH₂—, —CH₂—N(aryl)-CH₂—, —OCH₂CH₂O—, —OCH₂CH₂OCH₂CH₂O—, or —OCH₂CH₂OCH₂CH₂OCH₂CH₂O—.

The term “heteocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quatemized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

The term “heteroaryl” refers to an aryl group that includes one or more ring heteroatoms selected from nitrogen, oxygen, and sulfur. In some embodiments, heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 0 atoms, 0-1 P atoms, and 0-1 S atoms in the ring. In some embodiments, a heteroaryl contains 1-4 N atoms, 0-1 0 atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group comprises a partially reduced cycloalkyl or heterocycloalkyl group defined herein (e.g., 7,8-dihydroquinoline). In some embodiments, a heteroaryl group comprises a fully reduced cycloalkyl or heterocycloalkyl group defined herein (e.g., 5,6,7,8-tetrahydroquinoline). When heteroaryl comprises a cycloalkyl or heterocycloalkyl group, the heteroaryl is bonded to the rest of the molecule through a heteroaromatic ring carbon or hetero atom. A heteroaryl radical can be a monocyclic or polycyclic (e.g., bicyclic, tricyclic, or tetracyclic) ring system, which may include fused, spiro or bridged ring systems.

The term “optionally substituted” or “substituted” means that the referenced group is optionally substituted with one or more additional group(s) individually and independently selected from D, halogen, —CN, —NH2, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from D, halogen, —CN, —NH₂, —OH, —NH(CH₃), —N(CH₃)₂, —NH(cyclopropyl), —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

As used herein, “AJICAP™ technology,” “AJICAP™ methods,” and similar terms refer to systems and methods (currently produced by Ajinomoto Bio-Pharma Services (“Ajinomoto”)) for the site specific functionalization of antibodies and related molecules using affinity peptides to deliver the desired functionalization to the desired site. General protocols for the AJICAP™ methodology are found at least in PCT Publication No. WO2018199337A1, PCT Publication No. WO2019240288A1, PCT Publication No. WO2019240287A1, PCT Publication No. WO2020090979A1, Matsuda et al., Ma Pharmaceutics 2021, 18, 4058-4066, and Yamada et al., AJICAP: Affinity Peptide Mediated Regiodivergent Functionalization of Native Antibodies. Angew. Chem., Int. Ed. 2019, 58, 5592-5597, and in particular Examples 2-4 of US Patent Publication No. US20200190165A1. In some embodiments, such methodologies site specifically incorporate the desired functionalization at lysine residues at a position selected from position 246, position 248, position 288, position 290, and position 317 of an antibody Fc region (e.g., an IgG1 Fc region) (EU numbering). In some embodiments, the desired functionalization is incorporated at residue position 248 of an antibody Fc region (EU numbering). In some embodiments, position 248 corresponds to the 18th residue in a human IgG CH₂ region (EU numbering).

Composition AA refers to a modified IL-2 polypeptide having a sequence set forth in SEQ ID NO: 3 which contains a ˜0.5 kDa PEG group attached at residue Y45 and a second ˜0.5 kDa PEG group attached at residue F42Y.

Composition AB refers to a modified IL-2 polypeptide having a sequence set forth in SEQ ID NO: 3 which contains a ˜0.5 kDa PEG group attached at residue Y45 and a 0.5 kDa PEG group capped with an azide functionality to facilitate conjugations at residue F42Y. A cartoon image of Composition AB is shown in FIG. 1B. Composition AB and related modified IL-2 polypeptides are described in PCT Publication No. WO2021140416A2, which is hereby incorporated by reference as set forth in its entirety. The polymers attached to Composition AB act to disrupt Composition AB's interaction with the IL-2 receptor alpha subunit and bias the molecule in favor of IL-2 receptor beta subunit signaling, thus enhancing the ability of the IL-2 polypeptide to expand and/or stimulate T_{eff} cells in vivo compared to WT IL-2.

Composition AC refers to a modified IL-2 polypeptide having a sequence set forth in SEQ ID NO: 3 which contains ˜0.5 kDa PEG groups attached at residue F42Y and Y45. Composition AC contains an azide conjugation handle attached to the N-terminal A residue through a ˜0.5 kDa PEG (see Structure 7 provided herein) coupled via glutaric acid linker functionality.

Composition A refer to an anti-PD-1 antibody/IL-2 conjugate prepared from a reaction of Composition AB and anti-PD-1 antibodies Pembrolizumab or LZM-009. Composition A is formed from a reaction of the azide functionality of Composition AB with a DBCO functionality attached to residue K248 of the Fc region of Pembrolizumab (EU numbering). The DBCO functionality is added to Pembrolizumab using an affinity peptide system according to AJICAP technology from Ajinomoto. Composition A has a drug-antibody ratio of 1.

Composition B is formed from a reaction of the azide functionality of Composition AB with a DBCO functionality attached to residue K248 of the Fc region of Pembrolizumab (EU numbering). The DBCO functionality is added to Pembrolizumab using an affinity peptide system according to AJICAP technology from Ajinomoto. Composition B has a drug-antibody ratio of 1.5.

Composition C is formed from a reaction of the azide functionality of Composition AB with a DBCO functionality attached to residue K248 of the Fc region of Pembrolizumab (EU numbering). The DBCO functionality is added to Pembrolizumab using an affinity peptide system according to AJICAP technology from Ajinomoto. Composition C has a drug-antibody ratio of 2.

Composition D is formed from a reaction of the azide functionality of Composition AB with a DBCO functionality attached to residue K288 of the Fc region of Pembrolizumab (EU numbering). The DBCO functionality is added to Pembrolizumab using an affinity peptide system according to AJICAP technology from Ajinomoto. Composition D has a drug-antibody ratio of 1.

Composition E is formed from a reaction of the azide functionality of Composition AB with a DBCO functionality attached to residue K288 of the Fc region of Pembrolizumab (EU numbering). The DBCO functionality is added to Pembrolizumab using an affinity peptide system according to AJICAP technology from Ajinomoto. Composition E has a drug-antibody ratio of 2.

Composition F is formed from a reaction of the azide functionality of Composition AC with a DBCO functionality attached to residue K248 of the Fc region of Pembrolizumab (EU numbering). The DBCO functionality is added to Pembrolizumab using an affinity peptide system according to AJICAP technology from Ajinomoto. Composition F has a drug-antibody ratio of 1.

Composition G is formed from a reaction of the azide functionality of Composition AC with a DBCO functionality attached to residue K248 of the Fc region of Pembrolizumab (EU numbering). The DBCO functionality is added to Pembrolizumab using an affinity peptide system according to AJICAP technology from Ajinomoto. Composition G has a drug-antibody ratio of 2.

Composition H is formed from a reaction of the azide functionality of Composition AB with a DBCO functionality attached to residue K248 of the Fc region of LZM-009 (EU numbering). The DBCO functionality is added to LZM-009 using an affinity peptide system according to AJICAP technology from Ajinomoto. Composition H has a drug-antibody ratio of 1.

Composition I is formed from a reaction of the azide functionality of Composition AB with a DBCO functionality attached to residue K248 of the Fc region of LZM-009 (EU numbering). The DBCO functionality is added to LZM-009 using an affinity peptide system according to AJICAP technology from Ajinomoto. Composition I has a drug-antibody ratio of 2.

Composition J is formed from a reaction of the azide functionality of Composition AB with a DBCO functionality attached to residue K288 of the Fc region of LZM-009 (EU numbering). The DBCO functionality is added to LZM-009 using an affinity peptide system according to AJICAP technology from Ajinomoto. Composition J has a drug-antibody ratio of 1.

Composition K is formed from a reaction of the azide functionality of Composition AB with a DBCO functionality attached to residue K288 of the Fc region of LZM-009 (EU numbering). The DBCO functionality is added to LZM-009 using an affinity peptide system according to AJICAP technology from Ajinomoto. Composition K has a drug-antibody ratio of 2.

Composition N is formed from a reaction of the azide functionality of Composition AB with a DBCO functionality attached to residue K248 of the Fc region of Trastuzumab (EU numbering). The DBCO functionality is added to Trastuzumab using an affinity peptide system according to AJICAP technology from Ajinomoto. Composition N has a drug-antibody ratio of 1.6.

Composition O is formed from a reaction of the azide functionality of Composition AB with a DBCO functionality attached to residue K248 of the Fc region of Trastuzumab (EU numbering). The DBCO functionality is added to Trastuzumab using an affinity peptide system according to AJICAP technology from Ajinomoto. Composition O has a drug-antibody ratio of 1. An overview of all immunocytokine compositions is presented in the table below.

Overview of compositions

| | | | | IL-2 | IL-2 | | |
|---|---|---|---|---|---|---|---|
| | | VH | VL | polypeptide | polypeptide | | Conjugation |
| Composition | Antibody | Seq ID | Seq ID | Composition | Seq ID | DAR | site |
| A | Pembrolizumab | 46 | 47 | AB | 3 | 1 | K248 |
| B | Pembrolizumab | 46 | 47 | AB | 3 | 1.5 | K248 |
| C | Pembrolizumab | 46 | 47 | AB | 3 | 2 | K248 |
| D | Pembrolizumab | 46 | 47 | AB | 3 | 1 | K288 |
| E | Pembrolizumab | 46 | 47 | AB | 3 | 2 | K288 |
| F | Pembrolizumab | 46 | 47 | AC | 3 | 1 | K248 |
| G | Pembrolizumab | 46 | 47 | AC | 3 | 2 | K248 |
| H | LZM-009 | 76 | 77 | AB | 3 | 1 | K248 |
| I | LZM-009 | 76 | 77 | AB | 3 | 2 | K248 |
| J | LZM-009 | 76 | 77 | AB | 3 | 1 | K288 |
| K | LZM-009 | 76 | 77 | AB | 3 | 2 | K288 |
| N | Trastuzumab | 121 | 122 | AB | 3 | 1.6 | K248 |
| O | Trastuzumab | 121 | 122 | AB | 3 | 1 | K248 |

Anti-PD-1 Polypeptides Conjugated to Cytokines

Programmed cell death protein 1 (also known as PD-1 and CD279), is a cell surface receptor that plays an role in down-regulating the immune system and promoting self-tolerance by suppressing T cell inflammatory activity. PD-1 is an immune cell inhibitory molecule that is expressed on activated B cells, T cells, and myeloid cells. PD-1 represents an immune checkpoint and guards against autoimmunity via a dual mechanism of promoting apoptosis (programmed cell death) in antigen-specific T-cells in lymph nodes while reducing apoptosis in regulatory T cells. PD-1 is a member of the CD28/CTLA-4/ICOS costimulatory receptor family that delivers negative signals that affect T and B cell immunity. PD-1 is monomeric both in solution as well as on cell surface, in contrast to CTLA-4 and other family members that are all disulfide-linked homodimers. Signaling through the PD-1 inhibitory receptor upon binding its ligand, PD-L1, suppresses immune responses against autoantigens and tumors and plays a role in the maintenance of peripheral immune tolerance. The interaction between PD-1 and PD-L1 results in a decrease in tumor infiltrating lymphocytes, a decrease in T cell receptor mediated proliferation, and immune evasion by the cancerous cells. A non-limiting, exemplary, human PD-1 amino acid sequence is

(SEQ ID NO: 120)
MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLVVGVVGGLLGSLVLLVWVLAVICSRAARGTI
GARRTGQPLKEDPSAVPVFSVDYGELDFQWREKTPEPPVPCVPEQTEYAT
IVFPSGMGTSSPARRGSADGPRSAQPLRPEDGHCSWPL.

Provided herein are polypeptides, such as antibodies and anti-PD-1 antigen binding fragments, which bind to programmed cell death protein 1 (PD-1) which are conjugated to one or more cytokine molecules or derivatives thereof. The conjugates provided herein are effective for simultaneously delivering the cytokine and the polypeptide which selectively binds to PD-1 to a target cell, such as a CD8+T effector (T_{eff}) cell. This simultaneous delivery of both agents to the same cell has numerous benefits, including improved IL-2 polypeptide selectivity, enhance the therapeutic potential of IL-2, and potentially reduce the risk of side effects from administering IL-2 therapies.

The conjugate compositions provided herein utilize linkers to attach the polypeptides which bind to PD-1 to the cytokines, such as IL-2 polypeptides and derivatives thereof. In some embodiments, the linkers are attached to each moiety the polypeptide which selectively binds to PD-1 and the cytokine) at specific residues or a specific subset of residues. In some embodiments, the linkers are attached to each moiety in a site-selective manner, such that a population of the conjugate is substantially uniform. This can be accomplished in a variety of ways as provided herein, including by site-selectively adding reagents for a conjugation reaction to a moiety to be conjugated, synthesizing, or otherwise preparing a moiety to be conjugated with a desired reagent for a conjugation reaction, or a combination of these two approaches. Using these approaches, the sites of attachment (such as specific amino acid residues) of the linker to each moiety can be selected with precision. Additionally, these approaches allow a variety of linkers to be employed for the composition which are not limited to amino acid residues as is required for fusion proteins. This combination of linker choice and precision attachment to the moieties allows the linker to also, in some embodiments, perform the function of modulating the activity of one of the moieties, for example if the linker is attached to the cytokine at a position that interacts with a receptor of the cytokine.

Anti-PD-1 Polypeptides

In some embodiments, an anti-PD-1 polypeptide of the disclosure specifically binds to PD-1. An anti-PD-1 polypeptide selectively binds or preferentially binds to a target if it binds with greater affinity, avidity, more readily, and/or with greater duration than it binds to other substances. As such, “specific binding” or “preferential binding” does not necessarily require (although it can include) exclusive binding. Generally, but not necessarily, reference to specific binding means preferential binding where the affinity of the antibody, or antigen binding fragment thereof, is at least at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater than the affinity of the antibody for unrelated amino acid sequences. An anti-PD-1 polypeptide or an anti-PD-1 antigen binding fragment of the disclosure can block interaction of PD-1 with a ligand (e.g., PD-L1).

As used herein, the term “antibody” refers to an immunoglobulin (Ig), polypeptide, or a protein having a binding domain which is, or is homologous to, an antigen binding domain. The term further includes “antigen binding fragments” and other interchangeable terms for similar binding fragments as described below. Native antibodies and native immunoglobulins (Igs) are generally heterotetrameric glycoproteins of about 150,000 Daltons, composed of two identical light chains and two identical heavy chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (“VH”) followed by a number of constant domains (“CH”). Each light chain has a variable domain at one end (“VL”) and a constant domain (“CL”) at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

In some instances, an antibody or an antigen binding fragment comprises an isolated antibody or antigen binding fragment, a purified antibody or antigen binding fragment, a recombinant antibody or antigen binding fragment, a modified antibody or antigen binding fragment, or a synthetic antibody or antigen binding fragment.

Antibodies and antigen binding fragments herein can be partly or wholly synthetically produced. An antibody or antigen binding fragment can be a polypeptide or protein having a binding domain which can be, or can be homologous to, an antigen binding domain. In one instance, an antibody or an antigen binding fragment can be produced in an appropriate in vivo animal model and then isolated and/or purified.

Depending on the amino acid sequence of the constant domain of its heavy chains, immunoglobulins (Igs) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. An Ig or portion thereof can, in some cases, be a human Ig. In some instances, a C_{H}3 domain can be from an immunoglobulin. In some cases, a chain or a part of an antibody or antigen binding fragment, a modified antibody or antigen binding fragment, or a binding agent can be from an Ig. In such cases, an Ig can be IgG, an IgA, an IgD, an IgE, or an IgM, or is derived therefrom. In cases where the Ig is an IgG, it can be a subtype of IgG, wherein subtypes of IgG can include IgG1, an IgG2a, an IgG2b, an IgG3, or an IgG4. In some cases, a C_{H}3 domain can be from an immunoglobulin selected from the group consisting of an IgG, an IgA, an IgD, an IgE, and an IgM, or derived therefrom. In some embodiments, an antibody or antigen binding fragment described herein comprises an IgG or is derived therefrom. In some instances, an antibody or antigen binding fragment comprises an IgG1 or is derived therefrom. In some instances, an antibody or antigen binding fragment comprises an IgG4 or is derived therefrom. In some embodiments, an antibody or antigen binding fragment described herein comprises an IgM, is derived therefrom, or is a monomeric form of IgM. In some embodiments, an antibody or antigen binding fragment described herein comprises an IgE or is derived therefrom. In some embodiments, an antibody or antigen binding fragment described herein comprises an IgD or is derived therefrom. In some embodiments, an antibody or antigen binding fragment described herein comprises an IgA or is derived therefrom.

The “light chains” of antibodies (immunoglobulins) from any vertebrate species can be assigned to one of two clearly distinct types, called kappa (“κ” or “K”) or lambda (“λ”), based on the amino acid sequences of their constant domains.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (e.g., Kabat et al., Sequences of Proteins of Immunological Interest, (5th Ed., 1991, National Institutes of Health, Bethesda Md. (1991), pages 647-669; hereafter “Kabat”); and (2) an approach based on crystallographic studies of antigen-antibody complexes (Al-Iazikani et al. (1997) J. Molec. Biol. 273:927-948)). As used herein, a CDR may refer to CDRs defined by either approach or by a combination of both approaches.

With respect to antibodies, the term “variable domain” refers to the variable domains of antibodies that are used in the binding and specificity of each particular antibody for its particular antigen. However, the variability is not evenly distributed throughout the variable domains of antibodies. Rather, it is concentrated in three segments called hypervariable regions (also known as CDRs) in both the light chain and the heavy chain variable domains. More highly conserved portions of variable domains are called the “framework regions” or “FRs.” The variable domains of unmodified heavy and light chains each contain four FRs (FR1, FR2, FR3, and FR4), largely adopting a β-sheet configuration interspersed with three CDRs which form loops connecting and, in some cases, part of the β-sheet structure. The CDRs in each chain are held together in close proximity by the FRs and, with the CDRs from the other chain, contribute to the formation of the antigen binding site of antibodies (see, Kabat).

The terms “hypervariable region” and “CDR” when used herein, refer to the amino acid residues of an antibody which are responsible for antigen binding. The CDRs comprise amino acid residues from three sequence regions which bind in a complementary manner to an antigen and are known as CDR1, CDR2, and CDR3 for each of the V_{H} and V_{L} chains. In the light chain variable domain, the CDRs typically correspond to approximately residues 24-34 (CDRL1), 50-56 (CDRL2), and 89-97 (CDRL3), and in the heavy chain variable domain the CDRs typically correspond to approximately residues 31-35 (CDRH1), 50-65 (CDRH2), and 95-102 (CDRH3) according to Kabat. It is understood that the CDRs of different antibodies may contain insertions, thus the amino acid numbering may differ. The Kabat numbering system accounts for such insertions with a numbering scheme that utilizes letters attached to specific residues (e.g., 27A, 27B, 27C, 27D, 27E, and 27F of CDRL1 in the light chain) to reflect any insertions in the numberings between different antibodies. Alternatively, in the light chain variable domain, the CDRs typically correspond to approximately residues 26-32 (CDRL1), 50-52 (CDRL2), and 91-96 (CDRL3), and in the heavy chain variable domain, the CDRs typically correspond to approximately residues 26-32 (CDRH1), 53-55 (CDRH2), and 96-101 (CDRH3) according to Chothia and Lesk (J. Mol. Biol., 196: 901-917 (1987)).

As used herein, “framework region,” “FW,” or “FR” refers to framework amino acid residues that form a part of the antigen binding pocket or groove. In some embodiments, the framework residues form a loop that is a part of the antigen binding pocket or groove and the amino acids residues in the loop may or may not contact the antigen. Framework regions generally comprise the regions between the CDRs. In the light chain variable domain, the FRs typically correspond to approximately residues 0-23 (FRL1), 35-49 (FRL2), 57-88 (FRL3), and 98-109 and in the heavy chain variable domain the FRs typically correspond to approximately residues 0-30 (FRH1), 36-49 (FRH2), 66-94 (FRH3), and 103-133 according to Kabat. As discussed above with the Kabat numbering for the light chain, the heavy chain too accounts for insertions in a similar manner (e.g., 35A, 35B of CDRH1 in the heavy chain). Alternatively, in the light chain variable domain, the FRs typically correspond to approximately residues 0-25 (FRL1), 33-49 (FRL2) 53-90 (FRL3), and 97-109 (FRL4), and in the heavy chain variable domain, the FRs typically correspond to approximately residues 0-25 (FRH1), 33-52 (FRH2), 56-95 (FRH3), and 102-113 (FRH4) according to Chothia and Lesk, Id. The loop amino acids of a FR can be assessed and determined by inspection of the three-dimensional structure of an antibody heavy chain and/or antibody light chain. The three-dimensional structure can be analyzed for solvent accessible amino acid positions as such positions are likely to form a loop and/or provide antigen contact in an antibody variable domain. Some of the solvent accessible positions can tolerate amino acid sequence diversity and others (e.g., structural positions) are, generally, less diversified. The three-dimensional structure of the antibody variable domain can be derived from a crystal structure or protein modeling.

In the present disclosure, the following abbreviations (in the parentheses) are used in accordance with the customs, as necessary: heavy chain (H chain), light chain (L chain), heavy chain variable region (VH), light chain variable region (VL), complementarity determining region (CDR), first complementarity determining region (CDR1), second complementarity determining region (CDR2), third complementarity determining region (CDR3), heavy chain first complementarity determining region (VH CDR1), heavy chain second complementarity determining region (VH CDR2), heavy chain third complementarity determining region (VH CDR3), light chain first complementarity determining region (VL CDR1), light chain second complementarity determining region (VL CDR2), and light chain third complementarity determining region (VL CDR3).

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is generally defined to stretch from an amino acid residue at position Cys226, or from Pro230, to the carboxyl-terminus thereof. The numbering of the residues in the Fc region is that of the EU index as in Kabat. The Fc region of an immunoglobulin generally comprises two constant domains, C_{H}2 and C_{H}3.

“Antibodies” useful in the present disclosure encompass, but are not limited to, monoclonal antibodies, polyclonal antibodies, chimeric antibodies, bispecific antibodies, multispecific antibodies, heteroconjugate antibodies, humanized antibodies, human antibodies, grafted antibodies, deimmunized antibodies, mutants thereof, fusions thereof, immunoconjugates thereof, antigen binding fragments thereof, and/or any other modified configuration of the immunoglobulin molecule that comprises an antigen recognition site of the required specificity, including glycosylation variants of antibodies, amino acid sequence variants of antibodies, and covalently modified antibodies. In certain embodiments of the methods and conjugates provided herein, the antibody requires an Fc region to enable attachment of a linker between the antibody and the protein (e.g., attachment of the linker using an affinity peptide, such as in AJICAP′ technology).

In some instances, an antibody is a monoclonal antibody. As used herein, a “monoclonal antibody” refers to an antibody obtained from a population of substantially homogeneous antibodies, i.e., the individual antibodies comprising the population are identical except for possible naturally-occurring mutations that may be present in minor amounts. In contrast to polyclonal antibody preparations, which typically include different antibodies directed against different determinants (epitopes), each monoclonal antibody is directed against a single determinant on the antigen (epitope). The modifier “monoclonal” indicates the character of the antibody as being obtained from a substantially homogeneous population of antibodies and is not to be construed as requiring production of the antibody by any particular method.

In some instances, an antibody is a humanized antibody. As used herein, “humanized” antibodies refer to forms of non-human (e.g., murine) antibodies that are specific chimeric immunoglobulins, immunoglobulin chains, or fragments thereof that contain minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and biological activity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences but are included to further refine and optimize antibody performance. In general, a humanized antibody comprises substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in, for example, WO 99/58572. Other forms of humanized antibodies have one or more CDRs (one, two, three, four, five, or six) which are altered with respect to the original antibody, which are also termed one or more CDRs “derived from” one or more CDRs from the original antibody.

If needed, an antibody or an antigen binding fragment described herein can be assessed for immunogenicity and, as needed, be deimmunized (i.e., the antibody is made less immunoreactive by altering one or more T cell epitopes). As used herein, a “deimmunized antibody” means that one or more T cell epitopes in an antibody sequence have been modified such that a T cell response after administration of the antibody to a subject is reduced compared to an antibody that has not been deimmunized. Analysis of immunogenicity and T-cell epitopes present in the antibodies and antigen binding fragments described herein can be carried out via the use of software and specific databases. Exemplary software and databases include iTope™ developed by Antitope of Cambridge, England. iTope™, is an in silico technology for analysis of peptide binding to human MHC class II alleles. The iTope™ software predicts peptide binding to human MHC class II alleles and thereby provides an initial screen for the location of such “potential T cell epitopes.” iTope™ software predicts favorable interactions between amino acid side chains of a peptide and specific binding pockets within the binding grooves of 34 human MHC class II alleles. The location of key binding residues is achieved by the in silico generation of 9mer peptides that overlap by one amino acid spanning the test antibody variable region sequence. Each 9mer peptide can be tested against each of the 34 MHC class II allotypes and scored based on their potential “fit” and interactions with the MHC class II binding groove. Peptides that produce a high mean binding score (>0.55 in the iTope™ scoring function) against >50% of the MHC class II alleles are considered as potential T cell epitopes. In such regions, the core 9 amino acid sequence for peptide binding within the MHC class II groove is analyzed to determine the MHC class II pocket residues (P1, P4, P6, P7, and P9) and the possible T cell receptor (TCR) contact residues (P-1, P2, P3, P5, P8). After identification of any T-cell epitopes, amino acid residue changes, substitutions, additions, and/or deletions can be introduced to remove the identified T-cell epitope. Such changes can be made so as to preserve antibody structure and function while still removing the identified epitope. Exemplary changes can include, but are not limited to, conservative amino acid changes.

An antibody can be a human antibody. As used herein, a “human antibody” means an antibody having an amino acid sequence corresponding to that of an antibody produced by a human and/or that has been made using any suitable technique for making human antibodies. This definition of a human antibody includes antibodies comprising at least one human heavy chain polypeptide or at least one human light chain polypeptide. One such example is an antibody comprising murine light chain and human heavy chain polypeptides. In one embodiment, the human antibody is selected from a phage library, where that phage library expresses human antibodies. Human antibodies can also be made by introducing human immunoglobulin loci into transgenic animals, e.g., mice in which the endogenous immunoglobulin genes have been partially or completely inactivated. Alternatively, the human antibody may be prepared by immortalizing human B lymphocytes that produce an antibody directed against a target antigen (such B lymphocytes may be recovered from an individual or may have been immunized in vitro).

Any of the antibodies herein can be bispecific. Bispecific antibodies are antibodies that have binding specificities for at least two different antigens and can be prepared using the antibodies disclosed herein. Traditionally, the recombinant production of bispecific antibodies was based on the coexpression of two immunoglobulin heavy chain-light chain pairs, with the two heavy chains having different specificities. Bispecific antibodies can be composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations.

According to one approach to making bispecific antibodies, antibody variable domains with the desired binding specificities (antibody-antigen combining sites) are fused to immunoglobulin constant domain sequences. The fusion can be with an immunoglobulin heavy chain constant domain, comprising at least part of the hinge, CH₂ and CH₃ regions. The first heavy chain constant region (CH₁), containing the site necessary for light chain binding, can be present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In some instances, an antibody herein is a chimeric antibody. “Chimeric” forms of non-human (e.g., murine) antibodies include chimeric antibodies which contain minimal sequence derived from a non-human Ig. For the most part, chimeric antibodies are murine antibodies in which at least a portion of an immunoglobulin constant region (Fc), typically that of a human immunoglobulin, is inserted in place of the murine Fc. Chimeric or hybrid antibodies also may be prepared in vitro using suitable methods of synthetic protein chemistry, including those involving cross-linking agents. For example, immunotoxins may be constructed using a disulfide exchange reaction or by forming a thioether bond. Examples of suitable reagents for this purpose include iminothiolate and methyl-4-mercaptobutyrimidate.

Provided herein are antibodies and antigen binding fragments thereof, modified antibodies and antigen binding fragments thereof, and binding agents that specifically bind to one or more epitopes on one or more target antigens. In one instance, a binding agent selectively binds to an epitope on a single antigen. In another instance, a binding agent is bivalent and either selectively binds to two distinct epitopes on a single antigen or binds to two distinct epitopes on two distinct antigens. In another instance, a binding agent is multivalent (i.e., trivalent, quatravalent, etc.) and the binding agent binds to three or more distinct epitopes on a single antigen or binds to three or more distinct epitopes on two or more (multiple) antigens.

Antigen binding fragments of any of the antibodies herein are also contemplated. The terms “antigen binding portion of an antibody,” “antigen binding fragment,” “antigen binding domain,” “antibody fragment,” or a “functional fragment of an antibody” are used interchangeably herein to refer to one or more fragments of an antibody that retain the ability to specifically bind to an antigen. Representative antigen binding fragments include, but are not limited to, a Fab, a Fab′, a F(ab′)₂, a bispecific F(ab′)₂, a trispecific F(ab′)₂, a variable fragment (Fv), a single chain variable fragment (scFv), a dsFv, a bispecific scFv, a variable heavy domain, a variable light domain, a variable NAR domain, bispecific scFv, an AVIMER®, a minibody, a diabody, a bispecific diabody, triabody, a tetrabody, a minibody, a maxibody, a camelid, a VHH, a minibody, an intrabody, fusion proteins comprising an antibody portion (e.g., a domain antibody), a single chain binding polypeptide, a scFv-Fc, a Fab-Fc, a bispecific T cell engager (BiTE; two scFvs produced as a single polypeptide chain, where each scFv comprises an amino acid sequences a combination of CDRs or a combination of VL/VL described herein), a tetravalent tandem diabody (TandAb; an antibody fragment that is produced as a non-covalent homodimer folder in a head-to-tail arrangement, e.g., a TandAb comprising an scFv, where the scFv comprises an amino acid sequences a combination of CDRs or a combination of VL/VL described herein), a Dual-Affinity Re-targeting Antibody (DART; different scFvs joined by a stabilizing interchain disulphide bond), a bispecific antibody (bscAb; two single-chain Fv fragments joined via a glycine-serine linker), a single domain antibody (sdAb), a fusion protein, a bispecific disulfide-stabilized Fv antibody fragment (dsFv-dsFv; two different disulfide-stabilized Fv antibody fragments connected by flexible linker peptides). In certain embodiments of the invention, a full length antibody (e.g., an antigen binding fragment and an Fc region) are preferred.

Heteroconjugate polypeptides comprising two covalently joined antibodies or antigen binding fragments of antibodies are also within the scope of the disclosure. Suitable linkers may be used to multimerize binding agents. Non-limiting examples of linking peptides include, but are not limited to, (GS)ₙ (SEQ ID NO: 24), (GGS)ₙ (SEQ ID NO: 25), (GGGS)ₙ (SEQ ID NO: 26), (GGSG)ₙ (SEQ ID NO: 27), or (GGSGG)ₙ (SEQ ID NO: 28), (GGGGS)ₙ (SEQ ID NO: 29), wherein n is 1, 2, 3, 4, 5, 6, 7, 8, 9, or 10. For example, a linking peptide can be (GGGGS)₃ (SEQ ID NO: 30) or (GGGGS)₄ (SEQ ID NO: 31). In some embodiments, a linking peptide bridges approximately 3.5 nm between the carboxy terminus of one variable region and the amino terminus of the other variable region. Linkers of other sequences have been designed and used. Linkers can in turn be modified for additional functions, such as attachment of drugs or attachment to solid supports.

As used herein, the term “avidity” refers to the resistance of a complex of two or more agents to dissociation after dilution. Apparent affinities can be determined by methods such as an enzyme-linked immunosorbent assay (ELISA) or any other suitable technique. Avidities can be determined by methods such as a Scatchard analysis or any other suitable technique.

As used herein, the term “affinity” refers to the equilibrium constant for the reversible binding of two agents and is expressed as K_{D}. The binding affinity (K_{D}) of an antibody or antigen binding fragment herein can be less than 500 nM, 475 nM, 450 nM, 425 nM, 400 nM, 375 nM, 350 nM, 325 nM, 300 nM, 275 nM, 250 nM, 225 nM, 200 nM, 175 nM, 150 nM, 125 nM, 100 nM, 90 nM, 80 nM, 70 nM, 50 nM, 50 nM, 49 nM, 48 nM, 47 nM, 46 nM, 45 nM, 44 nM, 43 nM, 42 nM, 41 nM, 40 nM, 39 nM, 38 nM, 37 nM, 36 nM, 35 nM, 34 nM, 33 nM, 32 nM, 31 nM, 30 nM, 29 nM, 28 nM, 27 nM, 26 nM, 25 nM, 24 nM, 23 nM, 22 nM, 21 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, 1 nM, 990 pM, 980 pM, 970 pM, 960 pM, 950 pM, 940 pM, 930 pM, 920 pM, 910 pM, 900 pM, 890 pM, 880 pM, 870 pM, 860 pM, 850 pM, 840 pM, 830 pM, 820 pM, 810 pM, 800 pM, 790 pM, 780 pM, 770 pM, 760 pM, 750 pM, 740 pM, 730 pM, 720 pM, 710 pM, 700 pM, 690 pM, 680 pM, 670 pM, 660 pM, 650 pM, 640 pM, 630 pM, 620 pM, 610 pM, 600 pM, 590 pM, 580 pM, 570 pM, 560 pM, 550 pM, 540 pM, 530 pM, 520 pM, 510 pM, 500 pM, 490 pM, 480 pM, 470 pM, 460 pM, 450 pM, 440 pM, 430 pM, 420 pM, 410 pM, 400 pM, 390 pM, 380 pM, 370 pM, 360 pM, 350 pM, 340 pM, 330 pM, 320 pM, 310 pM, 300 pM, 290 pM, 280 pM, 270 pM, 260 pM, 250 pM, 240 pM, 230 pM, 220 pM, 210 pM, 200 pM, 190 pM, 180 pM, 170 pM, or any integer therebetween. Binding affinity may be determined using surface plasmon resonance (SPR), KINEXA® Biosensor, scintillation proximity assays, enzyme linked immunosorbent assay (ELISA), ORIGEN immunoassay (IGEN), fluorescence quenching, fluorescence transfer, yeast display, or any combination thereof. Binding affinity may also be screened using a suitable bioassay.

As used herein, the term “avidity” refers to the resistance of a complex of two or more agents to dissociation after dilution. Apparent affinities can be determined by methods such as an enzyme linked immunosorbent assay (ELISA) or any other technique familiar to one of skill in the art. Avidities can be determined by methods such as a Scatchard analysis or any other technique familiar to one of skill in the art.

Also provided herein are affinity matured antibodies. The following methods may be used for adjusting the affinity of an antibody and for characterizing a CDR. One way of characterizing a CDR of an antibody and/or altering (such as improving) the binding affinity of a polypeptide, such as an antibody, is termed “library scanning mutagenesis.” Generally, library scanning mutagenesis works as follows. One or more amino acid position in the CDR is replaced with two or more (such as 3, 4, 5, 6, 7, 8, 9, 10, 11, 12, 13, 14, 15, 16, 17, 18, 19, or 20) amino acids. This generates small libraries of clones (in some embodiments, one for every amino acid position that is analyzed), each with a complexity of two or more members (if two or more amino acids are substituted at every position). Generally, the library also includes a clone comprising the native (unsubstituted) amino acid. A small number of clones, for example, about 20-80 clones (depending on the complexity of the library), from each library can be screened for binding specificity or affinity to the target polypeptide (or other binding target), and candidates with increased, the same, decreased, or no binding are identified. Binding affinity may be determined using Biacore surface plasmon resonance analysis, which detects differences in binding affinity of about 2-fold or greater.

In some instances, an antibody or antigen binding fragment is bispecific or multispecific and can specifically bind to more than one antigen. In some cases, such a bispecific or multispecific antibody or antigen binding fragment can specifically bind to 2 or more different antigens. In some cases, a bispecific antibody or antigen binding fragment can be a bivalent antibody or antigen binding fragment. In some cases, a multi specific antibody or antigen binding fragment can be a bivalent antibody or antigen binding fragment, a trivalent antibody or antigen binding fragment, or a quadravalent antibody or antigen binding fragment.

An antibody or antigen binding fragment described herein can be isolated, purified, recombinant, or synthetic.

The antibodies described herein may be made by any suitable method. Antibodies can often be produced in large quantities, particularly when utilizing high level expression vectors.

In one embodiment, an anti-PD1 antibody or an anti-PD1 antigen binding fragment of the disclosure comprises a combination of a heavy chain variable region (VH) and a light chain variable region (VL) described herein. In another embodiment, an anti-PD1 antibody or an anti-PD1 antigen binding fragment of the disclosure comprises a combination of complementarity determining regions (VH CDR1, VH CDR2, VH CDR3, VL CDR1, VL CDR2, and VL CDR3) described herein. In one embodiment, an anti-PD-1 antibody or an anti-PD-1 antigen binding fragment of the disclosure comprises a modified Tislelizumab, Baizean, OKVO411B3N, BGB-A317, hu317-1/IgG4mt2, Sintilimab, Tyvyt, IBI-308, Toripalimab, TeRuiPuLi, Terepril, Tuoyi, JS-001, TAB-001, Camrelizumab, HR-301210, INCSHR-01210, SHR-1210, Cemiplimab, Cemiplimab-rwlc, LIBTAYO®, 6QVL057INT, H4H7798N, REGN-2810, SAR-439684, Lambrolizumab, Pembrolizumab, KEYTRUDA®, MK-3475, SCH-900475, h409A11, Nivolumab, Nivolumab BMS, OPDIVO®, BMS-936558, MDX-1106, ONO-4538, Prolgolimab, Forteca, BCD-100, Penpulimab, AK-105, Zimberelimab, AB-122, GLS-010, WBP-3055, Balstilimab, 1Q2QT5M7EO, AGEN-2034, AGEN-2034w, Genolimzumab, Geptanolimab, APL-501, CBT-501, GB-226, Dostarlimab, ANB-011, GSK-4057190A, POGVQ9A4S5, TSR-042, WBP-285, Serplulimab, IALX-10, CS-1003, Retifanlimab, 2Y3T5IF01Z, INCMGA-00012, INCMGA-0012, MGA-012, Sasanlimab, LZZOIC2EWP, PF-06801591, RN-888, Spartalizumab, NVP-LZV-184, PDR-001, QOG25L6Z8Z, Relatlimab/nivolumab, BMS-986213, Cetrelimab, JNJ-3283, JNJ-63723283, LYK98WP91F, Tebotelimab, MGD-013, BCD-217, BAT-1306, HX-008, MEDI-5752, JTX-4014, Cadonilimab, AK-104, BI-754091, Pidilizumab, CT-011, MDV-9300, YBL-006, AMG-256, RG-6279, RO-7284755, BH-2950, IBI-315, RG-6139, RO-7247669, ONO-4685, AK-112, 609-A, LY-3434172, T-3011, MAX-10181, AMG-404, MGD-019, INCB-086550, ONCR-177, LY-3462817, RG-7769, RO-7121661, F-520, XmAb-23104, Pd-1-pik, SG-001, 5-95016, Sym-021, LZM-009, Budigalimab, 6VDO4TY300, ABBV-181, PR-1648817, CC-90006, XmAb-20717, 2661380, AMP-224, B7-DCIg, EMB-02, ANB-030, PRS-332, [89Zr]Deferoxamide-Pembrolizumab, 89Zr-Df-Pembrolizumab, [89Zr]Df-Pembrolizumab, STI-1110, STI-A1110, CX-188, mPD-1 Pb-Tx, MCLA-134, 244C₈, ENUM 224C₈, ENUM C8, 388D4, ENUM 388D4, ENUM D4, MEDI0680, or AMP-514.

In one embodiment, an anti-PD-1 antibody or an anti-PD-1 antigen binding fragment of the disclosure comprises a modified Tislelizumab, Sintilimab, Toripalimab, Terepril, Camrelizumab, Cemiplimab, Pembrolizumab Nivolumab, Prolgolimab, Penpulimab, Zimberelimab, Balstilimab, Genolimzumab, Geptanolimab, Dostarlimab, Serplulimab, Retifanlimab, Sasanlimab, Spartalizumab, Cetrelimab, Tebotelimab, Cadonilimab, A Pidilizumab, LZM-009, or Budigalimab.

In some embodiments, the anti-PD-1 polypeptide is Nivolumab, Pembrolizumab, LZM-009, Dostarlimab, Sintilimab, Spartalizumab, Tislelizumab, or Cemiplimab. In some embodiment, the anti-PD-1 polypeptide is Dostarlimab, Sintilimab, Spartalizumab, or Tislelizumab. In some embodiments, the anti-PD-1 polypeptide is Nivolumab, Pembrolizumab, LZM-009, or Cemiplimab.

In some embodiments, the anti-PD-1 polypeptide is modified Pembrolizumab. In some embodiments, the anti-PD-1 polypeptide is modified with mAB3. In some embodiments, the anti-PD-1 polypeptide is modified with mAB4.

It is contemplated that generic or biosimilar versions of the named antibodies herein which share the same amino acid sequence as the indicated antibodies are also encompassed when the name of the antibody is used. In some embodiments, the anti-PD-1 antibody is a biosimilar of Tislelizumab, Sintilimab, Toripalimab, Terepril, Camrelizumab, Cemiplimab, Pembrolizumab Nivolumab, Prolgolimab, Penpulimab, Zimberelimab, Balstilimab, Genolimzumab, Geptanolimab, Dostarlimab, Serplulimab, Retifanlimab, Sasanlimab, Spartalizumab, Cetrelimab, Tebotelimab, Cadonilimab, A Pidilizumab, LZM-009, or Budigalimab. In some embodiments, the anti-PD-1 antibody is a biosimilar of any one of the antibodies provided herein.

TABLE 1 provides the sequences of exemplary anti-PD-1 polypeptides (e.g., anti-PD-1 antibodies) and anti-PD-1 antigen binding fragments that can be modified to prepare anti-PD-1 immunoconjugates. TABLE 1 also shows provides combinations of CDRs that can be utilized in a modified anti-PD-1 immunoconjugate. Reference to an anti-PD-1 polypeptide herein may alternatively refer to an anti-PD-1 antigen binding fragment.

TABLE 1

| Antibody or | | SEQ |
|---|---|---|
| Ag-binding | | ID |
| fragment | Sequence | NO |
| Tislelizumab, | QVQLQESGPGLVKPSETLSLTCTVSGFSLTSYGVHWIRQPPGKGL | 32 |
| Baizean, | EWIGVIYADGSTNYNPSLKSRVTISKDTSKNQVSLKLSSVTAADT | |
| 0KVO411B3N, | AVYYCARAYGNYWYIDVWGQGTTVTVSSASTKGPSVFPLAPCS | |
| BGB-A317, | RSTSESTAALGCLVKDYFPEPVTVSWNSGALTSGVHTFPAVLQSS | |
| hu317- | GLYSLSSVVTVPSSSLGTKTYTCNVDHKPSNTKVDKRVESKYGP | |
| 1/IgG4mt2 | PCPPCPAPPVAGGPSVFLFPPKPKDTLMISRTPEVTCVVVAVSQE | |
| VH | DPEVQFNWYVDGVEVHNAKTKPREEQFNSTYRVVSVLTVVHQD | |
| | WLNGKEYKCKVSNKGLPSSIEKTISKAKGQPREPQVYTLPPSQEE | |
| | MTKNQVSLTCLVKGFYPSDIAVEWESNGQPENNYKTTPPVLDSD | |
| | GSFFLYSKLTVDKSRWQEGNVFSCSVMHEALHNHYTQKSLSLSL | |
| | GK | |
| Tislelizumab, | DIVMTQSPDSLAVSLGERATINCKSSESVSNDVAWYQQKPGQPP | 33 |
| Baizean, | KLLINYAFHRFTGVPDRFSGSGYGTDFTLTISSLQAEDVAVYYCH | |
| 0KVO411B3N, | QAYSSPYTFGQGTKLEIKRTVAAPSVFIFPPSDEQLKSGTASVVCL | |
| BGB-A317, | LNNFYPREAKVQWKVDNALQSGNSQESVTEQDSKDSTYSLSSTL | |
| hu317- | TLSKADYEKHKVYACEVTHQGLSSPVTKSFNRGEC | |
| 1/IgG4mt2 | | |
| VL | | |
| Sintilimab, | QVQLVQSGAEVKKPGSSVKVSCKASGGTFSSYAISWVRQAPGQ | 34 |
| Tyvyt, IBI-308 | GLEWMGLIIPMFDTAGYAQKFQGRVAITVDESTSTAYMELSSLR | |
| VH | SEDTAVYYCARAEHSSTGTFDYWGQGTLVTVSSASTKGPSVFPL | |
| | APCSRSTSESTAALGCLVKDYFPEPVTVSWNSGALTSGVHTFPAV | |
| | LQSSGLYSLSSVVTVPSSSLGTKTYTCNVDHKPSNTKVDKRVESK | |
| | YGPPCPPCPAPEFLGGPSVFLFPPKPKDTLMISRTPEVTCVVVDVS | |
| | QEDPEVQFNWYVDGVEVHNAKTKPREEQFNSTYRVVSVLTVLH | |
| | QDWLNGKEYKCKVSNKGLPSSIEKTISKAKGQPREPQVYTLPPSQ | |
| | EEMTKNQVSLTCLVKGFYPSDIAVEWESNGQPENNYKTTPPVLD | |
| | SDGSFFLYSRLTVDKSRWQEGNVFSCSVMHEALHNHYTQKSLSL | |
| | SLGK | |
| Sintilimab, | DIQMTQSPSSVSASVGDRVTITCRASQGISSWLAWYQQKPGKAP | 35 |
| Tyvyt, IBI-308 | KLLISAASSLQSGVPSRFSGSGSGTDFTLTISSLQPEDFATYYCQQ | |
| VL | ANHLPFTFGGGTKVEIKRTVAAPSVFIFPPSDEQLKSGTASVVCLL | |
| | NNFYPREAKVQWKVDNALQSGNSQESVTEQDSKDSTYSLSSTLT | |
| | LSKADYEKHKVYACEVTHQGLSSPVTKSFNRGEC | |
| Toripalimab, | QGQLVQSGAEVKKPGASVKVSCKASGYTFTDYEMHWVRQAPIH | 36 |
| TeRuiPuLi, | GLEWIGVIESETGGTAYNQKFKGRVTITADKSTSTAYMELSSLRS | |
| Terepril, Tuoyi, | EDTAVYYCAREGITTVATTYYWYFDVWGQGTTVTVSSASTKGP | |
| JS-001, TAB- | SVFPLAPCSRSTSESTAALGCLVKDYFPEPVTVSWNSGALTSGVH | |
| 001 | TFPAVLQSSGLYSLSSVVTVPSSSLGTKTYTCNVDHKPSNTKVDK | |
| VH | RVESKYGPPCPPCPAPEFLGGPSVFLFPPKPKDTLMISRTPEVTCV | |
| | VVDVSQEDPEVQFNWYVDGVEVHNAKTKPREEQFNSTYRVVSV | |
| | LTVLHQDWLNGKEYKCKVSNKGLPSSIEKTISKAKGQPREPQVY | |
| | TLPPSQEEMTKNQVSLTCLVKGFYPSDIAVEWESNGQPENNYKT | |
| | TPPVLDSDGSFFLYSRLTVDKSRWQEGNVFSCSVMHEALHNHYT | |
| | QKSLSLSLGK | |
| Toripalimab, | DVVMTQSPLSLPVTLGQPASISCRSSQSIVHSNGNTYLEWYLQKP | 37 |
| TeRuiPuLi, | GQSPQLLIYKVSNRFSGVPDRFSGSGSGTDFTLKISRVEAEDVGV | |
| Terepril, Tuoyi, | YYCFQGSHVPLTFGQGTKLEIKRTVAAPSVFIFPPSDEQLKSGTAS | |
| JS-001, TAB- | VVCLLNNFYPREAKVQWKVDNALQSGNSQESVTEQDSKDSTYS | |
| 001 | LSSTLTLSKADYEKHKVYACEVTHQGLSSPVTKSFNRGEC | |
| VL | | |
| Camrelizumab, | EVQLVESGGGLVQPGGSLRLSCAASGFTFSSYMMSWVRQAPGK | 38 |
| HR-301210, | GLEWVATISGGGANTYYPDSVKGRFTISRDNAKNSLYLQMNSLR | |
| INCSHR-01210, | AEDTAVYYCARQLYYFDYWGQGTTVTVSSASTKGPSVFPLAPCS | |
| SHR-1210 | RSTSESTAALGCLVKDYFPEPVTVSWNSGALTSGVHTFPAVLQSS | |
| VH | GLYSLSSVVTVPSSSLGTKTYTCNVDHKPSNTKVDKRVESKYGP | |
| | PCPPCPAPEFLGGPSVFLFPPKPKDTLMISRTPEVTCVVVDVSQED | |
| | PEVQFNWYVDGVEVHNAKTKPREEQFNSTYRVVSVLTVLHQD | |
| | WLNGKEYKCKVSNKGLPSSIEKTISKAKGQPREPQVYTLPPSQEE | |
| | MTKNQVSLTCLVKGFYPSDIAVEWESNGQPENNYKTTPPVLDSD | |
| | GSFFLYSRLTVDKSRWQEGNVFSCSVMHEALHNHYTQKSLSLSL | |
| | GK | |
| Camrelizumab, | DIQMTQSPSSLSASVGDRVTITCLASQTIGTWLTWYQQKPGKAP | 39 |
| HR-301210, | KLLIYTATSLADGVPSRFSGSGSGTDFTLTISSLQPEDFATYYCQQ | |
| INCSHR-01210, | VYSIPWTFGGGTKVEIKRTVAAPSVFIFPPSDEQLKSGTASVVCLL | |
| SHR-1210 | NNFYPREAKVQWKVDNALQSGNSQESVTEQDSKDSTYSLSSTLT | |
| VL | LSKADYEKHKVYACEVTHQGLSSPVTKSFNRGEC | |
| Cemiplimab, | EVQLLESGGVLVQPGGSLRLSCAASGFTFSNFGMTWVRQAPGK | 40 |
| Cemiplimab- | GLEWVSGISGGGRDTYFADSVKGRFTISRDNSKNTLYLQMNSLK | |
| rwlc, | GEDTAVYYCVKWGNIYFDYWGQGTLVTVSSASTKGPSVFPLAP | |
| LIBTAYO ®, | CSRSTSESTAALGCLVKDYFPEPVTVSWNSGALTSGVHTFPAVL | |
| 6QVL057INT, | QSSGLYSLSSVVTVPSSSLGTKTYTCNVDHKPSNTKVDKRVESK | |
| H4H7798N, | YGPPCPPCPAPEFLGGPSVFLFPPKPKDTLMISRTPEVTCVVVDVS | |
| REGN-2810, | QEDPEVQFNWYVDGVEVHNAKTKPREEQFNSTYRVVSVLTVLH | |
| SAR-439684 | QDWLNGKEYKCKVSNKGLPSSIEKTISKAKGQPREPQVYTLPPSQ | |
| VH | EEMTKNQVSLTCLVKGFYPSDIAVEWESNGQPENNYKTTPPVLD | |
| | SDGSFFLYSRLTVDKSRWQEGNVFSCSVMHEALHNHYTQKSLSL | |
| | SLGK | |
| Cemiplimab, | DIQMTQSPSSLSASVGDSITITCRASLSINTFLNWYQQKPGKAPNL | 41 |
| Cemiplimab- | LIYAASSLHGGVPSRFSGSGSGTDFTLTIRTLQPEDFATYYCQQSS | |
| rwlc, | NTPFTFGPGTVVDFRRTVAAPSVFIFPPSDEQLKSGTASVVCLLN | |
| LIBTAYO ®, | NFYPREAKVQWKVDNALQSGNSQESVTEQDSKDSTYSLSSTLTL | |
| 6QVL057INT, | SKADYEKHKVYACEVTHQGLSSPVTKSFNRGEC | |
| H4H7798N, | (Disulfide bridge) | |
| REGN-2810, | H22-H96, H131-L214, H144-H200, H223-H′223, | |
| SAR-439684 | H226-H′226, H258-H318, H364-H422, H′22-H′96, | |
| VL | H′131-L′214, H′114-H′200, H′258-H′318, H′364- | |
| | H′422, L23-L88, L134-L194, L′23-L′88, L′134- | |
| | L′194) | |
| Lambrolizumab, | QVQLVQSGVEVKKPGASVKVSCKASGYTFTNYYMYWVRQAPG | 46 |
| Pembrolizumab, | QGLEWMGGINPSNGGTNFNEKFKNRVTLTTDSSTTTAYMELKSL | |
| KEYTRUDA ®, | QFDDTAVYYCARRDYRFDMGFDYWGQGTTVTVSSASTKGPSVF | |
| MK-3475, SCH- | PLAPCSRSTSESTAALGCLVKDYFPEPVTVSWNSGALTSGVHTFP | |
| 900475, | AVLQSSGLYSLSSVVTVPSSSLGTKTYTCNVDHKPSNTKVDKRV | |
| h409A11 | ESKYGPPCPPCPAPEFLGGPSVFLFPPKPKDTLMISRTPEVTCVVV | |
| VH | DVSQEDPEVQFNWYVDGVEVHNAKTKPREEQFNSTYRVVSVLT | |
| | VLHQDWLNGKEYKCKVSNKGLPSSIEKTISKAKGQPREPQVYTL | |
| | PPSQEEMTKNQVSLTCLVKGFYPSDIAVEWESNGQPENNYKTTP | |
| | PVLDSDGSFFLYSRLTVDKSRWQEGNVFSCSVMHEALHNHYTQ | |
| | KSLSLSLGK | |
| Lambrolizumab, | EIVLTQSPATLSLSPGERATLSCRASKGVSTSGYSYLHWYQQKPG | 47 |
| Pembrolizumab, | QAPRLLIYLASYLESGVPARFSGSGSGTDFTLTISSLEPEDFAVYY | |
| KEYTRUDA ®, | CQHSRDLPLTFGGGTKVEIKRTVAAPSVFIFPPSDEQLKSGTASVV | |
| MK-3475, SCH- | CLLNNFYPREAKVQWKVDNALQSGNSQESVTEQDSKDSTYSLSS | |
| 900475, | TLTLSKADYEKHKVYACEVTHQGLSSPVTKSFNRGEC | |
| h409A11 | | |
| VL | | |
| Lambrolizumab, | QVQLVQSGVEVKKPGASVKVSCKASGYTFTNYYMYWVRQAPG | 48 |
| Pembrolizumab, | QGLEWMGGFPSNGGTNFNEKFKNRVTLTTDSSTTTAYMELKSL | |
| KEYTRUDA ®, | QFDDTAVYYCARRDYRFDMGFDYWGQGTTVTVSS | |
| MK-3475, SCH- | (Disulfide bridge) | |
| 900475, | H22-H96, H134-L218, H147-H203, H226-H′226, | |
| h409A11 | H229-H′229, H261-H321, H367-H425, H′22-H′96, | |
| VH | H′134-L′218, H′147-H′203, H′261-H′321, H′367- | |
| | H′425, L23-L92, L138-L198, L′23-L′92, L′138- | |
| | L′198) | |
| Lambrolizumab, | EIVLTQSPATLSLSPGERATLSCRASKGVSTSGYSYLHWYQQKPG | 49 |
| Pembrolizumab, | QAPRLLIYLASYLESGVPARFSGSGSGTDFTLTISSLEPEDFAVYY | |
| KEYTRUDA ®, | CQHSRDLPLTFGGGTKVEIK | |
| MK-3475, SCH- | | |
| 900475, | | |
| h409A11 | | |
| VL | | |
| Nivolumab, | QVQLVESGGGVVQPGRSLRLDCKASGITFSNSGMHWVRQAPGK | 50 |
| Nivolumab | GLEWVAVIWYDGSKRYYADSVKGRFTISRDNSKNTLFLQMNSL | |
| BMS, | RAEDTAVYYCATNDDYWGQGTLVTVSS | |
| OPDIVO ®, | | |
| BMS-936558, | | |
| MDX-1106, | | |
| ONO-4538 | | |
| VH | | |
| Nivolumab, | EIVLTQSPATLSLSPGERATLSCRASQSVSSYLAWYQQKPGQAPR | 51 |
| Nivolumab | LLIYDASNRATGIPARFSGSGSGTDFTLTISSLEPEDFAVYYCQQS | |
| BMS, | SNWPRTFGQGTKVEIK | |
| OPDIVO ®, | (Disulfide bridge) | |
| BMS-936558, | H22-H96, H127-L214, H140-H196, H219-H′219, | |
| MDX-1106, | H222-H′222, H254-H314, H360-H418, H′22-H′96, | |
| ONO-4538 | H′127-L′214, H′140-H′196, H′254-H′314, H′360- | |
| VL | H′418, L-23-L88, L134-L194, L′23-L′88, | |
| | L′134-L′194) | |
| Prolgolimab, | QVQLVQSGGGLVQPGGSLRLSCAASGFTFSSYWMYWVRQVPGK | 52 |
| Forteca, BCD- | GLEWVSAIDTGGGRTYYADSVKGRFAISRVNAKNTMYLQMNSL | |
| 100 | RAEDTAVYYCARDEGGGTGWGVLKDWPYGLDAWGQGTLVTV | |
| VH | SSASTKGPSVFPLAPSSKSTSGGTAALGCLVKDYFPEPVTVSWNS | |
| | GALTSGVHTFPAVLQSSGLYSLSSVVTVPSSSLGTQTYICNVNHK | |
| | PSNTKVDKRVEPKSCDKTHTCPPCPAPEAAGGPSVFLFPPKPKDT | |
| | LMISRTPEVTCVVVDVSHEDPEVKFNWYVDGVEVHNAKTKPRE | |
| | EQYNSTYRVVSVLTVLHQDWLNGKEYKCKVSNKALPAPIEKTIS | |
| | KAKGQPREPQVYTLPPSREEMTKNQVSLTCLVKGFYPSDIAVEW | |
| | ESNGQPENNYKTTPPVLDSDGSFFLYSKLTVDKSRWQQGNVFSC | |
| | SVMHEALHNHYTQKSLSLSPGK | |
| Prolgolimab, | QPVLTQPLSVSVALGQTARITCGGNNIGSKNVHWYQQKPGQAPV | 53 |
| Forteca, BCD- | LVIYRDSNRPSGIPERFSGSNSGNTATLTISRAQAGDEADYYCQV | |
| 100 | WDSSTAVFGTGTKLTVLQRTVAAPSVFIFPPSDEQLKSGTASVVC | |
| VL | LLNNFYPREAKVQWKVDNALQSGNSQESVTEQDSKDSTYSLSST | |
| | LTLSKADYEKHKVYACEVTHQGLSSPVTKSFNRGEC | |
| Balstilimab, | QVQLVESGGGVVQPGRSLRLSCAASGFTFSSYGMHWVRQAPGK | 54 |
| 1Q2QT5M7EO, | GLEWVAVIWYDGSNKYYADSVKGRFTISRDNSKNTLYLQMNSL | |
| AGEN-2034, | RAEDTAVYYCASNGDHWGQGTLVTVSSASTKGPSVFPLAPCSRS | |
| AGEN-2034w | TSESTAALGCLVKDYFPEPVTVSWNSGALTSGVHTFPAVLQSSG | |
| VH | LYSLSSVVTVPSSSLGTKTYTCNVDHKPSNTKVDKRVESKYGPP | |
| | CPPCPAPEFLGGPSVFLFPPKPKDTLMISRTPEVTCVVVDVSQEDP | |
| | EVQFNWYVDGVEVHNAKTKPREEQFNSTYRVVSVLTVLHQDW | |
| | LNGKEYKCKVSNKGLPSSIEKTISKAKGQPREPQVYTLPPSQEEM | |
| | TKNQVSLTCLVKGFYPSDIAVEWESNGQPENNYKTTPPVLDSDG | |
| | SFFLYSRLTVDKSRWQEGNVFSCSVMHEALHNHYTQKSLSLSLG | |
| Balstilimab, | EIVMTQSPATLSVSPGERATLSCRASQSVSSNLAWYQQKPGQAP | 55 |
| 1Q2QT5M7EO, | RLLIYGASTRATGIPARFSGSGSGTEFTLTISSLQSEDFAVYYCQQ | |
| AGEN-2034, | YNNWPRTFGQGTKVEIKRTVAAPSVFIFPPSDEQLKSGTASVVCL | |
| AGEN-2034w | LNNFYPREAKVQWKVDNALQSGNSQESVTEQDSKDSTYSLSSTL | |
| VL | TLSKADYEKHKVYACEVTHQGLSSPVTKSFNRGEC | |
| Dostarlimab, | EVQLLESGGGLVQPGGSLRLSCAASGFTFSSYDMSWVRQAPGKG | 56 |
| ANB-011, | LEWVSTISGGGSYTYYQDSVKGRFTISRDNSKNTLYLQMNSLRA | |
| GSK- | EDTAVYYCASPYYAMDYWGQGTTVTVSSASTKGPSVFPLAPCS | |
| 4057190A, | RSTSESTAALGCLVKDYFPEPVTVSWNSGALTSGVHTFPAVLQSS | |
| P0GVQ9A4S5, | GLYSLSSVVTVPSSSLGTKTYTCNVDHKPSNTKVDKRVESKYGP | |
| TSR-042, WBP- | PCPPCPAPEFLGGPSVFLFPPKPKDTLMISRTPEVTCVVVDVSQED | |
| 285 | PEVQFNWYVDGVEVHNAKTKPREEQFNSTYRVVSVLTVLHQD | |
| VH | WLNGKEYKCKVSNKGLPSSIEKTISKAKGQPREPQVYTLPPSQEE | |
| | MTKNQVSLTCLVKGFYPSDIAVEWESNGQPENNYKTTPPVLDSD | |
| | GSFFLYSRLTVDKSRWQEGNVFSCSVMHEALHNHYTQKSLSLSL | |
| | GK | |
| Dostarlimab, | DIQLTQSPSFLSAYVGDRVTITCKASQDVGTAVAWYQQKPGKAP | 57 |
| ANB-011, | KLLIYWASTLHTGVPSRFSGSGSGTEFTLTISSLQPEDFATYYCQH | |
| GSK- | YSSYPWTFGQGTKLEIKRTVAAPSVFIFPPSDEQLKSGTASVVCLL | |
| 4057190A, | NNFYPREAKVQWKVDNALQSGNSQESVTEQDSKDSTYSLSSTLT | |
| P0GVQ9A4S5, | LSKADYEKHKVYACEVTHQGLSSPVTKSFNRGEC | |
| TSR-042, WBP- | | |
| 285 | | |
| VL | | |
| Serplulimab, | OVQLVESGGGLVKPGGSLRLSCAASGFTFSNYGMSWIROAPGKG | 58 |
| HLX-10 | LEWVSTISGGGSNIYYADSVKGRFTISRDNAKNSLYLQMNSLRA | |
| VH | EDTAVYYCVSYYYGIDFWGQGTSVTVSSASTKGPSVFPLAPCSR | |
| | STSESTAALGCLVKDYFPEPVTVSWNSGALTSGVHTFPAVLQSSG | |
| | LYSLSSVVTVPSSSLGTKTYTCNVDHKPSNTKVDKRVESKYGPP | |
| | CPPCPAPEFLGGPSVFLFPPKPKDTLMISRTPEVTCVVVDVSQEDP | |
| | EVQFNWYVDGVEVHNAKTKPREEQFNSTYRVVSVLTVLHQDW | |
| | LNGKEYKCKVSKGLPSSIEKTISKAKGQPREPQVYTLPPSQEEMT | |
| | KNQVSLTCLVKGFYPSDIAVEWESNGQPENNYKTTPPVLDSDGS | |
| | FFLYSRLTVDKSRWQEGNVFSCSVMHEALHNHYTQKSLSLSLGK | |
| Serplulimab, | DIQMTQSPSSLSASVGDRVTITCKASQDVTTAVAWYQQKPGKAP | 59 |
| HLX-10 | KLLIYWASTRHTGVPSRFSGSGSGTDFTLTISSLQPEDFATYYCOO | |
| VL | HYTIPWTFGGGTKLEIKRTVAAPSVFIFPPSDEQLKSGTASVVCLL | |
| | NNFYPREAKVQWKVDNALQSGNSQESVTEQDSKDSTYSLSSTLT | |
| | LSKADYEKHKVYACEVTHQGLSSPVTKSFNRGEC | |
| Retifanlimab, | QVQLVQSGAEVKKPGASVKVSCKASGYSFTSYWMNWVRQAPG | 60 |
| 2Y3T5IF01Z, | QGLEWIGVIHPSDSETWLDQKFKDRVTITVDKSTSTAYMELSSLR | |
| INCMGA- | SEDTAVYYCAREHYGTSPFAYWGQGTLVTVSSASTKGPSVFPLA | |
| 00012, | PCSRSTSESTAALGCLVKDYFPEPVTVSWNSGALTSGVHTFPAVL | |
| INCMGA-0012, | QSSGLYSLSSVVTVPSSSLGTKTYTCNVDHKPSNTKVDKRVESK | |
| MGA-012 | YGPPCPPCPAPEFLGGPSVFLFPPKPKDTLMISRTPEVTCVVVDVS | |
| VH | QEDPEVQFNWYVDGVEVHNAKTKPREEQFNSTYRVVSVLTVLH | |
| | QDWLNGKEYKCKVSNKGLPSSIEKTISKAKGQPREPQVYTLPPSQ | |
| | EEMTKNQVSLTCLVKGFYPSDIAVEWESNGQPENNYKTTPPVLD | |
| | SDGSFFLYSRLTVDKSRWQEGNVFSCSVMHEALHNHYTQKSLSL | |
| | SLG | |
| Retifanlimab, | EIVLTQSPATLSLSPGERATLSCRASESVDNYGMSFMNWFQQKP | 61 |
| 2Y3T5IF01Z, | GQPPKLLIHAASNQGSGVPSRFSGSGSGTDFTLTISSLEPEDFAVY | |
| INCMGA- | FCQQSKEVPYTFGGGTKVEIKRTVAAPSVFIFPPSDEQLKSGTASV | |
| 00012, | VCLLNNFYPREAKVQWKVDNALQSGNSQESVTEQDSKDSTYSL | |
| INCMGA-0012, | SSTLTLSKADYEKHKVYACEVTHQGLSSPVTKSFNRGEC | |
| MGA-012 | | |
| VL | | |
| Sasanlimab, | QVQLVQSGAEVKKPGASVKVSCKASGYTFTSYWINWVRQAPGQ | 62 |
| LZZ0IC2EWP, | GLEWMGNIYPGSSLTNYNEKFKNRVTMTRDTSTSTVYMELSSLR | |
| PF-06801591, | SEDTAVYYCARLSTGTFAYWGQGTLVTVSSASTKGPSVFPLAPC | |
| RN-888 | SRSTSESTAALGCLVKDYFPEPVTVSWNSGALTSGVHTFPAVLQS | |
| VH | SGLYSLSSVVTVPSSSLGTKTYTCNVDHKPSNTKVDKRVESKYG | |
| | PPCPPCPAPEFLGGPSVFLFPPKPKDTLMISRTPEVTCVVVDVSQE | |
| | DPEVQFNWYVDGVEVHNAKTKPREEQFNSTYRVVSVLTVLHQD | |
| | WLNGKEYKCKVSNKGLPSSIEKTISKAKGQPREPQVYTLPPSQEE | |
| | MTKNQVSLTCLVKGFYPSDIAVEWESNGQPENNYKTTPPVLDSD | |
| | GSFFLYSRLTVDKSRWQEGNVFSCSVMHEALHNHYTQKSLSLSL | |
| | GK | |
| Sasanlimab, | DIVMTQSPDSLAVSLGERATINCKSSQSLWDSGNQKNFLTWYQQ | 63 |
| LZZ0IC2EWP, | KPGQPPKLLIYWTSYRESGVPDRFSGSGSGTDFTLTISSLQAEDVA | |
| PF-06801591, | VYYCQNDYFYPHTFGGGTKVEIKRTVAAPSVFIFPPSDEQLKSGT | |
| RN-888 | ASVVCLLNNFYPREAKVQWKVDNALQSGNSQESVTEQDSKDST | |
| VL | YSLSSTLTLSKADYEKHKVYACEVTHQGLSSPVTKSFNRGEC | |
| Spartalizumab, | EVQLVQSGAEVKKPGESLRISCKGSGYTFTTYWMHWVRQATGQ | 64 |
| NVP-LZV-184, | GLEWMGNIYPGTGGSNFDEKFKNRVTITADKSTSTAYMELSSLR | |
| PDR-001, | SEDTAVYYCTRWTTGTGAYWGQGTTVTVSSASTKGPSVFPLAP | |
| QOG25L6Z8Z | CSRSTSESTAALGCLVKDYFPEPVTVSWNSGALTSGVHTFPAVL | |
| VH | QSSGLYSLSSVVTVPSSSLGTKTYTCNVDHKPSNTKVDKRVESK | |
| | YGPPCPPCPAPEFLGGPSVFLFPPKPKDTLMISRTPEVTCVVVDVS | |
| | QEDPEVQFNWYVDGVEVHNAKTKPREEQFNSTYRVVSVLTVLH | |
| | QDWLNGKEYKCKVSNKGLPSSIEKTISKAKGQPREPQVYTLPPSQ | |
| | EEMTKNQVSLTCLVKGFYPSDIAVEWESNGQPENNYKTTPPVLD | |
| | SDGSFFLYSRLTVDKSRWQEGNVFSCSVMHEALHNHYTQKSLSL | |
| | SLG | |
| Spartalizumab, | EIVLTQSPATLSLSPGERATLSCKSSQSLLDSGNQKNFLTWYQQK | 65 |
| NVP-LZV-184, | PGQAPRLLIYWASTRESGVPSRFSGSGSGTDFTFTISSLEAEDAAT | |
| PDR-001, | YYCQNDYSYPYTFGQGTKVEIKRTVAAPSVFIFPPSDEQLKSGTA | |
| QOG25L6Z8Z | SVVCLLNNFYPREAKVQWKVDNALQSGNSQESVTEQDSKDSTY | |
| VL | SLSSTLTLSKADYEKHKVYACEVTHQGLSSPVTKSFNRGEC | |
| Cetrelimab, | QVQLVQSGAEVKKPGSSVKVSCKASGGTFSSYAISWVRQAPGQ | 66 |
| JNJ-3283, JNJ- | GLEWMGGIIPIFDTANYAQKFQGRVTITADESTSTAYMELSSLRS | |
| 63723283, | EDTAVYYCARPGLAAAYDTGSLDYWGQGTLVTVSSASTKGPSV | |
| LYK98WP91F | FPLAPCSRSTSESTAALGCLVKDYFPEPVTVSWNSGALTSGVHTF | |
| VH | PAVLQSSGLYSLSSVVTVPSSSLGTKTYTCNVDHKPSNTKVDKR | |
| | VESKYGPPCPPCPAPEFLGGPSVFLFPPKPKDTLMISRTPEVTCVV | |
| | VDVSQEDPEVQFNWYVDGVEVHNAKTKPREEQFNSTYRVVSVL | |
| | TVLHQDWLNGKEYKCKVSNKGLPSSIEKTISKAKGQPREPQVYT | |
| | LPPSQEEMTKNQVSLTCLVKGFYPSDIAVEWESNGQPENNYKTT | |
| | PPVLDSDGSFFLYSRLTVDKSRWQEGNVFSCSVMHEALHNHYTQ | |
| | KSLSLSLGK | |
| Cetrelimab, | EIVLTQSPATLSLSPGERATLSCRASQSVRSYLAWYQQKPGQAPR | 67 |
| JNJ-3283, JNJ- | LLIYDASNRATGIPARFSGSGSGTDFTLTISSLEPEDFAVYYCQQR | |
| 63723283, | NYWPLTFGQGTKVEIKRTVAAPSVFIFPPSDEQLKSGTASVVCLL | |
| LYK98WP91F | NNFYPREAKVQWKVDNALQSGNSQESVTEQDSKDSTYSLSSTLT | |
| VL | LSKADYEKHKVYACEVTHQGLSSPVTKSFNRGEC | |
| Tebotelimab, | DIQMTQSPSSLSASVGDRVTITCRASQDVSSVVAWYQQKPGKAP | 68 |
| MGD-013 | KLLIYSASYRYTGVPSRFSGSGSGTDFTLTISSLQPEDFATYYCQQ | |
| VH | HYSTPWTFGGGTKLEIKGGGSGGGGQVQLVQSGAEVKKPGASV | |
| | KVSCKASGYSFTSYWMNWVRQAPGQGLEWIGVIHPSDSETWLD | |
| | QKFKDRVTITVDKSTSTAYMELSSLRSEDTAVYYCAREHYGTSP | |
| | FAYWGQGTLVTVSSGGCGGGEVAACEKEVA | |
| | ALEKEVAALEKEVAALEKESKYGPPCPPCPAPEFLGGPSVFLFPP | |
| | KPKDTLYITREPEVTCVVVDVSQEDPEVQFNWYVDGVEVHNAK | |
| | TKPREEQFNSTYRVVSVLTVLHQDWLNGKEYKCKVSNKGLPSSI | |
| | EKTISKAKGQPREPQVYTLPPSQEEMTKNQVSLTCLVKGFYPSDI | |
| | AVEWESNGQPENNYKTTPPVLDSDGSFFLYSRLTVDKSRWQEG | |
| | NVFSCSVMHEALHNHYTQKSLSLSLG | |
| Tebotelimab, | EIVLTQSPATLSLSPGERATLSCRASESVDNYGMSFMNWFQQKP | 69 |
| MGD-013 | GQPPKLLIHAASNQGSGVPSRFSGSGSGTDFTLTISSLEPEDFAVY | |
| VL | FCQQSKEVPYTFGGGTKVEIKGGGSGGGGQVQLVQSGAEVKKP | |
| | GASVKVSCKASGYTFTDYNMDWVRQAPGQGLEWMGDINPDNG | |
| | VTIYNQKFEGRVTMTTDTSTSTAYMELRSLRSDDTAVYYCAREA | |
| | DYFYFDYWGQGTTLTVSSGGCGGGKVAACKEKVAALKEKVAA | |
| | LKEKVAALKE | |
| Pidilizumab, | QVQLVQSGSELKKPGASVKISCKASGYTFTNYGMNWVRQAPGQ | 70 |
| CT-011, MDV- | GLQWMGWINTDSGESTYAEEFKGRFVFSLDTSVNTAYLQITSLT | |
| 9300 | AEDTGMYFCVRVGYDALDYWGQGTLVTVSSASTKGPSVFPLAP | |
| VH | SSKSTSGGTAALGCLVKDYFPEPVTVSWNSGALTSGVHTFPAVL | |
| | QSSGLYSLSSVVTVPSSSLGTQTYICNVNHKPSNTKVDKRVEPKS | |
| | CDKTHTCPPCPAPELLGGPSVFLFPPKPKDTLMISRTPEVTCVVV | |
| | DVSHEDPEVKFNWYVDGVEVHNAKTKPREEQYNSTYRVVSVLT | |
| | VLHQDWLNGKEYKCKVSNKALPAPIEKTISKAKGQPREPQVYTL | |
| | PPSREEMTKNQVSLTCLVKGFYPSDIAVEWESNGQPENNYKTTP | |
| | PVLDSDGSFFLYSKLTVDKSRWQQGNVFSCSVMHEALHNHYTQ | |
| | KSLSLSPGK | |
| Pidilizumab, | EIVLTQSPSSLSASVGDRVTITCSARSSVSYMHWFQQKPGKAPKL | 71 |
| CT-011, MDV- | WIYRTSNLASGVPSRFSGSGSGTSYCLTINSLQPEDFATYYCQQR | |
| 9300 | SSFPLTFGGGTKLEIKRTVAAPSVFIFPPSDEQLKSGTASVVCLLN | |
| VL | NFYPREAKVQWKVDNALQSGNSQESVTEQDSKDSTYSLSSTLTL | |
| | SKADYEKHKVYACEVTHQGLSSPVTKSFNRGEC | |
| SG-001 VH | QVQLVESGGGVVQPGRSLRLTCKASGLTFSSSGMHWVRQAPGK | 72 |
| | GLEWVAVIWYDGSKRYYADSVKGRFTISRDNSKNTLFLQMNSL | |
| | RAEDTAVYYCATNNDYWGQGTLVTVSS | |
| SG-001 VL | EIVLTQSPATLSLSPGERATLSCRASQSVSSYLAWYQQKPGQAPR | 73 |
| | LLIYTASNRATGIPARFSGSGSGTDFTLTISSLEPEDFAVYYCQQY | |
| | SNWPRTFGQGTKVEIK | |
| LZM-009 VH | EVQLQQSGPVLVKPGASVKMSCKASGYTFTSYYMYWVKQSHG | 74 |
| | KSLEWIGGVNPSNGGTNFNEKFKSKATLTVDKSSSTAYMELNSL | |
| | TSEDSAVYYCARRDYRYDMGFDYWGQGTTLTVSS | |
| LZM-009 VL | QIVLTQSPAIMSASPGEKVTMTCRASKGVSTSGYSYLHWYQQKP | 75 |
| | GSSPRLLIYLASYLESGVPVRFSGSGSGTSYSLTISRMEAEDAATY | |
| | YCQHSRELPLTFGTGTRLEIK | |
| LZM-009 VH | QVQLVQSGAEVKKPGASVKVSCKASGYTFTSYYMYWVRQAPG | 76 |
| | QGLEWMGGVNPSNGGTNFNEKFKSRVTITADKSTSTAYMELSSL | |
| | RSEDTAVYYCARRDYRYDMGFDYWGQGTTVTVSS | |
| LZM-009 VL | EIVLTQSPATLSLSPGERATISCRASKGVSTSGYSYLHWYQQKPG | 77 |
| | QAPRLLIYLASYLESGVPARFSGSGSGTDFTLTISSLEPEDFATYY | |
| | CQHSRELPLTFGTGTKVEIK | |
| Budigalimab, | EIQLVQSGAEVKKPGSSVKVSCKASGYTFTHYGMNWVRQAPGQ | 78 |
| 6VDO4TY3OO, | GLEWVGWVNTYTGEPTYADDFKGRLTFTLDTSTSTAYMELSSL | |
| ABBV-181, PR- | RSEDTAVYYCTREGEGLGFGDWGQGTTVTVSSASTKGPSVFPLA | |
| 1648817 | PSSKSTSGGTAALGCLVKDYFPEPVTVSWNSGALTSGVHTFPAV | |
| VH | LQSSGLYSLSSVVTVPSSSLGTQTYICNVNHKPSNTKVDKKVEPK | |
| | SCDKTHTCPPCPAPEAAGGPSVFLFPPKPKDTLMISRTPEVTCVV | |
| | VDVSHEDPEVKFNWYVDGVEVHNAKTKPREEQYNSTYRVVSVL | |
| | TVLHQDWLNGKEYKCKVSNKALPAPIEKTISKAKGQPREPQVYT | |
| | LPPSREEMTKNQVSLTCLVKGFYPSDIAVEWESNGQPENNYKTT | |
| | PPVLDSDGSFFLYSKLTVDKSRWQQGNVFSCSVMHEALHNHYT | |
| | QKSLSLSPGK | |
| Budigalimab, | DVVMTQSPLSLPVTPGEPASISCRSSQSIVHSHGDTYLEWYLQKP | 79 |
| 6VDO4TY3OO, | GQSPQLLIYKVSNRFSGVPDRFSGSGSGTDFTLKISRVEAEDVGV | |
| ABBV-181, PR- | YYCFQGSHIPVTFGQGTKLEIKRTVAAPSVFIFPPSDEQLKSGTAS | |
| 1648817 | VVCLLNNFYPREAKVQWKVDNALQSGNSQESVTEQDSKDSTYS | |
| VL | LSSTLTLSKADYEKHKVYACEVTHQGLSSPVTKSFNRGEC | |
| Lambrolizumab, | NYYMY | 80 |
| Pembrolizumab, | | |
| KEYTRUDA ®, | | |
| MK-3475, SCH- | | |
| 900475, | | |
| h409A11 | | |
| VH CDR1 | | |
| Lambrolizumab, | GINPSNGGTNFNEKFKN | 81 |
| Pembrolizumab, | | |
| KEYTRUDA ®, | | |
| MK-3475, SCH- | | |
| 900475, | | |
| h409A11 | | |
| VH CDR2 | | |
| Lambrolizumab, | RDYRFDMGFDY | 82 |
| Pembrolizumab, | | |
| KEYTRUDA ®, | | |
| MK-3475, SCH- | | |
| 900475, | | |
| h409A11 | | |
| VH CDR3 | | |
| Lambrolizumab, | RASKGVSTSGYSYLH | 83 |
| Pembrolizumab, | | |
| KEYTRUDA ®, | | |
| MK-3475, SCH- | | |
| 900475, | | |
| h409A11 | | |
| VL CDR1 | | |
| Lambrolizumab, | LASYLES | 84 |
| Pembrolizumab, | | |
| KEYTRUDA ® | | |
| MK-3475, SCH- | | |
| 900475, | | |
| h409A11 | | |
| VL CDR2 | | |
| Lambrolizumab, | QHSRDLPLT | 85 |
| Pembrolizumab, | | |
| KEYTRUDA ®, | | |
| MK-3475, SCH- | | |
| 900475, | | |
| h409A11 | | |
| VL CDR3 | | |
| Nivolumab, | NSGMH | 86 |
| Nivolumab | | |
| BMS, | | |
| OPDIVO ®, | | |
| BMS-936558, | | |
| MDX-1106, | | |
| ONO-4538 | | |
| VH CDR1 | | |
| Nivolumab, | VIWYDGSKRYYADSVKG | 87 |
| Nivolumab | | |
| BMS, | | |
| OPDIVO ®, | | |
| BMS-936558, | | |
| MDX-1106, | | |
| ONO-4538 | | |
| VH CDR2 | | |
| Nivolumab, | NDDY | 88 |
| Nivolumab | | |
| BMS, | | |
| OPDIVO ®, | | |
| BMS-936558, | | |
| MDX-1106, | | |
| ONO-4538 | | |
| VH CDR3 | | |
| Nivolumab, | RASQSVSSYLA | 89 |
| Nivolumab | | |
| BMS, | | |
| OPDIVO ®, | | |
| BMS-936558, | | |
| MDX-1106, | | |
| ONO-4538 | | |
| VL CDR1 | | |
| Nivolumab, | DASNRAT | 90 |
| Nivolumab | | |
| BMS, | | |
| OPDIVO ®, | | |
| BMS-936558, | | |
| MDX-1106, | | |
| ONO-4538 | | |
| VL CDR2 | | |
| Nivolumab, | QQSSNWPRT | 91 |
| Nivolumab | | |
| BMS, | | |
| OPDIVO ®, | | |
| BMS-936558, | | |
| MDX-1106, | | |
| ONO-4538 | | |
| VL CDR3 | | |
| Serplulimab, | FTFSNYGMS | 92 |
| HLX-10 | | |
| VH CDR1 | | |
| Serplulimab, | TISGGGSNIY | 93 |
| HLX-10 | | |
| VH CDR2 | | |
| Serplulimab, | VSYYYGIDF | 94 |
| HLX-10 | | |
| VH CDR3 | | |
| Serplulimab, | KASQDVTTAVA | 95 |
| HLX-10 | | |
| VL CDR1 | | |
| Serplulimab, | WASTRHT | 96 |
| HLX-10 | | |
| VL CDR2 | | |
| Serplulimab, | QQHYTIPWT | 97 |
| HLX-10 | | |
| VL CDR3 | | |
| SG-001 | GLTFSSSG | 98 |
| VH CDR1 | | |
| SG-001 | IWYDGSKR | 99 |
| VH CDR2 | | |
| SG-001 | ATNNDY | 100 |
| VH CDR3 | | |
| SG-001 | RASQSVSSYLA | 101 |
| VL CDR1 | | |
| SG-001 | TASNRAT | 102 |
| VL CDR2 | | |
| SG-001 | QQYSNWPRT | 103 |
| VL CDR3 | | |
| PD-1-Fc- | MQIPQAPWPWWAVLQLGWRPGWFLDSPDRPWNPPTFSPALLVV | 104 |
| OX40L (Code), | TEGDNATFTCSFSNTSESFVLNWYRMSPSNQTDKLAAFPEDRSQP | |
| SL-279252 | GQDCRFRVTQLPNGRDFHMSVVRARRNDSGTYLCGAISLAPKA | |
| (Code), TAK- | QIKESLRAELRVTERRAEVPTAHPSPSPRPAGQFQSKYGPPCPSCP | |
| 252 (Code) | APEFLGGPSVFLFPPKPKDTLMISRTPEVTCWVDVSQEDPEVQFN | |
| | WYVDGVEVHNAKTKPREEQFNSTYRVVSVLTVLHQDWLSGKE | |
| | YKCKVSSKGLPSSIEKTISNATGQPREPQVYTLPPSQEEMTKNQV | |
| | SLTCLVKGFYPSDIAVEWESNGQPENNYKTTPPVLDSDGSFFLYS | |
| | RLTVDKSSWQEGNVFSCSVMHEALHNHYTQKSLSLSLGKIEGR | |
| | MDQVSHRYPRIQSIKVQFTEYKKEKGFILTSQKEDEIMKVQNNSV | |
| | IINCDGFYLISLKGYFSQEVNISLHYQKDEEPLFQLKKVRSVNSLM | |
| | VASLTYKDKVYLNVTTDNTSLDDFHVNGGELILIHQNPGEFCVL | |
| | MQIPQAPWPWWAVLQLGWRPGWFLDSPDRPWNPPTFSPALLVV | |
| | TEGDNATFTCSFSNTSESFVLNWYRMSPSNQTDKLAAFPEDRSQP | |
| | GQDCRFRVTQLPNGRDFHMSVVRARRNDSGTYLCGAISLAPKA | |
| | QIKESLRAELRVTERRAEVPTAHPSPSPRPAGQFQ | |
| | QVSHRYPRIQSIKVQFTEYKKEKGFILTSQKEDEIMKVQNNSVIIN | |
| | CDGFYLISLKGYFSQEVNISLHYQKDEEPLFQLKKVRSVNSLMVA | |
| | SLTYKDKVYLNVTTDNTSLDDFHVNGGELILIHQNPGEFCVL | |

An anti-PD-1 polypeptide or an anti-PD-1 antigen binding fragment can comprise a VH having an amino acid sequence of any one of SEQ ID NOS: 32, 34, 36, 38, 40, 46, 48, 50, 52, 54, 56, 58, 60, 62, 64, 66, 68, 70, 72, 74, 76, and 78. An anti-PD-1 polypeptide or an anti-PD-1 antigen binding fragment can comprise a VH having an amino acid sequence of any one of SEQ ID NOS: 33, 35, 37, 39, 41, 47, 49, 51, 53, 55, 57, 59, 61, 63, 65, 67, 69, 71, 73, 75, 77, and 79.

In one instance, an anti-PD-1 polypeptide or an anti-PD-1 antigen binding fragment comprises a VH having an amino acid sequence of SEQ ID NO: 32, and a VL having an amino acid sequence of SEQ ID NO: 33. In another instance, an anti-PD-1 polypeptide or an anti-PD-1 antigen binding fragment comprises a VH having an amino acid sequence of SEQ ID NO: 34, and a VL having an amino acid sequence of SEQ ID NO: 35. In another instance, an anti-PD-1 polypeptide or an anti-PD-1 antigen binding fragment comprises a VH having an amino acid sequence of SEQ ID NO: 36, and a VL having an amino acid sequence of SEQ ID NO: 37. In another instance, an anti-PD-1 polypeptide or an anti-PD-1 antigen binding fragment comprises a VH having an amino acid sequence of SEQ ID NO: 38, and a VL having an amino acid sequence of SEQ ID NO: 39. In another instance, an anti-PD-1 polypeptide or an anti-PD-1 antigen binding fragment comprises a VH having an amino acid sequence of SEQ ID NO: 40, and a VL having an amino acid sequence of SEQ ID NO: 41. In another instance, an anti-PD-1 polypeptide or an anti-PD-1 antigen binding fragment comprises a VH having an amino acid sequence of SEQ ID NO: 46, and a VL having an amino acid sequence of SEQ ID NO: 47. In another instance, an anti-PD-1 polypeptide or an anti-PD-1 antigen binding fragment comprises a VH having an amino acid sequence of SEQ ID NO: 48, and a VL having an amino acid sequence of SEQ ID NO: 49. In another instance, an anti-PD-1 polypeptide or an anti-PD-1 antigen binding fragment comprises a VH having an amino acid sequence of SEQ ID NO: 50, and a VL having an amino acid sequence of SEQ ID NO: 51. In another instance, an anti-PD-1 polypeptide or an anti-PD-1 antigen binding fragment comprises a VH having an amino acid sequence of SEQ ID NO: 52, and a VL having an amino acid sequence of SEQ ID NO: 53. In another instance, an anti-PD-1 polypeptide or an anti-PD-1 antigen binding fragment comprises a VH having an amino acid sequence of SEQ ID NO: 54, and a VL having an amino acid sequence of SEQ ID NO: 55. In another instance, an anti-PD-1 polypeptide or an anti-PD-1 antigen binding fragment comprises a VH having an amino acid sequence of SEQ ID NO: 56, and a VL having an amino acid sequence of SEQ ID NO: 57. In another instance, an anti-PD-1 polypeptide or an anti-PD-1 antigen binding fragment comprises a VH having an amino acid sequence of SEQ ID NO: 58, and a VL having an amino acid sequence of SEQ ID NO: 59. In another instance, an anti-PD-1 polypeptide or an anti-PD-1 antigen binding fragment comprises a VH having an amino acid sequence of SEQ ID NO: 60, and a VL having an amino acid sequence of SEQ ID NO: 61. In another instance, an anti-PD-1 polypeptide or an anti-PD-1 antigen binding fragment comprises a VH having an amino acid sequence of SEQ ID NO: 62, and a VL having an amino acid sequence of SEQ ID NO: 63. In another instance, an anti-PD-1 polypeptide or an anti-PD-1 antigen binding fragment comprises a VH having an amino acid sequence of SEQ ID NO: 64, and a VL having an amino acid sequence of SEQ ID NO: 65. In another instance, an anti-PD-1 polypeptide or an anti-PD-1 antigen binding fragment comprises a VH having an amino acid sequence of SEQ ID NO: 66, and a VL having an amino acid sequence of SEQ ID NO: 67. In another instance, an anti-PD-1 polypeptide or an anti-PD-1 antigen binding fragment comprises a VH having an amino acid sequence of SEQ ID NO: 68, and a VL having an amino acid sequence of SEQ ID NO: 69. In another instance, an anti-PD-1 polypeptide or an anti-PD-1 antigen binding fragment comprises a VH having an amino acid sequence of SEQ ID NO: 70, and a VL having an amino acid sequence of SEQ ID NO: 71. In another instance, an anti-PD-1 polypeptide or an anti-PD-1 antigen binding fragment comprises a VH having an amino acid sequence of SEQ ID NO: 72, and a VL having an amino acid sequence of SEQ ID NO: 73. In another instance, an anti-PD-1 polypeptide or an anti-PD-1 antigen binding fragment comprises a VH having an amino acid sequence of SEQ ID NO: 74, and a VL having an amino acid sequence of SEQ ID NO: 75. In another instance, an anti-PD-1 polypeptide or an anti-PD-1 antigen binding fragment comprises a VH having an amino acid sequence of SEQ ID NO: 76, and a VL having an amino acid sequence of SEQ ID NO: 77. In another instance, an anti-PD-1 polypeptide or an anti-PD-1 antigen binding fragment comprises a VH having an amino acid sequence of SEQ ID NO: 78, and a VL having an amino acid sequence of SEQ ID NO: 79.

In one instance, an anti-PD-1 polypeptide or an anti-PD-1 antigen binding fragment comprises a VH CHR1 having an amino acid sequence of SEQ ID NO: 80, a VH CHR2 having an amino acid sequence of SEQ ID NO: 81, a VH CHR3 having an amino acid sequence of SEQ ID NO: 82, VL CHR1 having an amino acid sequence of SEQ ID NO: 83, a VL CHR2 having an amino acid sequence of SEQ ID NO: 84, and a VL CHR3 having an amino acid sequence of SEQ ID NO: 85. In one instance, an anti-PD-1 polypeptide or an anti-PD-1 antigen binding fragment comprises a VH CHR1 having an amino acid sequence of SEQ ID NO: 86, a VH CHR2 having an amino acid sequence of SEQ ID NO: 87, a VH CHR3 having an amino acid sequence of SEQ ID NO: 88, VL CHR1 having an amino acid sequence of SEQ ID NO: 89, a VL CHR2 having an amino acid sequence of SEQ ID NO: 90, and a VL CHR3 having an amino acid sequence of SEQ ID NO: 91. In one instance, an anti-PD-1 polypeptide or an anti-PD-1 antigen binding fragment comprises a VH CHR1 having an amino acid sequence of SEQ ID NO: 92, a VH CHR2 having an amino acid sequence of SEQ ID NO: 93, a VH CHR3 having an amino acid sequence of SEQ ID NO: 94, VL CHR1 having an amino acid sequence of SEQ ID NO: 95, a VL CHR2 having an amino acid sequence of SEQ ID NO: 96, and a VL CHR3 having an amino acid sequence of SEQ ID NO: 97. In one instance, an anti-PD-1 polypeptide or an anti-PD-1 antigen binding fragment comprises a VH CHR1 having an amino acid sequence of SEQ ID NO: 98, a VH CHR2 having an amino acid sequence of SEQ ID NO: 99, a VH CHR3 having an amino acid sequence of SEQ ID NO: 100, VL CHR1 having an amino acid sequence of SEQ ID NO: 101, a VL CHR2 having an amino acid sequence of SEQ ID NO: 102, and a VL CHR3 having an amino acid sequence of SEQ ID NO: 103.

In one instance, an anti-PD-1 polypeptide comprises a fusion protein. Such fusion protein can be, for example, a two-sided Fc fusion protein comprising the extracellular domain (ECD) of programmed cell death 1 (PD-1) and the ECD of tumor necrosis factor (ligand) superfamily member 4 (TNFSF4 or OX40L) fused via hinge-CH₂—CH₃ Fc domain of human IgG4, expressed in CHO-K1 cells, where the fusion protein has an exemplary amino acid sequence of SEQ ID NO: 104.

Also provided herein is an anti-HER2 antibody. An anti-HER2 antibody can be conjugated to an IL-2 polypeptide as provided herein. In some embodiments, the anti-HER2 antibody is Trastuzumab (Herceptin, Roche, Herclon, RG597, RO452317). Trastuzumab has a VH sequence of

(SEQ ID NO: 121)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
K.

The VL sequence of Trastuzumab is

(SEQ ID NO: 122)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

Modification to Fc Region

Disclosed herein are anti-PD-1 polypeptides, wherein the anti-PD-1 polypeptides comprise an Fc region, and the Fc region comprises at least one covalently linked chemical linker. In some embodiments, the chemical linker is covalently attached to an asparagine, glutamine, cysteine, or lysine residue. In some embodiments, the chemical linker is covalently attached to a lysine, or cysteine residue. In some embodiments, the chemical linker is covalently attached to a lysine residue. In some embodiments, the chemical linker is covalently attached to a constant region of the anti-PD-1 polypeptide. In some embodiments, the chemical linker is covalently attached to a constant region of the anti-PD-1 polypeptide.

In some embodiments, the anti-PD-1 polypeptide comprises an Fc region. In some embodiments, the Fc region is an IgG Fc region, an IgA Fc region, an IgD Fc region, an IgM Fc region, or an IgE Fc region. In some embodiments, the Fc region is an IgG Fc region, an IgA Fc region, or an IgD Fc region. In some embodiments, the Fc region is a human Fc region. In some embodiments, the Fc region is a humanized. Fc region. In some embodiments, the Fc region is an IgG Fc region. In some instances, an IgG Fc region is an IgG1 Fc region, an IgG2a Fc region, or an IgG4 Fc region. In some instances, an IgG Fc region is an IgG1 Fc region, an IgG2a Fc region, or an IgG4 Fc region.

One or more mutations may be introduced in an Fc region to reduce Fc-mediated effector functions of an antibody or antigen-binding fragment such as, for example, antibody-dependent cellular cytotoxicity (ADCC) and/or complement function. In some instances, a modified Fc comprises a humanized IgG4 kappa isotype that contains a S228P Fc mutation. In some instances, a modified Fc comprises a human IgG1 kappa where the heavy chain CH2 domain is engineered with a triple mutation such as, for example: (a) L238P, L239E, and P335S; or (2) K248; K288; and K317.

In some embodiments, the Fc region has an amino acid sequence at least 80%, at least 85%, at least 90%, at least 91%, at least 92%, at least 93%, at least 94%, at least 95%, at least 96%, at least 97%, at least 98%, or at least 99% identical to a sequence as set forth in SEQ ID NO: 105 (Pro Lys Ser Cys Asp Lys Thr His Thr Cys Pro Pro Cys Pro Ala Pro Glu Xaa Xaa Gly Xaa Pro Ser Val Phe Leu Phe Pro Pro Lys Pro Lys Asp Thr Leu Met Ile Ser Arg Thr Pro Glu Val Thr Cys Val Val Val Asp Val Ser His Glu Asp Pro Glu Val Lys Phe Asn Trp Tyr Val Asp Gly Val Glu Val His Asn Ala Lys Thr Lys Pro Arg Glu Glu Gln Tyr Asp Ser Thr Tyr Arg Val Val Ser Val Leu Thr Val Leu His Gln Asp Trp Leu Asn Gly Lys Glu Tyr Lys Cys Lys Val Ser Asn Lys Ala Leu Pro Ala Pro Ile Glu Lys Thr Ile Ser Lys Ala Lys Gly Gln Pro Arg Glu Pro Gln Val Tyr Thr Leu Pro Pro Ser Arg Xaa Glu Xaa Thr Lys Asn Gln Val Ser Leu Thr Cys Leu Val Lys Gly Phe Tyr Pro Ser Asp Ile Ala Val Glu Trp Glu Ser Asn Gly Gln Pro Glu Asn Asn Tyr Lys Xaa Thr Pro Pro Val Leu Asp Ser Asp Gly Ser Phe Phe Leu Tyr Ser Lys Leu Thr Val Asp Lys Ser Arg Trp Gln Gln Gly Asn Val Phe Ser Cys Ser Val Met His Glu Ala Leu His Asn His Tyr Thr Gln Lys Ser Leu Ser Leu Ser Pro Gly, where Xaa can be any naturally occurring amino acid).

In some embodiments, the Fc region comprises one or more mutations which make the Fc region susceptible to modification or conjugation at a particular residue, such as by incorporation of a cysteine residue at a position which does not contain a cysteine in SEQ ID NO: 105. Alternatively, the Fc region could be modified to incorporate a modified natural amino acid or an unnatural amino acid which comprises a conjugation handle, such as one connected to the modified natural amino acid or unnatural amino acid through a linker. In some embodiments, the Fc region does not comprise any mutations which facilitate the attachment of a linker to an additional cytokine (e.g., an IL-2, IL-7, or IL-18 polypeptide). In some embodiments, the chemical linker is attached to a native residue as set forth in SEQ ID NO: 105. In some embodiments, the chemical linker is attached to a native lysine residue of SEQ ID NO: 105.

In some embodiments, the chemical linker can be covalently attached to one amino acid residue of an Fc region of the anti-PD-1 polypeptide. In some embodiments, the chemical linker is covalently attached to a non-terminal residue of the Fc region. In some embodiments, the non-terminal residue is in the CH1, CH2, or CH3 region of the anti-PD-1 polypeptide. In some embodiments, the non-terminal residue is in the CH2 region of the anti-PD-1 polypeptide.

In some embodiments, the chemical linker is attached to the Fc region at an amino acid residue at any one of positions 10-90 of SEQ ID NO: 105. In some embodiments, the chemical linker is attached to the Fc region at an amino acid residue at any one of positions 10-20, 10-30, 10-40, 10-50, 10-60, 10-70, 1-80, 10-90, 10-100, 10-110, 10-120, 10-130, 10-140, 10-150, 10-160, 10-170, 10-180, 10-190, or 10-200 of SEQ ID NO: 105. In some embodiments, the chemical linker is attached to the Fc region at an amino acid residue at one of positions 10-30, 50-70, or 80-100 of SEQ ID NO: 105. In some embodiments, the chemical linker is attached to the Fc region at an amino acid residue at any one of positions 20-40, 65-85, or 90-110 of SEQ ID NO: 105. In some embodiments, the chemical linker is attached to the Fc region at an amino acid residue at one of positions 15-26, 55-65, or 85-90 of SEQ ID NO: 105. In some embodiments, the chemical linker is attached to the Fc region at an amino acid residue at any one of positions 25-35, 70-80, or 95-105 of SEQ ID NO: 105. In some embodiments, the chemical linker is attached to the Fc region at an amino acid residue at any one of positions 30, 32, 72, 74, 79 or 101 of SEQ ID NO: 105. In some embodiments, the chemical linker is attached to the Fc region at an amino acid residue at any one of positions K30, K32, K72, K74, Q79, or K101 of SEQ ID NO: 105. In some embodiments, the chemical linker is attached to the Fc region at amino acid residue 30 of SEQ ID NO: 105. In some embodiments, the chemical linker is attached to the Fc region at amino acid residue 32 of SEQ ID NO: 105. In some embodiments, the chemical linker is attached to the Fc region at amino acid residue 72 of SEQ ID NO: 105. In some embodiments, the chemical linker is attached to the Fc region at amino acid residue 74 of SEQ ID NO: 105. In some embodiments, the chemical linker is attached to the Fc region at amino acid residue 79 of SEQ ID NO: 105. In some embodiments, the chemical linker is attached to the Fc region at amino acid residue 101 of SEQ ID NO: 105.

In some embodiments, the chemical linker is covalently attached at an amino acid residue of the polypeptide which selectively binds a cancer or inflammatory associated antigen (e.g., an anti-PD-1 antibody) such that the function of the polypeptide is maintained (e.g., without denaturing the polypeptide). For example, when the polypeptide is an antibody such as a human IgG (e.g., human IgG1), exposed lysine residues exposed glutamine residues and exposed tyrosine residues are present at the following positions (refer to web site imgt. org/IMGTScientificChart/Numbering/Hu_IGHGnber.html by EU numbering). Exemplary exposed Lysine Residues: CH2 domain (position 246, position 248, position 274, position 288, position 290, position 317, position 320, position 322, and position 338) CH3 domain (position 360, position 414, and position 439). Exemplary exposed Glutamine Residues: CH2 domain (position 295). Exemplary exposed Tyrosine Residues: CH2 domain (position 278, position 296, and position 300) CH3 domain (position 436).

The human IgG, such as human IgG1, may also be modified with a lysine, glutamine, or tyrosine residue at any one of the positions listed above in order provide a residue which is ideally surface exposed for subsequent modification.

In some embodiments, the chemical linker is covalently attached at an amino acid residue in the constant region of an anti-PD-1 antibody. In some embodiments, the chemical linker is covalently attached at an amino acid residue in the CH1, CH2, or CH3 region. In some embodiments, the chemical inker is covalently attached at an amino acid residue in the CH2 region. In some embodiments, the chemical linker may be covalently attached to one residue selected from the following groups of residues following EU numbering in human IgG Fc: amino acid residues 1-478, amino acid residues 2-478, amino acid residues 1-477, amino acid residues 2-477, amino acid residues 10-467, amino acid residues 30-447, amino acid residues 50-427, amino acid residues 100-377, amino acid residues 150-327, amino acid residues 200-327, amino acid residues 240-327, and amino acid residues 240-320.

In some embodiments, the chemical linker is covalently attached to one lysine or glutamine residue of a human IgG Fc region. In some embodiments, the chemical linker is covalently attached at Lys 246 of an Fc region of the anti-PD-1 polypeptide, wherein amino acid residue position number is based on Eu numbering. In some embodiments, the chemical linker is covalently attached at Lys 248 of an Fc region of the anti-PD-1 polypeptide, wherein amino acid residue position number is based on Eu numbering. In some embodiments, the chemical linker is covalently attached at Lys 288 of an Fc region of the anti-PD-1 polypeptide, wherein amino acid residue position number is based on Eu numbering. In some embodiments, the chemical linker is covalently attached at Lys 290 of an Fc region of the anti-PD-1 polypeptide, wherein amino acid residue position number is based on Eu numbering. In some embodiments, the chemical linker is covalently attached at Gln 295 of an Fc region of the antibody polypeptide, wherein amino acid residue position number is based on Eu numbering. In some embodiments, the chemical linker is covalently attached at Lys 317 of the anti-PD-1 polypeptide, wherein amino acid residue position number is based on Eu numbering.

In some embodiments, the chemical linker can be covalently attached to an amino acid residue selected from a subset of amino acid residues. In some embodiments, the subset comprises two three, four, five, six, seven, eight, nine, or ten amino acid residues of an Fc region of the anti-PD-1 polypeptide. In some embodiments, the chemical linker can be covalently attached to one of two lysine residues of an Fc region of the anti-PD-1 polypeptide.

In some embodiments, the anti-PD-1 polypeptide will comprise two linkers covalently attached to the Fc region of the anti-PD-1 polypeptide. In some embodiments, each of the two linkers will be covalently attached to a different heavy chain of the anti-PD1 polypeptide. In some embodiments, each of the two linkers will be covalently attached to a different heavy chain of the anti-PD-1 polypeptide at a residue position which is the same. In some embodiments, each of the two linkers will be covalently attached to a different heavy chain of anti-PD-1 polypeptide at a residue position which is different. When the two linkers are covalently attached to residue positions which differ, any combination of the residue positions provided herein may be used in combination.

In some embodiments, a first chemical linker is covalently attached at Lys 248 of a first Fc region of the anti-PD-1 polypeptide, and a second chemical linker is covalently attached at Lys 288 of a second Fc region of the anti-PD-1 polypeptide, wherein residue position number is based on Eu numbering. In some embodiments, a first chemical linker is covalently attached at Lys 246 of a first Fc region of the anti-PD-1 polypeptide, and a second chemical linker is covalently attached at Lys 288 of a second Fc region of the anti-PD-1 polypeptide, wherein residue position number is based on Eu numbering. In some embodiments, a first chemical linker is covalently attached at Lys 248 of a first Fc region of the anti-PD-1 polypeptide, and a second chemical linker is covalently attached at Lys 317 of a second Fc region of the anti-PD-1 polypeptide, wherein residue position number is based on Eu numbering. In some embodiments, a first chemical linker is covalently attached at Lys 246 of a first Fc region of the anti-PD-1 polypeptide, and a second chemical linker is covalently attached at Lys 317 of a second Fc region of the anti-PD-1 polypeptide, wherein residue position number is based on Eu numbering. In some embodiments, a first chemical linker is covalently attached at Lys 288 of a first Fc region of the anti-PD-1 polypeptide, and a second chemical linker is covalently attached at Lys 317 of a second Fc region of the anti-PD-1 polypeptide, wherein residue position number is based on Eu numbering.

Method of Modifying an Fc Region

Also provided herein are method of preparing a modified Fc region of a polypeptide which selectively binds to programmed cell death protein 1 (PD-1), such as for the attachment of a linker, a conjugation handle, or the cytokine to the polypeptide which selectively binds to PD-1. A variety of methods for site-specific modification of Fc regions of antibodies or other polypeptides which bind to PD-1 are known in the art.

Modification with an Affinity Peptide Configured to Site-Specifically Attach Linker to the Antibody

In some embodiments, an Fc region is modified to incorporate a linker, a conjugation handle, or a combination thereof. In some embodiments, the modification is performed by contacting the Fc region with an affinity peptide bearing a payload configured to attach a linker or other group to the Fc region, such as at a specific residue of the Fc region. In some embodiments, the linker is attached using a reactive group (e.g., a N-hydroxysuccinimide ester) which forms a bond with a residue of the Fc region. In some embodiments, the affinity peptide comprises a cleavable linker. The cleavable linker is configured on the affinity peptide such that after the linker or other group is attached to the Fc region, the affinity peptide can be removed, leaving behind only the desired linker or other group attached to the Fc region. The linker or other group can then be used further to add attach additional groups, such as a cytokine or a linker attached to a cytokine, to the Fc region.

Non-limiting examples of such affinity peptides can be found at least in PCT Publication No. WO2018199337A1, PCT Publication No. WO2019240288A1, PCT Publication No. WO2019240287A1, and PCT Publication No. WO2020090979A1, each of which is incorporated by reference as if set forth herein in its entirety. In some embodiments, the affinity peptide is a peptide which has been modified to deliver the linker/conjugation handle payload one or more specific residues of the Fc region of the antibody. In some embodiments, the affinity peptide has at least 80%, 85%, 90%, 91%, 92%, 93%, 94%, 95%, 96%, 97%, 98%, 99%, or 100% sequence identify to a peptide selected from among (1) QETNPTENLYFQQKNMQCQRRFYEALHDPNLNEEQRNARIRSIRDDDC (SEQ ID NO: 106); (2) QTADNQKNMQCQRRFYEALEEDPNLNEEQRNARIRSIRDDCSQSANLLAEAQQLNDAQA PQA (SEQ ID NO: 107); (3) QETKNMQCQRRFYEALHDPNLNEEQRNARIRSIRDDDC (SEQ ID NO: 108); (4) QETFNKQCQRRFYEALHDPNLNEEQRNARIRSIRDDDC (SEQ ID NO: 109); (5) QETFNMQCQRRFYEALHDPNLNKEQRNARIRSIRDDDC (SEQ ID NO: 110); (6) QETFNMQCQRRFYEALHDPNLNEEQRNARIRSIKDDC (SEQ ID NO: 111); (7) QETMQCQRRFYEALHDPNLNEEQRNARIRSIKDDC (SEQ ID NO: 112); (8) QETQCQRRFYEALHDPNLNEEQRNARIRSIKDDC (SEQ ID NO: 113); (9) QETCQRRFYEALHDPNLNEEQRNARIRSIKDDC (SEQ ID NO: 114); (10) QETRGNCAYHKGQLVWCTYH (SEQ ID NO: 115); and (11) QETRGNCAYHKGQIIWCTYH (SEQ ID NO: 116), or a corresponding peptide which has been truncated at the N-terminus by one, two, three, four, or five residues. An exemplary affinity peptide with cleavable linker and conjugation handle payload capable of attaching the payload to residue K248 of an antibody as provided herein is shown below (SEQ ID NO: 155) (as reported in Matsuda et al, “Chemical Site-Specific Conjugation Platform to Improve the Pharmacokinetics and Therapeutic Index of Antibody-Drug Conjugates,” Mol. Pharmaceutics 2021, 18, 11, 4058-4066.

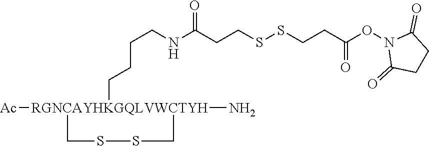

Alternative affinity peptides targeting alternative residues of the Fc region are described in the references cited above for AJICAP™ technology, and such affinity peptides can be used to attach the desired functionality to an alternative residue of the Fc region (e.g., K246, K288, etc.). For example, the disulfide group of the above affinity peptide could instead be replaced with a thioester to provide a sulfhydryl protecting group as a cleavable portion of the linking group (e.g., the relevant portion of the affinity peptide would have a structure of

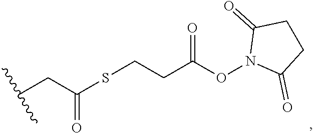 [SMILES: *CC(=O)SCCC(=O)ON1C(=O)CCC1=O]

or another of the cleavable linkers discussed below).

The affinity peptide of the disclosure can comprise a cleavable linker. In some embodiments, the cleavable linker of the affinity peptide connects the affinity peptide to the group which is to be attached to the Fc region and is configured such that the peptide can be cleaved after the group comprising the linker or conjugation handle has been attached. In some embodiments, the cleavable linker is a divalent group. In some embodiments, the cleavable linker can comprise a thioester group, an ester group, a sulfane group; a methanimine group; an oxyvinyl group; a thiopropanoate group; an ethane-1,2-diol group; an (imidazole-1-yl)methan-1-one group; a seleno ether group; a silylether group; a di-oxysilane group; an ether group; a di-oxymethane group; a tetraoxospiro[5.5]undecane group; an acetamidoethyl phosphoramidite group; a bis(methylthio)-pyrazolopyrazole-dione group; a 2-oxo-2-phenylethyl formate group; a 4-oxybenzylcarbamate group; a 2-(4-hydroxy-oxyphenyl)diazinyl)benzoic acid group; a 4-amino-2-(2-amino-2-oxoethyl)-4-oxobut-2-enoic acid group; a 2-(2-methylenehydrazineyl)pyridine group; an N′-methyleneformohydrazide group; or an isopropylcarbamate group, any of which is unsubstituted or substituted. Composition and points of attachment of the cleavable linker to the affinity peptide, as well as related methods of use, are described in, at least, PCT Publication No. WO2018199337A1, PCT Publication No. WO2019240288A1, PCT Publication No. WO2019240287A1, and PCT Publication No. WO2020090979A1.

In some embodiments, the cleavable linker is:

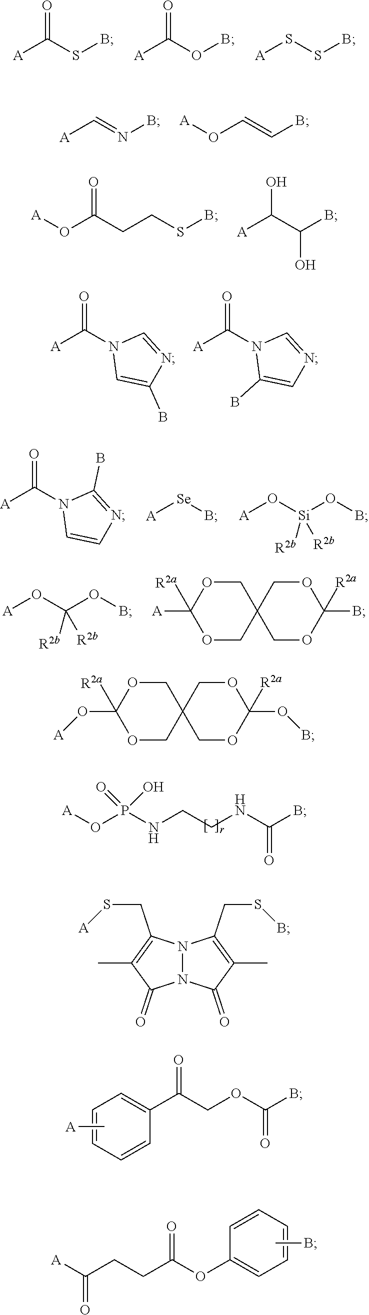

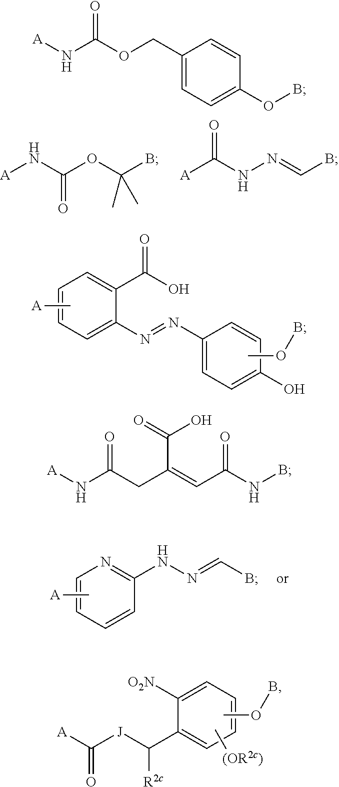

wherein:
one of A or B is a point of attachment the linker and the other of A or B is a point of attachment to the affinity peptide;
each R^{2a} is independently H or optionally substituted alkyl;
each R_{2b} is independently H or optionally substituted alkyl;
R^{2c} is a H or optionally substituted alkyl;
J is a methylene, a N, a S, a Si, or an O atom; and
r is 1, 2, 3, 4, 5, 6, 7, 8, 9, or 10.

The affinity peptide comprises a reactive group which is configured to enable the covalent attachment of the linker/conjugation handle to the Fc region. In some embodiments, the reactive group is selective for a functional group of a specific amino acid residue, such as a lysine residue, tyrosine residue, serine residue, cysteine residue, or an unnatural amino acid residue of the Fc region incorporated to facilitate the attachment of the linker. The reactive group may be any suitable functional group, such as an activated ester for reaction with a lysine (e.g., N-hydroxysuccinimide ester or a derivate thereof, a pentafluorophenyl ester, etc.) or a sulfhydryl reactive group for reaction with a cysteine (e.g., a Michael acceptor, such as an alpha-beta unsaturated carbonyl or a maleimide). In some embodiments, the reactive group is:

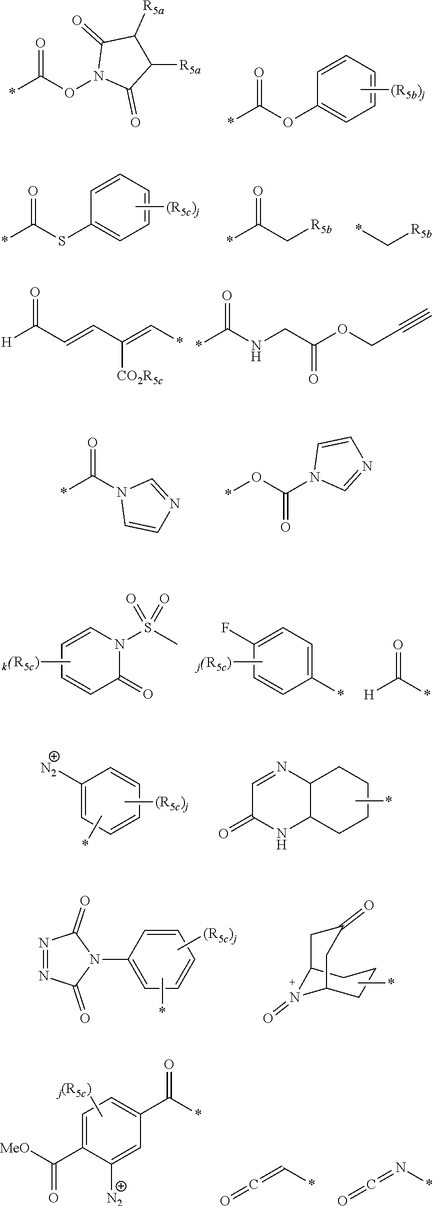

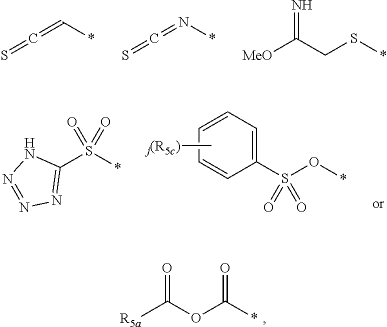

wherein:
each R^{5a}, R^{5b}, and R^{5c} is independently H, halogen, or optionally substituted alkyl;
each j is 1, 2, 3, 4, or 5; and
each k is 1, 2, 3, 4, or 5.

In some embodiments, the affinity peptide is used to deliver a reactive moiety to the desired amino acid residue such that the reactive moiety is exposed upon cleavage of the cleavable linker. By way of non-limiting example, the reactive group forms a covalent bond with a desired residue of the Fc region of the polypeptide which selectively binds to anti-PD-1 due to an interaction between the affinity peptide and the Fc region. Following this covalent bond formation, the cleavable linker is cleaved under appropriate conditions to reveal a reactive moiety (e.g., if the cleavable linker comprises a thioester, a free sulfhydryl group is attached to the Fc region following cleavage of the cleavable linker). This new reactive moiety can then be used to subsequently add an additional moiety, such as a conjugation handle, by way of reagent comprising the conjugation handle tethered to a sulfhydryl reactive group (e.g., alpha-halogenated carbonyl group, alpha-beta unsaturated carbonyl group, maleimide group, etc.).

In some embodiments, an affinity peptide is used to deliver a free sulfhydryl group to a lysine of the Fc region. In some embodiments, the free sulfhydryl group is then reacted with a bifunctional linking reagent to attach a new conjugation handle to the Fc region. In some embodiments, the new conjugation handle is then used to form the linker to the attached cytokine. In some embodiments, the new conjugation handle is an alkyne functional group. In some embodiments, the new conjugation handle is a DBCO functional group.

JOIN Exemplary bifunctional linking reagents useful for this purpose are of a formula A-B-C, wherein A is the sulfhydryl reactive conjugation handle (e.g., maleimide, α,β-unsaturated carbonyl, a-halogenated carbonyl), B is a lining group, and C is the new conjugation handle (e.g., an alkyne such as DBCO). Specific non-limiting examples of bifunctional linking reagents include

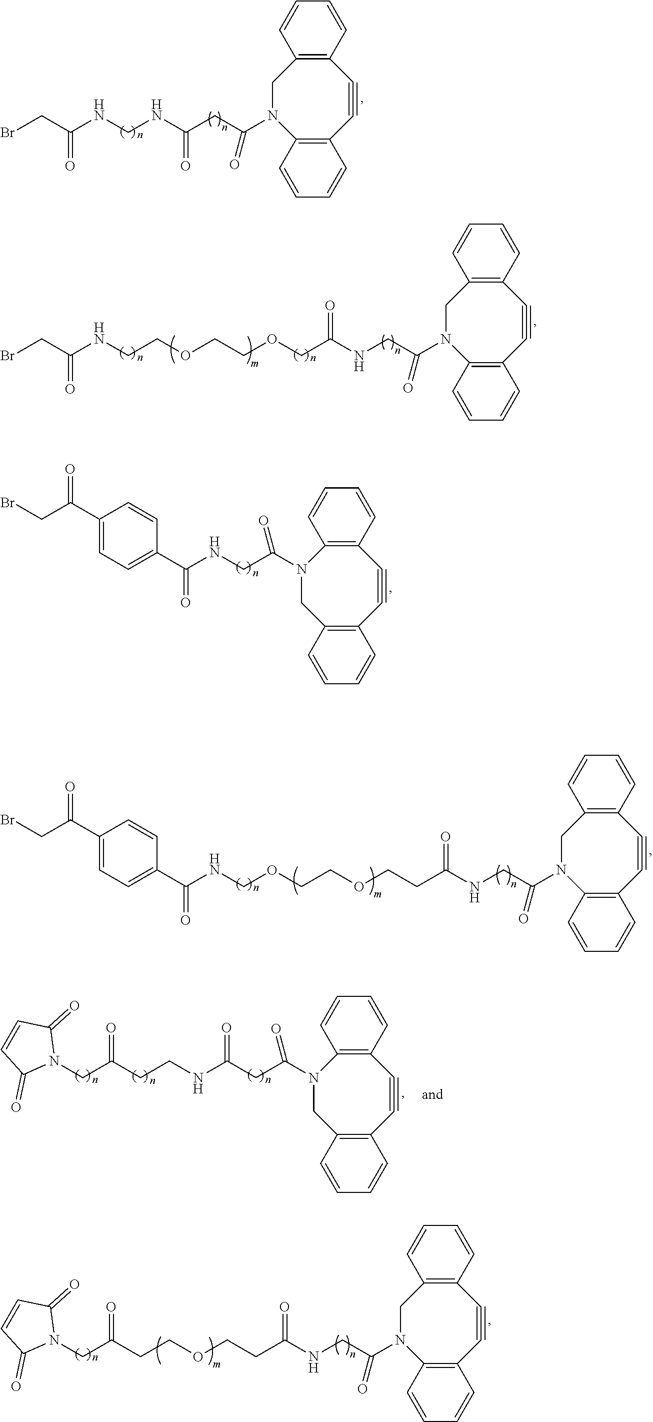

wherein each n is independently an integer from 1-6 and each m is independently an integer from 1-30, and related molecules (e.g., isomers).

Alternatively, the affinity peptide can be configured such that a conjugation handle is added to the Fc region (such as by a linker group) immediately after covalent bond formation between the reactive group and a residue of the Fc region. In such cases, the affinity peptide is cleaved and the conjugation handle is immediately ready for subsequent conjugation to the IL-2 polypeptide (or other cytokines).

Alternative Methods of Attachment—Enzyme Mediated

While the affinity peptide mediated modification of an Fc region of an antibody provide supra possesses many advantages over other methods which can be used to site-specifically modify the Fc region (e.g., ease of use, ability to rapidly generate many different antibody conjugates, ability to use many “off-the-shelf” commercial antibodies without the need to do time consuming protein engineering, etc.), other methods of performing the modification are also contemplated as being within the scope of the present disclosure.

In some embodiments, the present disclosure relates generally to transglutaminase-mediated site-specific antibody-drug conjugates (ADCs) comprising: 1) glutamine-containing tags, endogenous glutamines (e.g., native glutamines without engineering, such as glutamines in variable domains, CDRs, etc.), and/or endogenous glutamines made reactive by antibody engineering or an engineered transglutaminase; and 2) amine donor agents comprising amine donor units, linkers, and agent moieties. Non-limiting examples of such transglutaminase mediated site-specific modifications can be found at least in publications WO2020188061, US2022133904, US2019194641, US2021128743, U.S. Pat. No. 9,764,038, U.S. Ser. No. 10/675,359, U.S. Pat. No. 9,717,803, U.S. Ser. No. 10/434,180, U.S. Pat. No. 9,427,478, which are incorporated by reference as if set forth herein in their entirety.

In another aspect, the disclosure provides an engineered Fc-containing polypeptide conjugate comprising the formula: (Fc-containing polypeptide-T-A), wherein T is an acyl donor glutamine-containing tag engineered at a specific site, wherein A is an amine donor agent, wherein the amine donor agent is site-specifically conjugated to the acyl donor glutamine-containing tag at a carboxyl terminus, an amino terminus, or at an another site in the Fc-containing polypeptide, wherein the acyl donor glutamine-containing tag comprises an amino acid sequence XXQX, wherein X is any amino acid (e.g., X can be the same or different amino acid), and wherein the engineered Fc-containing polypeptide conjugate comprises an amino acid substitution from glutamine to asparagine at position 295 (Q295N; EU numbering scheme).

In some embodiments, the acyl donor glutamine-containing tag is not spatially adjacent to a reactive Lys (e.g., the ability to form a covalent bond as an amine donor in the presence of an acyl donor and a transglutaminase) in the polypeptide or the Fc-containing polypeptide. In some embodiments, the polypeptide or the Fc-containing polypeptide comprises an amino acid modification at the last amino acid position in the carboxyl terminus relative to a wild-type polypeptide at the same position. The amino acid modification can be an amino acid deletion, insertion, substitution, mutation, or any combination thereof.

In some embodiments, the polypeptide conjugate comprises a full length antibody heavy chain and an antibody light chain, wherein the acyl donor glutamine-containing tag is located at the carboxyl terminus of a heavy chain, a light chain, or both the heavy chain and the light chain.

In some embodiments, the polypeptide conjugate comprises an antibody, wherein the antibody is a monoclonal antibody, a polyclonal antibody, a human antibody, a humanized antibody, a chimeric antibody, a bispecific antibody, a minibody, a diabody, or an antibody fragment. In some embodiments, the antibody is an IgG.

In another aspect, described herein is a method for preparing an engineered Fc-containing polypeptide conjugate comprising the formula: (Fc-containing polypeptide-T-A), wherein T is an acyl donor glutamine-containing tag engineered at a specific site, wherein A is an amine donor agent, wherein the amine donor agent is site-specifically conjugated to the acyl donor glutamine-containing tag at a carboxyl terminus, an amino terminus, or at an another site in the Fc-containing polypeptide, wherein the acyl donor glutamine-containing tag comprises an amino acid sequence XXQX, wherein X is any amino acid (e.g., X can be the same or a different amino acid), and wherein the engineered Fc-containing polypeptide conjugate comprises an amino acid substitution from glutamine to asparagine at position 295 (Q295N; EU numbering scheme), comprising the steps of: a) providing an engineered (Fc-containing polypeptide)-T molecule comprising the Fc-containing polypeptide and the acyl donor glutamine-containing tag; b) contacting the amine donor agent with the engineered (Fc-containing polypeptide)-T molecule in the presence of a transglutaminase; and c) allowing the engineered (Fc-containing polypeptide)-T to covalently link to the amine donor agent to form the engineered Fc-containing polypeptide conjugate.

In another aspect, described herein is a method for preparing an engineered polypeptide conjugate comprising the formula: polypeptide-T-A, wherein T is an acyl donor glutamine-containing tag engineered at a specific site, wherein A is an amine donor agent, wherein the amine donor agent is site-specifically conjugated to the acyl donor glutamine-containing tag at a carboxyl terminus, an amino terminus, or at an another site in the polypeptide, and wherein the acyl donor glutamine-containing tag comprises an amino acid sequence LLQGPX (SEQ ID NO: 153), wherein X is A or P, or GGLLQGPP (SEQ ID NO: 154), comprising the steps of: a) providing an engineered polypeptide-T molecule comprising the polypeptide and the acyl donor glutamine-containing tag; b) contacting the amine donor agent with the engineered polypeptide-T molecule in the presence of a transglutaminase; and c) allowing the engineered polypeptide-T to covalently link to the amine donor agent to form the engineered Fc-containing polypeptide conjugate.

In some embodiments, the engineered polypeptide conjugate (e.g., the engineered Fc-containing polypeptide conjugate, the engineered Fab-containing polypeptide conjugate, or the engineered antibody conjugate) as described herein has conjugation efficiency of at least about 51%. In another aspect, the invention provides a pharmaceutical composition comprising the engineered polypeptide conjugate as described herein (e.g., the engineered Fc-containing polypeptide conjugate, the engineered Fab-containing polypeptide conjugate, or the engineered antibody conjugate) and a pharmaceutically acceptable excipient.

In some embodiments, described herein is a method for conjugating a moiety of interest (Z) to an antibody, comprising the steps of: (a) providing an antibody having (e.g., within the primary sequence of a constant region) at least one acceptor amino acid residue (e.g., a naturally occurring amino acid) that is reactive with a linking reagent (linker) in the presence of a coupling enzyme, e.g., a transamidase; and (b) reacting said antibody with a linking reagent (e.g., a linker comprising a primary amine) comprising a reactive group (R), optionally a protected reactive group or optionally an unprotected reactive group, in the presence of an enzyme capable of causing the formation of a covalent bond between the acceptor amino acid residue and the linking reagent (other than at the R moiety), under conditions sufficient to obtain an antibody comprising an acceptor amino acid residue linked (covalently) to a reactive group (R) via the linking reagent. Optionally, said acceptor residue of the antibody or antibody fragment is flanked at the +2 position by a non-aspartic acid residue. Optionally, the residue at the +2 position is a non-aspartic acid residue. In one embodiment, the residue at the +2 position is a non-aspartic acid, non-glutamine residue. In one embodiment, the residue at the +2 position is a non-aspartic acid, non-asparagine residue. In one embodiment, the residue at the +2 position is a non-negatively charged amino acid (an amino acid other than an aspartic acid or a glutamic acid). Optionally, the acceptor glutamine is in an Fc domain of an antibody heavy chain, optionally further-within the CH2 domain Optionally, the antibody is free of heavy chain N297-linked glycosylation. Optionally, the acceptor glutamine is at position 295 and the residue at the +2 position is the residue at position 297 (EU index numbering) of an antibody heavy chain.

In one aspect, described herein is a method for conjugating a moiety of interest (Z) to an antibody, comprising the steps of: (a) providing an antibody having at least one acceptor glutamine residue; and (b) reacting said antibody with a linker comprising a primary amine (a lysine-based linker) comprising a reactive group (R), preferably a protected reactive group, in the presence of a transglutaminase (TGase), under conditions sufficient to obtain an antibody comprising an acceptor glutamine linked (covalently) to a reactive group (R) via said linker. Optionally, said acceptor glutamine residue of the antibody or antibody fragment is flanked at the +2 position by a non-aspartic acid residue. Optionally, the residue at the +2 position is a non-aspartic acid residue. In one embodiment, the residue at the +2 position is a non-aspartic acid, non-glutamine residue. In one embodiment, the residue at the +2 position is a non-aspartic acid, non-asparagine residue. In one embodiment, the residue at the +2 position is a non-negatively charged amino acid (an amino acid other than an aspartic acid or a glutamic acid). Optionally, the acceptor glutamine is in an Fc domain of an antibody heavy chain, optionally further-within the CH2 domain Optionally, the antibody is free of heavy chain N297-linked glycosylation. Optionally, the acceptor glutamine is at position 295 and the residue at the +2 position is the residue at position 297 (EU index numbering) of an antibody heavy chain.

The antibody comprising an acceptor residue or acceptor glutamine residue linked to a reactive group (R) via a linker comprising a primary amine (a lysine-based linker) can thereafter be reacted with a reaction partner comprising a moiety of interest (Z) to generate an antibody comprising an acceptor residue or acceptor glutamine residue linked to a moiety of interest (Z) via the linker. Thus, in one embodiment, the method further comprises a step (c): reacting (i) an antibody of step b) comprising an acceptor glutamine linked to a reactive group (R) via a linker comprising a primary amine (a lysine-based linker), optionally immobilized on a solid support, with (ii) a compound comprising a moiety of interest (Z) and a reactive group (R′) capable of reacting with reactive group R, under conditions sufficient to obtain an antibody comprising an acceptor glutamine linked to a moiety of interest (Z) via a linker comprising a primary amine (a lysine-based linker). Preferably, said compound comprising a moiety of interest (Z) and a reactive group (R′) capable of reacting with reactive group R is provided at a less than 80 times, 40 times, 20 times, 10 times, 5 times or 4 molar equivalents to the antibody. In one embodiment, the antibody comprises two acceptor glutamines and the compound comprising a moiety of interest (Z) and a reactive group (R′) is provided at 10 or less molar equivalents to the antibody. In one embodiment, the antibody comprises two acceptor glutamines and the compound comprising a moiety of interest (Z) and a reactive group (R′) is provided at 5 or less molar equivalents to the antibody. In one embodiment, the antibody comprises four acceptor glutamines and the compound comprising a moiety of interest (Z) and a reactive group (R′) is provided at 20 or less molar equivalents to the antibody. In one embodiment, the antibody comprises four acceptor glutamines and the compound comprising a moiety of interest (Z) and a reactive group (R′) is provided at 10 or less molar equivalents to the antibody. In one embodiment, steps (b) and/or (c) are carried out in aqueous conditions. Optionally, step (c) comprises: immobilizing a sample of an antibody comprising a functionalized acceptor glutamine residue on a solid support to provide a sample comprising immobilized antibodies, reacting the sample comprising immobilized antibodies with a compound, optionally recovering any unreacted compound and re-introducing such recovered compound to the solid support for reaction with immobilized antibodies, and eluting the antibody conjugates to provide a composition comprising a Z moiety.

Conjugation Handle Chemistry

In some embodiments, the appropriately modified Fc region of the polypeptide which selectively binds to PD-1 will comprise a conjugation handle which is used to conjugate the polypeptide which selectively binds to PD-1 to an IL-2 polypeptide.

Any suitable reactive group capable of reacting with a complementary reactive group attached to the IL-2 polypeptide can be used as the conjugation handle. In some embodiments, the conjugation handle comprises a reagent for a Cu(I)-catalyzed or “copper-free” alkyne-azide triazole-forming reaction (e.g., strain promoted cycloadditions), the Staudinger ligation, inverse-electron-demand Diels-Alder (IEDDA) reaction, “photo-click” chemistry, tetrazine cycloadditions with trans-cycloctenes, or a metal-mediated process such as olefin metathesis and Suzuki-Miyaura or Sonogashira cross-coupling.

In some embodiments, the conjugation handle comprises a reagent for a “copper-free” alkyne azide triazole-forming reaction. Non-limiting examples of alkynes for said alkyne azide triazole forming reaction include cyclooctyne reagents (e.g., (1R,8S,9s)-Bicyclo[6.1.0]non-4-yn-9-ylmethanol containing reagents, dibenzocyclooctyne-amine reagents, difluorocyclooctynes, or derivatives thereof). In some embodiments, the alkyne functional group is attached to the Fc region. In some embodiments, the azide functional group is attached to the Fc region.

In some embodiments, the conjugation handle comprises a reactive group selected from azide, alkyne, tetrazine, halide, sulfhydryl, disulfide, maleimide, activated ester, alkene, aldehyde, ketone, imine, hydrazine, and hydrazide. In some embodiments, the IL-2 polypeptide comprises a reactive group complementary to the conjugation handle of the Fc region. In some embodiments, the conjugation handle and the complementary conjugation handle comprise “CLICK” chemistry reagents. Exemplary groups of click chemistry residue are shown in Hein et al., “Click Chemistry, A Powerful Tool for Pharmaceutical Sciences,” Pharmaceutical Research volume 25, pages 2216-2230 (2008); Thirumurugan et al., “Click Chemistry for Drug Development and Diverse Chemical—Biology Applications,” Chem. Rev. 2013, 113, 7, 4905-4979; US20160107999A1; U.S. Ser. No. 10/266,502B2; and US20190204330A1, each of which is incorporated by reference in its entirety.

Linker Structure

In some embodiments, the linker used to attach the polypeptide which selectively binds to PD-1 and the cytokine (such as the IL-2 polypeptide) comprises points of attachment at both moieties. The points of attachment can be any of the residues for facilitating the attachment as provided herein. The linker structure can be any suitable structure for creating the spatial attachment between the two moieties. In some embodiments, the linker provides covalent attachment of both moieties. In some embodiments, the linker is a chemical linker (e.g., not an expressed polypeptide as in a fusion protein).

In some embodiments, the linker comprises a polymer. In some embodiments, the linker comprises a water soluble polymer. In some embodiments, the linker comprises poly(alkylene oxide), polysaccharide, poly(vinyl pyrrolidone), poly(vinyl alcohol), polyoxazoline, poly(acryloylmorpholine), or a combination thereof. In some embodiments, the linker comprises poly(alkylene oxide). In some embodiments, the poly(alkylene oxide) is polyethylene glycol or polypropylene glycol, or a combination thereof. In some embodiments, the poly(alkylene oxide) is polyethylene glycol.

In some embodiments, the linker is a bifunctional linker. In some embodiments, the bifunctional linker comprises an amide group, an ester group, an ether group, a thioether group, or a carbonyl group. In some embodiments, the linker comprises a non-polymer linker. In some embodiments, the linker comprises a non-polymer, bifunctional linker. In some embodiments, the non-polymer, bifunctional linker comprises succinimidyl 4-(N-maleimidomethyl)cyclohexane-1-carboxylate; Maleimidocaproyl; Valine-citrulline; Allyl(4-methoxyphenyl)dimethylsilane; 6-(Allyloxycarbonylamino)-1-hexanol; 4-Aminobutyraldehyde diethyl acetal; or (E)-N-(2-Aminoethyl)-4-{2-[4-(3-azidopropoxy)phenyl]diazenyl}benzamide hydrochloride.

The linker can be branched or linear. In some embodiments, the linker is linear. In some embodiments, the linker is branched. In some embodiments, the linker comprises a linear portion (e.g., between the first point of attachment and the second point of attachment) of a chain of at least 10, 20, 50, 100, 500, 1000, 2000, 3000, or 5000 atoms. In some embodiments, the linker comprises a linear portion of a chain of at least 10, 20, 30, 40, or 50 atoms. In some embodiments, the linker comprises a linear portion of at least 10 atoms. In some embodiments, the linker is branched and comprises a linear portion of a chain of at least 10, 20, 50, 100, 500, 1000, 2000, 3000, or 5000 atoms.

In some embodiments, the linker has a molecular weight of about 200 Daltons to about 2000 Daltons. In some embodiments, the linker has a molecular weight of 200 Daltons to 100,000 Daltons. In some embodiments, the linker has a molecular weight of 200 Daltons to 500 Daltons, 200 Daltons to 750 Daltons, 200 Daltons to 1,000 Daltons, 200 Daltons to 5,000 Daltons, 200 Daltons to 10,000 Daltons, 200 Daltons to 20,000 Daltons, 200 Daltons to 50,000 Daltons, 200 Daltons to 100,000 Daltons, 500 Daltons to 750 Daltons, 500 Daltons to 1,000 Daltons, 500 Daltons to 5,000 Daltons, 500 Daltons to 10,000 Daltons, 500 Daltons to 20,000 Daltons, 500 Daltons to 50,000 Daltons, 500 Daltons to 100,000 Daltons, 750 Daltons to 1,000 Daltons, 750 Daltons to 5,000 Daltons, 750 Daltons to 10,000 Daltons, 750 Daltons to 20,000 Daltons, 750 Daltons to 50,000 Daltons, 750 Daltons to 100,000 Daltons, 1,000 Daltons to 5,000 Daltons, 1,000 Daltons to 10,000 Daltons, 1,000 Daltons to 20,000 Daltons, 1,000 Daltons to 50,000 Daltons, 1,000 Daltons to 100,000 Daltons, 5,000 Daltons to 10,000 Daltons, 5,000 Daltons to 20,000 Daltons, 5,000 Daltons to 50,000 Daltons, 5,000 Daltons to 100,000 Daltons, 10,000 Daltons to 20,000 Daltons, 10,000 Daltons to 50,000 Daltons, 10,000 Daltons to 100,000 Daltons, 20,000 Daltons to 50,000 Daltons, 20,000 Daltons to 100,000 Daltons, or 50,000 Daltons to 100,000 Daltons. In some embodiments, the linker has a molecular weight of 200 Daltons, 500 Daltons, 750 Daltons, 1,000 Daltons, 5,000 Daltons, 10,000 Daltons, 20,000 Daltons, 50,000 Daltons, or 100,000 Daltons. In some embodiments, the linker has a molecular weight of at least 200 Daltons, 500 Daltons, 750 Daltons, 1,000 Daltons, 5,000 Daltons, 10,000 Daltons, 20,000 Daltons, or 50,000 Daltons. In some embodiments, the linker has a molecular weight of at most 500 Daltons, 750 Daltons, 1,000 Daltons, 5,000 Daltons, 10,000 Daltons, 20,000 Daltons, 50,000 Daltons, or 100,000 Daltons. In a preferred embodiments, the linker has a molecular weight of less than 5000 Daltons, less than 4000 Daltons, less than 3000 Daltons, or less than 2000 Daltons, and the linker is monodisperse (e.g., for a population of conjugate compositions herein, there is a high degree of uniformity of the linker structure between the polypeptide which binds specifically to PD-L1 and the IL-2 polypeptide (or other cytokine).

In some embodiments, the linker comprises a reaction product one or more pairs of conjugation handles and a complementary conjugation handle thereof. In some embodiments, the reaction product comprises a triazole, a hydrazone, pyridazine, a sulfide, a disulfide, an amide, an ester, an ether, an oxime, an alkene, or any combination thereof. In some embodiments, the reaction product comprises a triazole. The reaction product can be separated from the first point of attachment and the second point of attachment by any portion of the linker. In some embodiments, the reaction product is substantially in the center of the linker. In some embodiments, the reaction product is substantially closer to one point of attachment than the other.

In some embodiments, the linker comprises a structure of Formula (X)

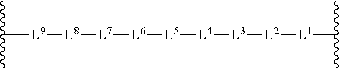

wherein each of L¹, L², L³, L⁴, L⁵, L⁶, L⁸, and L⁹ is independently —O—, —NR^{L}—, —N(R^{L})₂⁺—, —OP(═O)(OR^{L})O—, —S—, —S(═O)—, —S(═O)₂—, —C(═O)—, —C(═O)O—, —OC(═O)—, —OC(═O)O—, —OC(═O)NR^{L}—, —NR^{L}—C(═O)O—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}—, —(O—CH₂—CH₂)_{qb}—, —(CH₂—CH(CH₃)—O)_{qc}—, —(O—CH(CH₃)—CH₂)_{qd}—, a reaction product of a conjugation handle and a complementary conjugation handle, or absent;
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
each of qa, qb, qc and qd is independently an integer from 1-100,
wherein each

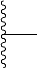

is a point of attachment to the polypeptide which selectively binds to PD-1 or the cytokine (e.g., the IL-2 polypeptide).

In some embodiments, the linker comprises a structure of Formula (X^{a})

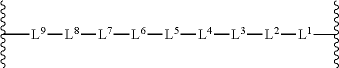

wherein each of L¹, L², L³, L⁴, L⁵, L⁶, L⁷, L⁸, and L⁹ is independently —O—, —NR^{L}—, —(C₁-C₆ alkylene)NR^{L}—, —NR^{L}(C₁-C₆ alkylene)-, —N(R^{L})₂+—, —(C₁-C₆ alkylene)N(R^{L})₂⁺—, —N(R^{L})₂⁺—(C₁-C₆ alkylene)-, —OP(═O)(OR^{L})O—, —S—, —(C₁-C₆ alkylene)S—, —S(C₁-C₆ alkylene)-, —S(═O)—, —S(═O)₂—, —C(═O)—, —(C₁-C₆ alkylene)C(═O)—, —C(═O) (C₁-C₆ alkylene)-, —C(═O)O—, —OC(═O)—, —OC(═O)O—, —C(═O)NR^{L}—, —C(═O)NR^{L}(C₁-C₆ alkylene)-, —(C₁-C₆ alkylene)C(═O)NR^{L}—, —NR^{L}C(═O)—, —(C₁-C₆ alkylene)NR^{L}C(═O)—, —NR^{L}C(═O)(C₁-C₆ alkylene)-, —OC(═O)NR^{L}—, —NR^{L}C(═O)O—, —NR^{L}C(═O)NR^{L}—, —NR^{L} s)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂-0)_{qa}—, —(O—CH₂—CH₂)_{qb}—, —(CH₂—CH(CH₃)-0)_{qc}—, —(O—CH(CH₃)—CH₂)_{qd}—, a reaction product of a conjugation handle and a complementary conjugation handle, or absent; (C₁-C₆ alkylene)
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
each of qa, qb, qc and qd is independently an integer from 1-100,
wherein each

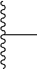

is a point of attachment to the polypeptide which selectively binds to PD-1 or the cytokine (e.g., the IL-2 polypeptide).

In some embodiments, the linker comprises a structure of Formula (X′)

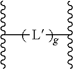

wherein each L′ is independently —O—, —NR^{L}—, —(C₁-C₆ alkylene)NR^{L}—, —NR^{L}(C₁-C₆ alkylene)-, —N(R^{L})₂⁺—, —(C₁-C₆ alkylene)N(R^{L})₂⁺—, —N(R^{L})₂+—(C₁-C₆ alkylene)-, —OP(═O)(OR^{L})O—, —S—, —(C₁-C₆ alkylene)S—, —S(C₁-C₆ alkylene)-, —S(═O)—, —S(═O)₂—, —C(═O)—, —(C₁-C₆ alkylene)C(═O)—, —C(═O) (C₁-C₆ alkylene)-, —C(═O)O—, —OC(═O)—, —OC(═O)O—, —C(═O)NR^{L}—, —C(═O)NR^{L}(C₁-C₆ alkylene)-, —(C₁-C₆ alkylene)C(═O)NR^{L}—, —NR^{L}C(═O)—, —(C₁-C₆ alkylene)NR^{L}C(═O)—, —NR^{L}C(═O)(C₁-C₆ alkylene)-, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}—, —(O—CH₂—CH₂)O—, —(CH₂—CH(CH₃)—O)_{qc}—, —(O—CH(CH₃)—CH₂)_{qd}—, a reaction product of a conjugation handle and a complementary conjugation handle, or absent; (C₁-C₆ alkylene);
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl;
each of qa, qb, qc and qd is independently an integer from 1-100, and,
g is an integer from 1-100,
wherein each

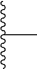

is a point of attachment to the modified IL-2 polypeptide or the antibody or antigen binding fragment.

In some embodiments, the linker of Formula (X) or of Formula (X^{a}) or of Formula (X′) comprises the structure:

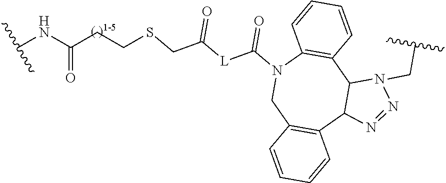

wherein

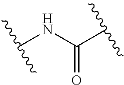 [SMILES: *NC(*)=O]

is the first point of attachment to a lysine residue of the polypeptide which selectively binds to PD-1;
L is a linking group; and

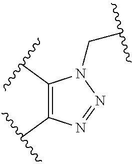 [SMILES: *Cn1nnc(*)c1*]

is a point of attachment to a linking group which connects to the first point of attachment,
or a regioisomer thereof.

In some embodiments, L has a structure

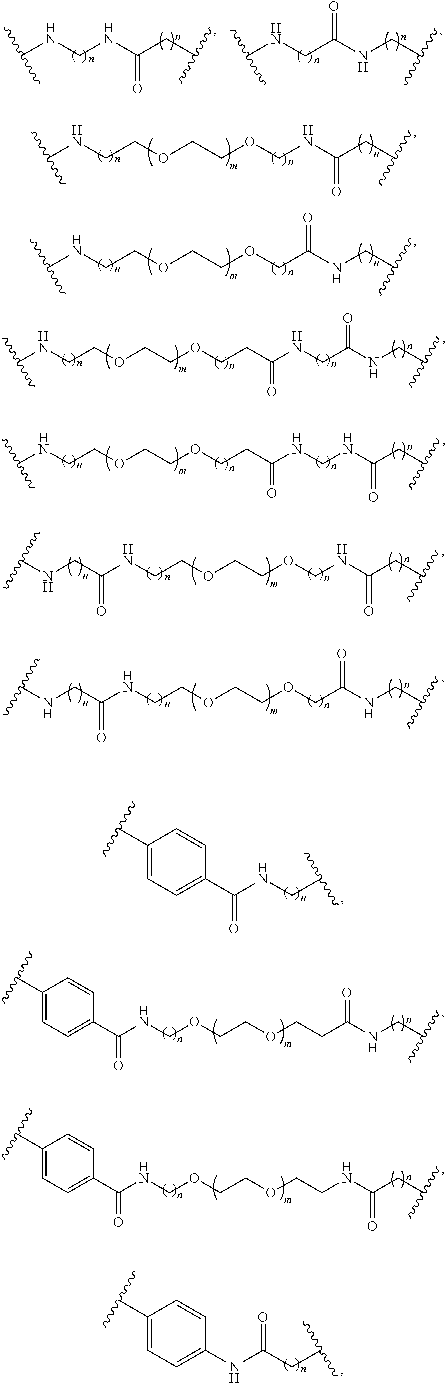

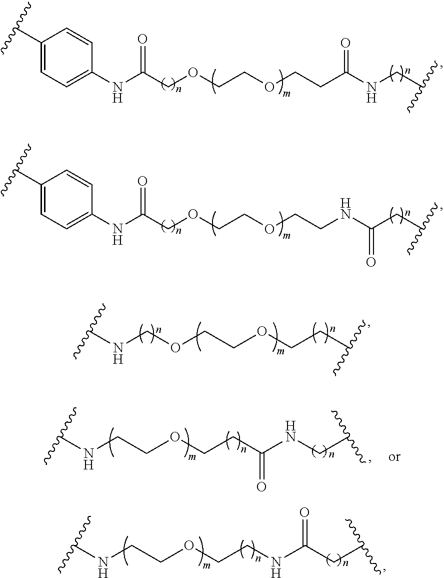

wherein each n is independently an integer from 1-6 and each m is an integer from 1-30. In some embodiments, each m is independently 2 or 3. In some embodiments, each m is an integer from 1-24, from 1-18, from 1-12, or from 1-6.

In some embodiments, the linker of Formula (X) or of Formula (X^{a}) or of Formula (X′) comprises the structure:

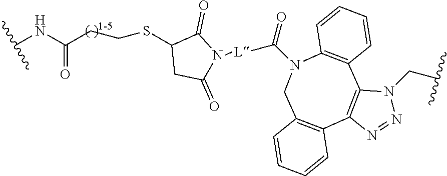

wherein

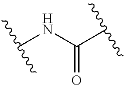 [SMILES: *NC(*)=O]

is the first point of attachment to a lysine residue of the polypeptide which selectively binds to PD-1;
L is a linking group; and

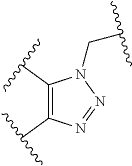 [SMILES: *Cn1nnc(*)c1*]

is a point of attachment to a linking group which connects to the first point of attachment,
or a regioisomer thereof.

In some embodiments, L″ has a structure

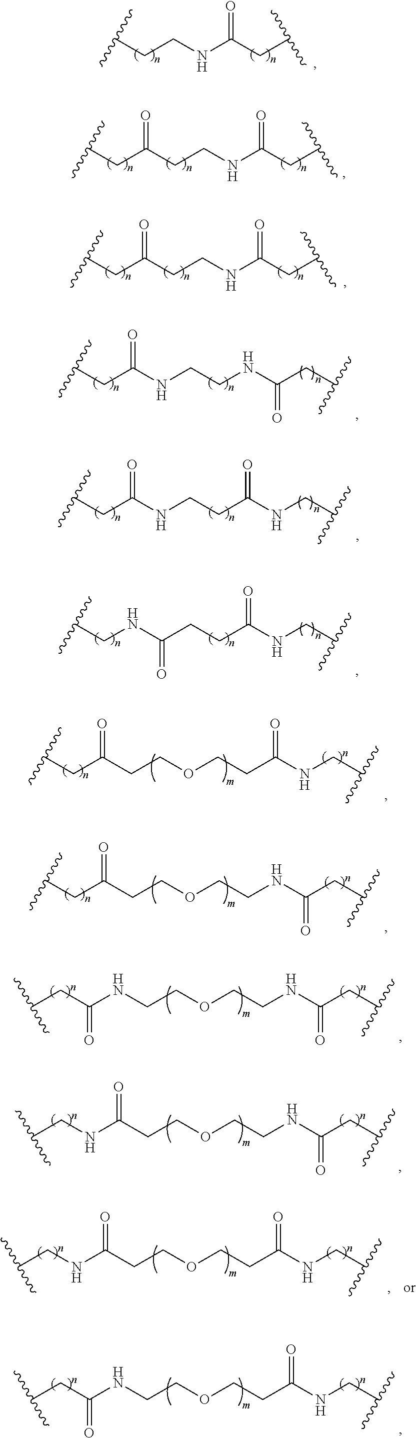

wherein each n is independently an integer from 1-6 and each m is independently an integer from 1-30. In some embodiments, each m is independently 2 or 3. In some embodiments, each m is an integer from 1-24, from 1-18, from 1-12, or from 1-6.

In some embodiments, L or L″ comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more subunits each independently selected from

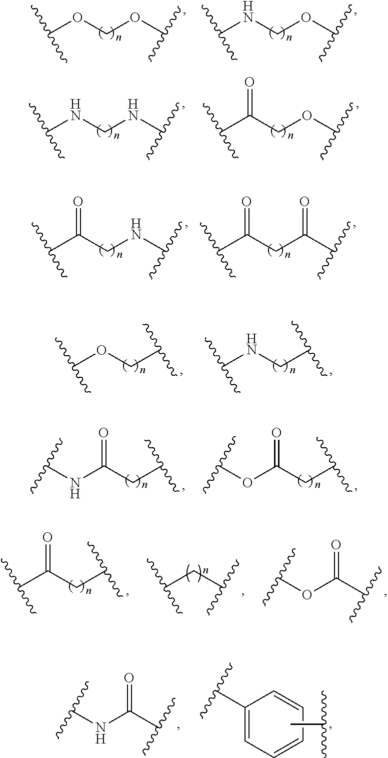

wherein each n is independently an integer from 1-30. In some embodiments, each n is independently an integer from 1-6. In some embodiments, L or L″ comprise 1, 2, 3, 4, 5, 6, 7, 8, 9, or 10 of the subunits.

In some embodiments, L or L″ is a structure of Formula (X″)

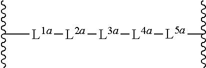

wherein each of L^{1a}, L^{2a}, L^{3a}, L^{4a}, L^{5a}, is independently —O—, —NR^{La}—, —(C₁-C₆ alkylene)NR^{La}—, NR^{La}(ci-C₆ alkylene)-, —N(R^{L})₂⁺—, —(C₁-C₆ alkylene)N(R^{La})₂+(C₁-C₆ alkylene)-, —N(R^{L})₂⁺—, —OP(═O)(OR^{La})O—, —S—, —(C₁-C₆ alkylene)S—, —S(C₁-C₆ alkylene)-, —S(═O)—, —S(═O)₂—, —C(═O)—, —(C₁-C₆ alkylene)C(═O)—, —C(═O)(C₁-C₆ alkylene)-, —C(═O)O—, —OC(═O)—, —OC(═O)O—, —C(═O)NR^{La}—, —C(═O)NR^{La}(C₁-C₆ alkylene)-, —(C₁-C₆ alkylene)C(═O)NR^{La}—, —NR^{La}C(═O)—, —(C₁-C₆ alkylene)NR^{La}C(═O)—, —NR^{La}C(═O)(C₁-C₆ alkylene)-, —OC(═O)NR^{La}—, —NR^{La}C(═O)O—, —NR^{La}C(═O)NR^{La}—, —NR^{La}C(═S)NR^{La}—, —CR^{La}═N—, —N═CR^{La}, —NR^{La}S(═O)₂—, —S(═O)₂NR^{LA}—, —C(═O)NR^{La}S(═O)₂—, —S(═O)₂NR^{La}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qe}—, —(O—CH₂—CH₂)_{qf}—, —(CH₂—CH(CH₃)—O)_{qg}—, —(O—CH(CH₃)—CH₂)_{qh}—, a reaction product of a conjugation handle and a complementary conjugation handle, or absent;
each R^{La} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
each of qe, qf, qg and qh is independently an integer from 1-100.

In some embodiments, L or L″ comprises a linear chain of 2 to 10, 2 to 15, 2 to 20, 2 to 25, or 2 to 30 atoms. In some embodiments, the linear chain comprises one or more alkyl groups (e.g., lower alkyl (C₁-C₄)), one or more aromatic groups (e.g., phenyl), one or more amide groups, one or more ether groups, one or more ester groups, or any combination thereof.

In some embodiments, the linking group which connects to the first point of attachment (e.g., the point of attachment to the cytokine) comprises poly(ethylene glycol). In some embodiments, the linking group comprises about 2 to about 30 poly(ethylene glycol) units. In some embodiments, the linking group which connects to the first point of attachment (e.g., the point of attachment to the cytokine) is a functionality attached to a cytokine provided herein which comprises an azide (e.g., the triazole is the reaction product of the azide).

In some embodiments, L is —O—, —N(R^{L})₂⁺—, —OP(═O)(OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)—NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}—, —(O—CH₂—CH₂)_{qb}—, —(CH₂—CH(CH₃)—O)_{qc}—, —(O—CH(CH₃)—CH₂)O—, wherein R^{L} hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and each of qa, qb, qc and qd is independently an integer from 1-100.

In some embodiments, each reaction product of a conjugation handle and a complementary conjugation handle independently comprises a triazole, a hydrazone, pyridazine, a sulfide, a disulfide, an amide, an ester, an ether, an oxime, or an alkene. In some embodiments, each reaction product of a conjugation handle and a complementary conjugation handle comprises a triazole. In some embodiments, each reaction product of a conjugation handle and a complementary

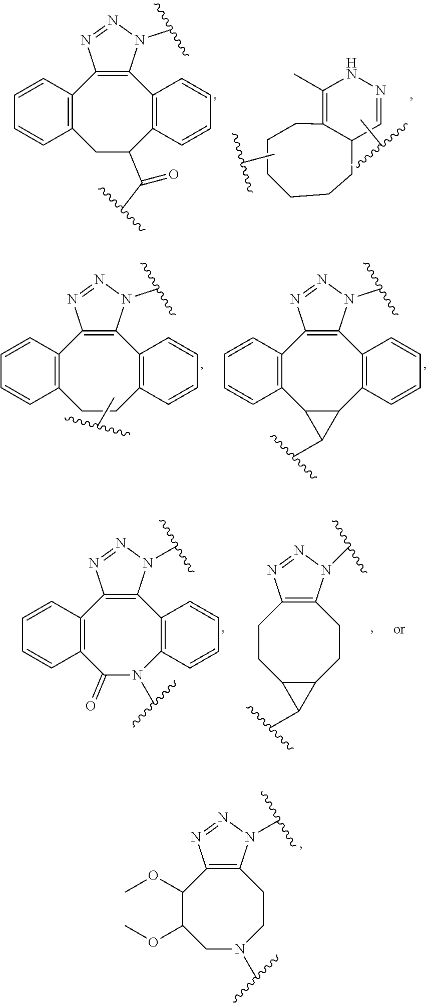

conjugation handle comprise a structure of

or a regioisomer or derivative thereof.

In some embodiments the linker is a cleavable linker. In some embodiments, the cleavable linker is cleaved at, near, or in a tumor microenvironment. In some embodiments, the tumor is mechanically or physically cleaved at, near, or in the tumor microenvironment. In some embodiments, the tumor is chemically cleaved at, near, or in a tumor microenvironment. In some embodiments, the cleavable linker is a reduction sensitive linker. In some embodiments, the cleavable linker is an oxidation sensitive linker. In some embodiments, the cleavable linker is cleaved as a result of pH at, near, or in the tumor microenvironment. In some embodiments, the linker by a tumor metabolite at, near, or in the tumor microenvironment. In some embodiments, the cleavable linker is cleaved by a protease at, near, or in the tumor microenvironment.

IL-2 Cytokines

Cytokines are proteins produced in the body that are important in cell signaling. Cytokines can modulate the immune system, and cytokine therapy utilizes the immunomodulatory properties of the molecules to enhance the immune system of a subject and kills cancer cells. Disclosed herein are anti-PD-1 polypeptides conjugated to cytokines, which can exhibit enhanced biological activity.

Interleukin-2 (IL-2) is a cytokine signaling molecule important in regulating the immune system. IL-2 is implicated in helping the immune system differentiate between foreign and endogenous cell types, thereby preventing the immune system from attacking a subject's own cells. IL-2 accomplishes its activity through interactions with IL-2 receptors (IL-2R) expressed by lymphocytes. Through these binding interactions, IL-2 can modulate a subject's populations of T-effector (T_{eff}) cells, natural killer (NK) cells, and regulatory T-cells (T_{reg}).

IL-2 has been used to treat cancer, both alone and in combination with other therapies. However, use of IL-2 as a treatment has been limited by the toxicity of IL-2, undesirable side effects such as vascular leak syndrome, and the short half-life of IL-2. Conjugation of IL-2 to an anti-PD-1 polypeptide of the disclosure can improve IL-2 polypeptide selectivity, enhance the therapeutic potential of IL-2, and potentially reduce the risk of side effects from administering IL-2 therapies.

The present disclosure describes anti-PD-1 polypeptides conjugated to modified interleukin-2 (IL-2) polypeptides and their use as therapeutic agents. Modified IL-2 polypeptides provided herein can be used as immunotherapies or as parts of other immunotherapy regimens. Such modified IL-2 polypeptides may display binding characteristics for the IL-2 receptor (IL-2R) that differ from wild-type IL-2. In one aspect, modified IL-2 polypeptides described herein have decreased affinity for the IL-2R aβγ complex (IL-2Ra). In some embodiments, the modified IL-2 polypeptides have an increased affinity for the IL-2R βγ complex (IL-2Rβ). In some embodiments, the binding affinity between the modified IL-2 polypeptides and IL-2Rβ is the same as or higher than the binding affinity between a wild-type IL-2 and IL-2Rβ. Non-limiting examples of IL-2 amino acid sequences to be utilized in embodiments described herein are provided below in Table 8.

In some embodiments of the instant disclosure, it is preferable that the IL-2 polypeptide is biased in favor of signaling through the IL-2 receptor beta subunit compared to wild type IL-2. In some embodiments, this is accomplished through one or both of a) inhibiting or diminishing binding of the IL-2 polypeptide to the IL-2 receptor alpha subunit (e.g., with a mutation at a residue contacting the alpha subunit, with addition of a polymer to the residue contacting the alpha subunit, or through attachment of the linker to the polypeptide which binds to PD-1 to the residue contacting the alpha subunit) and/or b) enhancing the binding of the IL-2 polypeptide to the beta subunit of the IL-2 receptor (e.g., with a mutation at a residue contacting the beta subunit which enhances binding). In some embodiments, the IL-2 polypeptide of the immunocytokine composition provided herein is biased towards the IL-2 receptor beta subunit compared to wild type IL-2. Non-limiting examples of IL-2 polypeptides with modifications which are biased towards IL-2 receptor beta signaling are described in, for example, PCT Publication Nos. WO2021140416A2, WO2012065086A1, WO2019028419A1, WO2012107417A1, WO2018119114A1, WO2012062228A2, WO2019104092A1, WO2012088446A1, and WO2015164815A1, each of which is hereby incorporated by reference as if set forth herein in its entirety.

Points of Attachment of Chemical Linkers to IL-2 Polypeptides

Provided herein are compositions comprising polypeptides, such as antibodies, which bind to PD-1 that are connected to IL-2 polypeptides by a chemical linker. As discussed supra, the chemical linker can be attached to the anti-PD-1 polypeptide at any of the positions provide herein. The second point of attachment of the linker is attached to an IL-2 polypeptide as provided herein.

In some embodiments, the chemical linker is attached to the IL-2 polypeptide at an amino acid residue. In some embodiments, the chemical linker is attached at an amino acid residue corresponding to any one of amino acid residues 1-133 of SEQ ID NO: 1. In some embodiments, the chemical linker is attached at a non-terminal amino acid residue (e.g., any one of amino acid residues 2-132 of SEQ ID NO: 1, or any one of amino acid residues 1-133 of SEQ ID NO: 1, wherein either the N-terminus or C-terminus has been extended by one or more amino acid residues). In some embodiments, the chemical linker is attached at a non-terminal amino acid residue of the IL-2 polypeptide, wherein the IL-2 polypeptide comprises either an N-terminal truncation or a C-terminal truncation relative to SEQ ID NO: 1.

In some embodiments, the chemical linker is attached to the IL-2 polypeptide at an amino acid residue which interacts with an IL-2 receptor (IL-2R) protein or subunit. In some embodiments, the chemical linker is attached at an amino acid residue which interacts with the IL-2R alpha subunit (IL-2Ra), the IL-2R beta subunit (IL-2Rβ), or the IL-2R gamma subunit (IL-2R□). In some embodiments, the chemical linker is attached at an amino acid residue which interacts with the IL-2R alpha subunit (IL-2Ra). In some embodiments, the chemical linker is attached at an amino acid residue which interacts with the IL-2R beta subunit (IL-2Rβ). In some embodiments, the chemical linker is attached at an amino acid residue which interacts with the IL-2R gamma subunit (IL-2Ry).

In some embodiments, the point of attachment to the IL-2 polypeptide is selected such that the interaction of the IL-2 polypeptide with at least one IL-2 receptor subunit is decreased or blocked. In some embodiments, the point of attachment is selected such that interaction of the IL-2 polypeptide with the IL-2Ra is reduced or blocked. In some embodiments, the point of attachment is selected such that interaction of the IL-2 polypeptide with the IL-2Rβ is reduced.

In some embodiments, the linker is attached to the IL-2 polypeptide at a residue which disrupts binding of the IL-2 polypeptide with the IL-2 receptor alpha subunit (IL-2Ra). Examples of these residues include residues 3, 5, 34, 35, 36, 37, 38, 40, 41, 42, 43, 44, 45, 60, 61, 62, 63, 64, 65, 67, 68, 69, 71, 72, 103, 104, 105, and 107, as described in, for example, PCT Pub. Nos. WO2019028419A1, WO2020056066A1, WO2021140416A2, and WO2021216478A1 each of which is hereby incorporated by reference as if set forth in its entirety.

In some embodiments, the linker is attached to the IL-2 polypeptide at an amino acid residue at any one of positions 1-110, wherein residue position numbering of the modified IL-2 polypeptide is based on SEQ ID NO: 1 as a reference sequence. In some embodiments, the linker is attached to the IL-2 polypeptide at an amino acid residue at any one of positions 1-10, 1-20, 1-30, 30-50, 30-70, 30-100, 40-50, 40-70, 40-100, or 40-110. In some embodiments, the linker is attached to the IL-2 polypeptide at an amino acid residue at any one of positions 1, 35, 37, 38, 41, 42, 43, 44, 45, 60, 61, 62, 64, 65, 68, 69, 71, 72, 104, 105, and 107, wherein amino acid residue position numbering of the modified IL-2 polypeptide is based on SEQ ID NO: 1 as a reference sequence. In some embodiments, the linker is attached to the IL-2 polypeptide at an amino acid residue at any one of positions 1, 35, 37, 38, 41, 42, 43, 44, 45, 60, 61, 62, 64, 65, 68, 69, 71, 72, 104, 105, and 107, wherein amino acid residue position numbering of the modified IL-2 polypeptide is based on SEQ ID NO: 1 as a reference sequence. In some embodiments, the linker is attached to the IL-2 polypeptide at an amino acid residue at any one of positions 1, 35, 37, 38, 41, 42, 43, 44, 60, 61, 62, 64, 65, 68, 69, 71, 72, 104, 105, and 107, wherein amino acid residue position numbering of the modified IL-2 polypeptide is based on SEQ ID NO: 1 as a reference sequence. In some embodiments, the linker is attached to the IL-2 polypeptide at an amino acid residue at any one of positions 1, 35, 37, 38, 41, 43, 44, 60, 61, 62, 64, 65, 68, 69, 71, 72, 104, 105, and 107, wherein amino acid residue position numbering of the modified IL-2 polypeptide is based on SEQ ID NO: 1 as a reference sequence In some embodiments, the linker is attached to the IL-2 polypeptide at an amino acid residue at any one of positions 1, 35, 37, 38, 39, 40, 41, 42, 43, 44, 45, or 46. In some embodiments, the linker is attached to the IL-2 polypeptide at an amino acid residue at any one of positions 1, 41, 42, 43, 44, and 45, wherein amino acid residue position numbering of the modified IL-2 polypeptide is based on SEQ ID NO: 1 as a reference sequence. In some embodiments, the linker is attached at amino acid residue 1, 42 or 45. In some embodiments, the linker is attached at amino acid residue 1. In some embodiments, the linker is attached at amino acid residue 42. In some embodiments, the linker is attached at amino acid residue 45.

In some embodiments, the linker is attached to a residue which is a natural amino acid residue of an IL-2 polypeptide as set forth in SEQ ID NO: 1. In some embodiments, the linker is attached to an amino acid residue which is a modified version of the natural amino acid residue of an IL-2 polypeptide as set forth in SEQ ID NO: 1. Non-limiting examples of such modification include incorporation or attachment of a conjugation handle to the natural amino acid residue (including through a linker), or attachment of the chemical linker to the natural amino acid using any compatible method. In some embodiments, the linker is attached to an amino acid residue which is a substituted amino acid residue compared to the IL-2 polypeptide of SEQ ID NO: 1. The substitution can be for a naturally occurring amino acid which is more amenable to attachment of additional functional groups (e.g., aspartic acid, cysteine, glutamic acid, lysine, serine, threonine, or tyrosine), a derivative of modified version of any naturally occurring amino acid, or any unnatural amino acid (e.g., an amino acid containing a desired reactive group, such as a CLICK chemistry reagent such as an azide, alkyne, etc.). Non-limiting examples of amino acids which can be substituted include, but are not limited to, -alpha-(9-Fluorenylmethyloxycarbonyl)-L-biphenylalanine (Fmoc-L-Bip-OH) and N-alpha-(9-Fluorenylmethyloxycarbonyl)-O-benzyl-L-tyrosine (Fmoc-L-Tyr(Bzl)-OH. Exemplary non-canonical amino acids include p-acetyl-L-phenylalanine, p-iodo-L-phenylalanine, p-methoxyphenylalanine, O-methyl-L-tyrosine, p-propargyloxyphenylalanine, p-propargyl-phenylalanine, L-3-(2-naphthyl)alanine, 3-methyl-phenylalanine, O-4-allyl-L-tyrosine, 4-propyl-L-tyrosine, tri-O-acetyl-GlcNAcp-serine, L-Dopa, fluorinated phenylalanine, isopropyl-L-phenylalanine, p-azido-L-phenylalanine, p-acyl-L-phenylalanine, p-benzoyl-L-phenylalanine, p-Boronophenylalanine, O-propargyltyrosine, L-phosphoserine, phosphonoserine, phosphonotyrosine, p-bromophenylalanine, selenocysteine, p-amino-L-phenylalanine, isopropyl-L-phenylalanine, azido-lysine (AzK), an analogue of a tyrosine amino acid; an analogue of a glutamine amino acid; an analogue of a phenylalanine amino acid; an analogue of a serine amino acid; an analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, a β-amino acid; a cyclic amino acid other than proline or histidine; an aromatic amino acid other than phenylalanine, tyrosine or tryptophan; or a combination thereof. In some embodiments, the non-canonical amino acids are selected from β-amino acids, homoamino acids, cyclic amino acids and amino acids with derivatized side chains. In some embodiments, the non-canonical amino acids comprise β-alanine, β-aminopropionic acid, piperidinic acid, aminocaprioic acid, aminoheptanoic acid, aminopimelic acid, desmosine, diaminopimelic acid, N^{α}-ethylglycine, N^{α}-ethylaspargine, hydroxylysine, allo-hydroxylysine, isodesmosine, allo-isoleucine, ω-methylarginine, N^{α}-methylglycine, N^{α}-methylisoleucine, N^{α}-methylvaline, γ-carboxyglutamate, ε-N,N,N-trimethyllysine, ε-N-acetyllysine, O-phosphoserine, N^{α}-acetylserine, N^{α}-formylmethionine, 3-methylhistidine, 5-hydroxylysine, and/or other similar amino acids.

In some embodiments, the linker is attached at an unnatural amino acid residue. In some embodiments, the unnatural amino acid residue comprises a conjugation handle. In some embodiments, the conjugation handle facilitates the addition of the linker to the modified IL-2 polypeptide. The conjugation handle can be any of the conjugation handles provided herein. In some embodiments, the linker is covalently attached site-specifically to the unnatural amino acid. Non-limiting examples of amino acid residues comprising conjugation handles can be found, for example, in PCT Pub. Nos. WO2015054658A1, WO2014036492A1, and WO2021133839A1 WO2006069246A2, and WO2007079130A2, each of which is incorporated by reference as if set forth in its entirety.

In some embodiments, the linker is attached to an amino acid residue which has been substituted with a natural amino acid. In some embodiments, the linker is attached to an amino acid residue which has been substituted with a cysteine, lysine, or tyrosine residue. In some embodiments, the linker is attached to a residue which has been substituted with a cysteine residue. In some embodiments, the linker is attached to an amino acid residue which has been substituted with a lysine residue. In some embodiments, the linker is attached to an amino acid residue which has been substituted with a tyrosine residue.

In some embodiments, the linker is attached to the amino acid residue at the N-terminal, A1, K35, F42Y, K43, F44Y, or Y45. In some embodiments, the linker is attached to the amino acid residue at the N-terminal, A1, F42Y or Y45. In some embodiments, the linker is attached to the amino terminal residue. In some embodiments, the linker is attached to amino acid residue A1. In some embodiments, the linker is attached to amino acid residue F42Y. In some embodiments, the linker is attached to amino acid residue Y45.

Modifications to IL-2 Polypeptides

In some embodiments, the modified IL-2 polypeptides described herein contain one or more modified amino acid residues. Such modifications can take the form of mutations of a wild type IL-2 polypeptide such as the amino acid sequence of SEQ ID NO: 1, addition and/or deletion of amino acids from the sequence of SEQ ID NO: 1, or the addition of moieties to amino acid residues. In some embodiments, the modified IL-2 polypeptide described herein contains a deletion of the first amino acid from the sequence of SEQ ID NO: 1. In some embodiments, the modified IL-2 polypeptide described herein comprises a C125S mutation, using the sequence of SEQ ID NO: 1 as a reference sequence. Moieties which can be added to amino acid residues include, but are not limited to, polymers, linkers, spacers, and combinations thereof. When added to certain amino acid residues, these moieties can modulate the activity or other properties of the modified IL-2 polypeptide compared to wild-type IL-2. In some embodiments, the modified IL-2 polypeptides comprise two modifications in the range of amino acid residues 35-46. In some embodiments, one modification is in the range of amino acid residues 40-43. In some embodiments, one modification is at amino acid residue 42. In some embodiments, one modification is in the range of amino acid residues 44-46. In some embodiments, one modification is at amino acid residue 45.

In some embodiments, the modified IL-2 polypeptides described herein contain one or more polymers. For example, the addition of polymers to certain amino acid residues can have the effect of disrupting the binding interaction of the modified IL-2 polypeptide with IL-2R, particularly the aβγ complex. In some embodiments, residues to which polymers are added to disrupt this interaction include F42 and Y45. In some embodiments, the polymer added to residue 42 or 45 also acts as the linker between the IL-2 polypeptide and the polypeptide which binds to PD-1.

In some embodiments, the polymers are water-soluble polymers, such as polyethylene glycol (PEG) polymers. The F42 residue can be mutated to another residue to facilitate the addition of the PEG polymer (or the linker), for example to a tyrosine residue. Polymers may be added to either one or both of residues F42 and Y45, or mutants thereof. These polymers may be either in the form of a linker between the IL-2 polypeptide and the polypeptide which selectively binds to PD-1 or may be an additional polymer in addition to the linker. In some embodiments, the modified IL-2 polypeptide comprises one or more amino acid mutations selected from TABLE 2.

TABLE 2

| WT IL-2 Residue | WT IL-2 | |
|---|---|---|
| Number* | Residue | Mutations |
| 31 | Y | H, F, A |
| 35 | K | R, D, I, L, M, N, P, Q, T, Y |
| 36 | L | A, D, E, F, G, H, I, K, M, N, |
| | | P, R, S, W, Y |
| 38 | R | A, D, G, K, N, P, S, Y |
| 40 | L | D, G, N, S, Y |
| 41 | T | E, G, Y |
| 42 | F | A, D, E, G, I, K, L, N, Q, R, |
| | | S, T, V, Y |
| 43 | K | H, Y |
| 44 | F | K, Y |
| 45 | Y | A, D, E, G, K, L, N, Q, R, S, |
| | | T, V |
| 46 | M | I, Y |
| 61 | E | K, M, R, Y |
| 62 | E | D, L, T, Y |
| 64 | K | D, E, G, L, Q, R, Y |
| 65 | P | D, E, F, G, H, I, K, L, N, Q, |
| | | R, S, T, V, W, Y |
| 66 | L | A, F, Y |
| 67 | E | A, Y |
| 68 | E | V, Y |
| 72 | L | A, D, E, G, K, N, Q, R, S, T, Y |
| 74 | Q | P, G, A |
| 88 | N | D, E, A |
| 125 | C | S |

*Residue position numbering based on SEQ ID NO: 1 as a reference sequence.

In some embodiments, a modified IL-2 polypeptide provided herein comprises one or more amino acid mutations selected from TABLE 3.

TABLE 3

| WT IL-2 | | |
|---|---|---|
| Residue | WT IL-2 | |
| Number* | Residue | Mutations |
| 20 | D | T, Y |
| 31 | Y | H, F, A |
| 35 | K | R, D, I, L, M, N, P, Q, T Y |
| 38 | R | A, D, G, K, N, P, S, Y |
| 42 | F | A, D, E, G, I, K, L, N, Q, R, S, T, V, Y |
| 43 | K | H, Y |
| 45 | Y | A, D, E, G, K, L, N, Q, R, S, T, V, Y |
| 62 | E | D, L, T, Y |
| 65 | P | D, E, F, G, H, I, K, L, N, Q, R, S, T, V, W, Y |
| 68 | E | V, Y |
| 72 | L | A, D, E, G, K, N, Q, R, S, T, Y |
| 74 | Q | P, G, A |
| 88 | N | D, E, A |
| 125 | C | S |

*Residue position numbering based on SEQ ID NO: 1 as a reference sequence.

In some embodiments, a modified IL-2 polypeptide provided herein comprises one or more polymers selected from TABLE 4.

TABLE 4

| Polymer | |
|---|---|
| Identifier | |
| and | |
| Approx. | |
| Molecular | |
| Weight | Polymer Structure |
| Formula D 500 Da | 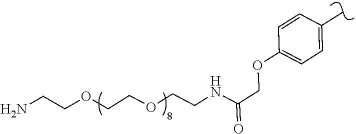 |

In some embodiments, a modified IL-2 polypeptide provided herein comprises mutations and polymers as provided in TABLE 5. In some embodiments, one or more of the polymers of table is replaced with or comprises a portion of the linker which is attached to the polypeptide which binds to PD-1.

TABLE 5

| | Polymer Residue | |
|---|---|---|
| Mutation* | Location | Polymer |
| F42Y | 42, 45 | Formula D (Residues 42, 45) |
| None | 45 | Formula D |
| F42A | 45 | Formula D |
| F42Y, L72G | 42, 45 | Formula D (Residues 42, 45) |
| F42Y, P65Y | 42, 65 | Formula D (Residues 42, 65) |
| F42Y, P65Y | 42, 45, 65 | Formula D (Residues 42, 45, 65) |
| R38Y, F42Y, | 38, 42, 45, | Formula D (Residues 38, 42, 45, |
| E62Y, E68Y | 62, 68 | 62, 68) |
| F42Y, L72Y | 42, 45, 72 | Formula D (Residues 42, 45, 75) |
| F42Y, Y45K | 42 | Formula D |
| F42A | 45 | Formula D |
| L72G | 45 | Formula D |
| F42Y | 42, 45 | Formula D (Residue 42), linker to |
| | | polypeptide which selectively binds |
| | | to PD-1 (Residue 45) |
| F42Y | 42, 45 | Formula D (Residues 45), linker to |
| | | polypeptide which selectively binds |
| | | to PD-1 (Residue 42) |

*Residue position numbering based on SEQ ID NO: 1 as a reference sequence.

In some instances, the modified IL-2 polypeptides described herein may be recombinant. The modified IL-2 polypeptides described herein may also be synthesized chemically rather than expressed as recombinant polypeptides. Synthetic IL-2 polypeptides have been described, at least in PCT Publication No WO2021140416A2, US Patent Application Publication No US20190023760A1, and Asahina et al., Angew. Chem. Int. Ed. 2015, 54, 8226-8230, each of which is incorporated by reference as if set forth herein in its entirety. The modified IL-2 polypeptides can be made by synthesizing one or more fragments of the full-length modified IL-2 polypeptides, ligating the fragments together, and folding the ligated full-length polypeptide. In some embodiments, the modified IL-2 polypeptide comprises an F42Y mutation in the amino acid sequence, a first PEG polymer of about 500 Da covalently attached to amino acid residue F42Y, a second PEG polymer of about 500 Da covalently attached to amino acid residue Y45, and an optional third PEG polymer of about 6 kDa covalently attached to the N-terminus of the modified IL-2 polypeptide. In some embodiments, the PEG polymer comprises a portion of the linker which attached the IL-2 polypeptide to the polypeptide which binds to PD-1.

In some embodiments, the chemically synthesized IL-2 polypeptide comprises a conjugation handle attached to one or more residues to facilitate attachment of the linker to the polypeptide which selectively binds to PD-1. The conjugation handle may be any such conjugation handle provided herein and may be attached at any residue to which the linker may be attached. In some embodiments, the conjugation handle is attached to residue 42 or 45 of the IL-2 polypeptide. In some embodiments, the conjugation handle comprises an azide or an alkyne. Alternatively, in some embodiments, the conjugation handle is incorporated into an unnatural or modified natural amino acid of a recombinant IL-2 polypeptide. Recombinant IL-2 polypeptides with unnatural amino acids can be made using methods as described in, for example, Patent Cooperation Treaty Publication Nos. WO2016115168, WO2002085923, WO2005019415, and WO2005003294.

In some embodiments, the modified IL-2 polypeptides enhance T-effector (T_{eff}) or natural killer (NK) cell proliferation when administered to a subject. In some embodiments, the modified IL-2 polypeptides enhance T_{eff} or NK cell proliferation while preventing preferential activation of regulatory T cells (T_{reg}) when administered to a subject. In some embodiments, the modified IL-2 polypeptides increase CD8+T and NK cells. In some embodiments, the modified IL-2 polypeptides produce a T_{eff}/T_{reg} ratio of close to 1 when administered to a subject.

In one aspect, described herein is a modified polypeptide that comprises a modified interleukin-2 (IL-2) polypeptide, wherein the modified IL-2 polypeptide comprises a covalently attached first polymer. Described herein is a modified polypeptide comprising a modified interleukin-2 (IL-2) polypeptide, wherein the modified IL-2 polypeptide comprises a first polymer covalently attached at residue F42Y, and wherein residue position numbering of the modified IL-2 polypeptide is based on SEQ ID NO: 1 as a reference sequence. In some embodiments, the first polymer is the same as linker which attaches the IL-2 polypeptide and the polypeptide which selectively binds to PD-1. In some embodiments, the first polymer is an additional polymer which is distinct from the linker. In another aspect, described herein is a modified polypeptide, comprising: a modified interleukin-2 (IL-2) polypeptide, wherein the modified IL-2 polypeptide exhibits a reduced functional activity on cells expressing the high affinity heterotrimeric IL-2 receptor (IL-2Rα/β/γ) and a greater functional activity on cells expressing the intermediate affinity heterodimeric IL-2 receptor (IL-2Rβ/γ) as measured by half maximal effective concentration (EC50) in an agonist assay on primary Tregs (expressing IL-2Rα/β/γ receptor) and resting CD8+ Teff (expressing IL-2Rβ/γ receptor), and wherein a ratio of the EC50 value of the modified IL-2 polypeptide on IL-2Rβ over the EC50 value of the modified IL-2 polypeptide on IL-2Rα is below 3:1. In some instances, the modified IL-2 polypeptide is a modified IL-2 polypeptide described herein, a modified IL-2 polypeptide provided in Table 8 or Table 5, a modified IL-2 polypeptide having a mutation provided in Table 2 or Table 3, and/or a modified IL-2 polypeptide having a polymer provided in Table 4.

Biological Activity

In some embodiments, an immunoconjugate comprising of a modified IL-2 polypeptide conjugated to an anti-PD-1 polypeptide as provided herein shows enhanced affinity for immune cells expressing high levels of PD-1 (CD279) located within tumors (e.g., tumor infiltrating lymphocytes (TIL)) or tumor draining lymph nodes while exhibiting reduced affinity for immune cells in the periphery expressing low or moderate levels of surface PD-1.

In some embodiments, the immunoconjugate comprising a modified IL-2 polypeptide conjugated to an anti-PD-1 polypeptide exhibits enhanced exposure within tumors or tumor draining lymph nodes compared to exposure in plasma compared to non-targeted IL-2 polypeptide or non-targeted IL-2 immunoconjugate. In some embodiments, the ratio of exposure within tumors or tumor draining lymph nodes over exposure in plasma or serum of PD1-IL2 immunoconjugate is at least 2-fold, 5-fold, 10-fold, 20-fold, 30-fold, 40-fold or higher as compared to a non-targeted IL-2 immunoconjugate or unconjugated IL-2 polypeptide. In some embodiments, the half-life of a PD1-IL2 immunoconjugate within tumors or tumor draining lymph nodes is 10-fold to 100-fold higher compared to its half-life in plasma or serum. In some embodiments the ratio of exposure within tumors or tumor draining lymph nodes over exposure in plasma or serum of a PD1-IL2 immunoconjugate is 10-fold to 100-fold, 20-fold to 100-fold, 30-fold to 100-fold, 40-fold to 100-fold, 20-fold to 75-fold, 30-fold to 75-fold, 40-fold to 100-fold, or 40-fold to 75-fold higher as compared to a non-targeted IL-2 immunoconjugate or unconjugated IL-2 polypeptide.

In some embodiments, the PD1-IL2 immunoconjugate exhibits an enhanced ratio of tumoral or tumor draining lymph node exposure over plasma or serum exposure compared to an IL-2 immunoconjugate or IL-2 polypeptide not targeting PD-1. In some embodiments, a ratio of tumoral or tumor draining lymph node exposure over plasma or serum exposure of an anti-PD1-IL-2 immunoconjugate is at least 2-fold, 5-fold, 10-fold, 20-fold, 30-fold, 40-fold higher as compared to an IL-2 immunoconjugate or IL-2 polypeptide not targeting PD-1. In some embodiments, a ratio of tumoral or tumor draining lymph node exposure over plasma or serum exposure of an anti-PD1-IL-2 immunoconjugate is 10-fold to 100-fold higher as compared to an IL-2 immunoconjugate or IL-2 polypeptide not targeting PD-1. In some embodiments, a ratio of tumoral or tumor draining lymph node exposure over plasma or serum exposure of the PD1-IL2 immunoconjugate is 10-fold to 100-fold, 20-fold to 100-fold, 30-fold to 100-fold, 40-fold to 100-fold, 20-fold to 75-fold, 30-fold to 75-fold, 40-fold to 100-fold, or 40-fold to 75-fold higher as compared to a non-targeted IL-2 immunoconjugate or IL-2 polypeptide.

In some embodiments, a ratio of expansion of immune cell populations within tumors (e.g., tumor infiltrating lymphocytes (TIL)) and within tumor draining lymph nodes over the expansion of immune cell populations in other tissues (e.g., immune cells of the same type in other tissues) induced by a PD1-IL2 immunoconjugate is at least 1.5, 2, 2.5, 3, 3.5, 4, 4.5, 5 or more. In some embodiments, a ratio of expansion of immune cell populations within tumors and tumor draining lymph nodes over the expansion of immune cell populations in other tissues induced by a PD1-IL2 immunoconjugate is from about 1.5 to 10, about 2 to 10, about 2.5 to 10, about 3 to 10, about 1.5 to 8, about 2 to 8, about 2.5 to 8, about 3 to 8, about 1.5 to 6, about 2 to 6, about 2.5 to 6, or about 3 to 6. In some embodiments, the immune cell population is at least one selected from naïve CD8+ cells, CD4+ helper cells, CD8+ central memory cells, CD8+ effector memory cells, NK cells, NKT cells, or any combination thereof. In some embodiments, the ratio is measured at a specified time post-administration (e.g., 6 hours, 12 hours, 24 hours, 2 days, 3 days, 4 days, 5 days, 6 days, or 7 days).

In some embodiments, the PD1-IL2 immunoconjugate comprising of a modified IL-2 polypeptide conjugated to an anti-PD-1 polypeptide shows enhanced potency due to cis-signaling of the modified IL-2 polypeptide on cells expressing high levels of PD-1 as compared to cells expressing no or only moderate levels of PD-1. In some embodiments, for a PD1-IL2 immunoconjugate the ratio of the EC50 value of IL-2 pathway engagement (pSTAT5 assay) in cells expressing no or only moderate levels of PD-1 over the EC50 value of IL-2 pathway engagement (pSTAT5 assay) in cells expressing high levels of PD-1 is at last 10, at least 50, at least 100, at least 250, at least 500, at least 750, at least 1000, at least 1500, at least 2000, at least 2500, or at least 3000. In some embodiments, for a PD1-IL2 immunoconjugate the ratio of the EC50 value of IL-2 pathway engagement (pSTAT5 assay) in cells expressing no or only moderate levels of PD-1 over the EC50 value of IL-2 pathway engagement (pSTAT5 assay) in cells expressing high levels of PD-1 is at least 10-fold, at least 50-fold, at least 100-fold, at least 200-fold, at least 300-fold, at least 400-fold, at least 500-fold, at least 600-fold, at least 700-fold, at least 800-fold, at least 900-fold, or at least 1000-fold as compared to an IL-2 immunoconjugate or IL-2 polypeptide not targeting PD-1.

In some embodiments, the modified IL-2 polypeptides display activity which differs from a wild type IL-2. These modified biological activities provided herein below apply, in some embodiments, to the IL-2 polypeptide alone (e.g., not conjugated or otherwise attached to the polypeptide which binds to PD-1) as well as when the IL-2 polypeptide is conjugated or otherwise to the polypeptide which binds to PD-1 (e.g., the modified biological activity is retained upon conjugation or attachment). Thus, when a modified IL-2 polypeptide is described herein as having an indicated activity, it is also contemplated that immunocytokine compositions provided herein (e.g., the IL-2 polypeptide attached to the polypeptide which binds to PD-1) has the same activity.

In some embodiments, a modified IL-2 polypeptide described herein is capable of expanding CD4+ helper cell, CD8+ central memory cell, CD8+ effector memory cell, naïve CD8+ cell, Natural Killer (NK) cell, Natural killer T (NKT) cell populations, or a combination thereof. In some instances, the modified IL-2 polypeptide is a modified IL-2 polypeptide described herein, a modified IL-2 polypeptide provided in Table 8 or Table 5, a modified IL-2 polypeptide having a mutation provided in Table 2 or Table 3, and/or a modified IL-2 polypeptide having a polymer provided in Table 4.

In some embodiments, a modified IL-2 polypeptide described herein expands a cell population of effector T cells (T_{eff} cells). In some embodiments, the modified IL-2 polypeptide expands a cell population of T_{eff} cells by at least 1%, at least 2%, at least 5%, at least 10%, at least 15%, at least 20%, at least 30%, at least 40%, at least 50%, at least 100%, or at least 200% when the modified IL-2 polypeptide is in contact with the population. In some embodiments, the modified IL-2 polypeptide expands a cell population of T_{eff} cells by at least 20% when the modified IL-2 polypeptide is in contact with the population. In some embodiments, the modified IL-2 polypeptide expands a cell population of T_{eff} cells by at least 30% when the modified IL-2 polypeptide is in contact with the population. In some embodiments, the modified IL-2 polypeptide expands a cell population of T_{eff} cells by at least 40% when the modified IL-2 polypeptide is in contact with the population. In some embodiments, the modified IL-2 polypeptide expands a cell population of T_{eff} cells by at least 50% when the modified IL-2 polypeptide is in contact with the population. In some embodiments, the modified IL-2 polypeptide expands a cell population of T_{eff} cells by at least 100% when the modified IL-2 polypeptide is in contact with the population. In some embodiments, the modified IL-2 polypeptide expands a cell population of T_{eff} cells by at least 200% when the modified IL-2 polypeptide is in contact with the population.

In some embodiments, a modified IL-2 polypeptide described herein expands a cell population of effector T cells (T_{eff} cells). In some embodiments, the modified IL-2 polypeptide expands a cell population of T_{eff} cells by at most 5%, at most 10%, at most 20%, at most 30%, at most 40%, at most 50%, at most 75%, at most 100%, or at most 500% when the modified IL-2 polypeptide is in contact with the population. In some embodiments, the modified IL-2 polypeptide expands a cell population of T_{eff} cells by at most 5%, when the modified IL-2 polypeptide is in contact with the population. In some embodiments, the modified IL-2 polypeptide expands a cell population of T_{eff} cells by at most 20%, when the modified IL-2 polypeptide is in contact with the population. In some embodiments, the modified IL-2 polypeptide expands a cell population of T_{eff} cells by at most 50%, when the modified IL-2 polypeptide is in contact with the population. In some embodiments, the modified IL-2 polypeptide expands a cell population of T_{eff} cells by at most 100%, when the modified IL-2 polypeptide is in contact with the population. In some embodiments, the modified IL-2 polypeptide expands a cell population of T_{eff} cells by at most 500%, when the modified IL-2 polypeptide is in contact with the population.

In some embodiments, a ratio of cell population expansion of T_{eff} cells over cell population expansion of T_{reg} cells expanded by a modified IL-2 polypeptide described herein is from about 0.1 to about 15, from about 0.5 to about 10, from about 0.75 to about 5, or from about 1 to about 2. In some embodiments, a ratio of cell population expansion of T_{eff} cells over cell population expansion of T_{reg} cells expanded by the modified IL-2 polypeptide is from 0.1 to 15. In some embodiments, a ratio of cell population expansion of T_{eff} cells over cell population expansion of T_{reg} cells expanded by the modified IL-2 polypeptide is from 0.1 to 0.5, from 0.1 to 0.75, from 0.1 to 1, from 0.1 to 2, from 0.1 to 5, from 0.1 to 10, from 0.1 to 15, from 0.5 to 0.75, from 0.5 to 1, from 0.5 to 2, from 0.5 to 5, from 0.5 to 10, from 0.5 to 15, from 0.75 to 1, 0.75 to 2, from 0.75 to 5, from 0.75 to 10, from 0.75 to 15, from 1 to 2, from 1 to 5, from 1 to 10, from 1 to 15, from 2 to 5, from 2 to 10, from 2 to 15, from 5 to 10, from 5 to 15, from 10 to 15, or any numbers or ranges therebetween. In some embodiments, a ratio of cell population expansion of T_{eff} cells over cell population expansion of T_{reg} cells expanded by the modified IL-2 polypeptide is about 0.1, 0.5, 0.75, 1, 2, 5, 10, or 15. In some embodiments, a ratio of cell population expansion of T_{eff} cells over cell population expansion of T_{reg} cells expanded by the modified IL-2 polypeptide is at least 0.1, 0.5, 0.75, 1, 2, 5, or 10. In some embodiments, a ratio of cell population expansion of T_{eff} cells over cell population expansion of T_{reg} cells expanded by the modified IL-2 polypeptide is at most 0.5, 0.75, 1, 2, 5, 10, or 15.

In some embodiments, a cell population expanded by a modified IL-2 polypeptide provided herein is an in vitro cell population, an in vivo cell population, or an ex vivo cell population. In some embodiments, the cell population is an in vitro cell population. In some embodiments, the cell population is an in vivo cell population. In some embodiments, the cell population is an ex vivo cell population. The cell population may be a population of CD4+ helper cells, CD8+ central memory cells, CD8+ effector memory cells, naïve CD8+ cells, Natural Killer (NK) cells, Natural killer T (NKT) cells, or a combination thereof.

In some embodiments, the levels of cells are measured 1 hour after injection of the modified IL-2 polypeptide. In some embodiments, the levels of cells are measured 2 hours after injection of the modified IL-2 polypeptide. In some embodiments, the levels of cells are measured 4 hours after injection of the modified IL-2 polypeptide. In some embodiments, the levels of cells are measured 30 minutes after injection of the modified IL-2 polypeptide (e.g., for an in vitro experiment). In some embodiments, the level of cells are measured at extended time points (e.g., 6 h, 12 h, 24 h, 72 h, 96 h, 120 h, 144 h, 168 h, etc.), particularly for in vivo experiments.

In some embodiments, an immunoconjugate composition provided herein (e.g., a polypeptide which binds to PD-1 (e.g., an anti-PD-1 antibody such as Pembrolizumab or LZM-009) attached to an IL-2 polypeptide through a linker) maintains binding affinity associated with at least one of the components after formation of the linkage between the two groups. For example, in an immunoconjugate composition comprising an anti-PD-1 antibody or antigen binding fragment linked to an IL-2 polypeptide, in some embodiments the anti-PD-1 antibody or antigen binding fragment thereof retains binding to one or more Fc receptors. In some embodiments, the composition displays binding to one or more Fc receptors which is reduced by no more than about 5-fold, no more than about 10-fold, no more than about 15-fold, or no more than about 20-fold compared to the unconjugated antibody. In some embodiments, the one or more Fc receptors is the FcRn receptor, the FcγRI receptor (CD64), the FcγRIIa receptor (CD32a), the FcγRIIβ receptor (CD32β), the FcγRIII receptor (CD16a), or any combination thereof. In some embodiments, binding of the composition to each of the FcRn receptor, the FcγRI receptor (CD64), the FcγRIIa receptor (CD32α), and the FcγRIIβ receptor (CD32β), the FcγRIII receptor (CD16a), is reduced by no more than about 10-fold compared to the unconjugated antibody.

In some embodiments, binding of the polypeptide which binds to PD-1 (e.g., the antibody) to PD-1 is substantially unaffected by the conjugation with the IL-2 polypeptide. In some embodiments, the binding of the polypeptide to PD-1 is reduced by no more than about 5% compared to the unconjugated antibody.

Site-Specific Modification

In some embodiments, a modified IL-2 polypeptide described herein comprises one or more modifications at one or more amino acid residues. In some embodiments, the residue position numbering of the modified IL-2 polypeptide is based on SEQ ID NO: 1 as a reference sequence. In some embodiments, the amino acid residue position numbering of the modified IL-2 polypeptide is based on a wild-type human IL-2 polypeptide as a reference sequence. In some instances, the modified IL-2 polypeptide is a modified IL-2 polypeptide described herein, a modified IL-2 polypeptide provided in Table 8 or Table 5, a modified IL-2 polypeptide having a mutation provided in Table 2 or Table 3, and/or a modified IL-2 polypeptide having a polymer provided in Table 4.

Modifications to the polypeptides described herein encompass mutations, addition of various functionalities, deletion of amino acids, addition of amino acids, or any other alteration of the wild-type version of the protein or protein fragment. Functionalities which may be added to polypeptides include polymers, linkers, alkyl groups, detectable molecules such as chromophores or fluorophores, reactive functional groups, or any combination thereof. In some embodiments, functionalities are added to individual amino acids of the polypeptides. In some embodiments, functionalities are added site-specifically to the polypeptides. In some embodiments, the functionality comprises at least a portion of the linker used to attach the IL-2 polypeptide to the polypeptide which selectively binds to PD-1.

In some embodiments, a modified IL-2 polypeptide described herein comprises a modification at an amino acid residue from the region of residues 35-46, wherein the residue numbering is based on SEQ ID NO: 1. In some embodiments, the modification is at K35, L36, T37, R38, M39, L40, T41, F42, K43, F44, Y45, or M46. In some embodiments, the modification is at F42. In some embodiments, the modification is at Y45. In some embodiments, the modified IL-2 polypeptide comprises a modification at the N-terminal residue. In some embodiments, the modified IL-2 polypeptide comprises a C125S mutation. In some embodiments, the modified IL-2 polypeptide comprises an A1 deletion. In some embodiments, the modification comprises attachment of the linker which is used to attach the IL-2 polypeptide to the polypeptide which selectively binds to PD-1.

In some embodiments, a modified IL-2 polypeptide described herein comprises a first polymer covalently attached at an amino acid residue in any of residues 35-46, wherein amino acid residue position numbering of the modified IL-2 polypeptide is based on SEQ ID NO: 1 as a reference sequence. In some embodiments, the modified IL-2 polypeptide comprises a first polymer covalently attached at an amino acid residue in any of residues 39-43. In some embodiments, the modified IL-2 polypeptide comprises a first polymer covalently attached at amino acid residue F42. In some embodiments, the modified IL-2 polypeptide comprises a first polymer covalently attached at amino acid residue F42Y. In some embodiments, the modified IL-2 polypeptide comprises a first polymer covalently attached at an amino acid residue in any of residues 44-46. In some embodiments, the modified IL-2 polypeptide comprises a first polymer covalently attached at amino acid residue Y45. In some embodiments, the first polymer is part of the linker which attaches the IL-2 polypeptide to the polypeptide which selectively binds to PD-1. In some embodiments, the first polymer is a separate modification from the linker which attached the IL-2 polypeptide to the polypeptide which selectively binds to PD-1.

In some embodiments, a modified IL-2 polypeptide described herein comprises one or more PEGylated tyrosine located at an amino acid residue in the region from amino acid residue 35 to amino acid residue 45. In some embodiments, the one or more PEGylated tyrosine is located at amino acid residue 42, amino acid residue 45, or both. In some embodiments, the one or more PEGylated tyrosine is located at amino acid residue 42. In some embodiments, the one or more PEGylated tyrosine is located at amino acid residue 45. In some embodiments, the one or more PEGylated tyrosine is located at both amino acid residue 42 and amino acid residue 45. In some embodiments, the modified IL-2 polypeptide comprises two PEGylated tyrosines, each independently having a structure of Formula (I). A non-limiting set of modified IL-2 polypeptides provided herein with various linker points of attachment and polymers as provided herein is shown in Table 7 below.

TABLE 7

| IL-2 | Linker Point | Polymer 1 Point | Polymer 2 Point |
|---|---|---|---|
| Construct | of Attachment | of Attachment | of Attachment |
| A | N-terminus | Residue 42 | Residue 45 |
| B | N-terminus | Residue 42 | None |
| C | N-terminus | Residue 45 | None |
| D | Residue 42 | Residue 45 | None |
| E | Residue 42 | N-terminus | Residue 45 |
| F | Residue 42 | N-terminus | None |
| G | Residue 45 | Residue 42 | None |
| H | Residue 45 | N-terminus | Residue 42 |
| I | Residue 45 | N-terminus | None |
| J | N-terminus | Residue 65 | None |
| K | Residue 65 | N-terminus | None |

*Residue position numbering based on SEQ ID NO: 1 as a reference sequence

In some embodiments, a modified IL-2 polypeptide provided herein is synthetic. In one aspect, disclosed herein is a modified IL-2 polypeptide comprising one or more amino acid substitutions. In some embodiments, the modified IL-2 polypeptide comprises F42Y and Y45. In some embodiments, the modified IL-2 polypeptide comprises a homoserine (Hse) residue located in any one of amino acid residues 35-45. In some embodiments, the modified IL-2 polypeptide comprises a Hse residue located in any one of amino acid residues 61-81. In some embodiments, the modified IL-2 polypeptide comprises a Hse residue located in any one of amino acid residues 94-114. In some embodiments, the modified IL-2 polypeptide comprises 1, 2, 3, or more Hse residues. In some embodiments, the modified IL-2 polypeptide comprises Hse41, Hse71, Hse104, or a combination thereof. In some embodiments, the modified IL-2 polypeptide comprises Hse41, Hse71, and Hse104. In some embodiments, the modified IL-2 polypeptide comprises at least two amino acid substitutions, wherein the at least two amino acid substitutions are selected from (a) a homoserine (Hse) residue located in any one of amino acid residues 35-45; (b) a homoserine residue located in any one of amino acid residues 61-81; and (c) a homoserine residue located in any one of amino acid residues 94-114. In some embodiments, the modified IL-2 polypeptide comprises Hse41 and Hse71. In some embodiments, the modified IL-2 polypeptide comprises Hse41 and Hse104. In some embodiments, the modified IL-2 polypeptide comprises Hse71 and Hse104. In some embodiments, the modified IL-2 polypeptide comprises Hse41. In some embodiments, the modified IL-2 polypeptide comprises Hse71. In some embodiments, the modified IL-2 polypeptide comprises Hse104. In some embodiments, the modified IL-2 polypeptide comprises 1, 2, 3, or more norleucine (Nle) residues. In some embodiments, the modified IL-2 polypeptide comprises a Nle residue located in any one of residues 18-28. In some embodiments, the modified IL-2 polypeptide comprises one or more Nle residues located in any one of amino acid residues 34-50. In some embodiments, the modified IL-2 polypeptide comprises a Nle residue located in any one of amino acid residues 20-60. In some embodiments, the modified IL-2 polypeptide comprises three Nle substitutions. In some embodiments, the modified IL-2 polypeptide comprises Nle23, Nle39, and Nle46. In some embodiments, the modified IL-2 polypeptide comprises SEQ ID NO: 3. In some embodiments, the modified IL-2 polypeptide comprises SEQ ID NO: 3 with an A1 deletion. In some embodiments, the modified IL-2 polypeptide comprises SEQ ID NO: 4. In some embodiments, the modified IL-2 polypeptide comprises an A1 deletion. In some embodiments, the modified IL-2 polypeptide comprises SEQ ID NO: 4 with an A1 deletion.

In some embodiments, a modified IL-2 polypeptide provided herein comprises an amino acid sequence of any one of SEQ ID NOs: 3-23 provided in Table 8. In some embodiments, the modified IL-2 polypeptide comprises an amino acid sequence at least 80%, 85%, 90%, 95%, 99%, or 100% identical to the sequence of any one of SEQ ID NOs: 3-23. In some embodiments, the modified IL-2 polypeptide comprises an amino acid sequence of SEQ ID NO: 3. In some embodiments, the modified IL-2 polypeptide comprises an amino acid sequence at least 80%, 85%, 90%, 95%, 99%, or 100% identical to the sequence of SEQ ID NO: 3. In some embodiments, the modified IL-2 polypeptide comprises an amino acid sequence of SEQ ID NO: 4. In some embodiments, the modified IL-2 polypeptide comprises an amino acid sequence at least 80%, 85%, 90%, 95%, 99, or 100% identical to the sequence of SEQ ID NO: 4. In some embodiments, the modified IL-2 polypeptide comprises an amino acid sequence of SEQ ID NO: 9. In some embodiments, the modified IL-2 polypeptide comprises an amino acid sequence at least 80%, 85%, 90%, 95%, 99%, or 100% identical to the sequence of SEQ ID NO: 9. In some embodiments, the modified IL-2 polypeptide comprises an amino acid sequence of SEQ ID NO: 10. In some embodiments, the modified IL-2 polypeptide comprises an amino acid sequence at least 80%, 85%, 90%, 95%, 99%, or 100% identical to the sequence of SEQ ID NO: 10. In some embodiments, the modified IL-2 polypeptide comprises an amino acid sequence of SEQ ID NO: 11. In some embodiments, the modified IL-2 polypeptide comprises an amino acid sequence at least 80%, 85%, 90%, 95%, 99%, or 100% identical to the sequence of SEQ ID NO: 11. In some embodiments, the modified IL-2 polypeptide comprises an amino acid sequence of SEQ ID NO: 12. In some embodiments, the modified IL-2 polypeptide comprises an amino acid sequence at least 80%, 85%, 90%, 95%, 99%, or 100% identical to the sequence of SEQ ID NO: 12. In some embodiments, the modified IL-2 polypeptide comprises an amino acid sequence of SEQ ID NO: 13. In some embodiments, the modified IL-2 polypeptide comprises an amino acid sequence at least 80%, 85%, 90%, 95%, 99%, or 100% identical to the sequence of SEQ ID NO: 13. In some embodiments, the modified IL-2 polypeptide comprises an amino acid sequence of SEQ ID NO: 14. In some embodiments, the modified IL-2 polypeptide comprises an amino acid sequence at least 80%, 85%, 90%, 95%, 99%, or 100% identical to the sequence of SEQ ID NO: 14. In some embodiments, the modified IL-2 polypeptide comprises an amino acid sequence of SEQ ID NO: 15. In some embodiments, the modified IL-2 polypeptide comprises an amino acid sequence at least 80%, 85%, 90%, 95%, 99%, or 100% identical to the sequence of SEQ ID NO: 15. In some embodiments, the modified IL-2 polypeptide comprises an amino acid sequence of SEQ ID NO: 17. In some embodiments, the modified IL-2 polypeptide comprises an amino acid sequence at least 80%, 85%, 90%, 95%, 99%, or 100% identical to the sequence of SEQ ID NO: 17. In some embodiments, the modified IL-2 polypeptide comprises an amino acid sequence of SEQ ID NO: 18. In some embodiments, the modified IL-2 polypeptide comprises an amino acid sequence at least 80%, 85%, 90%, 95%, 99%, or 100% identical to the sequence of SEQ ID NO: 18. In some embodiments, the modified IL-2 polypeptide comprises an amino acid sequence of SEQ ID NO: 19. In some embodiments, the modified IL-2 polypeptide comprises an amino acid sequence at least 80%, 85%, 90%, 95%, 99%, or 100% identical to the sequence of SEQ ID NO: 19. In some embodiments, the modified IL-2 polypeptide comprises an amino acid sequence of SEQ ID NO: 20. In some embodiments, the modified IL-2 polypeptide comprises an amino acid sequence at least 80%, 85%, 90%, 95%, 99%, or 100% identical to the sequence of SEQ ID NO: 20. In some embodiments, the modified IL-2 polypeptide comprises an amino acid sequence of SEQ ID NO: 21. In some embodiments, the modified IL-2 polypeptide comprises an amino acid sequence at least 80%, 85%, 90%, 95%, 99%, or 100% identical to the sequence of SEQ ID NO: 21. In some embodiments, the modified IL-2 polypeptide comprises an amino acid sequence of SEQ ID NO: 22. In some embodiments, the modified IL-2 polypeptide comprises an amino acid sequence at least 80%, 85%, 90%, 95%, 99%, or 100% identical to the sequence of SEQ ID NO: 22. In some embodiments, the modified IL-2 polypeptide comprises an amino acid sequence of SEQ ID NO: 23. In some embodiments, the modified IL-2 polypeptide comprises an amino acid sequence at least 80%, 85%, 90%, 95%, 99%, or 100% identical to the sequence of SEQ ID NO: 23.

In some embodiments, a modified IL-2 polypeptide described herein comprises at least 3, at least 4, at least 5, at least 6, at least 7, or at least 9 amino acid substitutions. In some embodiments, the modified IL-2 polypeptide comprises 3 to 9 amino acid substitutions. In some embodiments, the modified IL-2 polypeptide comprises 3 or 4 amino acid substitutions, 3 to 5 amino acid substitutions, 3 to 6 amino acid substitutions, 3 to 7 amino acid substitutions, 3 to 9 amino acid substitutions, 4 or 5 amino acid substitutions, 4 to 6 amino acid substitutions, 4 to 7 amino acid substitutions, 4 to 9 amino acid substitutions, 5 or 6 amino acid substitutions, 5 to 7 amino acid substitutions, 5 to 9 amino acid substitutions, 6 or 7 amino acid substitutions, 6 to 9 amino acid substitutions, or 7 to 9 amino acid substitutions. In some embodiments, the modified IL-2 polypeptide comprises 3 amino acid substitutions, 4 amino acid substitutions, 5 amino acid substitutions, 6 amino acid substitutions, 7 amino acid substitutions, or 9 amino acid substitutions. In some embodiments, the modified IL-2 polypeptide comprises at most 4 amino acid substitutions, 5 amino acid substitutions, 6 amino acid substitutions, 7 amino acid substitutions, or 9 amino acid substitutions. In some embodiments, one or more of the amino acid substitutions are selected from Table 2. In some embodiments, one or more of the amino acid substitutions are selected from Table 3. In some instances, the modified IL-2 polypeptide is a modified IL-2 polypeptide described herein, a modified IL-2 polypeptide provided in Table 8 or Table 5, a modified IL-2 polypeptide having a mutation provided in Table 2 or Table 3, and/or a modified IL-2 polypeptide having a polymer provided in Table 4.

In some embodiments, a modified IL-2 polypeptide described herein comprises a second modification. In some embodiments, the modified IL-2 polypeptide comprises a third modification. In some embodiments, the modified IL-2 polypeptide comprises a second and a third modification.

In some embodiments, a modified IL-2 polypeptide described herein comprises at least about 80%, at least about 85%, at least about 90%, at least about 95%, at least about 96%, at least about 97%, at least about 98%, at least about 99%, or 100% sequence identity to SEQ ID NO: 3. In some embodiments, the sequence identity is measured by protein-protein BLAST algorithm using parameters of Matrix BLOSUM62, Gap Costs Existence:11, Extension:1, and Compositional Adjustments Conditional Compositional Score Matrix Adjustment.

A modified IL-2 polypeptide as described herein can comprise one or more non-canonical amino acids. In some embodiments, in some cases, Tyr 45 and/or Phe 42 are substituted with non-canonical amino acids. In some embodiments, one or more amino acids located at positions provided in Table 2 and/or Table 3 are substituted with one or more non-canonical amino acids. Non-canonical amino acids include, but are not limited to N-alpha-(9-Fluorenylmethyloxycarbonyl)-L-biphenylalanine (Fmoc-L-Bip-OH) and N-alpha-(9-Fluorenylmethyloxycarbonyl)-O-benzyl-L-tyrosine (Fmoc-L-Tyr(Bzl)-OH. Exemplary non-canonical amino acids include p-acetyl-L-phenylalanine, p-iodo-L-phenylalanine, p-methoxyphenylalanine, O-methyl-L-tyrosine, p-propargyloxyphenylalanine, p-propargyl-phenylalanine, L-3-(2-naphthyl)alanine, 3-methyl-phenylalanine, O-4-allyl-L-tyrosine, 4-propyl-L-tyrosine, tri-O-acetyl-GlcNAcp-serine, L-Dopa, fluorinated phenylalanine, isopropyl-L-phenylalanine, p-azido-L-phenylalanine, p-acyl-L-phenylalanine, p-benzoyl-L-phenylalanine, p-Boronophenylalanine, O-propargyltyrosine, L-phosphoserine, phosphonoserine, phosphonotyrosine, p-bromophenylalanine, selenocysteine, p-amino-L-phenylalanine, isopropyl-L-phenylalanine, azido-lysine (AzK), an analogue of a tyrosine amino acid; an analogue of a glutamine amino acid; an analogue of a phenylalanine amino acid; an analogue of a serine amino acid; an analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, a β-amino acid; a cyclic amino acid other than proline or histidine; an aromatic amino acid other than phenylalanine, tyrosine or tryptophan; or a combination thereof. In some embodiments, the non-canonical amino acids are selected from β-amino acids, homoamino acids, cyclic amino acids and amino acids with derivatized side chains. In some embodiments, the non-canonical amino acids comprise β-alanine, β-aminopropionic acid, piperidinic acid, aminocaprioic acid, aminoheptanoic acid, aminopimelic acid, desmosine, diaminopimelic acid, N^{α}-ethylglycine, N^{α}-ethylaspargine, hydroxylysine, allo-hydroxylysine, isodesmosine, allo-isoleucine, w-methylarginine, N^{α}-methylglycine, N^{α}-methylisoleucine, N^{α}-methylvaline, γ-carboxyglutamate, ε-N,N,N-trimethyllysine, E-N-acetyllysine, 0-phosphoserine, N^{α}-acetylserine, N^{α}-formylmethionine, 3-methylhistidine, 5-hydroxylysine, and/or other similar amino acids. In some embodiments, Tyr 45 and/or Phe 42 are substituted with modified tyrosine residues. In some embodiments, the modified tyrosine residues comprise an amino, azide, allyl, ester, and/or amide functional groups. In some embodiments, the modified tyrosine residue at position 42 or 45 is used as the point of attachment for the linker which attaches the IL-2 polypeptide to the polypeptide which selectively binds to PD-1. In some embodiments, the modified tyrosine residues at positions 42 and/or 45 have a structure built from precursors Structure 1, Structure 2, Structure 3, Structure 4, or Structure 5, wherein Structure 1 is:

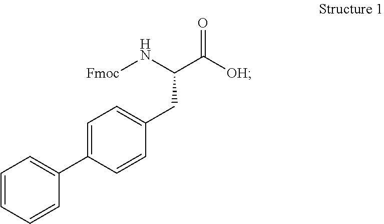

Structure 2 is:

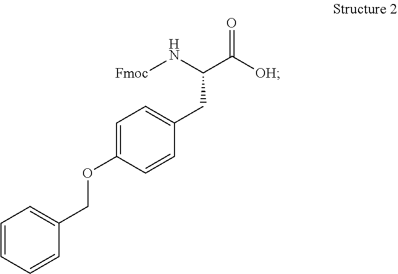

Structure 3 is:

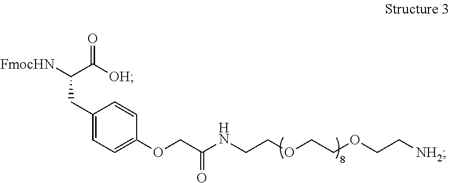

Structure 4 is:

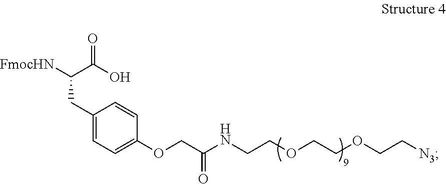

and Structure 5 is:

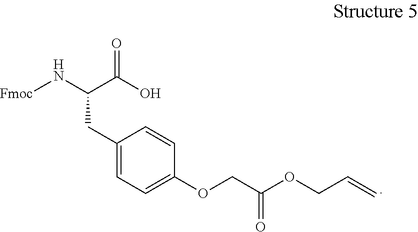

Polymers

In some embodiments, a herein described modified IL-2 polypeptide comprises one or more polymers covalently attached thereon. In some embodiments, the described modified IL-2 polypeptide comprises 1, 2, 3, 4, 5, 6, 7, 8, 9, 10, or more polymers covalently attached to the modified IL-2 polypeptide. In some embodiments, the described modified IL-2 polypeptide comprises a first polymer. In some embodiments, the first polymer comprises at least a portion of the linker which attached the IL-2 polypeptide to the polypeptide which selectively binds to PD-1. In some instances, the modified IL-2 polypeptide is a modified IL-2 polypeptide described herein, a modified IL-2 polypeptide provided in Table 4, a modified IL-2 polypeptide having a mutation provided in Table 2 or Table 3, and/or a modified IL-2 polypeptide having a polymer provided in Table 3.

In some embodiments, the first polymer comprises a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer is poly(alkylene oxide). In some embodiments, the water-soluble polymer is polysaccharide. In some embodiments, the water-soluble polymer is poly(ethylene oxide).

In some embodiments, a modified IL-2 polypeptide described herein comprises a first polymer covalently attached to the N-terminus of the IL-2 polypeptide. In some embodiments, the modified IL-2 polypeptide comprises a second polymer covalently attached thereto. In some embodiments, the modified IL-2 polypeptide comprises a second and a third polymer covalently attached thereto. In some embodiments, the second polymer is covalently attached to amino acid residue 42 or 45, wherein amino acid residue position numbering of the modified IL-2 polypeptide is based on SEQ ID NO: 1 as a reference sequence. In some embodiments, the second polymer is covalently attached to amino acid residue F42Y or Y45, wherein the amino acid residue position numbering of the modified IL-2 polypeptide is based on SEQ ID NO: 1 as a reference sequence. In some embodiments, the second and third polymers are covalently attached to amino acid residue 42 and 45, wherein amino acid residue position numbering of the modified IL-2 polypeptide is based on SEQ ID NO: 1 as a reference sequence. In some embodiments, the second and third polymers are covalently attached to amino acid residue F42Y and Y45, wherein amino acid residue position numbering of the modified IL-2 polypeptide is based on SEQ ID NO: 1 as a reference sequence. In some embodiments, at least one of the first, second, or third polymers comprises at least a portion of the linker used to attach the IL-2 polypeptide to the polypeptide which selectively binds to PD-1.

In some embodiments, the attached polymer such as the first polymer has a weight average molecular weight of about 120 Daltons to about 1,000 Daltons. In some embodiments, the polymer has a weight average molecular weight of about 120 Daltons to about 250 Daltons, about 120 Daltons to about 300 Daltons, about 120 Daltons to about 400 Daltons, about 120 Daltons to about 500 Daltons, about 120 Daltons to about 1,000 Daltons, about 250 Daltons to about 300 Daltons, about 250 Daltons to about 400 Daltons, about 250 Daltons to about 500 Daltons, about 250 Daltons to about 1,000 Daltons, about 300 Daltons to about 400 Daltons, about 300 Daltons to about 500 Daltons, about 300 Daltons to about 1,000 Daltons, about 400 Daltons to about 500 Daltons, about 400 Daltons to about 1,000 Daltons, or about 500 Daltons to about 1,000 Daltons. In some embodiments, the polymer has a weight average molecular weight of about 120 Daltons, about 250 Daltons, about 300 Daltons, about 400 Daltons, about 500 Daltons, or about 1,000 Daltons. In some embodiments, the polymer has a weight average molecular weight of at least about 120 Daltons, about 250 Daltons, about 300 Daltons, about 400 Daltons, or about 500 Daltons. In some embodiments, the polymer has a weight average molecular weight of at most about 250 Daltons, about 300 Daltons, about 400 Daltons, about 500 Daltons, or about 1,000 Daltons.

In some embodiments, the attached polymer such as the first polymer comprises a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer is poly(alkylene oxide) such as polyethylene glycol (e.g., polyethylene oxide). In some embodiments, the water-soluble polymer is polyethylene glycol. In some embodiments, the water-soluble polymer comprises modified poly(alkylene oxide). In some embodiments, the modified poly(alkylene oxide) comprises one or more linker groups. In some embodiments, the one or more linker groups comprise bifunctional linkers such as an amide group, an ester group, an ether group, a thioether group, a carbonyl group and alike. In some embodiments, the one or more linker groups comprise an amide linker group. In some embodiments, the modified poly(alkylene oxide) comprises one or more spacer groups. In some embodiments, the spacer groups comprise a substituted or unsubstituted C₁-C₆ alkylene group. In some embodiments, the spacer groups comprise —CH₂—, —CH₂CH₂—, or —CH₂CH₂CH₂—. In some embodiments, the linker group is the product of a biorthogonal reaction (e.g., biocompatible and selective reactions). In some embodiments, the bioorthogonal reaction is a Cu(I)-catalyzed or “copper-free” alkyne-azide triazole-forming reaction, the Staudinger ligation, inverse-electron-demand Diels-Alder (IEDDA) reaction, “photo-click” chemistry, or a metal-mediated process such as olefin metathesis and Suzuki-Miyaura or Sonogashira cross-coupling. In some embodiments, the first polymer is attached to the IL-2 polypeptide via click chemistry. In some embodiments, the first polymer comprises at least a portion of the linker which attaches the IL-2 polypeptide to the polypeptide which selectively binds to PD-1.

In some embodiments, a modified IL-2 polypeptide provided herein comprises a reaction group that facilitates the conjugation of the modified IL-2 polypeptide with a derivatized molecule or moiety such as an antibody and a polymer. In some embodiments, the reaction group comprises one or more of: carboxylic acid derived active esters, mixed anhydrides, acyl halides, acyl azides, alkyl halides, N-maleimides, imino esters, isocyanates, and isothiocyanates. In some embodiments, the reaction group comprises azides. In some embodiments, the reaction group forms a part of the linker which attaches the IL-2 polypeptide to the polypeptide which selectively binds to PD-1.

In some embodiments, a modified IL-2 polypeptide provided herein comprises a chemical reagent covalently attached to an amino acid residue. In some embodiments, the chemical reagent comprises a bioorthogonal reagent. In some embodiments, the chemical reagent comprises an azide. In some embodiments, the chemical reagent comprises an alkyne. In some embodiments, the chemical reagent is attached at an amino acid residue from 35-46, wherein the amino acid residue position numbering is based on SEQ ID NO: 1 as a reference sequence. In some embodiments, the chemical reagent is attached at an amino acid residue from 39-43, wherein the amino acid residue position numbering is based on SEQ ID NO: 1 as a reference sequence. In some embodiments, the chemical reagent is attached at amino acid residue 42, wherein the amino acid residue position numbering is based on SEQ ID NO: 1 as a reference sequence. In some embodiments, the chemical reagent is attached at amino acid residue F42Y, wherein the amino acid residue position numbering is based on SEQ ID NO: 1 as a reference sequence. In some embodiments, the chemical reagent is attached at an amino acid residue from 44-46, wherein the amino acid residue position numbering is based on SEQ ID NO: 1 as a reference sequence. In some embodiments, the chemical reagent is attached at amino acid residue 45, wherein the amino acid residue position numbering is based on SEQ ID NO: 1 as a reference sequence. In some embodiments, the chemical reagent is attached at any of the amino acid residues indicated in Table 2 or Table 3. In some embodiments, the chemical reagent forms a part of the linker which attaches the IL-2 polypeptide to the polypeptide which selectively binds to PD-1.

In some embodiments, the water-soluble polymer comprises from 1 to 10 polyethylene glycol chains.

In some embodiments, a modified IL-2 polypeptide described herein further comprises a second polymer covalently attached to the modified IL-2 polypeptide. In some embodiments, the second polymer is covalently attached at an amino acid residue region from residue 40 to residue 50. In some embodiments, the second polymer is covalently attached at amino acid residue Y45. In some embodiments, the second polymer is covalently attached to the N-terminus of the modified IL-2 polypeptide. In some embodiments, second polymer comprises at least a portion of the linker which attaches the IL-2 polypeptide to the polypeptide which selectively binds to PD-1.

In some embodiments, the second polymer has a weight average molecular weight of about 120 Daltons to about 1,000 Daltons. In some embodiments, the second polymer has a weight average molecular weight of about 120 Daltons to about 250 Daltons, about 120 Daltons to about 300 Daltons, about 120 Daltons to about 400 Daltons, about 120 Daltons to about 500 Daltons, about 120 Daltons to about 1,000 Daltons, about 250 Daltons to about 300 Daltons, about 250 Daltons to about 400 Daltons, about 250 Daltons to about 500 Daltons, about 250 Daltons to about 1,000 Daltons, about 300 Daltons to about 400 Daltons, about 300 Daltons to about 500 Daltons, about 300 Daltons to about 1,000 Daltons, about 400 Daltons to about 500 Daltons, about 400 Daltons to about 1,000 Daltons, or about 500 Daltons to about 1,000 Daltons. In some embodiments, the second polymer has a weight average molecular weight of about 120 Daltons, about 250 Daltons, about 300 Daltons, about 400 Daltons, about 500 Daltons, or about 1,000 Daltons. In some embodiments, the second polymer has a weight average molecular weight of at least about 120 Daltons, about 250 Daltons, about 300 Daltons, about 400 Daltons, or about 500 Daltons. In some embodiments, the second polymer has a weight average molecular weight of at most about 250 Daltons, about 300 Daltons, about 400 Daltons, about 500 Daltons, or about 1,000 Daltons.

In some embodiments, the second polymer comprises a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer is poly(alkylene oxide). In some embodiments, the water-soluble polymer is poly(ethylene oxide). In some embodiments, the second polymer is attached to the IL-2 polypeptide via click chemistry. In some embodiments, the second polymer comprises at least a portion of the linker which attaches the IL-2 polypeptide to the polypeptide which selectively binds to PD-1.

In some embodiments, the second water-soluble polymer comprises from 1 to 10 polyethylene glycol chains.

In some embodiments, a modified IL-2 polypeptide described herein further comprises a third polymer covalently attached to the modified IL-2 polypeptide. In some embodiments, the third polymer is covalently attached at an amino acid residue region from amino acid residue 40 to amino acid residue 50. In some embodiments, the third polymer is covalently attached at amino acid residue Y45. In some embodiments, the third polymer is covalently attached to the N-terminus of the modified IL-2 polypeptide.

In some embodiments, each polymer comprises a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, each water-soluble polymer is poly(alkylene oxide). In some embodiments, each water-soluble polymer is polyethylene glycol.

In some embodiments, each of the first polymer and the second polymer independently comprises from 1 to 5 polyethylene glycol chains. In some embodiments, each of the first polymer and the second polymer independently comprise single polyethylene glycol chains.

In some embodiments, each of the polyethylene glycol chains is independently linear or branched. In some embodiments, each of the polyethylene glycol chains is a linear polyethylene glycol. In some embodiments, each of the polyethylene glycol chains is a branched polyethylene glycol. For example, in some embodiments, each of the first and the second polymers comprises a linear polyethylene glycol chain.

In some embodiments, each of the polyethylene glycol chains is independently terminally capped with a hydroxy, an alkyl, an alkoxy, an amido, or an amino group. In some embodiments, each of the polyethylene glycol chains is independently terminally capped with an amino group. In some embodiments, each of the polyethylene glycol chains is independently terminally capped with an amido group. In some embodiments, each of the polyethylene glycol chains is independently terminally capped with an alkoxy group. In some embodiments, each of the polyethylene glycol chains is independently terminally capped with an alkyl group. In some embodiments, each of the polyethylene glycol chains is independently terminally capped with a hydroxy group.

In some embodiments, the modified IL-2 polypeptide comprises one or more PEGylated tyrosine having a structure of formula (I),

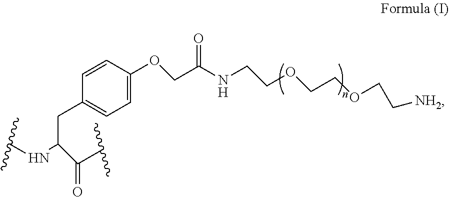

wherein n is an integer selected from 4 to 30. In some embodiments, n is 4 to 6, 4 to 8, 4 to 10, 4 to 15, 4 to 20, 4 to 25, 4 to 30, 6 to 8, 6 to 10, 6 to 15, 6 to 20, 6 to 25, 6 to 30, 8 to 10, 8 to 15, 8 to 20, 8 to 25, 8 to 30, 10 to 15, 10 to 20, 10 to 25, 10 to 30, 15 to 20, 15 to 25, 15 to 30, 20 to 25, 20 to 30, or 25 to 30. In some embodiments, n is 4, 6, 8, 10, 15, 20, 25, or 30. In some embodiments, n is at least 4, 6, 8, 10, 15, 20, or 25. In some embodiments, n is at most 6, 8, 10, 15, 20, 25, or 30. In one aspect, a modified IL-2 polypeptide as described herein comprises one or two water-soluble polymers covalently attached at one or two amino acid residues. For example, in some embodiments, the modified IL-2 polypeptide comprises one or two water-soluble polymers having the characteristics and attachment sites as shown in Table 6.

TABLE 6
Exemplary Polypeptides Structures and Water-soluble Polymer Characteristics

| Exemplary | | |
|---|---|---|
| Polypeptide | Characteristics of water-soluble | Characteristics of water-soluble |
| structures | polymer attached at residue 45 | polymer attached at residue 42 |
| 1 | Linear; | Linear; |
| | Mw: from about 200 to about 1000 Da | Mw: from about 200 to about 1000 Da |
| 2 | Linear; | None |
| | Mw: from about 200 to about 1000 Da | |
| 3 | None | Linear; |
| | | Mw: from about 200 to about 1000 Da |
| 4 | Linear; | Linear; |
| | Mw: from about 200 to about 1000 Da, | Mw: from about 200 to about 1000 Da |
| | optionally acts as linker to polypeptide | |
| | which selectively binds to PD-1 | |
| 5 | Linear; | Linear; Mw: from about 200 to about 1000 |
| | Mw: from about 200 to about 1000 Da | Da, optionally acts as linker to polypeptide |
| | | which selectively binds to PD-1 |
| 6 | None | Linear; |
| | | Mw: from about 200 to about 1000 Da |
| 7 | Linear; Mw: from about 200 to about | None |
| | 1000 Da, optionally acts as linker to | |
| | polypeptide which selectively binds to | |
| | PD-1 | |

In some embodiments, a water-soluble polymer that can be attached to a modified IL-2 polypeptide comprises a structure of Formula (D):

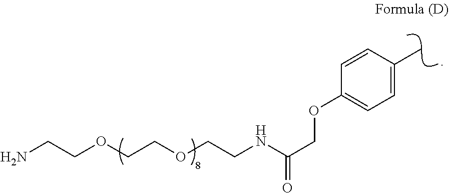

In some embodiments, the polymers are synthesized from suitable precursor materials. In some embodiments, the polymers are synthesized from the precursor materials of, Structure 6, Structure 7, Structure 8, or Structure 9, wherein Structure 6 is:

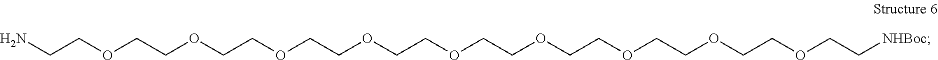 [SMILES: CC(C)(C)OC(=O)NCCOCCOCCOCCOCCOCCOCCOCCOCCOCCN]

Structure 7 is:

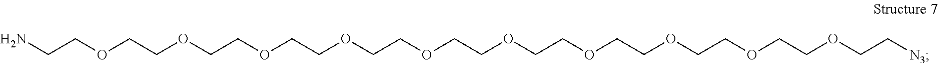 [SMILES: [N-]=[N+]=NCCOCCOCCOCCOCCOCCOCCOCCOCCOCCOCCN]

Structure 8 is:

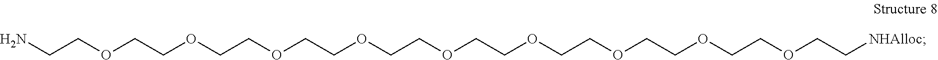

and Structure 9 is:

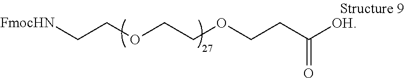

Orthogonal Payloads

The anti-PD-1-IL-2 immunoconjugates of the disclosure can comprise dual orthogonal payloads. In one non-limiting instance, the anti-PD-1-IL-2 immunoconjugates can comprise an anti-PD-1 polypeptide, one modified IL-2 polypeptide, and one payload that linked to the anti-PD-1 polypeptide by a chemical orthogonal linking group. The orthogonal payload can be an amino acid, amino acid derivative, peptide, protein, cytokine, alkyl group, aryl or heteroaryl group, therapeutic small molecule drug, polyethylene glycol (PEG) moiety, lipid, sugar, biotin, biotin derivative, deoxyribonucleic acid (DNA), ribonucleic acid (RNA), or peptide nucleic acid (PNA), any of which is substituted, unsubstituted, modified, or unmodified. In some embodiments, the orthogonal payload is a therapeutic small molecule. In some embodiments, the orthogonal payload is a PEG moiety. In some embodiments, the orthogonal payload is an additional cytokine, for example, IL-7 or IL-18. In one exemplary instance, human IL-7 has an amino acid sequence of DCDIEGKDGKQYESVLMVSIDQLLDSMKEIGSNCLNNEFNFFKRHICDANKEGMFLFRA ARKLRQFLKMNSTGDFDLEILLKVSEGTTILLNCTGQVKGRKPAALGEAQPTKSLEENKS LKEQKKLNDLCFLKRLLQEIKTCWNKILMGTKEH (SEQ ID NO: 117), or is a modified IL-7. In one exemplary instance, human IL-18 has an amino acid sequence of

(SEQ ID NO: 118)
YFIAEDDENLESDYFGKLESKLSVIRNLNDQVLFIDQGNRPLFEDMTDSD
CRDNAPRTIFIISMYKDSQPRGMAVTISVKCEKISTLSCENKIISFKEMN
PPDNIKDTKSDIIFFQRSVPGHDNKMQFESSSYEGYFLACEKERDLFKLI
LKKEDELGDRSIMFTVQNED,

or is a modified IL-18. In some instances, a conjugation handle can be added at one or more of Cys68, Glu69, Lys70 of IL-18.

Pharmaceutical Compositions

In one aspect, described herein is a pharmaceutical composition comprising: a polypeptide which selectively binds to PD-1 linked to a modified IL-2 polypeptide described herein; and a pharmaceutically acceptable carrier or excipient. In some embodiments, the pharmaceutical composition further comprises one or more excipients, wherein the one or more excipients include, but are not limited to, a carbohydrate, an inorganic salt, an antioxidant, a surfactant, a buffer, or any combination thereof. In some embodiments the pharmaceutical composition further comprises one, two, three, four, five, six, seven, eight, nine, ten, or more excipients, wherein the one or more excipients include, but are not limited to, a carbohydrate, an inorganic salt, an antioxidant, a surfactant, a buffer, or any combination thereof.

In some embodiments, the pharmaceutical composition further comprises a carbohydrate. In certain embodiments, the carbohydrate is selected from the group consisting of fructose, maltose, galactose, glucose, D-mannose, sorbose, lactose, sucrose, trehalose, cellobiose raffinose, melezitose, maltodextrins, dextrans, starches, mannitol, xylitol, maltitol, lactitol, xylitol, sorbitol (glucitol), pyranosyl sorbitol, myoinositol, cyclodextrins, and combinations thereof.

Alternately, or in addition, the pharmaceutical composition further comprises an inorganic salt. In certain embodiments, the inoragnic salt is selected from the group consisting of sodium chloride, potassium chloride, magnesium chloride, calcium chloride, sodium phosphate, potassium phosphate, sodium sulfate, or combinations thereof.

Alternately, or in addition, the pharmaceutical composition comprises an antioxidant. In certain embodiments, the antioxidant is selected from the group consisting of ascorbyl palmitate, butylated hydroxyanisole, butylated hydroxytoluene, potassium metabisulfite, propyl gallate, sodium metabisulfite, sodium thiosulfate, vitamin E, 3,4-dihydroxybenzoic acid, and combinations thereof.

Alternately, or in addition, the pharmaceutical composition further comprises a surfactant. In certain embodiments, the surfactant is selected from the group consisting of polysorbates, sorbitan esters, lipids, phospholipids, phosphatidylethanolamines, fatty acids, fatty acid esters, steroids, EDTA, zinc, and combinations thereof.

Alternately, or in addition, the pharmaceutical composition further comprises a buffer. In certain embodiments, the buffer is selected from the group consisting of citric acid, sodium phosphate, potassium phosphate, acetic acid, ethanolamine, histidine, amino acids, tartaric acid, succinic acid, fumaric acid, lactic acid, tris, HEPES, or combinations thereof.

In some embodiments, the pharmaceutical composition is formulated for parenteral or enteral administration. In some embodiments, the pharmaceutical composition is formulated for intravenous (IV) or subcutaneous (SQ) administration. In some embodiments, the pharmaceutical composition is in a lyophilized form.

In one aspect, described herein is a liquid or lyophilized composition that comprises a described a polypeptide which selectively binds to PD-1 linked to a modified IL-2 polypeptide. In some embodiments, the polypeptide which selectively binds to PD-1 linked to the modified IL-2 polypeptide modified IL-2 polypeptide is a lyophilized powder. In some embodiments, the lyophilized powder is resuspended in a buffer solution. In some embodiments, the buffer solution comprises a buffer, a sugar, a salt, a surfactant, or any combination thereof. In some embodiments, the buffer solution comprises a phosphate salt. In some embodiments, the phosphate salt is sodium Na₂HPO₄. In some embodiments, the salt is sodium chloride. In some embodiments, the buffer solution comprises phosphate buffered saline. In some embodiments, the buffer solution comprises mannitol. In some embodiments, the lyophilized powder is suspended in a solution comprising about 10 mM Na₂HPO₄ buffer, about 0.022% SDS, and about 50 mg/mL mannitol, and having a pH of about 7.5.

Dosage Forms

The polypeptide which selectively binds to PD-1 linked to the modified IL-2 polypeptides described herein can be in a variety of dosage forms. In some embodiments, polypeptide which selectively binds to PD-1 linked to the modified IL-2 polypeptide is dosed as a reconstituted lyophilized powder. In some embodiments, the polypeptide which selectively binds to PD-1 linked to the modified IL-2 polypeptide is dosed as a suspension. In some embodiments, the polypeptide which selectively binds to PD-1 linked to the modified IL-2 polypeptide is dosed as a solution. In some embodiments, the polypeptide which selectively binds to PD-1 linked to the modified IL-2 polypeptide is dosed as an injectable solution. In some embodiments, the polypeptide which selectively binds to PD-1 linked to the modified IL-2 polypeptides is dosed as an IV solution. In some embodiments, the polypeptide which selectively binds to PD-1 linked to the modified IL-2 polypeptide is administered by subcutaneous or intramuscular administration.

Methods of Treatment

In one aspect, described herein, is a method of treating cancer in a subject in need thereof, comprising: administering to the subject an effective amount of a polypeptide which selectively binds to PD-1 linked to a modified IL-2 polypeptide or a pharmaceutical composition as described herein. In some embodiments, the cancer is a solid cancer. A cancer or tumor can be, for example, a primary cancer or tumor or a metastatic cancer or tumor. Cancers and tumors to be treated include, but are not limited to, a melanoma, a lung cancer (e.g., a non-small cell lung cancer (NSCLC), a small cell lung cancer (SCLC), etc.), a carcinoma (e.g., a cutaneous squamous cell carcinoma (CSCC), a urothelial carcinoma (UC), a renal cell carcinoma (RCC), a hepatocellular carcinoma (HCC), a head and neck squamous cell carcinoma (HNSCC), an esophageal squamous cell carcinoma (ESCC), a gastroesophageal junction (GEJ) carcinoma, an endometrial carcinoma (EC), a Merkel cell carcinoma (MCC), etc.), a bladder cancer (BC), a microsatellite instability high (MSI-H)/mismatch repair-deficient (dMMR) solid tumor (e.g., a colorectal cancer (CRC)), a tumor mutation burden high (TMB-H) solid tumor, a triple-negative breast cancer (TNBC), a gastric cancer (GC), a cervical cancer (CC), a pleural mesothelioma (PM), classical Hodgkin's lymphoma (cHL), or a primary mediastinal large B cell lymphoma (PMBCL).

Combination therapies with one or more additional active agents are contemplated herein. In some embodiments, the second therapeutic agent is selected based on tumor type, tumor tissue of origin, tumor stage, or mutations in genes expressed by the tumor. For example, an anti-PD-1 antibody can be administered in combination with one or more of the following: a chemotherapeutic agent, an immune checkpoint inhibitor, an immune agonist, a biologic cancer agent, a low molecular weight anti-cancer agent, a synthetic peptide anti-cancer agent, an anti-cancer protein degrading agent, a cancer-specific agent, a cytokine therapy, an anti-angiogenic drug, a drug that targets cancer metabolism, an antibody that marks a cancer cell surface for destruction, an antibody-drug conjugate, a cell therapy, a commonly used anti-neoplastic agent, a CAR-T therapy, an oncolytic virus, a non-drug therapy, a neurotransmission blocker, or a neuronal growth factor blocker.

In some embodiments, the cancer is a solid cancer. In some embodiments, the solid cancer is adrenal cancer, anal cancer, bile duct cancer, bladder cancer, bone cancer, brain cancer, breast cancer, carcinoid cancer, cervical cancer, colorectal cancer, esophageal cancer, eye cancer, gallbladder cancer, gastrointestinal stromal tumor, germ cell cancer, head and neck cancer, kidney cancer, liver cancer, lung cancer, nasal cavity and paranasal sinus cancer, nasopharyngeal cancer, neuroblastoma, neuroendocrine cancer, oral cancer, oropharyngeal cancer, ovarian cancer, pancreatic cancer, pediatric cancer, penile cancer, pituitary cancer, prostate cancer, skin cancer, soft tissue cancer, spinal cord cancer, stomach cancer, testicular cancer, thymus cancer, thyroid cancer, ureteral cancer, uterine cancer, vaginal cancer, or vulvar cancer.

In some embodiments, the cancer is a blood cancer. In some embodiments, the blood cancer is leukemia, non-Hodgkin lymphoma, Hodgkin lymphoma, an AIDS-related lymphoma, multiple myeloma, plasmacytoma, post-transplantation lymphoproliferative disorder, or Waldenstrom macroglobulinemia.

An effective response is achieved when the subject experiences partial or total alleviation or reduction of signs or symptoms of illness, reduction of tumor burden, prolonging of time to increased tumor burden (progression of tumor), and specifically includes, without limitation, prolongation of survival. The expected progression-free survival times may be measured in months to years, depending on prognostic factors including the number of relapses, stage of disease, and other factors. Prolonging survival includes without limitation times of at least 1 month (mo), about at least 2 mos., about at least 3 mos., about at least 4 mos., about at least 6 mos., about at least 1 year, about at least 2 years, about at least 3 years, about at least 4 years, about at least 5 years, etc. Overall or progression-free survival can be also measured in months to years. Alternatively, an effective response may be that a subject's symptoms or cancer burden remain static and do not worsen. Further indications of treatment of indications are described in more detail below. In some instances, a cancer or tumor is reduced by at least 2%, 5%, 10%, 15%, 20%, 25%, 30%, 35%, 40%, 45%, 50%, 55%, 60%, 65%, 70%, 75%, 80%, 85%, 90%, 95%, or 100%.

In some embodiments, the polypeptide which selectively binds to PD-1 linked to the modified IL-2 polypeptide is administered in a single dose of the effective amount of the modified IL-2 polypeptide, including further embodiments in which (i) the polypeptide which selectively binds to PD-1 linked to the modified IL-2 polypeptide is administered once a day; or (ii) the polypeptide which selectively binds to PD-1 linked to the modified IL-2 polypeptide is administered to the subject multiple times over the span of one day. In some embodiments, the polypeptide which selectively binds to PD-1 linked to the modified IL-2 polypeptide is administered daily, every other day, 3 times a week, once a week, every 2 weeks, every 3 weeks, every 4 weeks, every 5 weeks, every 3 days, every 4 days, every 5 days, every 6 days, bi-weekly, 3 times a week, 4 times a week, 5 times a week, 6 times a week, once a month, twice a month, 3 times a month, once every 2 months, once every 3 months, once every 4 months, once every 5 months, or once every 6 months. Administration includes, but is not limited to, injection by any suitable route (e.g., parenteral, enteral, intravenous, subcutaneous, etc.). In preferred embodiments, the composition is administered weekly, every two weeks, every three weeks, or every four weeks.

Methods of Manufacturing

In one aspect, described herein, is a method of making a composition, comprising providing a polypeptide which selectively binds to PD-1, wherein the polypeptide which selectively binds to PD-1 comprises a reactive group (e.g., a conjugation handle), contacting the reactive group with a complementary reactive group attached to a cytokine, and forming the composition. The resulting composition is any of the compositions provided herein.

In some embodiments, the polypeptide which selectively binds to PD-1 is an antibody or an antigen binding fragment thereof. In some embodiments, providing the antibody comprising the reactive group comprises attaching the reactive group to the antibody. In some embodiments, the reactive group is added site-specifically. In some embodiments, attaching the reactive group to the antibody comprises contacting the antibody with an affinity group comprising a reactive functionality which forms a bond with a specific residue of the antibody. In some embodiments, attaching the reactive group to the antibody comprises contacting the antibody with an enzyme. In some embodiments, the enzyme is configured to site-specifically attach the reactive group to a specific residue of the antibody. In some embodiments, the enzyme is glycosylation enzyme or a transglutaminase enzyme.

In some embodiments, the method further comprises attaching the complementary reactive group to the cytokine. In some embodiments, attaching the complementary reactive group to the cytokine comprises chemically synthesizing the cytokine.

In some embodiments, the method comprises making a modified IL-2 polypeptide. In some embodiments, the method of making a modified IL-2 polypeptide comprises synthesizing two or more fragments of the modified IL-2 polypeptide and ligating the fragments. In some embodiments, the method of making the modified IL-2 polypeptide comprises a. synthesizing two or more fragments of the modified IL-2 polypeptide, b. ligating the fragments; and c. folding the ligated fragments.

In some embodiments, the two or more fragments of the modified IL-2 polypeptide are synthesized chemically. In some embodiments, the two or more fragments of the modified IL-2 polypeptide are synthesized by solid phase peptide synthesis. In some embodiments, the two or more fragments of the modified IL-2 polypeptide are synthesized on an automated peptide synthesizer.

In some embodiments, the modified IL-2 polypeptide is ligated from 2, 3, 4, 5, 6, 7, 8, 9, 10, or more peptide fragments. In some embodiments, the modified peptide is ligated from 2 peptide fragments. In some embodiments, the modified IL-2 polypeptide is ligated from 3 peptide fragments. In some embodiments, the modified IL-2 polypeptide is ligated from 4 peptide fragments. In some embodiments, the modified IL-2 polypeptide is ligated from 2 to 10 peptide fragments.

In some embodiments, the two or more fragments of the modified IL-2 polypeptide are ligated together. In some embodiments, three or more fragments of the modified IL-2 polypeptide are ligated in a sequential fashion. In some embodiments, three or more fragments of the modified IL-2 polypeptide are ligated in a one-pot reaction.

In some embodiments, ligated fragments are folded. In some embodiments, folding comprises forming one or more disulfide bonds within the modified IL-2 polypeptide. In some embodiments, the ligated fragments are subjected to a folding process. In some embodiments, the ligated fragments are folding using methods well known in the art. In some embodiments, the ligated polypeptide or the folded polypeptide are further modified by attaching one or more polymers thereto. In some embodiments, the ligated polypeptide or the folded polypeptide are further modified by PEGylation. In some embodiments, the modified IL-2 polypeptide is synthetic.

Sequences (SEQ ID NOS) of IL-2 Polypeptides

TABLE 8

| | SEQ | |
|---|---|---|
| | ID | |
| Substitutions | NO | Sequence |
| None (WT) | 1 | APTSSSTKKTQLQLEHLLLDLQMILNGINNYKNPKLTRML |
| | | TFKFYMPKKATELKHLQCLEEELKPLEEVLNLAQSKNFH |
| | | LRPRDLISNINVIVLELKGSETTFMCEYADETATIVEFLNR |
| | | WITFCQSIISTLT |
| ΔA1, C125S | 2 | PTSSSTKKTQLQLEHLLLDLQMILNGINNYKNPKLTRMLT |
| (Aldesleukin) | | FKFYMPKKATELKHLQCLEEELKPLEEVLNLAQSKNFHL |
| | | RPRDLISNINVIVLELKGSETTFMCEYADETATIVEFLNRW |
| | | ITFSQSIISTLT |
| M23Nle, | 3 | APTSSSTKKTQLQLEHLLLDLQ(Nle)ILNGINNYKNPKLTR |
| M39Nle | | (Nle)L(Hse)YKFY(Nle)PKKATELKHLQCLEEELKPLEEVL |
| T41Hse | | (Hse)LAQSKNFHLRPRDLISNINVIVLELKGSETTF(Hse) |
| F42Y, | | CEYADETATIVEFLNRWITFSQSIISTLT |
| M46Nle, | | |
| N71Hse, | | |
| M104Hse, | | |
| C125S | | |
| M23Nle, | 4 | APTSSSTKKTQLQLEHLLLDLQ(Nle)ILNGINNYKNPKLTR |
| M39Nle, | | (Nle)L(Hse)FKFY(Nle)PKKATELKHLQCLEEELKPLEEVL |
| T41Hse, | | (Hse)LAQSKNFHLRPRDLISNINVIVLELKGSETTF(Hse) |
| M46Hse, | | CEYADETATIVEFLNRWITFSQSIISTLT |
| N71Hse, | | |
| M104Hse, | | |
| C125S | | |
| M23Nle, | 5 | APTSSSTKKTQLQLEHLLLDLQ(Nle)ILNGINNYKNPKLTR |
| M39Nle, | | (Nle)L(Hse)YKFY(Nle)PKKATELKHLQCLEEELKPLEEVL |
| T41Hse, | | (Hse)YAQSKNFHLRPRDLISNINVIVLELKGSETTF(Hse) |
| F42Y, | | CEYADETATIVEFLNRWITFSQSIISTLT |
| M46Nle, | | |
| N71Hse, | | |
| L72Y, | | |
| M104Hse, | | |
| C125S | | |
| M23Nle, | 6 | APTSSSTKKTQLQLEHLLLDLQ(Nle)ILNGINNYKNPKLTR |
| M39Nle, | | (Nle)L(Hse)FKFY(Nle)PKKATELKHLQCLEEELKPLEEVL |
| T41Hse, | | (Hse)YAQSKNFHLRPRDLISNINVIVLELKGSETTF(Hse) |
| M46Nle, | | CEYADETATIVEFLNRWITFSQSIISTLT |
| N71Hse, | | |
| L72Y, | | |
| M104Hse, | | |
| C125S | | |
| M23Nle, | 7 | APTSSSTKKTQLQLEHLLLDLQ(Nle)ILNGINNYKNPKLTR |
| M39Nle, | | (Nle)L(Hse)YKFY(Nle)PKKATELKHLQCLEEELKPLEEVL |
| T41Hse, | | (Hse)GAQSKNFHLRPRDLISNINVIVLELKGSETTF(Hse) |
| F42Y, | | CEYADETATIVEFLNRWITFSQSIISTLT |
| M46Nle, | | |
| N71Hse, | | |
| L72G, | | |
| M104Hse, | | |
| C125S | | |
| M23Nle, | 8 | APTSSSTKKTQLQLEHLLLDLQ(Nle)ILNGINNYKNPKLTR |
| M39Nle, | | (Nle)L(Hse)YKFY(Nle)PKKATELKHLQCLEEELKYLEEVL |
| T41Hse, | | (Hse)LAQSKNFHLRPRDLISNINVIVLELKGSETTF(Hse) |
| F42Y, | | CEYADETATIVEFLNRWITFSQSIISTLT |
| M46Nle, | | |
| P65Y, | | |
| N71Hse, | | |
| M104Hse, | | |
| C125S | | |
| M23Nle, | 9 | APTSSSTKKTQLQLEHLLLDLQ(Nle)ILNGINNYKNPKLTR |
| M39Nle, | | (Nle)L(Hse)FKFY(Nle)PKKATELKHLQCLEEELKYLEEVL |
| T41Hse, | | (Hse)LAQSKNFHLRPRDLISNINVIVLELKGSETTF(Hse) |
| M46Nle, | | CEYADETATIVEFLNRWITFSQSIISTLT |
| P65Y, | | |
| N71Hse, | | |
| M104Hse, | | |
| C125S | | |
| M23Nle, | 10 | APTSSSTKKTQLQLEHLLLDLQ(Nle)ILNGINNYKNPKLTY |
| R38Y, | | (Nle)L(Hse)YKFY(Nle)PKKATELKHLQCLEEYLKYLEEVL |
| M39Nle, | | (Hse)LAQSKNFHLRPRDLISNINVIVLELKGSETTF(Hse) |
| T41Hse, | | CEYADETATIVEFLNRWITFSQSIISTLT |
| F42Y, | | |
| M46Nle, | | |
| E62Y, | | |
| P65Y, | | |
| N71Hse, | | |
| M104Hse, | | |
| C125S | | |
| M23Nle, | 11 | APTSSSTKKTQLQLEHLLLDLQ(Nle)ILNGINNYKNPKLTR |
| M39Nle, | | (Nle)L(Hse)FKFY(Nle)PKKATELKHLQCLEEYLKYLEEVL |
| T41Hse, | | (Hse)LAQSKNFHLRPRDLISNINVIVLELKGSETTF(Hse) |
| M46Nle, | | CEYADETATIVEFLNRWITFSQSIISTLT |
| H64Nle, | | |
| E62Y, | | |
| P65Y, | | |
| N71Hse, | | |
| M104Hse, | | |
| C125S | | |
| M23Nle, | 12 | APTSSSTKKTQLQLEHLLLDLQ(Nle)ILNGINNYKNPKLTR |
| M39Nle, | | (Nle)L(Hse)FKFY(Nle)PKKATELKHLQCLEEYLKPLEEVL |
| T41Hse, | | (Hse)LAQSKNFHLRPRDLISNINVIVLELKGSETTF(Hse) |
| M46Nle, | | CEYADETATIVEFLNRWITFSQSIISTLT |
| E62Y, | | |
| N71Hse, | | |
| M104Hse, | | |
| C125S | | |
| M23Nle, | 13 | APTSSSTKKTQLQLEHLLLDLQ(Nle)ILNGINNYKNPKLTR |
| M39Nle, | | (Nle)L(Hse)FYFY(Nle)PKKATELKHLQCLEEELKPLEEVL |
| T41Hse, | | (Hse)LAQSKNFHLRPRDLISNINVIVLELKGSETTF(Hse) |
| K43Y, | | CEYADETATIVEFLNRWITFSQSIISTLT |
| M46Nle, | | |
| N71Hse, | | |
| M104Hse, | | |
| C125S | | |
| M23Nle, | 14 | APTSSSTKKTQLQLEHLLLDLQ(Nle)ILNGINNYKNPKLTR |
| M39Nle, | | (Nle)L(Hse)FKYY(Nle)PKKATELKHLQCLEEELKPLEEVL |
| T41Hse, | | (Hse)LAQSKNFHLRPRDLISNINVIVLELKGSETTF(Hse) |
| F44Y, | | CEYADETATIVEFLNRWITFSQSIISTLT |
| M46Nle, | | |
| N71Hse, | | |
| M104Hse, | | |
| C125S | | |
| M23Nle, | 15 | APTSSSTKKTQLQLEHLLLDLQ(Nle)ILNGINNYKNPYLTR |
| K35Y, | | (Nle)L(Hse)FKFY(Nle)PKKATELKHLQCLEEELKPLEEVL |
| M39Nle, | | (Hse)LAQSKNFHLRPRDLISNINVIVLELKGSETTF(Hse) |
| T41Hse, | | CEYADETATIVEFLNRWITFSQSIISTLT |
| M46Nle, | | |
| N71Hse, | | |
| M104Hse, | | |
| C125S | | |
| H16Y, | 16 | APTSSSTKKTQLQLEYLLLDLQ(Nle)ILNGINNYKNPKLTR |
| M23Nle, | | (Nle)L(Hse)FKFY(Nle)PKKATELKHLQCLEEELKPLEEVL |
| M39Nle, | | (Hse)LAQSKNFHLRPRDLISNINVIVLELKGSETTF(Hse) |
| T41Hse, | | CEYADETATIVEFLNRWITFSQSIISTLT |
| M46Hse, | | |
| N71Hse, | | |
| M104Hse, | | |
| C125S | | |
| H16Y, | 17 | APTSSSTKKTQLQLEYLLLDLQ(Nle)ILNGINNYKNPKLTR |
| M23Nle, | | (Nle)L(Hse)YKFY(Nle)PKKATELKHLQCLEEELKPLEEVL |
| M39Nle, | | (Hse)LAQSKNFHLRPRDLISNINVIVLELKGSETTF(Hse) |
| T41Hse, | | CEYADETATIVEFLNRWITFSQSIISTLT |
| F41Y, | | |
| M45Nle, | | |
| N71Hse, | | |
| M104Hse, | | |
| C125S | | |
| D20Y, | 18 | APTSSSTKKTQLQLEHLLLYLQ(Nle)ILNGINNYKNPKLTR |
| M23Nle, | | (Nle)L(Hse)FKFY(Nle)PKKATELKHLQCLEEELKPLEEVL |
| M39Nle, | | (Hse)LAQSKNFHLRPRDLISNINVIVLELKGSETTF(Hse) |
| T41Hse, | | CEYADETATIVEFLNRWITFSQSIISTLT |
| M46Nle, | | |
| N71Hse, | | |
| M104Hse, | | |
| C125S | | |
| D20Y, | 19 | APTSSSTKKTQLQLEHLLLYLQ(Nle)ILNGINNYKNPKLTR |
| M23Nle, | | (Nle)L(Hse)YKFY(Nle)PKKATELKHLQCLEEELKPLEEVL |
| M39Nle, | | (Hse)LAQSKNFHLRPRDLISNINVIVLELKGSETTF(Hse) |
| T41Hse, | | CEYADETATIVEFLNRWITFSQSIISTLT |
| F42Y, | | |
| M46Nle, | | |
| N71Hse, | | |
| M104Hse, | | |
| C125S | | |
| M23Nle, | 20 | APTSSSTKKTQLQLEHLLLDLQ(Nle)ILNGINNYKNPYLTR |
| K35Y, | | (Nle)L(Hse)YKFY(Nle)PKKATELKHLQCLEEELKPLEEVL |
| M39Nle, | | (Hse)LAQSKNFHLRPRDLISNINVIVLELKGSETTF(Hse) |
| T41Hse, | | CEYADETATIVEFLNRWITFSQSIISTLT |
| F42Y, | | |
| M46Nle, | | |
| N71Hse, | | |
| M104Hse, | | |
| C125S | | |
| M23Nle, | 21 | APTSSSTKKTQLQLEHLLLDLQ(Nle)ILNGINNYKNPKLTR |
| M39Nle, | | (Nle)L(Hse)YKFY(Nle)PKKATELKHLQCLEEYLKPLEEVL |
| T41Hse, | | (Hse)LAQSKNFHLRPRDLISNINVIVLELKGSETTF(Hse) |
| F42Y, | | CEYADETATIVEFLNRWITFSQSIISTLT |
| M46Nle, | | |
| E62Y, | | |
| N71Hse, | | |
| M104Hse, | | |
| C125S | | |
| M23Nle, | 22 | APTSSSTKKTQLQLEHLLLDLQ(Nle)ILNGINNYKNPKLTR |
| M39Nle, | | (Nle)L(Hse)YKFY(Nle)PKKATELKHLQCLEEYLKYLEEVL |
| T41Hse, | | (Hse)LAQSKNFHLRPRDLISNINVIVLELKGSETTF(Hse) |
| F42Y, | | CEYADETATIVEFLNRWITFSQSIISTLT |
| M46Nle, | | |
| E62Y, | | |
| P65Y, | | |
| N71Hse, | | |
| M104Hse, | | |
| C125S | | |
| M23Nle, | 23 | APTSSSTKKTQLQLEHLLLDLQ(Nle)ILNGINNYKNPKLTR |
| M39Nle, | | (Nle)L(Hse)FKFY(Nle)PKKATELKHLQCLEEELKPLEEVL |
| T41Hse, | | (Hse)LAQSKNFHLRPRDLISNINVIVLELKGSETTF(Hse) |
| M46Nle, | | CEYADETATIVEFLNRWITFCQSIISTLT |
| N71Hse, | | |
| M104Hse | | |

In Table 8 above, Nle is a norleucine residue and Hse is a homoserine residue.

Although the present disclosure and its advantages have been described in detail, it should be understood that various changes, substitutions and alterations can be made herein without departing from the spirit and scope of the disclosure as defined in the appended claims.

The present disclosure is further illustrated in the following Examples which are given for illustration purposes only and are not intended to limit the disclosure in any way.

### EXAMPLES

### Example 1: Preparation of Pembrolizumab-(IL-2 Polypeptide) Immunoconjugates

A modified Pembrolizumab polypeptide is prepared utilizing methods described in Examples 2-4 of US Patent Application No. US20200190165A1. Briefly, an Ajicapped (AJICAP™ by Ajinomoto Bio-Pharma Services, which is described at least in in PCT Publication No. WO2018199337A1, PCT Publication No. WO2019240288A1, PCT Publication No. WO2019240287A1, PCT Publication No. WO2020090979A1, Matsuda et al., Mol. Pharmaceutics 2021, 18, 4058-4066, and Yamada et al., AJICAP: Affinity Peptide Mediated Regiodivergent Functionalization of Native Antibodies. Angew. Chem., Int. Ed. 2019, 58, 5592-5597, and in particular Examples 2-4 of US Patent Publication No. US20200190165A1) Pembrolizumab polypeptide with a DBCO conjugation group was reacted with (IL-2 polypeptide) azide polypeptide for 48 hours at room temperature. The crude Pembrolizumab-(IL-2 polypeptide) immunoconjugates were separated using hydrophobic interaction chromatography, ion exchange chromatography, size exclusion chromatography, trapping by DBCO-PEG, protein A chromatography, or DBCO resin column purification.

An exemplary process for the AJICAP™ methodology is as follows: modified antibody (e.g., an anti-PD-1 antibody such as Pembrolizumab of LZM-009) comprising a DBCO conjugation handle is prepared using a protocol modified from Examples 2-4 of US Patent Publication No. US20200190165A1. Briefly, the anti-PD1 antibody with a free sulfhydryl group attached to a lysine residue side chain in the Fc region is prepared by contacting the antibody with an affinity peptide configured to deliver a protected version of the sulfhydryl group (e.g., a thioester or reducible disulfide) to the lysine residue. An exemplary peptide capable of performing this reaction is shown below (SEQ ID NO: 155), as reported in Matsuda et al., Mol. Pharmaceutics 2021, 18, 4058-4066, which selectively attached the sulfhydryl group via the NHS ester at residue K248 of the Fc region of the antibody:

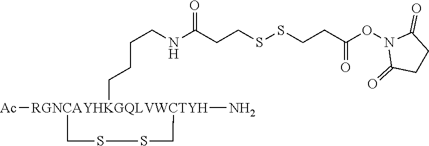

Alternative affinity peptides targeting alternative residues of the Fc region are described in the references cited above for AJICAP™ technology, and such affinity peptides can be used to attach the desired functionality to an alternative residue of the Fc region (e.g., K246, K288, etc.). For example, the disulfide group of the above affinity peptide could instead be replaced with a thioester to provide a sulfhydryl protecting group (e.g., the relevant portion of the affinity peptide would have a structure of

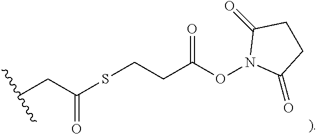 [SMILES: *CC(=O)SCCC(=O)ON1C(=O)CCC1=O]

The protecting group is then removed to reveal the free sulfhydryl (e.g., by hydrolysis of thioester or reduction of a disulfide with TCEP). The free sulfhydryl is then reacted with a bifunctional reagent comprising a bromoacetamide or bromoketone group connected to the DBCO conjugation handle through a linking group (e.g., bromoacetamido-dPEG®₄-amido-DBCO). The method can be used to produce an antibody with one DBCO group present (DAR1) and/or two DBCO groups attached to the antibody (DAR2, one DBCO group linked to each Fc of the antibody). The desired azide modified IL-2 polypeptide (e.g., Composition AB) is then reacted with the DBCO modified antibody to produce the immunocytokine.

In another embodiment, antibody comprising a single DBCO conjugation handle is prepared by first reacting excess anti-PD-1 antibody with appropriately loaded affinity peptide to introduce a single sulfhydryl after appropriate removal of protecting group (e.g., disulfide reduction or thioester cleavage). A bifunctional linking group with a sulfhydryl reactive conjugation handle and DBCO conjugation handle (e.g., bromoacetamido-dPEG® 4-amido-DBCO) is then reacted with the single sulfhydryl to produce the single DBCO containing antibody. The single DBCO containing antibody is then conjugated with a suitable azide containing IL-2 (e.g., Composition AB) to achieve an anti-PD-1-IL-2 immunoconjugate with a DAR of 1.

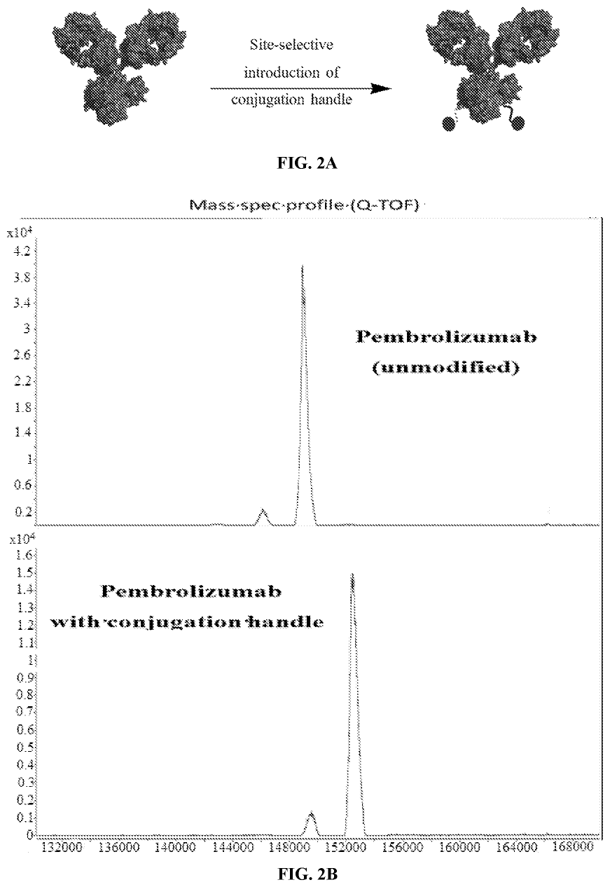

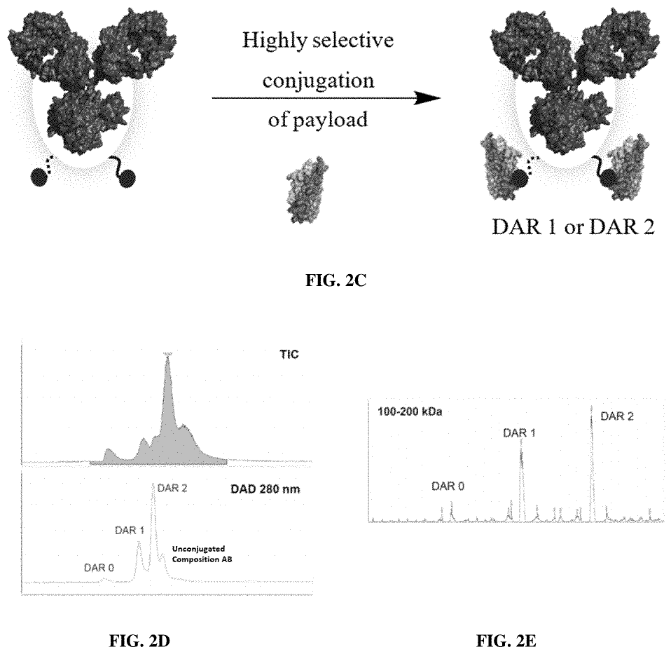

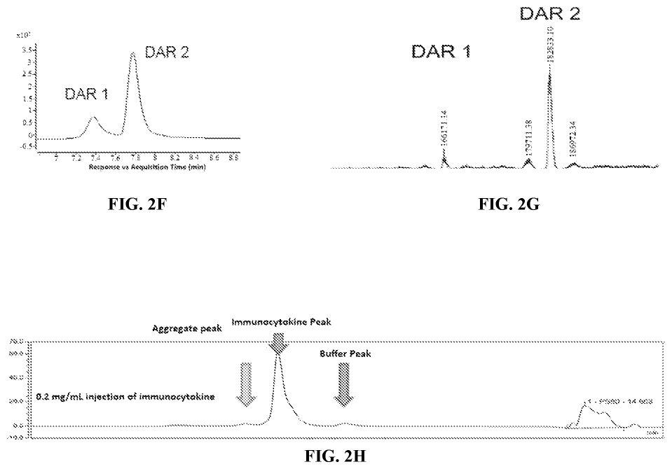

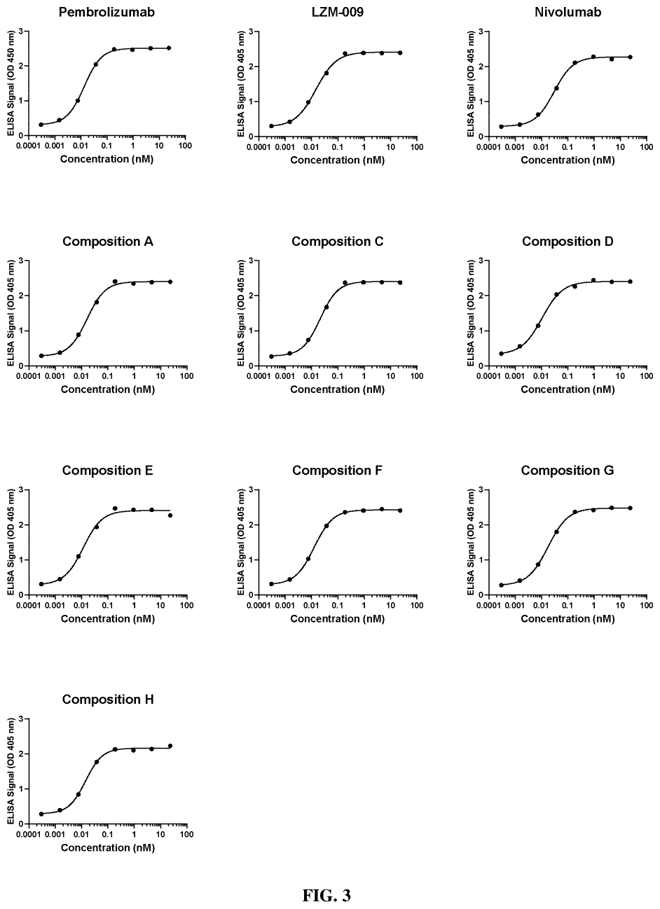

FIG. 2A shows site-selective modification of anti-PD1 antibody by chemical modification technology to introduce one or two conjugation handles. FIG. 2B shows Q-TOF mass spectra of unmodified Pembrolizumab and Pembrolizumab with conjugation to DBCO conjugation handle using AJICAP technology. FIG. 2C shows site-selective conjugation of IL2 cytokine to generate a PD1-IL2 with DAR1 or DAR 2. Populations of PD1-IL2s with mixed DAR between 1 and 2 can also be prepared. FIG. 2D shows TIC chromatogram (top) and intact RP-HPLC (bottom) profile of crude Pembrolizumab and Composition AB conjugation reaction mixture. DAR0 represents Pembrolizumab with no IL-2 conjugated, DAR1 represents Pembrolizumab with 1 IL-2 conjugated, and DAR2 represents Pembrolizumab with 2 IL-2 conjugated. FIG. 2E shows Q-TOF mass spec profile of crude Pembrolizumab and (Composition AB) conjugation reaction mixture showing the formation of DAR1 and DAR 2 species. FIG. 2F shows representative RP-HPLC chromatogram of the purified PD1-IL2, FIG. 2G shows representative Q-TOF mass spectra of the purified PD1-IL2, and FIG. 211 shows representative analytical SEC of the purified PD1-IL2 (Composition B).

Table 9 below summarizes various immunocytokines prepared according to the methods provided herein or analogous methods described elsewhere.

TABLE 9
Characteristics of immunocytokines

| | | | | | | Aggregates |
|---|---|---|---|---|---|---|
| | | IL-2 | | Conjugation | Endotoxin | (SEC- |
| Composition | Antibody | polypeptide | DAR | site | (EU/ml) | HPLC) |
| A | Pembrolizumab | Composition | 1 | K248 | <5 | 1.5% |
| | | AB | | | | |
| B | Pembrolizumab | Composition | 1.5 | K248 | 2.5 | 0% |
| | | AB | | | | |
| C | Pembrolizumab | Composition | 2 | K248 | 8.9 | 0.9% |
| | | AB | | | | |
| D | Pembrolizumab | Composition | 1 | K288 | <5 | 1.78% |
| | | AB | | | | |
| E | Pembrolizumab | Composition | 2 | K288 | <5.5 | 1.02% |
| | | AB | | | | |
| F | Pembrolizumab | Composition | 1 | K248 | 69.7 | 2.53% |
| | | AC | | | | |
| G | Pembrolizumab | Composition | 2 | K248 | 75.6 | 2.65% |
| | | AC | | | | |
| H | LZM-009 | Composition | 1 | K248 | <5.0 | 2.1% |
| | | AB | | | | |
| I | LZM-009 | Composition | 2 | K248 | 17.5 | 1.7% |
| | | AB | | | | |
| J | LZM-009 | Composition | 1 | K288 | 7.81 | 1.48% |
| | | AB | | | | |
| K | LZM-009 | Composition | 2 | K288 | <0.006 | <5% |
| | | AB | | | | |
| N | Trastuzumab | Composition | 1.6 | K248 | 11.3 | 0.2% |
| | | AB | | | | |
| 0 | Trastuzumab | Composition | 1 | K248 | <0.5 | <0.1% |
| | | AB | | | | |

### Example 2: PD-1 Binding ELISA Assay (FIG. 3)

The interaction of the unmodified and of conjugated anti-PD1 antibodies with PD-1 (CD279) were measured by ELISA assay. For these studies, Corning high-binding half-area plates (Fisher Scientific, Reinach, Switzerland) were coated overnight at 4° C. with 25 μl of unmodified or conjugated anti-PD1 antibodies at 2.5 μg/ml in PBS. Plates were then washed four times with 100 μl of PBS-0.02% Tween20. Plate surfaces were blocked with 25 μl of PBS-0.02% Tween20-1% BSA at 37° C. during 1 h. Plates were then washed four times with 100 μl of PBS-0.02% Tween20. Twenty-five microliters of recombinant biotinylated PD1/CD279 protein from Biolegend (789406, London, United Kingdom) were added in five-fold serial dilutions starting at 23 nM down to 0.0003 nM in PBS-0.02% Tween20-0.1% BSA and incubated at 37° C. during 2 h. Plates were then washed four times with 100 μl of PBS-0.02% Tween20. Twenty-five microliters of Streptavidin-Horseradish peroxidase (#RABHRP3, Merck, Buchs, Switzerland) diluted at 1:500 in PBS-0.02% Tween20-0.1% BSA were added to each well and incubated at Room Temperature during 30 min. Plates were then washed four times with 100 μl of PBS-0.02% Tween20. Fifty microliters of TMB substrate reagent (#CL07, Merck, Buchs, Switzerland) were added to each well and incubated at 37° C. during 5 min. After 5 min at 37° C., Horseradish peroxidase reaction was stopped by adding 50 μl/well of 0.5M H₂SO₄ stop solution. ELISA signal was then measured at 450 nm on an EnSpire plate reader from Perkin Elmer (Schwerzenbach, Switzerland).

TABLE 10
KD values of the interaction of immunocytokines
with PD-1 as measured by ELISA

| | | | | Conjugation | |
|---|---|---|---|---|---|
| | | | | site (Eu | |
| | | IL-2 | | numbering of | KD |
| Composition | Antibody | polypeptide | DAR | Fc region) | (μM) |
| — | Pembrolizumab | — | 0 | — | |
| A | Pembrolizumab | Composition AB | 1 | K248 | 60 |
| B | Pembrolizumab | Composition AB | 1.5 | K248 | NT |
| C | Pembrolizumab | Composition AB | 2 | K248 | 56 |
| D | Pembrolizumab | Composition AB | 1 | K288 | 31 |
| E | Pembrolizumab | Composition AB | 2 | K288 | 29 |
| F | Pembrolizumab | Composition AC | 1 | K248 | 36 |
| G | Pembrolizumab | Composition AC | 2 | K248 | 41 |
| | LZM-009 | — | 0 | — | 35 |
| H | LZM-009 | Composition AB | 1 | K248 | 37 |
| I | LZM-009 | Composition AB | 2 | K248 | NT |
| J | LZM-009 | Composition AB | 1 | K288 | 27 |
| K | LZM-009 | Composition AB | 2 | K288 | 41 |
| — | Nivolumab/Opdivo | — | 0 | — | 66 |

NT: Not Tested

FIG. 3 shows plots describing the ability of the unmodified and of conjugated anti-PD1 antibodies to bind to PD1/CD279 ligand, with the figure showing ELISA signal on the y-axis and dosage of the biotinylated PD-L1 protein on the x-axis. The unconjugated reference antibody and the conjugated antibodies tested in this figure are composition PEMBROLIZUMAB, NIVOLUMAB, LZM-009, and Composition A, C, D, E, F, G, and H, respectively.

PD-1/PD-L1 blockade bioassay: A PD-1/PD-L1 blockade bioassay was used to determine the ability of the Pembrolizumab-(IL-2 polypeptide) immunoconjugates to block PD-1/PD-L¹ interactions.

The ability of the unmodified and of conjugated anti-PD1 antibodies to interfere with PD1/PDL1 pathway was measured using the PD-1/PD-L1 Blockade Bioassay from Promega (Cat. #J1250, Madison, Wis., USA). PD-1/PD-L1 Blockade Bioassay is a bioluminescent cell-based assay based on the co-culture of effector cells with target cells mimicking an immunological synapse. Jurkat T cells expressing human PD-1 and a luciferase reporter driven by a NEAT response element (NEAT-RE) are activated by CHO-K1 cells expressing human PD-L1 and an engineered cell surface protein designed to activate Jurkat's cognate TCRs. Concurrent interaction PD-1/PD-L1 inhibits TCR signaling and represses NEAT-RE-mediated luminescence. Addition of either an anti-PD-1 or anti-PD-L1 antibody that blocks the PD-1/PD-L1 interaction releases the inhibitory signal, restoring TCR activation and resulting in a gain of signal of NFAT-RE luminescent reporter.

Briefly, PD-L1 aAPC/CHO-K1 Target cells were plated in white tissue culture −96 wells plates and cultured overnight at 37° C./5% CO₂. Test molecules were measured in four-fold serial dilutions starting at 1 uM down to 0.002 nM and pre-incubated on target cells for 10 min before the addition of freshly thawed PD-1 Jurkat effector cells. After 6 h at 37° C./5% CO₂, activity NFAT-RE luminescent reporter was evaluated by the addition of Bio-Glo reagent and measured on an EnSpire plate reader (1 sec/well) from Perkin Elmer (Schwerzenbach, Switzerland).

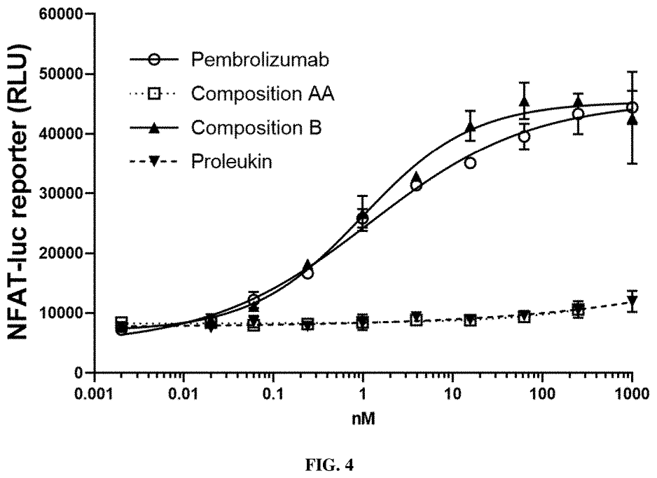

FIG. 4 shows plots describing the ability of the unmodified and of conjugated anti-PD1 antibodies to block the PD1/PDL1 pathway, with the figure showing mean luminescence intensity of effector cells NFAT-RE reporter on the y-axis and dosage of the unmodified and of conjugated anti-PD1 antibodies on the x-axis. The unconjugated reference antibody and the conjugated antibodies tested in this figure are Pembrolizumab and Composition B respectively. The modified IL-2 polypeptides tested in this figure are Proleukin and Composition AA.

The interaction of the unmodified and of conjugated anti-PD1 antibodies with the human neonatal Fc receptor (FcRn) at pH 6 was measured using the AlphaLISA® Human FcRn Binding Kit (AL3095C) from Perkin Elmer (Schwerzenbach, Switzerland). The AlphaLISA® detection of FcRn and IgG binding uses IgG coated AlphaLISA® acceptor beads to interact with biotinylated human FcRn captured on Streptavidin-coated donor beads. When reference IgG binds to FcRn, donor and acceptor beads come into proximity enabling the transfer of singlet oxygen that trigger a cascade of energy transfer reactions in the acceptor beads, resulting in a sharp peak of light emission at 615 nm. Addition of a free IgG antibodies into the AlphaLISA® mixture creates a competition for the binding of FcRn to the reference antibody resulting in a loss of signal.

Briefly, test molecules were measured in serial dilutions starting at 5 uM down to 64 pM and incubated with AlphaLISA® reaction mixture consisting of 800 nM of recombinant biotinylated human FcRn, 40 μg/ml of human IgG conjugated Acceptor beads, and 40 μg/ml of Streptavidin coated Donor beads in pH 6 MES buffer. After 90 min at 23° C. in the dark, AlphaLISA® signal was measured on an EnSpire plate reader (Excitation at 680 nm, Emission at 615 nm) from Perkin Elmer (Schwerzenbach, Switzerland).

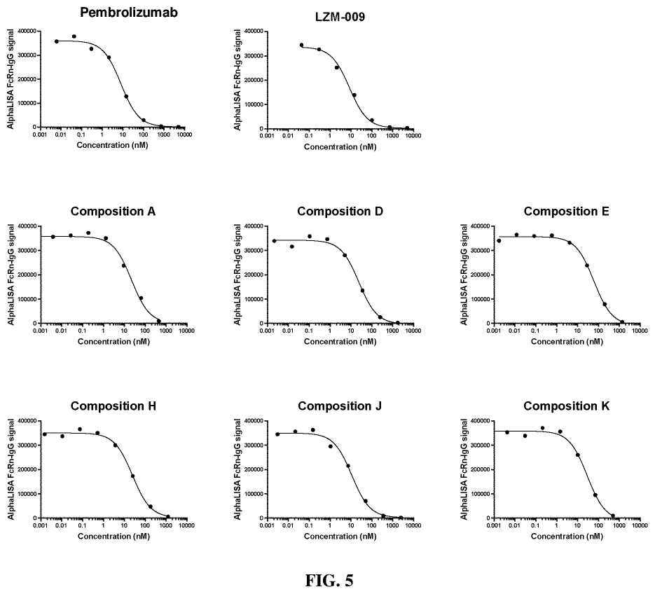

FIG. 5 shows plots describing the ability of the unmodified and of conjugated anti-PD1 antibodies to bind to human neonatal Fc receptor (FcRn) at pH 6, with the figure showing mean AlphaLISA® FcRn-IgG signal on the y-axis and dosage of the unmodified and of conjugated anti-PD1 antibodies on the x-axis. The unconjugated reference antibody and the conjugated antibodies tested in this figure are Pembrolizumab, LZM-009, and Composition A, D, E, H, J, K respectively.

TABLE 11
KD values of the interaction of immunocytokines with
human FcRn receptor at pH 6 as measured by alphaLISA

| | | IL-2 | | Conjugation | KD |
|---|---|---|---|---|---|
| Composition | Antibody | polypeptide | DAR | site | (nM) |
| — | Pembrolizumab | — | 0 | — | 7.31 |
| A | Pembrolizumab | Composition | 1 | K248 | 26.31 |
| | | AB | | | |
| B | Pembrolizumab | Composition | 1.5 | K248 | 27.80 |
| | | AB | | | |
| C | Pembrolizumab | Composition | 2 | K248 | 85.80 |
| | | AB | | | |
| D | Pembrolizumab | Composition | 1 | K288 | 18.56 |
| | | AB | | | |
| E | Pembrolizumab | Composition | 2 | K288 | 48.04 |
| | | AB | | | |
| F | Pembrolizumab | Composition | 1 | K248 | NT |
| | | AC | | | |
| G | Pembrolizumab | Composition | 2 | K248 | NT |
| | | AC | | | |
| | LZM-009 | — | 0 | — | 15.27 |
| H | LZM-009 | Composition | 1 | K248 | 25.27 |
| | | AB | | | |
| I | LZM-009 | Composition | 2 | K248 | NT |
| | | AB | | | |
| J | LZM-009 | Composition | 1 | K288 | 10.69 |
| | | AB | | | |
| K | LZM-009 | Composition | 1.9 | K288 | 27.11 |
| | | AB | | | |

NT: Not Tested.

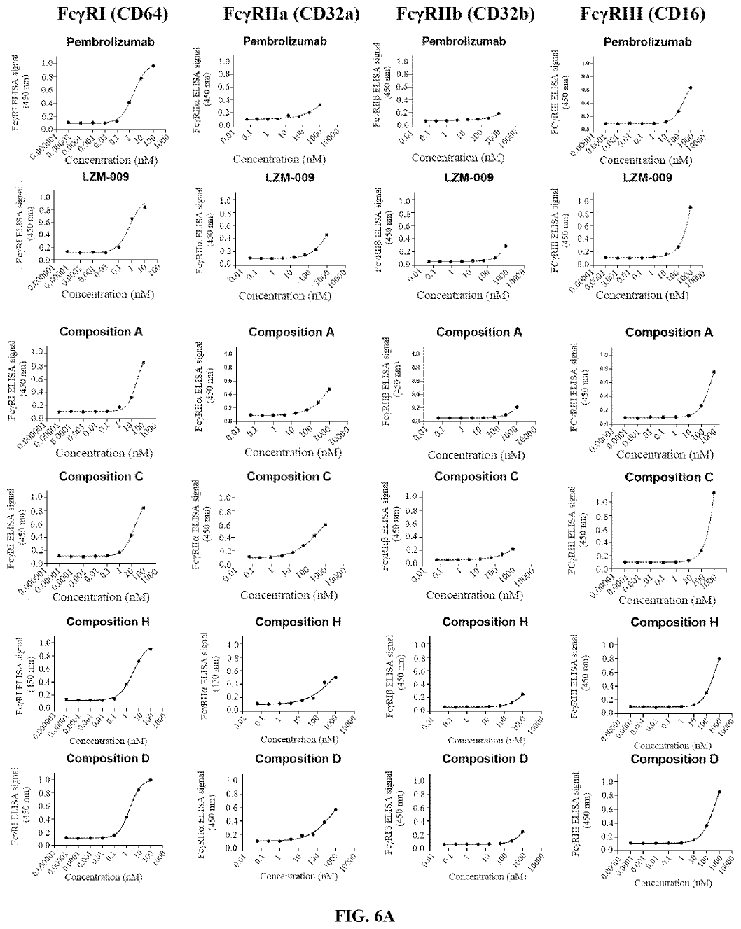

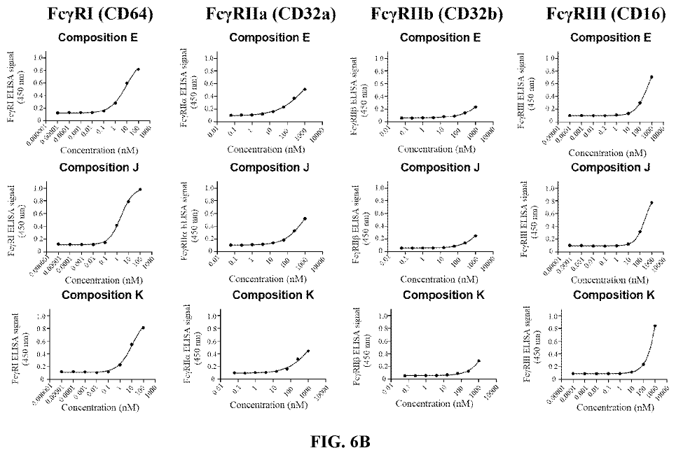

### Example 3: Human FcγR Binding Assay (FIGS. 6)

The interaction of the unmodified and of conjugated anti-PD1 antibodies with human Fc gamma receptors I (FcγRI/CD64), with human Fc gamma receptors Ha (FcγRIIa/CD32a), with inhibitory human Fc gamma receptors IIb (FcγRIIb/CD32b), and with human Fc gamma receptors III FcγR3a/CD16 were measured by ELISA. Briefly, Corning high-binding half-area plates (Fisher Scientific, Reinach, Switzerland) were coated overnight at 4° C. with 25 μl of unmodified and of conjugated anti-PD1 antibodies at 2.5 μg/ml in PBS. Plates were then washed four times with 100 μl of PBS-0.02% Tween20. Plates surfaces were blocked with 25 μl of PBS-0.02% Tween20-1% BSA at 37° C. during 1 h. Plates were then washed four times with 100 μl of PBS-0.02% Tween20. Then twenty-five microliters of either recombinant Human Fc gamma RI/CD64 Protein (R&D systems, 1257-FC-050, CF), recombinant Human Fc gamma RIIA/CD32a (H167) Protein (R&D systems, 9595-CD-050, CF), recombinant Human Fc gamma RIIB/CD32b Avi-tag Protein (R&D systems, AVI1875-050, CF), or recombinant Human Fc gamma RIIIA/CD16a Protein (R&D systems, 4325-FC-050; CF) were added in five-fold serial dilutions ranging from 1000 nM to 0.001 nM into PBS-0.02% Tween20-0.1% BSA and incubated at 37° C. during 2 h. Plates were then washed four times with 100 μl of PB S-0.02% Tween20. Twenty-five microliters of Streptavidin-Horseradish peroxidase (#RABHRP3, Merck, Buchs, Switzerland) diluted at 1:500 into PBS-0.02% Tween20-0.1% BSA were added to each well and incubated at Room Temperature during 30 min. Plates were then washed four times with 100 μl of PBS-0.02% Tween20. Fifty microliters of TMB substrate reagent (#CL07, Merck, Buchs, Switzerland) were added to each well and incubated at 37° C. during 5 min. After 5 min at 37° C., Horseradish peroxidase reaction was stopped by adding 50 μl/well of 0.5M H2SO4 stop solution. ELISA signal was then measured at 450 nm on an EnSpire plate reader from Perkin Elmer (Schwerzenbach, Switzerland). After 90 min at 23° C. in the dark,

TABLE 12
KD values of the interaction of immunocytokines with human Fcγ receptor as measured by ELISA

| | | | | | FcgRI | FcgRIIa | FcgRIIb | FcgRIIIa |
|---|---|---|---|---|---|---|---|---|
| | | IL-2 | | Conjugation | CD64 | CD32a | CD32b | CD16 |
| Composition | Antibody | polypeptide | DAR | site | (nM) | (nM) | (nM) | (nM) |
| | Pembrolizumab | — | 0 | — | 0.488 | 160.2 | 209.3 | 289 |
| A | Pembrolizumab | Composition | 1 | K248 | 1.265 | 313.7 | 744.5 | 339.5 |
| | | AB | | | | | | |
| B | Pembrolizumab | Composition | 1.5 | K248 | NT | NT | NT | NT |
| | | AB | | | | | | |
| C | Pembrolizumab | Composition | 2 | K248 | 18.57 | 314.2 | 780.2 | 390 |
| | | AB | | | | | | |
| D | Pembrolizumab | Composition | 1 | K288 | 1.261 | 488.9 | 2082.3 | 609.05 |
| | | AB | | | | | | |
| E | Pembrolizumab | Composition | 2 | K288 | 6.128 | 767.9 | 5084.6 | 1055.5 |
| | | AB | | | | | | |
| F | Pembrolizumab | Composition | 1 | K248 | NT | NT | NT | NT |
| | | AC | | | | | | |
| G | Pembrolizumab | Composition | 2 | K248 | NT | NT | NT | NT |
| | | AC | | | | | | |
| | LZM-009 | — | 0 | — | 0.248 | 904.6 | 543.6 | 921.5 |
| H | LZM-009 | Composition | 1 | K248 | 3.81 | 1142 | 4709 | 481.1 |
| | | AB | | | | | | |
| I | LZM-009 | Composition | 2 | K248 | NT | NT | NT | NT |
| | | AB | | | | | | |
| J | LZM-009 | Composition | 1 | K288 | 2.298 | 1179 | 6683 | 272.5 |
| | | AB | | | | | | |
| K | LZM-009 | Composition | 1.9 | K288 | 11.99 | 1873 | 3341 | 5407 |
| | | AB | | | | | | |

NT: Not Tested.

FIG. 6A shows plots describing the ability of the unmodified and of conjugated anti-PD1 antibodies to bind to human Fc gamma receptor I (CD64), human Fc gamma receptor IIa (CD32a), human Fc gamma receptor IIb (CD32b), and to human Fc gamma receptor Ma (CD16) with the figure showing mean ELISA signal on the y-axis and dosage of the unmodified and of conjugated anti-PD1 antibodies on the x-axis. The unconjugated reference antibody are Pembrolizumab, LZM-009, and Composition A and the conjugated antibodies tested in this figure are Compositions C, D, and H.

FIG. 6B shows plots describing the ability of the unmodified and of conjugated anti-PD1 antibodies to bind to human Fc gamma receptor I (CD64), human Fc gamma receptor IIa (CD32a), human Fc gamma receptor IIb (CD32b), and to human Fc gamma receptor Ma (CD16) with the figure showing mean ELISA signal on the y-axis and dosage of the unmodified and of conjugated anti-PD1 antibodies on the x-axis. The conjugated antibodies tested in this figure are Compositions E, J, and K.

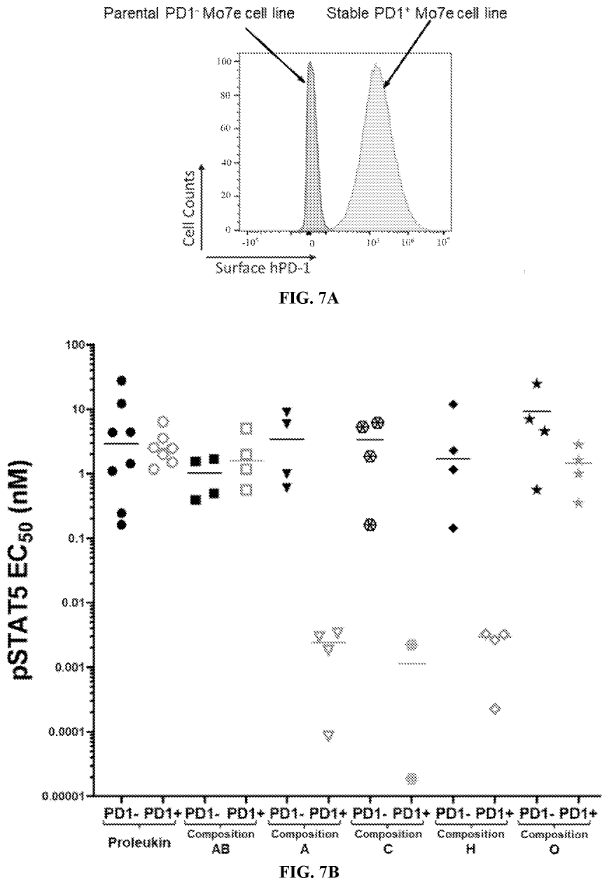

### Example 4: IL2-Induced pStat5 Activation in PD1^{positive} vs PD1^{negative} Mo7e Cells (FIGS. 7A-B)

A human Mo7e cell line stably expressing human PD-1 was established. Briefly, 250×10⁵ Mo7e cells were transduced with Lentiviral Particle carrying human PD1 gene (PDCD1 NM 005018; Origene, CAT #: RC210364 L3V) at MOI (Multiplicity of Infection) of 4. Spinfection was performed at 1260 g during 90 min in the presence of 5 μg/ml of Polybrene and 10 mM of HEPES in complete culture media (RPMI, 20% FBS, 10 ng/ml GM-CSF) at 37° C. Five days after transduction, puromycin at 0.75 μg/ml was added to select for PD-1 positive cells. Stable and homogenous expression of PD-1 was verified by surface staining.

An experiment was performed to determine the effect of various IL-2 polypeptides on PD-1 negative (parental non-transduced strain) and on PD-1 expressing (transduced) Mo7e cells. Cells were distributed at 100,000 cells per well and stimulated with 10-fold serial dilutions of modified IL-2 polypeptides unconjugated and conjugated to anti-PD1 antibody with a starting concentration of 949 nM down to 10 pM, for 40 min at 37° C./5% CO₂. After incubation, cells were fixed and permeabilized using the Transcription Factor Phospho Buffer kit (BD Biosciences) followed by a surface and intracellular immunostaining for PD-1 and pStat5 to enable cell identification and measure of levels of Stat5 (signal transducer and activator of transcription 5) phosphorylation. The FACS (fluorescence activated cell sorting) measurement was done with a Quanteon Flow Cytometer from Acea.

FIG. 7A shows plots describing the level of surface expression of PD-1/CD279 on parental non-transduced Mo7e (PDF) and stably transduced (PD1⁺) Mo7e cells.

FIG. 7B shows plots describing the effect of the modified IL-2 polypeptides unconjugated and conjugated to the anti-PD1 antibody on the inducement of IL-2 signaling pathway on PD1^{negative} (black filled symbols) and PD1^{positive} (grey open symbols) Mole cells in vitro, with the figure showing mean EC₅₀ values for phosphorylated signal transducer and activator of transcription 5 (pSTAT5) on the y-axis and dosage of modified IL-2 polypeptide and immunocytokines on the x-axis. The modified IL-2 polypeptides tested in this figure are Proleukin, and Composition AB. The immunocytokines tested in this figure are Composition A, C, H, L and Her2-targeted immunocytokine Composition O (Trastuzumab antibody conjugated to IL-2 polypeptide) as control.

TABLE 13
EC₅₀ values of the STAT5 phosphorylation assay in PD1⁻ and PD1⁺ Mo7e cells

| | | | | | EC₅₀ | EC₅₀ | PD1⁻/ |
|---|---|---|---|---|---|---|---|
| | | IL-2 | | Conjugation | PD1⁻ | PD1⁺ | PD1⁺ |
| Composition | Antibody | polypeptide | DAR | site | cells (nM) | cells (nM) | ratio |
| Proleukin | — | | — | — | 2.925 | 2.484 | |
| AB | — | Composition | — | — | 1.03 | 1.582 | 0.65 |
| | | AB | | | | | |
| AC | — | Composition | — | — | NT | NT | NT |
| | | AC | | | | | |
| A | Pembrolizumab | Composition | 1 | K248 | 3.437 | 0.0024 | 1424 |
| | | AB | | | | | |
| B | Pembrolizumab | Composition | 1.5 | K248 | NT | NT | NT |
| | | AB | | | | | |
| C | Pembrolizumab | Composition | 2 | K248 | 3.589 | 0.0011 | 3175 |
| | | AB | | | | | |
| D | Pembrolizumab | Composition | 1 | K288 | NT | NT | NT |
| | | AB | | | | | |
| E | Pembrolizumab | Composition | 2 | K288 | NT | NT | NT |
| | | AB | | | | | |
| F | Pembrolizumab | Composition | 1 | K248 | NT | NT | NT |
| | | AC | | | | | |
| G | Pembrolizumab | Composition | 2 | K248 | NT | NT | NT |
| | | AC | | | | | |
| H | LZM-009 | Composition | 1 | K248 | 1.722 | 0.003 | 581 |
| | | AB | | | | | |
| I | LZM-009 | Composition | 2 | K248 | NT | NT | NT |
| | | AB | | | | | |
| J | LZM-009 | Composition | 1 | K288 | NT | NT | NT |
| | | AB | | | | | |
| K | LZM-009 | Composition | 1.9 | K288 | NT | NT | NT |
| | | AB | | | | | |
| O | Trastuzumab | Composition | 1 | K248 | 5.781 | 1.308 | 1.62 |
| | | AB | | | | | |

NT: Not Tested

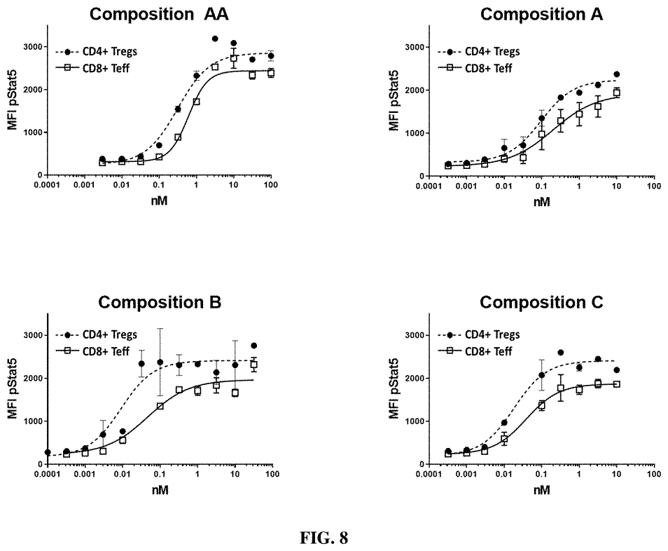

### Example 5: IL2-Induced pStat5 Activation in Primary T-Cells (FIGS. 8-10)

An experiment was performed to determine the effect of various IL-2 polypeptides on human T-cell populations. Primary pan T-cells (CD4+ T cells, CD8+ T cells, and Tregs) were obtained from healthy donor buffy coat by peripheral blood mononuclear cell (PBMC) purification using ficoll gradient centrifugation followed by negative isolation with magnetic beads and then cryopreserved until use. Pan T-cells were thawed, allowed them to recover overnight in T-cell medium (RPMI 10% FCS, 1% Glutamin, 1% NEAA, 25 μM βMeoH, 1% NaPyrovate) and after two washing steps with PBS cells were resuspended in PBS. When indicated, cells are pre-incubated during 20 min at 37° C./with 100 nM of unconjugated anti-PD1 antibody Pembrolizumab. Cells were then distributed at 200′000 cells per well and stimulated with 3.16-fold serial dilutions of modified IL-2 polypeptides unconjugated and conjugated to anti-PD1 antibody with a starting concentration of 316 nM down to 3 pM, for 40 min at 37° C./5% CO₂. After incubation, cells were fixed and permeabilized using the Transcription Factor Phospho Buffer kit followed by a surface and intracellular immunostaining for CD4, CD8, CD25, FoxP3, CD45RA and pStat5 to enable cell subsets identification and measure of levels of Stat5 (signal transducer and activator of transcription 5) phosphorylation. The FACS (fluorescence activated cell sorting) measurement was done either with a NovoCyte or a Quanteon Flow Cytometer from Acea.

pStat5 MFI (medium fluorescence intensity) signal for the following T-cell subsets were plotted against concentrations of wild type or of modified IL-2 polypeptides. Half maximal effective concentration (EC₅₀) was calculated based on a variable slope, four parameter analysis using GraphPad PRISM software.

Gating strategy for T-cell subsets identification

| | |
|---|---|
| T-Reg | CD4+, CD25^{Hi}, FoxP3+ |
| CD8 Teff | CD8+ |
| Naïve CD8 Teff | CD8+, CD45RA+ |
| Memory CD8 Teff | CD8+, CD45RA− |
| CD4 conv | CD4+, FoxP3− |

TABLE 14
EC₅₀ values of the STAT5 phosphorylation assay in primary human T-cells

| | | | | | EC₅₀ | EC₅₀ | CD8/ |
|---|---|---|---|---|---|---|---|
| | | IL-2 | | Conjugation | Tregs | CD8 | Treg |
| Composition | Antibody | polypeptide | DAR | site | (nM) | (nM) | ratio |
| Proleukin | — | | — | — | | | |
| AB | — | Composition AB | — | — | 0.385 | 0.818 | 2.1 |
| AC | — | Composition AC | — | — | 0.386 | 0.941 | 2.4 |
| A | Pembrolizumab | Composition AB | 1 | K248 | 0.411 | 2.287 | 5.6 |
| B | Pembrolizumab | Composition AB | 1.5 | K248 | 0.228 | 1.013 | 4.4 |
| C | Pembrolizumab | Composition AB | 2 | K248 | 0.154 | 0.833 | 5.4 |
| D | Pembrolizumab | Composition AB | 1 | K288 | 0.081 | 0.996 | 12.3 |
| E | Pembrolizumab | Composition AB | 2 | K288 | NT | NT | NT |
| F | Pembrolizumab | Composition AC | 1 | K248 | 7.775 | 10.72 | 1.8 |
| G | Pembrolizumab | Composition AC | 2 | K248 | 0.229 | 2.211 | 9.7 |
| H | LZM-009 | Composition AB | 1 | K248 | 0.098 | 0.864 | 8.8 |
| I | LZM-009 | Composition AB | 2 | K248 | NT | NT | NT |
| J | LZM-009 | Composition AB | 1 | K288 | NT | NT | NT |
| K | LZM-009 | Composition AB | 1.9 | K288 | NT | NT | NT |

FIG. 8 shows plots describing the effect of the modified IL-2 polypeptides unconjugated and conjugated to the anti-PD1 antibody on the inducement of T_{eff} and T_{reg} cells in an in vitro sample of human T-cells, with the figure showing mean fluorescence intensity for phosphorylated signal transducer and activator of transcription 5 (pSTAT5) on the y-axis and dosage of modified IL-2 polypeptide and immunocytokines on the x-axis. The modified IL-2 polypeptide tested in this figure is Composition AA. The immunocytokines tested in this figure are Compositions A, B, and C.

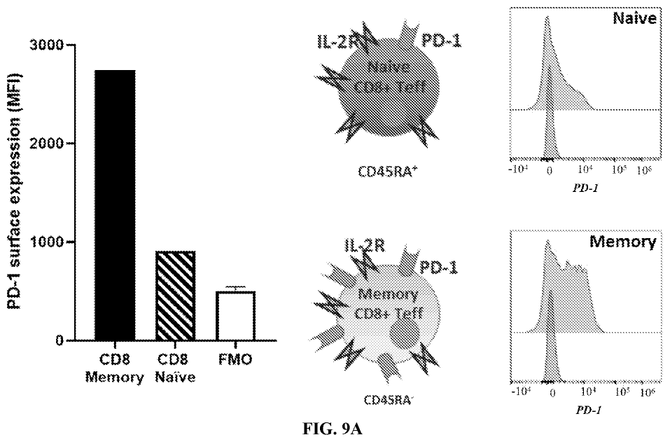

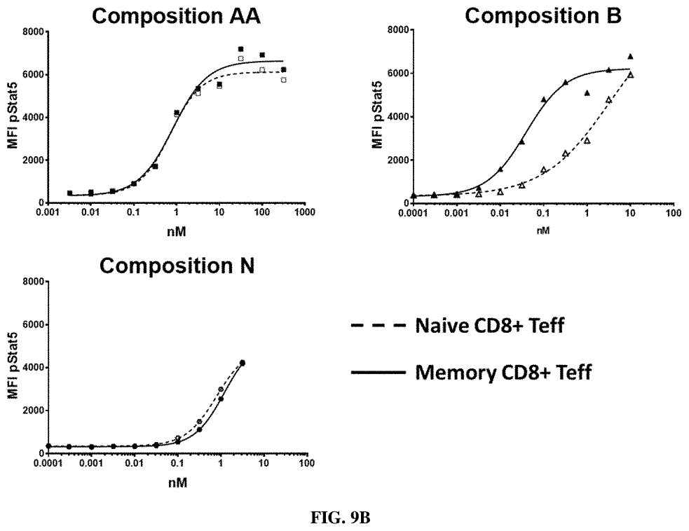

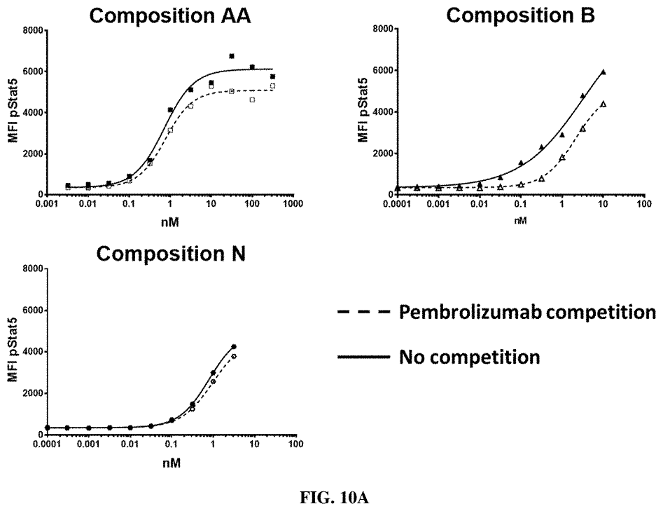

FIG. 9A shows plots describing the level of surface expression of PD-1/CD279 on resting memory (CD45RA−) and naïve (CD45RA+) CD8+ T_{eff} cells freshly isolate from peripheral blood of healthy donors.

FIG. 9B shows plots describing the effect of the modified IL-2 polypeptides unconjugated and conjugated to the anti-PD1 antibody on the inducement of on resting memory (CD45RA−) and naïve (CD45RA+) CD8+ T_{eff} cells in an in vitro sample of human T-cells, with the figure showing mean fluorescence intensity for phosphorylated signal transducer and activator of transcription 5 (pSTAT5) on the y-axis and dosage of modified IL-2 polypeptide and immunocytokines on the x-axis. The modified IL-2 polypeptide tested in this figure is Composition AA and the immunocytokines tested in this figure are Composition B and immunocytokine composition N (Trastuzumab antibody conjugated to IL-2 polypeptide) as a control.

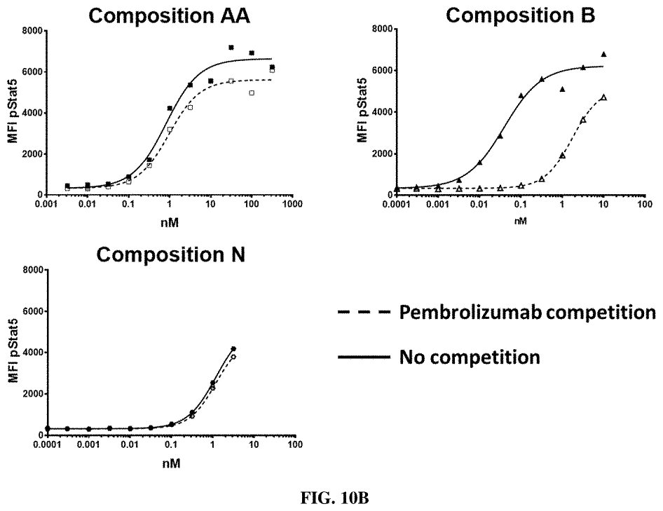

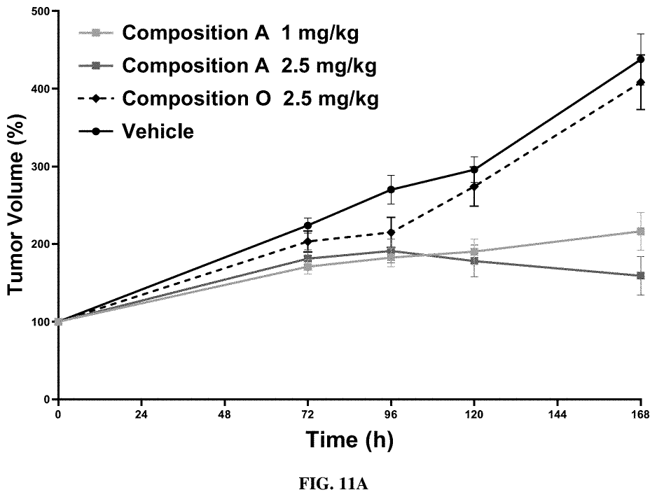

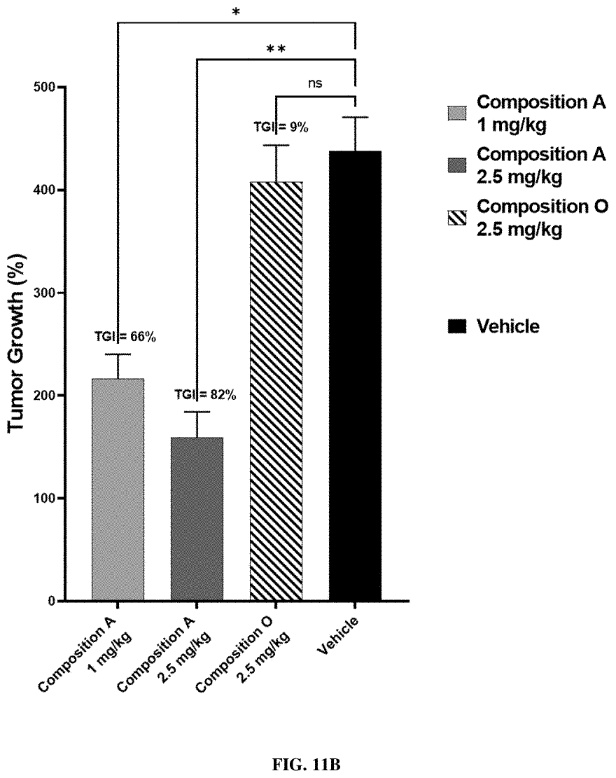

FIG. 10A shows plots measuring the effect of the modified IL-2 polypeptides unconjugated and conjugated to the anti-PD1 antibody on the inducement of resting naïve (CD45RA+) CD8+ T_{eff} cells in an in vitro sample of human T-cells in the presence or absence of excess amounts of unconjugated anti-PD1 antibody Pembrolizumab, with the figure showing mean fluorescence intensity for phosphorylated signal transducer and activator of transcription 5 (pSTAT5) on the y-axis and dosage of modified IL-2 polypeptide and immunocytokines on the x-axis. The modified IL-2 polypeptide tested in this figure is Composition AA and the immunocytokines tested in this figure are Composition B and Her2-targeted immunocytokine Composition N (Trastuzumab antibody conjugated to IL-2 polypeptide) as a control.

FIG. 10B shows plots measuring the effect of the modified IL-2 polypeptides unconjugated and conjugated to the anti-PD1 antibody on the inducement of resting memory (CD45RA−) CD8+ T_{eff} cells in an in vitro sample of human T-cells in the presence or absence of excess amounts of unconjugated anti-PD1 antibody Pembrolizumab, with the figure showing mean fluorescence intensity for phosphorylated signal transducer and activator of transcription 5 (pSTAT5) on the y-axis and dosage of modified IL-2 polypeptide and immunocytokines on the x-axis. The modified IL-2 polypeptide tested in this figure is Composition AA and the immunocytokines tested in this figure are Composition B and Her2-targeted immunocytokine Composition N (Trastuzumab antibody conjugated to IL-2 polypeptide) as a control.

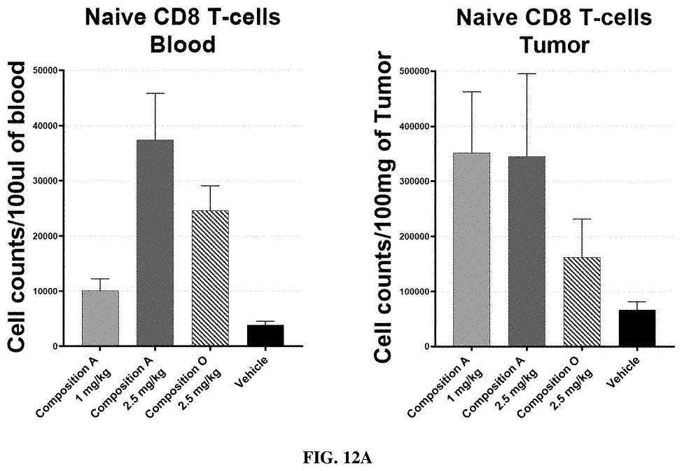

### Example 6: PK/PD Study in Tumor Bearing Mice (FIGS. 11-13)

An in vivo PK/PD study was performed in mice. Naïve, 6-8 weeks old, BALB/c-hPD1 female mice (GemPharmatech Co, Ltd, Nanjing, China) were inoculated subcutaneously at the left flank with wild type CT26 tumor cells (3×10⁵) in 0.1 mL of PBS for tumor development. The animals were randomized (using an Excel-based randomization software performing stratified randomization based upon tumor volumes), and treatment started when the average tumor volume reached approximately 186 mm³. Animals treated with Composition A received a single 10 mL/kg bolus intravenous (i.v.) injection of 1, and 2.5 mg/kg of PD-1 antibody conjugated with modified IL-2 polypeptide. Animals treated with control Her2-targeted immunocytokine Composition O (Trastuzumab antibody conjugated to IL-2 polypeptide) received a single 10 mL/kg bolus intravenous (i.v.) injection of 2.5 mg/kg of anti-Her2 antibody conjugated with modified IL-2 polypeptide. After inoculation, the animals were checked daily for morbidity and mortality. At the time, animals were checked for effects on tumor growth and normal behavior such as mobility, food and water consumption, body weight gain/loss (body weights were measured twice weekly), eye/hair matting and any other abnormal effect. Tumor sizes were measured three times a week in two dimensions using a caliper, and the volume was expressed in mm³ using the formula: V=0.5 a×b² where a and b are the long and short diameters of the tumor, respectively. Death and observed clinical signs were recorded on the basis of the numbers of animals within each subset.

Pharmacokinetic study included 9 time points (5 min, 1 h, 6 h, 12 h, 24 h, 72 h, 96 h, 120 h, 168 h) with 3 mice sampled per time points. At indicated time points, blood samples were collected in the presence of EDTA either via tail vein sampling or via cardiac puncture (end-point). In addition, 72 h, 96 h, 120 h, 168 h after injection 3 mice per group were sacrificed and tumor samples were collected.

Fresh tumor sample from each mouse was minced individually and digested with mixed enzymes in C tubes. C tubes were attached onto the sleeves of the Gentle MACS Dissociator before running the program “m_imptumor_01_01” one time. C tubes were then incubated for 30 minutes at 37° C., followed by another round of program “m_imptumor_01_01”. Digested tissues were filtered through 70 μm cell strainers. Cells were washed twice with DPBS before staining.

Collected mouse blood in the presence of EDTA was immediately centrifuged sample tube at 4,000 rpm for 5 min at 4° C. Collected plasma (supernatant) and stored at −80° C. until Bioanalysis. Then mixed one volume of cell pellet with 20 volumes of 1× Red Blood Cell Lysis Solution. Then incubated for 3 min and centrifuged. If the blood wasn't lysed well, it would be suspended with 2 mL 1× Red Blood Cell Lysis Solution again and incubated for 3 min.

Washed cells twice with DPBS. 100 μL of resuspended cells (in concentration of 10 million/mL) per test were seeded on 96 V-hole plate. After centrifugation, the cells were suspended with 100 μL DPBS. BV510 live/dead was added and incubated for 30 min at 4° C. in the dark. Cells were washed twice with DPBS. After extracellular antibodies incubation, washed cells twice with staining buffer, fixed and permeabilized for 30 min. Added purified rat anti-mouse CD16/CD32 for 5 min incubation and stained cells according to the procedures indicated in specific intracellular antibodies specification. Then washed the cells twice and suspend with 200 μL staining buffer. Stained cells were analyzed by BD Fortessa X20 Flow Cytometer.

TABLE 15
Staining was performed with the following antibody panel.

| # | Fluorochrome | Antibody | Supplier | Cat# | Clone |
|---|---|---|---|---|---|
| 1 | BUV395 | CD8 | BD | 563786 | 53-6.7 |
| 2 | BUV737 | F4/80 | BD | 749283 | T45-2342 |
| 3 | BV421 | CD62L | BioLegend | 104436 | MEL-14 |
| 4 | BV510 | Live/Dead | Invitrogen | L34957 | — |
| 5 | BV605 | MHCII | BioLegend | 107639 | M5/114.15.2 |
| 6 | BV785 | muPD-L1 | BioLegend | 124331 | 10F.9G2 |
| 7 | BV785 | Isotype | BioLegend | 400647 | RTK4530 |
| 8 | FITC | CD45 | BD | 553080 | 30-F11 |
| 9 | PerCP-Cy5.5 | CD44 | BD | 560570 | IM7 |
| 10 | PE | CD206 | eBioscience | 12-2061- | MR6F3 |
| | | | | 82 | |
| 11 | PE-CF594 | CD49b | BD | 562453 | DX5 |
| 12 | PE-Cy7 | CD4 | BioLegend | 100422 | GK1.5 |
| 13 | APC | hPD-1 | BioLegend | 621610 | A17188B |
| 14 | APC | Isotype | BioLegend | 400322 | MPC-11 |
| 15 | AF700 | CD11b | BD | 557960 | MI/70 |
| 16 | APC-Cy7 | CD3 | BioLegend | 100222 | 17A2 |

FIG. 11A shows a plot describing the effect of PD-1 targeted and untargeted immunocytokines on the growth of CT26 syngeneic colon carcinoma tumors in hPD1 humanized BALB/c mice. The immunocytokine tested in this figure is Composition A tested as a single agent at 1, and 2.5 mg/kg after a single injection schedule. Control Her2-targeted immunocytokine Composition O (Trastuzumab antibody conjugated to IL-2 polypeptide) was also tested at 2.5 mg/kg. (mean±SEM).

FIG. 11B shows a bar chart describing the effect PD-1 targeted and untargeted immunocytokines on the growth of CT26 syngeneic colon carcinoma tumors in hPD1 humanized BALB/c mice 7 days after treatment. The immunocytokine tested in this figure is Composition A tested as a single agent at 1 and 2.5 mg/kg after a single injection schedule. Control Her2-targeted immunocytokine Composition O (Trastuzumab antibody conjugated to IL-2 polypeptide) was also tested at 2.5 mg/kg. (mean±SEM; **one-way ANOVA P-value<0.001).

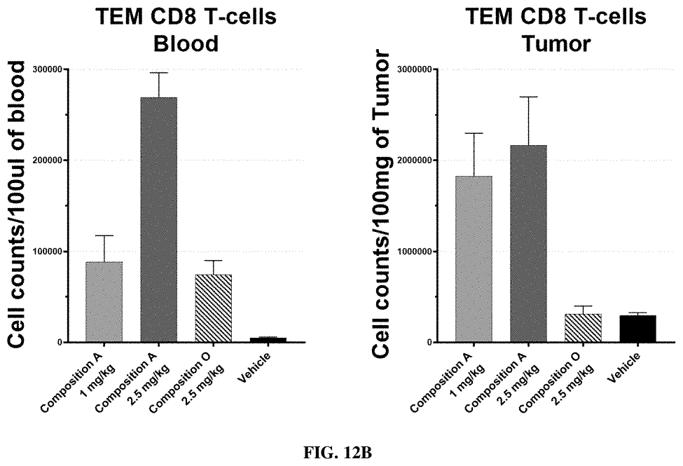

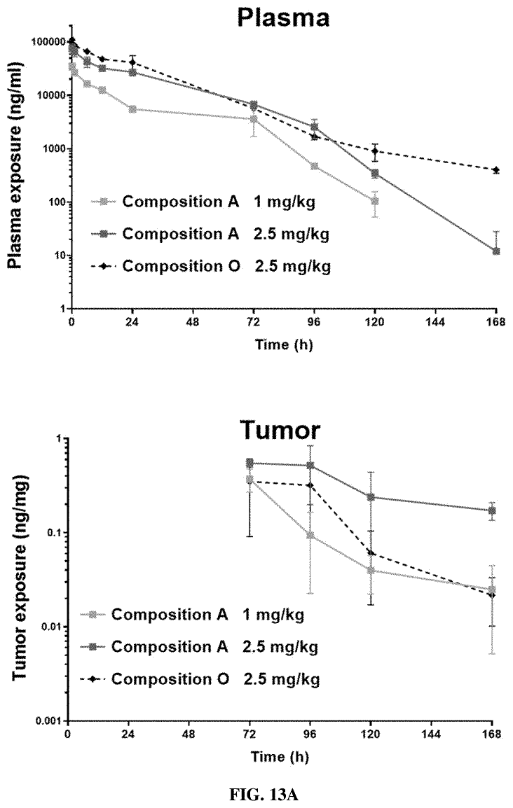

FIG. 12A shows a plot describing the effect of PD-1 targeted and untargeted immunocytokines on the expansion of naïve (CD62L^{high} CD44^{low}) CD8+ T-cells in the blood and tumors of CT26 tumor bearing hPD1 humanized BALB/c mice 7 days after treatment. The immunocytokine tested in this figure is Composition A tested as a single agent at 1, and 2.5 mg/kg after a single injection schedule. Control Her2-targeted immunocytokine Composition O (Trastuzumab antibody conjugated to IL-2 polypeptide) was also tested at 2.5 mg/kg. (n=3; mean±SEM).

FIG. 12B shows a plot describing the effect of PD-1 targeted and untargeted immunocytokines on the expansion of effector memory (CD62L^{negative} CD44^{high}) CD8+ T-cells in the blood and tumors of CT26 tumor bearing hPD1 humanized BALB/c mice 7 days after treatment. The immunocytokine tested in this figure is Composition A tested as a single agent at 1, and 2.5 mg/kg after a single injection schedule. Control Her2-targeted immunocytokine Composition O (Trastuzumab antibody conjugated to IL-2 polypeptide) was also tested at 2.5 mg/kg. (n=3; mean±SEM).

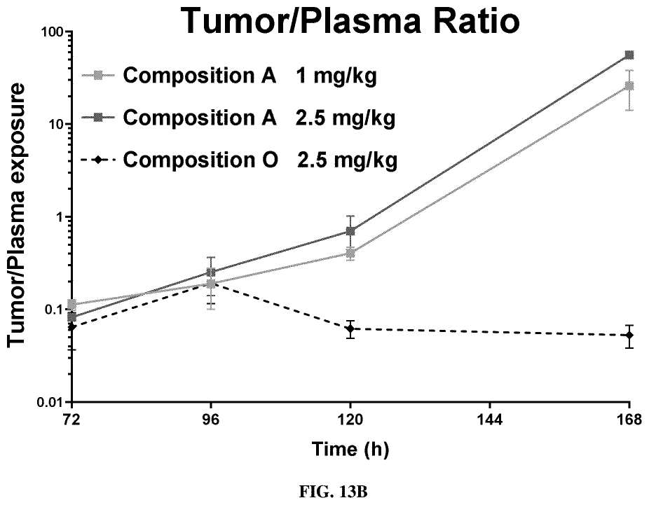

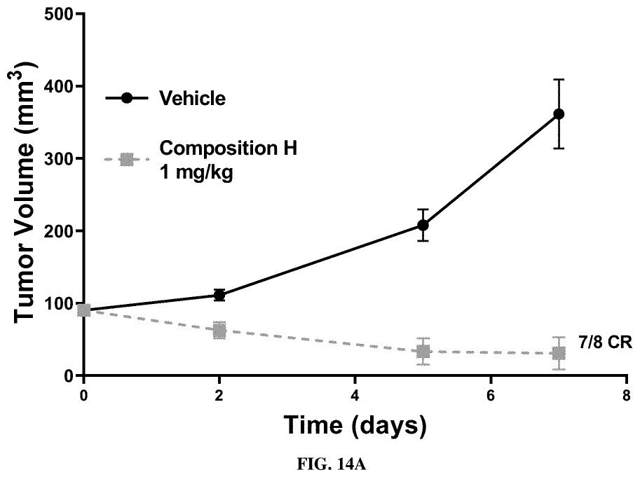

FIG. 13A shows a plot describing the effect of PD-1 targeting and untargeting of immunocytokines on their persistence in the blood and tumors of CT26 tumor bearing hPD1 humanized BALB/c mice, with the figure showing plasma or tumor concentration of PD-1 targeted and control immunocytokines on the y-axis and time on the x-axis. The immunocytokine tested in this figure is Composition A tested as a single agent at 1, and 2.5 mg/kg after a single injection schedule. Control Her2-targeted immunocytokine Composition O (Trastuzumab antibody conjugated to IL-2 polypeptide) was also tested at 2.5 mg/kg. (n=3; mean±SD).

FIG. 13B shows a plot describing the effect of PD-1 targeting and untargeting of immunocytokines on their persistence in the tumors as compared to blood of CT26 tumor bearing hPD1 humanized BALB/c mice, with the figure showing the ratio of tumor/plasma concentrations of PD-1 targeted and control immunocytokines on the y-axis and time on the x-axis. The immunocytokine tested in this figure is Composition A tested as a single agent at 1, and 2.5 mg/kg after a single injection schedule. Control Her2-targeted immunocytokine Composition O (Trastuzumab antibody conjugated to IL-2 polypeptide) was also tested at 2.5 mg/kg. (n=3; mean±SEM).

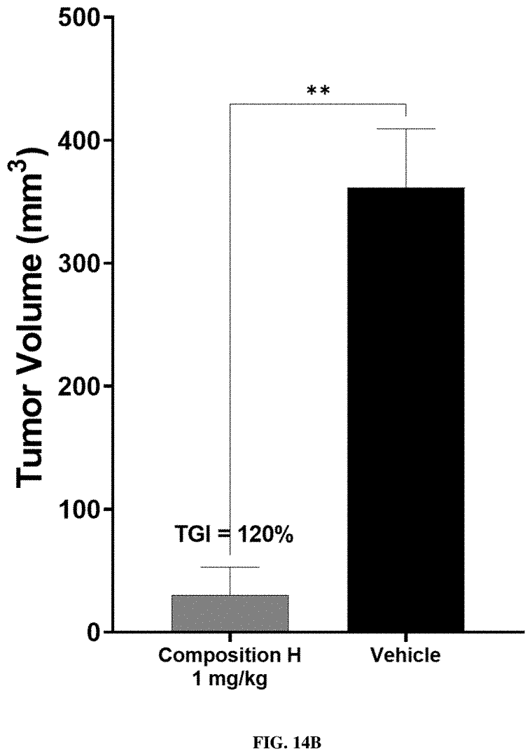

### Example 7: Efficacy Study (FIG. 14A-B)

An in vivo efficacy study was performed in mice. Naïve, 6-8 weeks old, C57BL/6-hPD1 female mice (GemPharmatech Co, Ltd, Nanjing, China) were inoculated subcutaneously at the right upper flank with MC38 tumor cells (3×10⁵) in 0.1 mL of PBS for tumor development. The animals were randomized (using an Excel-based randomization software performing stratified randomization based upon tumor volumes), and treatment started when the average tumor volume reached approximately 90 mm³. Animals treated with Composition H received a single 10 mL/kg bolus intravenous (i.v.) of 1 mg/kg of PD-1 antibody conjugated with modified IL-2 polypeptide. After inoculation, the animals were checked daily for morbidity and mortality. At the time, animals were checked for effects on tumor growth and normal behavior such as mobility, food and water consumption, body weight gain/loss (body weights were measured twice weekly), eye/hair matting and any other abnormal effect. The major endpoints were delayed tumor growth or complete tumor regression. Tumor sizes were measured three times a week in two dimensions using a caliper, and the volume was expressed in mm³ using the formula: V=0.5 a×b² where a and b are the long and short diameters of the tumor, respectively. Death and observed clinical signs were recorded on the basis of the numbers of animals within each subset.

FIG. 14A shows a plot describing the effect of a single injection of conjugated anti-PD1 antibody on the growth of MC38 syngeneic colon carcinoma tumors in hPD1 C57BL/6 mice. The immunocytokine tested in this figure is Composition H tested as a single agent at 1 mg/kg as a single i.v. injection. (n=8; mean±SEM).

FIG. 14B shows a bar chart describing the effect of a single injection of conjugated anti-PD1 antibody on the growth of MC38 syngeneic colon carcinoma tumors in hPD1 C₅₇BL/6 mice after 7 days of treatment. The immunocytokine tested in this figure is Composition H tested as a single agent at 1 mg/kg a single i.v. injection. (n=8 animals; mean±SEM; **one-way ANOVA P-value<0.005).

### Example 8: Synthesis of Composition AB

Modified IL-2 polypeptide Composition AB containing azido-PEG attached at residue F42Y, PEG group at Y45, and having an amino acid sequence of SEQ ID NO: 3, was synthesized by ligating individual peptides synthesized using solid phase peptide synthesis (SPPS). Individual peptides were synthesized on an automated peptide synthesizer using the methods described below. Related modified IL-2s provided herein were synthesized using analogous protocols.

Commercially available reagents were purchased from Sigma-Aldrich, Acros, Merck or TCI Europe and used without further purification. Fmoc amino acids with suitable side-chain protecting groups for solid phase peptide synthesis were purchased from Novabiochem, Christof Senn Laboratories AG or PeptART and they were used as supplied. The polyethylene glycol derivatives used for peptide synthesis were purchased by Polypure. HPLC grade CH₃CN from Sigma Aldrich was used for analytical and preparative HPLC purification.

High resolution mass spectra (FTMS) for peptides and proteins were measured on a Bruker solariX (9.4T magnet) equipped with a dual ESI/MALDI-FTICR source using 4-hydroxy-α-cyanocinnamic acid (HCCA) as matrix. CD spectra were recorded with a Jasco J-715 spectrometer with a 1.0 mm path length cell. Spectra were collected at 25° C. in continuous scanning mode with standard sensitivity (100 mdeg), 0.5 nm data pitch, 50 nm/min scanning speed, 1 nm bandwidth and 5 accumulations.

Peptides and proteins fragments were analyzed and purified by reverse phase high performance liquid chromatography (RP-HPLC). The peptide analysis and reaction monitoring were performed on analytical Jasco instruments with dual pumps, mixer and in-line degasser, autosampler, a variable wavelength UV detector (simultaneous monitoring of the eluent at 220 nm and 254 nm) and an injector fitted with a 100 μl injection loop. The purification of the peptide fragments was performed on a Gilson preparative instrument with 20 mL injection loop. In both cases, the mobile phase was MilliQ-H₂O with 0.1% TFA (Buffer A) and HPLC grade CH₃CN with 0.1% TFA (Buffer B). Analytical HPLC was performed on bioZen™ Intact C4 column (3.6 μm, 150×4.6 mm) or Shiseido Capcell Pak MG III (5 μm, 150×4.6 mm) column with a flow rate of 1 mL/min. Preparative HPLC was performed on a Shiseido Capcell Pak UG80 C18 column (5 μm, 50 mm I.D.×250 mm) at a flow rate of 40 mL/min.

The peptide segments were synthesized on a Syro I or a CS Bio 136X peptide synthesizers using Fmoc SPPS chemistry. The following Fmoc amino acids with side-chain protection groups were used: Fmoc-Ala-OH, Fmoc-Arg(Pbf)-OH, Fmoc-Asn(Trt)-OH, Fmoc-Asp(OtBu)-OH, Fmoc-Cys(Acm), Fmoc-Gln(Trt)-OH, Fmoc-Glu(OtBu)-OH, Fmoc-Gly-OH, Fmoc-His(1-Trt)-OH, Fmoc-Ile-OH, Fmoc-Leu-OH, Fmoc-Lys(Boc)-OH, Fmoc-Nle-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr(tBu)-OH, Fmoc-Val-OH. Fmoc-pseudoproline dipeptides were incorporated in the synthesis where necessary. Fmoc deprotections were performed with 20% piperidine in DMF (2×8 min), and monitored by UV at 304 nm with a feedback loop to ensure complete Fmoc removal. Couplings were performed with Fmoc-amino acid (3.0-5.0 equiv to resin substitution), HCTU or HATU (2.9-4.9 equiv) as coupling reagents and DIPEA or NMM (6-10 equiv) in DMF at room temperature or at 50° C. After pre-activating for 3 min, the solution was transferred and allowed to react with the peptide on-resin for either 30 min or 2 h depending on the amino acid. In some cases, double couplings were required. After coupling, the resin was treated with 20% acetic anhydride in DMF for capping any unreacted free amine. LiCl washes were performed where required. The allylester deprotection was performed using phenylsilane (24 equiv) and Palladium(0) tetrakis (triphenylphosphine) (0.5 equiv) in anhydrous dichloromethane.

The synthesis of the peptide segments by SPPS was monitored by microcleavage and analysis of the corresponding resin. The peptides were cleaved from the resin using a mixture of 95:2.5:2.5 TFA:DODT:H₂O (α-ketoacid segments synthesized on α-ketoacid resins) or 95:2.5:2.5 TFA:TIPS:H₂O (peptide synthesized on 2-cholorotrityl polystyrene resin) for 2 h. The resin was filtered off and the filtrate was evaporated and treated with cold diethyl ether, triturated and centrifuged. Ether layer was carefully decanted and the residue was resuspended in diethyl ether, triturated and centrifuged. Ether washings were repeated twice.

### 1.1 Synthesis of Composition AB Variant of IL-2

### Synthesis of IL-2 (1-39)-Leu-α-ketoacid

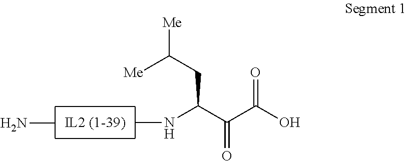

IL2 (1-39)-Leu-α-ketoacid (See SEQ ID NO: 3) was synthesized on Rink-amide resin pre-loaded with protected Fmoc-α-Leu-ketoacid with a substitution capacity of 0.25 mmol/g. To do so, Fmoc-Rink Amide MBHA resin (4 g) was pre-swelled in DMF for 15 min and Fmoc-deprotection was performed. Fmoc-Leucine-protected-α-ketoacid (795 mg, 1 mmol, 1.00 equiv.) was dissolved in 40 mL DMF and pre-activated with HATU (361 mg, 0.95 mmol, 0.95 equiv.) and DIPEA (348 μL, 2 mmol, 2.00 equiv.). The coupling was allowed to proceed for 6 h at room temperature. Then, the resin was capped followed by Fmoc-deprotection. The synthesis of the segment was performed on 0.250 mmol scale up to Ala1 by automated Fmoc SPPS using the procedure described in the general methods section. The progress of the peptide synthesis was monitored by performing a microcleavage and analysis using a mixture of (95:2.5:2.5) TFA:DODT:H₂O for 1.5 h. HPLC analysis were performed on a C18 column at 60° C. The peptide was cleaved from the resin using a mixture of 95:2.5:2.5 TFA:DODT:H₂O (15 mL/g resin) for 2 h, following the procedure described in the general methods. Purification of crude IL2 (1-39) was performed by preparative HPLC using Shiseido capcell pak C18 column (50×250 mm) with a gradient of 30 to 80% CH₃CN with 0.1% TFA in 30 min. The pure product fractions were pooled and lyophilized to obtain 650 mg of the pure IL2 (1-39) 1-39)-Leu-α-ketoacid (69% yield for peptide synthesis, resin cleavage and purification steps). Analytical HPLC and ESI-HRMS were used to confirm the purity and exact mass of the product. m/z calculated for C₂₀₄H₃₄₆N₅₆O₆₁ [M]: 4556.5694; measured: 4556.5783.

### Synthesis of Opr-IL2 (42-69) Photoprotected-Leu-α-Ketoacid of Composition AB

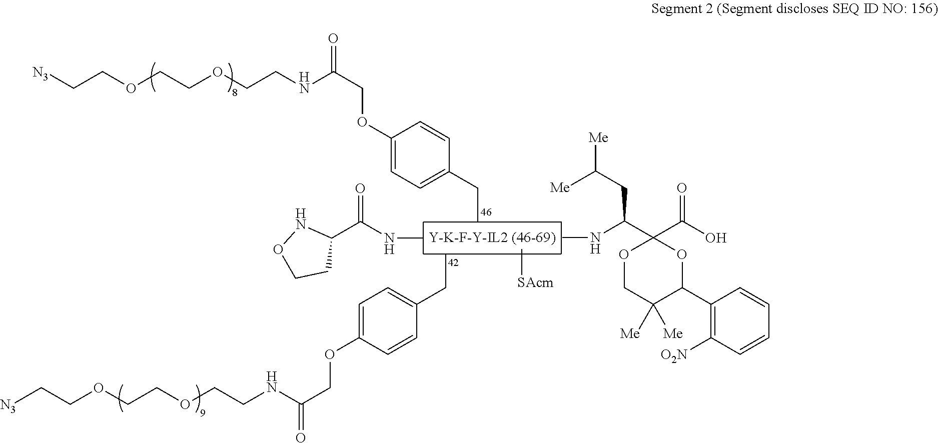

Opr-IL2 (42-69) (See SEQ ID NO: 3) photoprotected-Leu-α-ketoacid segment was prepared on Rink Amide MBHA resin preloaded with Fmoc-Leucine-photoprotected-α-ketoacid with a substitution capacity of 0.25 mmol/g. To do so, 4 g of Fmoc-Rink Amide MBHA resin were swelled with DMF for 15 min and Fmoc-deprotection was performed. Fmoc-Leucine-photoprotected-α-ketoacid (795 mg, 1 mmol, 1.00 equiv.) was dissolved in 40 mL DMF and preactivated with HATU (361 mg, 0.95 mmol, 0.95 equiv.) and DIPEA (348 μL, 2 mmol, 2.00 equiv.). The reaction was stirred for 6 h at room temperature. Then, the resin was capped, followed by Fmoc-deprotection. The synthesis of the segment was performed up to Nle46 on 0.151 mmol scale by automated Fmoc SPPS using the procedure described in the general methods section. Cys (Acm)-OH (10 equiv relative to the resin) was used for the coupling of Cys58 by symmetric anhydride method using DIC (5 equiv relative to resin) for 2 h at rt. The preformed amino acid Fmoc-Tyr(Ac0.5 kDa PEG)-OH (3 equiv) was coupled in position 45 by single coupling using HATU (2.9 equiv) and DIPEA (6 equiv). Phe44 and Lys43 were coupled by automated SPPS, followed by the manual coupling of Fmoc Tyr-allylacetate and Boc-5-(S)-Oxaproline in positions 42 and 41, respectively. The allyl ester deprotection was performed following established standard conditions using phenylsilane (449 μL, 3.6 mmol, 24 equiv) and Pd(Ph₃)₄ (87 mg, 0.075 mmol, 0.5 equiv) for 30 min at rt. After deprotection, O-(2-Aminoethyl)-O′-(2-azidoethyl) nonaethylene glycol (237 mg, 0,450 mmol, 3 equiv) was coupled at 50° C. for 1.5 h. The progress of the peptide synthesis was monitored by performing a microcleavage and analysis using a mixture of (95:2.5:2.5) TFA:DODT:H₂O for 1.5 h. HPLC analysis were performed on a C18 column at 60° C. The peptide was cleaved from the resin using a mixture of 95:2.5:2.5 TFA:DODT:H₂O (15 mL/g resin) for 2 h, following the procedure described in the general methods. The cold ether:pentane mixture (1:1) was used to treat and wash the crude peptide. Purification of crude IL2 (42-69) was performed by preparative HPLC using Shiseido capcell pak C18 column (50×250 mm) with a two step gradient: firstly, 10 to 30% CH₃CN in MQ-H₂O with 0.1% TFA in 5 min, then 30 to 60% CH₃CN in MQ-H₂O with 0.1% TFA in 30 min. The pure product fractions were pooled and lyophilized to obtain 117.4 mg of the pure IL2 (42-69) (16% yield for peptide synthesis, resin cleavage and purification steps). Analytical HPLC and ESI-HRMS were used to confirm the purity and exact mass of the product. m/z calculated for C₂₃₀H₃₇₆N₄₆O₇₄S [M]: 4998.6794; measured 4998.6749.

### Synthesis of Fmoc-Opr IL2 (72-102)-Phe-α-Ketoacid of Composition AB

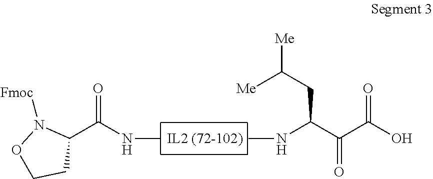

Fmoc-Opr IL2 (72-102)-Phenylalanine-α-ketoacid was synthesized on Rink Amide ChemMatrix resin pre-loaded with Fmoc-Phe-protected-α-ketoacid with a substitution capacity of ˜0.25 mmol/g. The synthesis was performed on 0.588 mmol scale by automated Fmoc SPPS up to Ala73 using HCTU as the coupling reagent. Coupling of residue 72, Fmoc-Leu was done with HATU as the coupling reagent. The coupling was repeated additional two times at 45° C. to ensure complete coupling. Fmoc-5-oxaproline (3.00 equiv to resin) was manually coupled to the free amine using HATU (2.95 equiv to resin) and NMM (6.00 equiv to resin) for 2 h at rt. The progress of the peptide synthesis was monitored by performing a microcleavage and analysis using a mixture of (95:2.5:2.5) TFA:DODT:H₂O for 2 h. HPLC analysis were performed on a C18 column at 60° C. The peptide was cleaved from resin using a mixture of 95:2.5:2.5 TFA:DODT:H₂O (15 mL/g resin) for 2.0 h. Purification of crude segment was performed by preparative HPLC using Shiseido Capcell Pak C18 column (50×250 mm) preheated at 60° C., with a gradient of 20 to 75% CH₃CN with 0.1% TFA in 30 min. The pure product fractions were pooled and lyophilized to obtain Fmoc-Opr IL2 (72-102)-Phe-α-ketoacid in >98% purity (147.9 mg, 6% yield for synthesis, cleavage and purification steps). Analytical HPLC and ESI-HRMS were used to confirm the purity and exact mass of the product. m/z calculated for C₁₈₄H₂₈₅N₄₇O₅₃ [M]: 4001.1051; measured 4001.1227.

### Synthesis of Opr-IL2 (105-133)

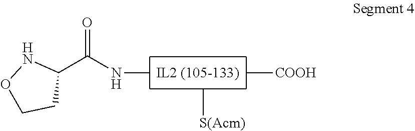

Opr-IL2 (105-133) was synthesized on 2-Chlorotrityl-resin pre-loaded with Fmoc-Thr-OH with a substitution capacity of 0.25 mmol/g. After capping (diisopropylethylamine, methanol), the synthesis was performed on 0.34 mmol scale (1.5 g of resin) by automated Fmoc SPPS up to Glu106. Cys (Acm)-OH (10 equiv relative to the resin) was used for the coupling of Cys105 by symmetric anhydride method using DIC (5 equiv relative to resin) for 2 h at rt. Then, Boc-5-oxaproline (2.00 equiv to resin) was coupled to the free amine on-resin using HATU (1.95 equiv) and NMM (4 equiv). The progress of the peptide synthesis was monitored by performing a microcleavage and analysis using a mixture of (95:2.5:2.5) TFA:TIPS:H₂O for 1.5 h. HPLC analysis were performed on a C18 column at 60° C. The peptide was cleaved from resin using a mixture of 95:2.5:2.5 TFA:TIPS:H₂O (15 mL/g resin) for 2.0 h. Purification of crude Opr-IL2(105-133) was performed by preparative HPLC using Shiseido Capcell Pak C4 column (50×250 mm) preheated at 60° C., with a gradient of 10 to 65% CH₃CN with 0.1% TFA in 10 min, then 65 to 95% CH₃CN with 0.1% TFA in 20 min. The pure product fractions were pooled and lyophilized to obtain Opr-IL2(105-133) in >98% purity (108.5 mg, 9% yield for synthesis, cleavage and purification steps. Analytical HPLC and ESI-HRMS were used to confirm the purity and exact mass of the product. m/z calculated for C₁₅₈H₂₄₂N₃₇O₅₂S [M+H]: 3521.7145; found 3521.7140.

### Synthesis of IL2-Seg12 of Composition AB by KAHA Ligation

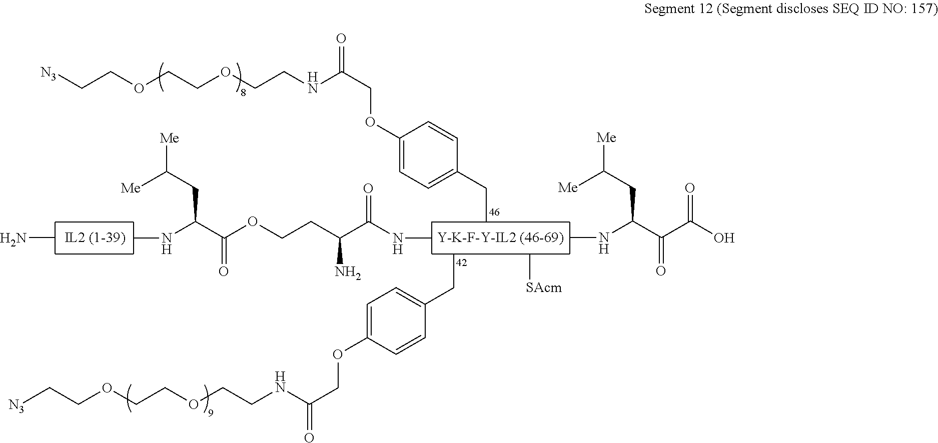

KAHA ligation: Seg1 (44 mg, 9.6 μmol, 1.2 equiv) and Seg2 (40 mg, 8.0 μmol, 1 equiv) were dissolved in DMSO:H₂O (9:1) containing 0.1 M oxalic acid (400 μL, 20 mM) and allowed to react at 60° C. for 20 h. The ligation vial was protected from light by wrapping it in aluminum foil. The progress of the KAHA ligation was monitored by uHPLC using a Phenomenex C18 column (150×4.6 mm) at 60° C. with CH₃CN/H₂O containing 0.1% TFA as mobile phase, with a gradient of 5 to 95% CH₃CN in 7 min.

Photo-deprotection and purification: After completion of the ligation the mixture was diluted ˜20 times (8 mL) with CH₃CN/H₂O (1:1) containing 0.1% TFA and irradiated at a wavelength of 365 nm for 1 h. The completion of photolysis reaction was confirmed by injecting a sample on uHPLC using previously described method. The photo-deprotected sample was purified by preparative HPLC using a Shiseido Capcell Pack UG80 C18 column (50×250 mm) kept at 60° C., with a 2-step gradient: double gradient of CH₃CN in water with 0.1% TFA: 10 to 35% in 5 min, then 35 to 65% in 35 min, with a flow of 40 mL/min with CH₃CN and MQ-H₂O containing 0.1% TFA as the eluents. The fractions containing the product were pooled and lyophilized to give pure Seg12 (25.4 mg, 40% yield for ligation and purification steps). m/z calculated for C₄₂₂H₇₀₉N₁₀₁O₁₃₀S [M]: 9304.1694; measured 9304.1639.

KAHA ligation for the preparation of IL2-Seg34 of Composition AB by KAHA ligation

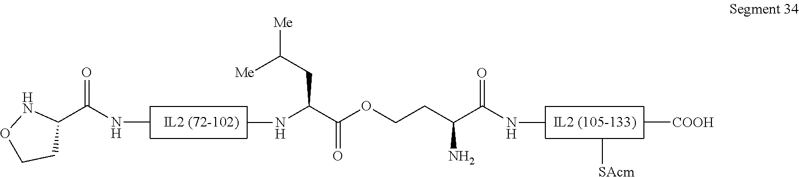

Ligation: Seg3 (136 mg, 34 μmol, 1.2 equiv) and Seg4 (100 mg, 28.40 μmol, 1 equiv) were dissolved in DMSO/H₂O (9:1) containing 0.1 M oxalic acid (1.8 mL, 15 mM) and allowed to react for 16 h at 60° C. The progress of the KAHA ligation was monitored by uHPLC using a Phenomenex C18 column (150×4.56 mm) at 60° C. using CH₃CN/H₂O containing 0.1% TFA as mobile phase, with a gradient of 30 to 70% CH₃CN in 7 min.

Fmoc deprotection and purification: After completion of ligation, the reaction mixture was diluted with DMSO (6 mL), 5% of diethylamine (300 μL) was added and the reaction mixture was shaken for 7 min at room temperature. To prepare the sample for purification, it was diluted with DMSO (4 mL) containing TFA (300 μL).

The sample was purified by preparative HPLC on a Shiseido Capcell Pack UG80 C18 column (50×250 mm) kept at 60° C., using a gradient of 30 to 70% CH₃CN in water with 0.1% TFA in 35 min, with a flow of 40 mL/min. The fractions containing the product were pooled and lyophilized to give pure Seg34 (43.4 mg after ligation and purification, 21% yield). Analytical HPLC and ESI-HRMS were used to confirm the purity and exact mass of the product. m/z calculated for C₃₂₆H₅₁₆N₈₄O₁₀₁S [M]: 7255.7545; measured: 7255.7653.

Final KAHA ligation for the preparation of IL2 linear protein Composition AB by KAHA ligation.

Ligation: Seg12 (59.2 mg, 6.35 μmol, 1.2 equiv) and Seg34 (38.5 mg, 5.3 μmol, 1 equiv) were dissolved in DMSO/H₂O (9:1) containing 0.1 M oxalic acid (423 μL, 15 mM) and the ligation was allowed to proceed for 24 h at 60° C. The progress of the KAHA ligation was monitored by analytical HPLC using a Shiseido Capcell Pak UG80 C18 column (250×4.6 mm) at 60° C. and CH₃CN/H₂O containing 0.1% TFA as mobile phase, with a gradient of 30 to 95% CH₃CN in 14 min.

Purification: After completion of ligation, the reaction mixture was diluted with 150 μL DMSO followed by further dilution with a mixture of (1:1) CH₃CN:H₂O containing 0.1% TFA (7 mL). The sample was purified by injecting on a preparative HPLC using a Shiseido Capcell Pack UG80 C18 column (50×250 mm) preheated at 60° C., with a 2-step gradient: 10 to 40% in 5 min and 40 to 80% in 35 min, flow rate: 40 mL/min with CH₃CN and MQ-H₂O containing 0.1% TFA as the eluents. The fractions containing the product were pooled and lyophilized to give pure COMPOSITION AB linear protein with Acm (42.3 mg, 48% yield for ligation and purification steps. Analytical HPLC and ESI-HRMS were used to confirm the purity and exact mass of the product. m/z calculated for C₇₄₇H₁₂₂₅N₁₈₅O₂₂₉S₂ [M]: 16515.9340; measured 16515.9008.

Acm deprotection: The peptide IL2 linear protein with Acm (35.4 mg, 2.14 μmol) was dissolved in AcOH/H₂O (1:1) (8.6 mL, 0.25 mM) and 86 mg AgOAc (1% m/v) were added to the solution. The mixture was shaken for 2.5 h at 50° C. protected from light. After completion of reaction as ascertained by HPLC, the sample was diluted with CH₃CN:H₂O (1:1) containing 0.1% TFA, and purified by preparative HPLC using a Shiseido CapCell Pak UG80 C₁₈ column (20×250 mm) kept at 60° C. A 2-step gradient was used for purification: 10 to 40% in 5 min and 40 to 95% in 30 min, flow rate: 10 mL/min, with CH₃CN and MQ-H₂O containing 0.1% TFA as the eluents. The fractions containing the product were pooled and lyophilized to give pure IL2 linear protein (26.1 mg, 74% yield for deprotection and purification steps). m/z calculated for C₇₄₁H₁₂₁₅N₁₈₃O₂₂₇S₂ [M]: 16373.8597; measured: 16373.8253

Synthesis of folded IL-2 Composition AB

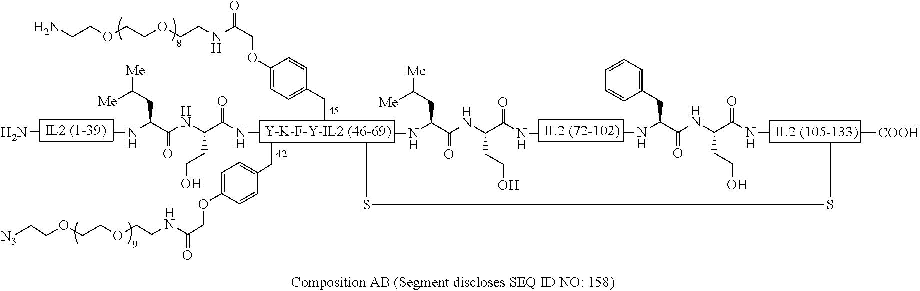

Rearrangement of linear protein: the linear protein (20 mg, 1.221 μmol) was dissolved in aqueous 6M Gu·HCl containing 0.1 M Tris and 30 mM reduced glutathione (81 mL, 15 protein concentration), which was adjusted to pH 8.0 by solution of 6M aq. HCl. The mixture was gently shaken at 50° C. for 2 h and monitored by analytical reverse phase HPLC using a bioZen™ 3.6 μm Intact C₄ column (150×4.6 mm) at 25° C., with a gradient of 30 to 95% CH₃CN in MQ-H₂O with 0.1% TFA in 18 min, flow rate: 1.0 mL/min.

Folding of the linear rearranged protein: the previous solution was cooled to room temperature and 3-fold diluted with a second buffer solution (240 mL) containing 0.1 M Tris and 1.5 mM oxidized glutathione at pH 8.0. the mixture was stored at room temperature and monitored by analytical HPLC using a bioZen™ 3.6 μm Intact C4 column (150×4.6 mm) at 25° C., with a gradient of 30 to 95% acetonitrile with 0.1% TFA in 18 min, flow rate: 1.0 mL/min. After 20 h, the folding solution was acidified with 10% aqueous TFA to ˜pH 3 and purified on preparative HPLC, using a Shiseido Proteonavi C4 column (20×250 mm) with a two-step gradient of 5 to 40 to 95% acetonitrile with 0.1% TFA in 60 min, flow rate: 10.0 mL/min. The fractions containing the folded IL2 protein were pooled together and lyophilized. The Purity and identity of the pure folded protein (3.5 mg, 18% yield) was further confirmed by analytical RP-HPLC and high-resolution ESI mass spectrometry. m/z calculated for C₇₄₁H₁₂₁₃N₁₈₃O₂₂₇S₂ [M]:16371.8441; measured: 16371.8107, confirming successful synthesis of Composition AB.

1. A composition comprising:
a polypeptide which selectively binds to programmed cell death protein 1 (PD-1);
a modified IL-2 polypeptide; and
a linker, wherein the linker comprises:
a first point of attachment covalently attached to the modified IL-2 polypeptide; and
a second point of attachment covalently attached to the polypeptide which selectively binds to PD-1;
wherein at least one of the first point of attachment or the second point of attachment is to a non-terminal residue of the polypeptide to which it is attached.

2-3. (canceled)

4. The composition of claim 1, wherein the first point of attachment is at a residue in the region of amino acid residues 30-110 of the modified IL-2 polypeptide, wherein amino acid residue position numbering of the IL-2 polypeptide is based on SEQ ID NO: 1 as a reference sequence.

5. The composition of claim 1, wherein the first point of attachment is at an amino acid residue selected from the group consisting of amino acid residue 35, 37, 38, 41, 42, 43, 44, 45, 60, 61, 62, 64, 65, 68, 69, 71, 72, 104, 105, and 107, wherein amino acid residue position numbering of the modified IL-2 polypeptide is based on SEQ ID NO: 1 as a reference sequence.

6. The composition of claim 1, wherein the first point of attachment is at amino acid residue 42 or 45, wherein amino acid residue position numbering of the modified IL-2 polypeptide is based on SEQ ID NO: 1 as a reference sequence.

7. The composition of claim 1, wherein the first point of attachment is at amino acid residue F42Y of the modified IL-2 polypeptide, wherein amino acid residue position numbering of the modified IL-2 polypeptide is based on SEQ ID NO: 1 as a reference sequence.

8. The composition of claim 7, wherein the IL-2 polypeptide comprises a non-linker polymer covalently attached thereto.

9. The composition of claim 8, wherein the non-linker polymer is attached at residue Y45.

10. The composition of claim 1, wherein the polypeptide which selectively binds to PD-1 is an anti-PD-1 antibody or an antigen binding fragment.

11-15. (canceled)

16. The composition of claim 10, wherein the second point of attachment is at an Fc region of the polypeptide which specifically binds to PD-1 at a position of a K246 amino acid residue, a K248 amino acid residue, a K288 amino acid residue, a K317 amino acid residue, or a combination thereof (Eu numbering).

17. The composition of claim 16, wherein the second point of attachment is at the K248 amino acid residue.

18. The composition of claim 1, wherein the polypeptide which selectively binds to PD-1 is a monoclonal antibody.

19. (canceled)

20. The composition of claim 10, wherein the polypeptide which selectively binds to PD-1 comprises an IgG.

21. The composition of claim 20, wherein the IgG is an IgG1, an IgG4, or is derived therefrom.

22. (canceled)

23. The composition of claim 1, wherein the polypeptide which selectively binds to PD-1 comprises Nivolumab, Pembrolizumab, LZM-009, Dostarlimab, Sintilimab, Spartalizumab, Tislelizumab, Zimberelimab, Cetrelimab, or Cemiplimab.

24-25. (canceled)

26. The composition of claim 1, wherein the second point of attachment is at a non-terminal amino acid residue of the polypeptide which selectively binds to PD-1.

27. The composition of claim 1, wherein the linker comprises a polymer.

28-31. (canceled)

32. The composition of claim 1, wherein the linker comprises a structure 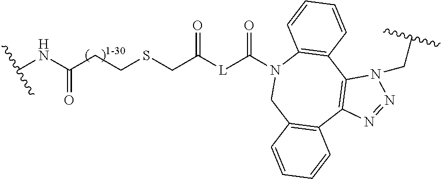
wherein 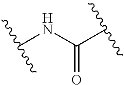 [SMILES: *NC(*)=O]
is the first point of attachment to a lysine residue of the polypeptide which selectively binds to PD-1;
L is a linking group; and 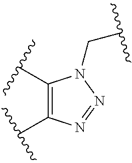 [SMILES: *Cn1nnc(*)c1*]
is a point of attachment to a linking group which connects to the first point of attachment.

33. The composition of claim 1, wherein the modified IL-2 polypeptide comprises a non-linker polymer covalently attached thereto.

34. The composition of claim 33, wherein the non-linker polymer is attached at an amino acid residue selected from the group consisting of amino acid residue 35, 37, 38, 41, 42, 43, 44, 45, 60, 61, 62, 64, 65, 68, 69, 71, 72, 104, 105, and 107.

35-86. (canceled)

87. The composition of claim 1, wherein the modified IL-2 polypeptide comprises an amino acid sequence of SEQ ID NO: 3.

88-103. (canceled)
